(19) United States
(12) Patent Application Publication
(10) Pub. No.: US 2022/0281860 A1
(43) Pub. Date: Sep. 8, 2022

(54) COMPOUNDS AND METHODS OF TREATING RNA-MEDIATED DISEASES

(71) Applicant: Arrakis Therapeutics, Inc., Waltham, MA (US)

(72) Inventors: Russell C. PETTER, Stow, MA (US); James Gregory BARSOUM, Lexington, MA (US)

(21) Appl. No.: 17/644,986

(22) Filed: Dec. 17, 2021

Related U.S. Application Data
(63) Continuation of application No. 16/074,232, filed on Jul. 31, 2018, now abandoned.
(60) Provisional application No. 62/289,671, filed on Feb. 1, 2016.

(51) Int. Cl. C07D 413/14; C07C 233/78; C07C 237/48; C07D 207/16; C07D 213/30; C07D 213/38; C07D 217/02; C07D 231/12; C07D 233/64; C07D 249/06; C07D 263/32; C07D 307/44; C07D 307/52; C07D 401/04; C07D 401/10; C07D 401/14; C07D 403/10; C07D 403/14; C07D 405/04; C07D 405/10; C07D 413/12; C07D 417/06; C07D 471/04; C07D 487/04; C07D 487/08; C07H 5/06; C07H 21/02; C12N 15/10

(52) U.S. Cl. CPC C07D 413/14; C07C 233/78; C07C 237/48; C07D 207/16; C07D 213/30; C07D 213/38; C07D 217/02; C07D 231/12; C07D 233/64; C07D 249/06; C07D 263/32; C07D 307/44; C07D 307/52; C07D 401/04; C07D 401/10; C07D 401/14; C07D 403/10; C07D 403/14; C07D 405/04; C07D 405/10; C07D 413/12; C07D 417/06; C07D 471/04; C07D 487/04; C07D 487/08; C07H 5/06; C07H 21/02; C12N 15/1034

(57) ABSTRACT

The present invention provides compounds, compositions thereof, and methods of using the same.

### CROSS-REFERENCE TO RELATED APPLICATIONS

This application claims the benefit of U.S. Provisional Application No. 62/289,671, filed on Feb. 1, 2016, the entirety of which is hereby incorporated by reference.

### SEQUENCE LISTING

The instant application contains a Sequence Listing which has been submitted electronically in ASCII format and is hereby incorporated by reference in its entirety. Said ASCII copy, created on Mar. 22, 2017, is named 394457-001WO(151019)_SL.txt and is 55,272 bytes in size.

### TECHNICAL FIELD OF THE INVENTION

The present invention relates to compounds and methods useful for modulating the biology of RNA transcripts to treat various diseases and conditions. The invention also provides methods of identifying RNA transcripts that bind compounds and are thus druggable, screening drug candidates and methods of determining drug binding sites and/or reactive site(s) on a target RNA.

### BACKGROUND OF THE INVENTION

Ribonucleic acids (RNAs) have been conventionally considered mere transient intermediaries between genes and proteins, whereby a protein-coding section of deoxyribonucleic acid (DNA) is transcribed into RNA that is then translated into a protein. RNA was thought to lack defined tertiary structure, and even where tertiary structure was present it was believed to be largely irrelevant to the RNA's function as a transient messenger. This understanding has been challenged by the recognition that RNA, including non-coding RNA (ncRNA), plays a multitude of critical regulatory roles in the cell and that RNA can have complex and defined tertiary structure.

All mammalian diseases are ultimately mediated by the transcriptome. Insofar as messenger mRNA (mRNA) is part of the transcriptome, and all protein expression derives from mRNAs, there is the potential to intervene in protein-mediated diseases by modulating the expression of the relevant protein and by, in turn, modulating the translation of the corresponding upstream mRNA. But mRNA is only a small portion of the transcriptome: other transcribed RNAs also regulate cellular biology either directly by the structure and function of RNA structures (e.g., ribonucleoproteins) as well as via protein expression and action, including (but not limited to) miRNA, lncRNA, lincRNA, snoRNA, snRNA, scaRNA, piRNA, ceRNA, and pseudo-genes. Drugs that intervene at this level have the potential of modulating any and all cellular processes. Existing therapeutic modalities such as antisense RNA or siRNA, in most cases, have yet to overcome significant challenges such as drug delivery, absorption, distribution to target organs, pharmacokinetics, and cell penetration. In contrast, small molecules have a long history of successfully surmounting these barriers and these qualities, which make them suitable as drugs, are readily optimized through a series of analogues to overcome such challenges. In sharp contrast, there are no validated, general methods of screening small molecules for binding to RNA targets in general, much less inside cells. The application of small molecules as ligands for RNA that yield therapeutic benefit has received little to no attention from the drug discovery community.

Targeting the RNA transcriptome with small molecule modulators represents an untapped therapeutic approach to treat a variety of RNA-mediated diseases. Accordingly, there remains a need to develop small-molecule RNA modulators useful as therapeutic agents.

### BRIEF DESCRIPTION OF THE FIGURES

FIG. 1 shows the basic steps of the hook and click (PEARL-seq; Proximity-Enhanced Activation of RNA Ligation) method. A small molecule ligand binds to a target RNA structure (here, a stem-loop feature), a modifying moiety attached to the small molecule (R^{mod}) forms a covalent bond to a proximate 2′-OH of the target RNA, and subsequent denaturing and sequencing reveals the location of the modification.

FIG. 2 shows general structures for the three broad types of compounds described herein: Type I, Type II, and Type III, which differ in the presence or location of the optional click-ready group. (RNA ligand=small-molecule binder to folded RNA; X=linkages; tethers=connects RNA ligand with RNA warhead; RNA warhead=range of electrophiles that acylate or sulfonylate 2′-OH groups on riboses; Click Grp.=a click-ready group that enables pull-down and focused assays, including sequencing.)

FIG. 3 shows general structures for the three broad types of RNA conjugates described herein: Type I, Type II, and Type III, which differ in the presence or location of the optional click-ready group. The target RNA is covalently conjugated to the RNA warhead, or modifying moiety, via a covalent bond to one of the 2′-OH groups on a ribose of the target RNA.

FIG. 4 shows a scheme of an exemplary hook and click compound (here, a theophylline tethered to a modifying moiety comprising a pyridine bearing a carbonyl(imidazolyl) acylating group and an azidomethyl click-ready group) binding to a target RNA, acylating (“hooking”) it, and then undergoing a click reaction with a 4-dibenzocyclooctynol (DIBO) group bound to biotin for use in a further pull-down procedure with avidin or other biotin-binding protein.

FIG. 5 shows a generalized scheme for assembling the components of a Type I compound joined by amide bonds.

FIG. 6 shows a generalized scheme for assembling the components of a Type II compound joined by amide bonds.

FIG. 7 shows a generalized scheme for assembling the components of a Type III compound joined by amide bonds.

FIG. 8 shows a generalized scheme for assembling the components of a Type I compound joined by amide bonds (directionality reversed relative to FIG. 5).

FIG. 9 shows a generalized scheme for assembling the components of a Type II compound joined by amide bonds (directionality reversed relative to FIG. 6).

FIG. 10 shows a generalized scheme for assembling the components of a Type III compound joined by amide bonds (directionality reversed relative to FIG. 7).

FIG. 11 shows a generalized scheme for assembling the components of a Type I compound joined by an amide bond between the RNA ligand and the tether and an ether bond between the tether and the RNA warhead (modifier moiety).

FIG. 12 shows a generalized scheme for assembling the components of a Type II compound joined by an amide bond between the RNA ligand and the tether and an ether bond between the tether and the RNA warhead (modifier moiety).

FIG. 13 shows a generalized scheme for assembling the components of a Type III compound joined by an amide bond between the RNA ligand and the tether and an ether bond between the tether and the RNA warhead (modifier moiety).

FIG. 14 shows a generalized scheme for assembling the components of a Type I compound joined by an ether between the RNA ligand and the tether and an amide between the tether and the RNA warhead (modifier moiety).

FIG. 15 shows a generalized scheme for assembling the components of a Type II compound joined by an ether between the RNA ligand and the tether and an amide between the tether and the RNA warhead (modifier moiety).

FIG. 16 shows a generalized scheme for assembling the components of a Type III compound joined by an ether between the RNA ligand and the tether and an amide between the tether and the RNA warhead (modifier moiety).

FIG. 17 shows a generalized scheme for assembling the components of a Type I compound joined by an amide between the RNA ligand and the tether and an ether between the tether and the RNA warhead (modifier moiety).

FIG. 18 shows a generalized scheme for assembling the components of a Type II compound joined by an amide between the RNA ligand and the tether and an ether between the tether and the RNA warhead (modifier moiety).

FIG. 19 shows a generalized scheme for assembling the components of a Type III compound joined by an amide between the RNA ligand and the tether and an ether between the tether and the RNA warhead (modifier moiety).

FIG. 20 shows a generalized scheme for assembling the components of a Type I compound joined by an ether between the RNA ligand and the tether and an amide between the tether and the RNA warhead (modifier moiety).

FIG. 21 shows a generalized scheme for assembling the components of a Type II compound joined by an ether between the RNA ligand and the tether and an amide between the tether and the RNA warhead (modifier moiety).

FIG. 22 shows a generalized scheme for assembling the components of a Type III compound joined by an ether between the RNA ligand and the tether and an amide between the tether and the RNA warhead (modifier moiety).

FIG. 23 shows a generalized scheme for assembling the components of a Type I compound joined by an ether between the RNA ligand and the tether and an ether between the tether and the RNA warhead (modifier moiety).

FIG. 24 shows a generalized scheme for assembling the components of a Type II compound joined by an ether between the RNA ligand and the tether and an ether between the tether and the RNA warhead (modifier moiety).

FIG. 25 shows a generalized scheme for assembling the components of a Type III compound joined by an ether between the RNA ligand and the tether and an ether between the tether and the RNA warhead (modifier moiety).

FIG. 26 shows a generalized scheme for assembling the components of a Type I compound joined by an amide between the RNA ligand and the tether and an amide between the tether and the RNA warhead (modifier moiety). This approach employs a diacid tether, i.e. a tether bearing a carboxylic acid on each end.

FIG. 27 shows a generalized scheme for assembling the components of a Type II compound joined by an amide between the RNA ligand and the tether and an amide between the tether and the RNA warhead (modifier moiety). This approach employs a diacid tether, i.e. a tether bearing a carboxylic acid on each end.

FIG. 28 shows a generalized scheme for assembling the components of a Type III compound joined by an amide between the RNA ligand and the tether and an amide between the tether and the RNA warhead (modifier moiety). This approach employs a diacid tether, i.e. a tether bearing a carboxylic acid on each end.

FIG. 29 shows a generalized scheme for assembling the components of a Type I compound joined by an amide between the RNA ligand and the tether and an amide between the tether and the RNA warhead (modifier moiety). This approach employs a diamine tether, i.e. a tether bearing an amino group on each end.

FIG. 30 shows a generalized scheme for assembling the components of a Type II compound joined by an amide between the RNA ligand and the tether and an amide between the tether and the RNA warhead (modifier moiety). This approach employs a diamine tether, i.e. a tether bearing an amino group on each end.

FIG. 31 shows a generalized scheme for assembling the components of a Type III compound joined by an amide between the RNA ligand and the tether and an amide between the tether and the RNA warhead (modifier moiety). This approach employs a diamine tether, i.e. a tether bearing an amino group on each end.

FIG. 32 shows points of attachment for the tethering group on the structure of tetracycline.

FIG. 33 shows points of attachment for the tethering group on the structures of theophylline, triptycene, linezolid, and anthracene-maleimide Diels-Alder adduct small molecule ligands.

FIG. 34 shows points of attachment for the tethering group on the structures of SMN2 ligands.

FIG. 35 shows points of attachment for the tethering group on the structures of the aminoglycoside kanamycin A.

FIG. 36 shows points of attachment for the tethering group on the structure of Ribocil.

FIG. 37 shows structures of theophylline ligands with points of attachment for the tethering groups.

FIG. 38 shows structures of tetracycline ligands with points of attachment for the tethering groups.

FIG. 39 shows structures of triptycene ligands with points of attachment for the tethering groups.

FIG. 40 shows structures of triptycene ligands with points of attachment for the tethering groups. X=CH, N, or C—OH; Y=CH or N; R1, R2, R3=each independently selected from halo, —OH, —OMe, —NH₂, —NH-(optionally substituted C₁₋₁₀ aliphatic), optionally substituted C₁₋₁₀ aliphatic, or other described tethering groups. The modifier moiety may be attached at any position on R1, R2, or R3, or at the other functional groups on the above structures.

FIG. 41 shows structures of anthracene-maleimide Diels-Alder adduct ligands with points of attachment for the tethering groups. Note: The corresponding structures having the succinimido group in the opposite stereochemical orientation may also be prepared. Each R is independently selected from halo, —OH, —OMe, —NH₂, —NH-(optionally substituted C₁₋₁₀ aliphatic), optionally substituted C₁₋₁₀ aliphatic, or other described tethering groups. The modifier moiety may be attached at any position on R, or at the other functional groups on the above structures.

FIG. 42 shows structures of ribocil ligands with points of attachment for the tethering groups.

FIG. 43 shows structures of SMN2 ligands with points of attachment for the tethering groups.

FIG. 44 shows structures of linezolid and tedizolid ligands with points of attachment for the tethering groups.

FIG. 45 shows structures of exemplary click-ready groups.

FIG. 46 shows exemplary tethering groups for linking RNA ligands and modifying moieties.

FIG. 47 shows further examples of tethering groups.

FIG. 48 shows further examples of tethering groups.

FIG. 49 shows further examples of tethering groups.

FIG. 50 shows further examples of tethering groups.

FIG. 51 shows further examples of tethering groups.

FIG. 52 shows further examples of tethering groups.

FIG. 53 shows further examples of tethering groups.

FIG. 54 shows exemplary broad classes of modifying groups that may be used to form a covalent adduct with a RNA 2′-OH.

FIG. 55 shows exemplary classes of lactone and lactam modifying groups that may be used to form a covalent adduct with a RNA 2′-OH.

FIG. 56 shows exemplary classes of arenecarbonyl imidazole modifying groups that may be used to form a covalent adduct with a RNA 2′-OH.

FIG. 57 shows exemplary classes of arenecarbonyl phenyl ester modifying groups that may be used to form a covalent adduct with a RNA 2′-OH.

FIG. 58 shows structures of sulfonyl-based modifying groups. The top three structures are specific agents known to sulfonylate catalytic site serines in serine proteases. The remaining structures are exemplary classes of sulfonyl fluoride modifying groups that may be used to form a covalent adduct with a RNA 2′-OH. Note: Specific agents capable of sulfonylating catalytic serines in serine proteases. Hydrolysis half-lives can be found at JBC 1982, 257, 5077; JACS 1974, 96, 233; Anal. Biochem. 1978, 86, 574; JOC 1970, 35, 1825; BBA 1979, 568, 11. See also: JACS 1968, 90, 1860; JACS 1975, 97, 4121; Bioorg. Chem. 1981, 10, 118; Bioorg. Chem. 1981, 10, 133.

FIG. 59 shows exemplary classes of furancarbonyl phenyl ester modifying groups that may be used to form a covalent adduct with a RNA 2′-OH.

FIG. 60 exemplary classes of furancarbonyl phenyl ester modifying groups that may be used to form a covalent adduct with a RNA 2′-OH.

FIG. 61 shows exemplary classes of arenecarbonyl phenyl ester modifying groups that may be used to form a covalent adduct with a RNA 2′-OH.

FIG. 62 shows exemplary classes of arenecarbonyl phenyl ester modifying groups that may be used to form a covalent adduct with a RNA 2′-OH.

FIG. 63 shows exemplary classes of isatoic anhydride modifying groups that may be used to form a covalent adduct with a RNA 2′-OH.

FIG. 64 shows exemplary classes of beta-lactone modifying groups that may be used to form a covalent adduct with a RNA 2′-OH.

FIG. 65 shows exemplary classes of beta-lactam modifying groups that may be used to form a covalent adduct with a RNA 2′-OH.

FIGS. 66A-66B show exemplary triptycene-based hook compounds (small molecule ligand+tethering group+modifying group).

FIG. 67 shows exemplary theophylline-based hook compounds (small molecule ligand+tethering group+modifying group).

FIG. 68 shows exemplary theophylline-based hook and click compounds (small molecule ligand+tethering group+modifying group+click-ready group).

FIG. 69 shows exemplary pull-down moieties, which include biotin and a group capable of reacting with a click-ready group.

FIG. 70 shows exemplary compounds comprising tetracycline as the small molecule ligand together with various exemplary tethering groups and modifying moieties.

FIG. 71 shows exemplary compounds comprising a substituted triptycene as the small molecule ligand together with various exemplary tethering groups and modifying moieties, with some also including click-ready groups.

FIG. 72 shows exemplary compounds comprising a substituted triptycene as the small molecule ligand together with various exemplary tethering groups, modifying moieties, and click-ready groups.

FIG. 73 shows exemplary compounds comprising SMN2 transcript-binding compounds as the small molecule ligand together with various exemplary tethering groups, modifying moieties, and click-ready groups.

FIGS. 74A-74B show shows exemplary compounds comprising ribocil as the small molecule ligand together with various exemplary tethering groups, modifying moieties, and click-ready groups.

FIG. 75 shows exemplary compounds comprising a substituted triptycene as the small molecule ligand together with various exemplary tethering groups and modifying moieties, with some also including click-ready groups.

FIG. 76 shows the basic steps of the SHAPE method (SHAPE=Selective 2′-hydroxyl acylation analyzed by primer extension; MaP=Mutational profiling). First, RNA is exposed to a SHAPE reagent that reacts at the 2′-OH groups of relatively accessible nucleotides to form a covalent adduct. The modified RNA is isolated and reverse transcribed. The reverse transcriptase “reads through” the chemical adducts in the RNA and incorporates a nucleotide noncomplementary to the original sequence (red) into the cDNA. Sequencing by any massively parallel approach assembles a profile of the mutations. Sequencing reads are compared with a reference sequence and mutation rates at each nucleotide are determined, corrected for background, and normalized, producing the SHAPE reactivity profile. SHAPE reactivities correlate with secondary structures, can reveal competing and alternative structures, or quantify effects on local nucleotide accessability.

FIG. 77 shows reaction schemes for accessing several theophylline small molecule ligands that include attachment points for the tethering group.

FIG. 78 shows reaction schemes for accessing several theophylline small molecule ligands that include attachment points for the tethering group.

FIG. 79 shows reaction schemes for accessing several theophylline small molecule ligands that include attachment points for the tethering group.

FIG. 80 shows reaction schemes for accessing several theophylline small molecule ligands that include attachment points for the tethering group.

FIG. 81 shows reaction schemes for accessing several tetracycline small molecule ligands that include attachment points for the tethering group.

FIG. 82 shows reaction schemes for accessing several tetracycline small molecule ligands that include attachment points for the tethering group.

FIGS. 83A-83B show reaction schemes for accessing several tetracycline small molecule ligands that include attachment points for the tethering group.

FIG. 84 shows reaction schemes for accessing several tetracycline small molecule ligands that include attachment points for the tethering group.

FIGS. 85A-85B show reaction schemes for accessing several triptycene small molecule ligands that include attachment points for the tethering group.

FIG. 86 shows reaction schemes for accessing several triptycene small molecule ligands that include attachment points for the tethering group.

FIGS. 87A-87B show reaction schemes for accessing several triptycene small molecule ligands that include attachment points for the tethering group.

FIG. 88 shows reaction schemes for accessing several triptycene small molecule ligands that include attachment points for the tethering group.

FIG. 89 shows reaction schemes for accessing several triptycene small molecule ligands that include attachment points for the tethering group.

FIG. 90 shows reaction schemes for accessing several triptycene small molecule ligands that include attachment points for the tethering group.

FIG. 91 shows reaction schemes for accessing several triptycene small molecule ligands that include attachment points for the tethering group.

FIG. 92 shows reaction schemes for accessing several triptycene small molecule ligands that include attachment points for the tethering group.

FIGS. 93A-93B show reaction schemes for accessing several tetracycline small molecule ligands that include a tethering group and modifying moiety.

FIGS. 94A-94B show reaction schemes for accessing several triptycene small molecule ligands that include a tethering group and modifying moiety.

FIG. 95 shows possible ambiguity that may arise in the described methods and ways of disambiguating sequence data from proximity-induced modification of 2′-OH RNA riboses. Because one ligand-binding event may yield modification of riboses that are remote in terms of the RNA primary sequence but proximal in the folded structure, there are two or more possible ligand binding sites. Data from SHAPE-MaP and/or SAR of the tethering group can resolve the ambiguities. SHAPE-MaP and RING-MaP can determine the actual, un-liganded structure of the RNA. Different tethering group lengths and other features will cause the SHAPE modification patterns to respond differently, resolving the ambiguity.

FIG. 96 shows a scheme for parallel synthesis of a library of hook compounds.

FIGS. 97A-97B show a synthetic route for compound ARK-132.

FIGS. 98A-98D show a synthetic route for compound ARK-134.

FIGS. 99A-99C show a synthetic route for compounds ARK-135 and ARK-136.

FIG. 100 shows a synthetic route for compound ARK-188.

FIG. 101 shows a synthetic route for compound ARK-190.

FIG. 102 shows a synthetic route for compound ARK-191.

FIG. 103 shows a synthetic route for compound ARK-195.

FIG. 104 shows a synthetic route for compound ARK-197.

FIGS. 105A-105B show a synthetic route for compounds based on the ribocil scaffold.

FIG. 106 shows a calibration experiment to determine the dependence of fluorescence on the concentration of 3WJ RNA constructs.

FIG. 107 shows the results of a fluorescence quenching experiment of compounds Ark000007 and Ark000008 with two RNA 3WJ constructs at various concentrations.

FIG. 108 shows likely structures for the following three RNA 3WJ constructs, with a putative binding site for small molecule ligands shown as a triangle: A) RNA3WJ_1.0.0_5IB_3FAM (cis 3WJ with one unpaired nucleotide) (SEQ ID NO: 31); B) Split3WJ.1_up_5IB+Split3WJ.1_down_3FAM (trans 3WJ as 1:1 mix) (SEQ ID NOS 52-53, respectively, in order of appearance); and C) Split3WJ.2 up_5IB+Split3WJ.2_down_3FAM (trans 3WJ as 1:1 mix) (SEQ ID NOS 54-55, respectively, in order of appearance).

FIG. 109 shows fluorescence quenching data measuring interaction of compounds Ark0000013 and Ark0000014 with the following RNA constructs: A) RNA3WJ_1.0.0_5IB_3FAM (cis 3WJ with one unpaired nucleotide); B) Split3WJ.1 up_5IB+Split3WJ.1_down_3FAM (trans 3WJ as 1:1 mix); and C) Split3WJ.2 up_5IB+Split3WJ.2_down_3FAM (trans 3WJ as 1:1 mix).

FIG. 110 shows thermal shift data for compounds Ark000007 and Ark000008 tested with the 3WJ_0.0.0_5IB_3FAM RNA construct. Data analysis shows significant effect for Ark000007 with melting temperature shift of ˜5° C. (i.e. from 61.2° C. to 65.6° C.). In contrast, only a very small effect for Ark000008 was observed. These data suggest that the presence of Ark000007 increases stability of the 3WJ.

FIG. 111 shows thermal shift data for Ark0000013 and Ark0000014 in the presence of RNA3WJ_1.0.0_5IB_3FAM (cis 3WJ with one unpaired nucleotide).

FIG. 112 shows thermal shift data for Ark0000013 and Ark0000014 in the presence of Split3WJ.1_up_5IB+Split3WJ.1_down_3FAM.

FIG. 113 shows thermal shift data for Ark0000013 and Ark0000014 in the presence of Split3WJ.2_up_5IB+Split3WJ.2_down_3FAM.

FIG. 114 shows the structure of CPNQ, assigned proton resonances, NMR spectrum, and epitope mapping results.

FIG. 115 shows the structure of HP-AC008002-E01, assigned proton resonances, NMR spectrum, and epitope mapping results. The scaled STD effect was plotted onto the molecule according to the preliminary assignments. The data suggests for both RNA constructs that protons of the pyridine ring are in closer proximity to RNA than the benzene ring. The aliphatic CH₂ group could not be observed due to buffer signal overlap in that region.

FIG. 116 shows the structure of HP-AC0008001-E02, assigned proton resonances, NMR spectrum, and epitope mapping results. The scaled STD effect was plotted onto the molecule according to the preliminary assignments. The data suggest for both RNA constructs that aromatic protons closest to the heterocycle are in closer proximity to RNA protons. Aliphatic proton resonances could not be assessed by STD due to direct saturation artifacts/buffer signal overlap in that region (epitope mapping by WaterLOGSY).

FIG. 117 shows the structure of HP-AT005003-C03, assigned proton resonances, NMR spectrum, and epitope mapping results. The scaled STD effect was plotted onto the molecule according to the preliminary assignments. Due to signal overlap no individual assignment of the CH₂ groups was possible. The data suggest for both RNA constructs that protons of the furan moiety are in closer proximity to RNA protons than the phenyl.

FIGS. 118A-118E show steps for the production of Illumina small RNA-Seq library preparation using T4 RNA ligase 1 adenylated adapters (SEQ ID NOS 31, 42, 56, 43, 57, 44, 57, 58, 45, 59, 58, 46, 60, 49, 48, 46, 61, 62, 46, 61 and 62, respectively, in order of appearance).

FIGS. 119A-119E show steps for the production of Illumina small RNA-Seq library preparation using T4 RNA ligase 1 adenylated adapters (SEQ ID NOS 63, 42, 64, 64, 43, 65, 44, 65, 58, 45, 59, 58, 46, 47, 49, 48, 46, 59, 62, 46, 59 and 62, respectively, in order of appearance).

FIG. 120 shows PAGE analysis of RNA target sequences for use in DEL experiments. The gel lanes show: 1: HTT17CAG in NMR buffer; 2: Before incubation with Neutravidin resin; 3: Supernatant after incubation with Neutravidin resin; 4: RNA after incubation with DEL compounds for 1 hour at RT. The RNA was recovered after heat release from the resin.

FIG. 121 shows exemplary steps of a Surface Plasmon Resonance (SPR) method for use in the present invention.

FIGS. 122A-122B show exemplary steps of a Surface Plasmon Resonance (SPR) method for use in the present invention.

### DETAILED DESCRIPTION OF CERTAIN EMBODIMENTS

### 1. General Description of Certain Embodiments of the Invention; Definitions

RNA Targets and Association with Diseases and Disorders

The vast majority of molecular targets that have been addressed therapeutically are proteins. However, it is now understood that a variety of RNA molecules play important regulatory roles in both healthy and diseased cells. While only 1-2% of the human genome codes for proteins, it is now known that the majority of the genome is transcribed (Carninci et al., Science 309:1559-1563; 2005). Thus, the noncoding transcripts (the noncoding transcriptome) represent a large group of new therapeutic targets. Noncoding RNAs such as microRNA (miRNA) and long noncoding RNA (lncRNA) regulate transcription, splicing, mRNA stability/decay, and translation. In addition, the noncoding regions of mRNA such as the 5′ untranslated regions (5′ UTR), the 3′ UTR, and introns can play regulatory roles in affecting mRNA expression levels, alternative splicing, translational efficiency, and mRNA and protein subcellular localization. RNA secondary and tertiary structures are critical for these regulatory activities.

Remarkably, GWAS studies have shown that there are far more single nucleotide polymorphisms (SNPs) associated with human disease in the noncoding transcriptome relative to the coding transcripts (Maurano et al., Science 337:1190-1195; 2012). Therefore, the therapeutic targeting of noncoding RNAs and noncoding regions of mRNA can yield novel agents to treat to previously intractable human diseases.

Current therapeutic approaches to interdict mRNA require methods such as gene therapy (Naldini, Nature 2015, 526, 351-360), genome editing (Cox et al., Nature Medicine 2015, 21, 121-131), or a wide range of oligonucleotide technologies (antisense, RNAi, etc.) (Bennett & Swayze, Annu. Rev. Pharmacol. Toxicol. 2010, 50, 259-293). Oligonucleotides modulate the action of RNA via canonical base/base hybridization. The appeal of this approach is that the basic pharmacophore of an oligonucleotide can be defined in a straightforward fashion from the sequence subject to interdiction. Each of these therapeutic modalities suffers from substantial technical, clinical, and regulatory challenges. Some limitations of oligonucleotides as therapeutics (e.g. antisense, RNAi) include unfavorable pharmacokinetics, lack of oral bioavailability, and lack of blood-brain-barrier penetration, with the latter precluding delivery to the brain or spinal cord after parenteral drug administration for the treatment of neurological diseases. In addition, oligonucleotides are not taken up effectively into solid tumors without a complex delivery system such as lipid nanoparticles. Lastly, a vast majority of the oligonucleotides that are taken up into cells and tissues remain in a non-functional compartment such as endosomes, and only a small fraction of the material escapes to gain access to the cytosol and/or nucleus where the target is located.

“Traditional” small molecules can be optimized to exhibit excellent absorption from the gut, excellent distribution to target organs, and excellent cell penetration. The present invention contemplates use of “traditional” (i.e., “Lipinski-compliant” (Lipinski et al., Adv. Drug Deliv. Rev. 2001, 46, 3-26) small molecules with favorable drug properties that bind and modulate the activity of a target RNA. Accordingly, in one aspect, the present invention provides a method of identifying a small molecule that binds to and modulates the function of a target RNA, comprising the steps of: screening one or more disclosed compounds for binding to the target RNA and analyzing the results by an RNA binding assay disclosed herein. In some embodiments, the screening method uses a screening library to identify new RNA targets. In some embodiments, the target RNA is selected from a mRNA or a noncoding RNA. In some embodiments, the RNA binding assay identifies the location in the primary sequence of the binding site(s) on the target RNA. In some embodiments, the small molecule is Lipinski-compliant.

Targeting mRNA

Within mRNAs, noncoding regions can affect the level of mRNA and protein expression. Briefly, these include IRES and upstream open reading frames (uORF) that affect translation efficiency, intronic sequences that affect splicing efficiency and alternative splicing patterns, 3′ UTR sequences that affect mRNA and protein localization, and elements that control mRNA decay and half-life. Therapeutic modulation of these RNA elements can have beneficial effects. Also, mRNAs may contain expansions of simple repeat sequences such as trinucleotide repeats. These repeat expansion containing RNAs can be toxic and have been observed to drive disease pathology, particularly in certain neurological and musculoskeletal diseases (see Gatchel & Zoghbi, Nature Rev. Gen. 2005, 6, 743-755). In addition, splicing can be modulated to skip exons having mutations that introduce stop codons in order to relieve premature termination during translation.

Small molecules can be used to modulate splicing of pre-mRNA for therapeutic benefit in a variety of settings. One example is spinal muscular atrophy (SMA). SMA is a consequence of insufficient amounts of the survival of motor neuron (SMN) protein. Humans have two versions of the SMN gene, SMN1 and SMN2. SMA patients have a mutated SMN1 gene and thus rely solely on SMN2 for their SMN protein. The SMN2 gene has a silent mutation in exon 7 that causes inefficient splicing such that exon 7 is skipped in the majority of SMN2 transcripts, leading to the generation of a defective protein that is rapidly degraded in cells, thus limiting the amount of SMN protein produced from this locus. A small molecule that promotes the efficient inclusion of exon 7 during the splicing of SMN2 transcripts would be an effective treatment for SMA (Palacino et al., Nature Chem. Biol., 2015, 11, 511-517). Accordingly, in one aspect, the present invention provides a method of identifying a small molecule that modulates the splicing of a target pre-mRNA to treat a disease or disorder, comprising the steps of: screening one or more disclosed compounds for binding to the target pre-mRNA; and analyzing the results by an RNA binding assay disclosed herein. In some embodiments, the pre-mRNA is an SMN2 transcript. In some embodiments, the disease or disorder is spinal muscular atrophy (SMA).

Even in cases in which defective splicing does not cause the disease, alteration of splicing patterns can be used to correct the disease. Nonsense mutations leading to premature translational termination can be eliminated by exon skipping if the exon sequences are in-frame. This can create a protein that is at least partially functional. One example of the use of exon skipping is the dystrophin gene in Duchenne muscular dystrophy (DMD). A variety of different mutations leading to premature termination codons in DMD patients can be eliminated by exon skipping promoted by oligonucleotides (reviewed in Fairclough et al., Nature Rev. Gen., 2013, 14, 373-378). Small molecules that bind RNA structures and affect splicing are expected to have a similar effect. Accordingly, in one aspect, the present invention provides a method of identifying a small molecule that modulates the splicing pattern of a target pre-mRNA to treat a disease or disorder, comprising the steps of: screening one or more disclosed compounds for binding to the target pre-mRNA; and analyzing the results by an RNA binding assay disclosed herein. In some embodiments, the pre-mRNA is a dystrophin gene transcript. In some embodiments, the small molecule promotes exon skipping to eliminate premature translational termination. In some embodiments, the disease or disorder is Duchenne muscular dystrophy (DMD).

Lastly, the expression of an mRNA and its translation products could be affected by targeting noncoding sequences and structures in the 5′ and 3′ UTRs. For instance, RNA structures in the 5′ UTR can affect translational efficiency. RNA structures such as hairpins in the 5′ UTR have been shown to affect translation. In general, RNA structures are believed to play a critical role in translation of mRNA. Two examples of these are internal ribosome entry sites (IRES) and upstream open reading frames (uORF) that can affect the level of translation of the main open reading frame (Komar and Hatzoglou, Frontiers Oncol. 5:233, 2015; Weingarten-Gabbay et al., Science 351:pii:aad4939, 2016; Calvo et al., Proc. Natl. Acad. Sci. USA 106:7507-7512; Le Quesne et al., J. Pathol. 220:140-151, 2010; Barbosa et al., PLOS Genetics 9:e10035529, 2013). For example, nearly half of all human mRNAs have uORFs, and many of these reduce the translation of the main ORF. Small molecules targeting these RNAs could be used to modulate specific protein levels for therapeutic benefit. Accordingly, in one aspect, the present invention provides a method of producing a small molecule that modulates the expression or translation efficiency of a target pre-mRNA or mRNA to treat a disease or disorder, comprising the steps of: screening one or more disclosed compounds for binding to the target pre-mRNA or mRNA; and analyzing the results by an RNA binding assay disclosed herein. In some embodiments, the small molecule binding site is a 5′ UTR, internal ribosome entry site, or upstream open reading frame.

The present invention contemplates the use of small molecules to up- or down-regulate the expression of specific proteins based on targeting their cognate mRNAs. Accordingly, the present invention provides methods of modulating the downstream protein expression associated with a target mRNA with a small molecule, wherein the small molecule is identified according to the screening methods disclosed herein. In another aspect, the present invention provides a method of producing a small molecule that modulates the downstream protein expression associated with a target mRNA to treat a disease or disorder, comprising the steps of: screening one or more disclosed compounds for binding to the target mRNA; and analyzing the results by an RNA binding assay disclosed herein.

In some embodiments, the present invention provides a method of treating a disease or disorder mediated by mRNA, comprising the step of administering to a patient in need thereof a compound of the present invention. Such compounds are described in detail herein.

Targeting Regulatory RNA

The largest set of RNA targets is RNA that is transcribed but not translated into protein, termed “non-coding RNA”. Non-coding RNA is highly conserved and the many varieties of non-coding RNA play a wide range of regulatory functions. The term “non-coding RNA,” as used herein, includes but is not limited to micro-RNA (miRNA), long non-coding RNA (lncRNA), long intergenic non-coding RNA (lincRNA), Piwi-interacting RNA (piRNA), competing endogenous RNA (ceRNA), and pseudo-genes. Each of these sub-categories of non-coding RNA offers a large number of RNA targets with significant therapeutic potential. Accordingly, in some embodiments, the present invention provides methods of treating a disease mediated by non-coding RNA. In some embodiments, the disease is caused by a miRNA, lncRNA, lincRNA, piRNA, ceRNA, or pseudo-gene. In another aspect, the present invention provides a method of producing a small molecule that modulates the activity of a target non-coding RNA to treat a disease or disorder, comprising the steps of: screening one or more disclosed compounds for binding to the target non-coding RNA; and analyzing the results by an RNA binding assay disclosed herein. In some embodiments, the target non-coding RNA is a miRNA, lncRNA, lincRNA, piRNA, ceRNA, or pseudo-gene.

miRNA are short double-strand RNAs that regulate gene expression (see Elliott & Ladomery, Molecular Biology of RNA, 2^{nd} Ed.). Each miRNA can affect the expression of many human genes. There are nearly 2,000 miRNAs in humans. These RNAs regulate many biological processes, including cell differentiation, cell fate, motility, survival, and function. miRNA expression levels vary between different tissues, cell types, and disease settings. They are frequently aberrantly expressed in tumors versus normal tissue, and their activity may play significant roles in cancer (for reviews, see Croce, Nature Rev. Genet. 10:704-714, 2009; Dykxhoorn Cancer Res. 70:6401-6406, 2010). miRNAs have been shown to regulate oncogenes and tumor suppressors and themselves can act as oncogenes or tumor suppressors. Some have been shown to promote epithelial-mesenchymal transition (EMT) and cancer cell invasiveness and metastasis. In the case of oncogenic miRNAs, their inhibition could be an effective anti-cancer treatment. Accordingly, in one aspect, the present invention provides a method of producing a small molecule that modulates the activity of a target miRNA to treat a disease or disorder, comprising the steps of: screening one or more disclosed compounds for binding to the target miRNA; and analyzing the results by an RNA binding assay disclosed herein. In some embodiments, the miRNA regulates an oncogene or tumor suppressor, or acts as an oncogene or tumor suppressor. In some embodiments, the disease is cancer. In some embodiments, the cancer is a solid tumor.

There are multiple oncogenic miRNA that could be therapeutically targeted including miR-155, miR-17-92, miR-19, miR-21, and miR-10b (see Stahlhut & Slack, Genome Med. 2013, 5, 111). miR-155 plays pathological roles in inflammation, hypertension, heart failure, and cancer. In cancer, miR-155 triggers oncogenic cascades and apoptosis resistance, as well as increasing cancer cell invasiveness. Altered expression of miR-155 has been described in multiple cancers, reflecting staging, progress and treatment outcomes. Cancers in which miR-155 over-expression has been reported are breast cancer, thyroid carcinoma, colon cancer, cervical cancer, and lung cancer. It is reported to play a role in drug resistance in breast cancer. miR-17˜92 (also called Oncomir-1) is a polycistronic 1 kb primary transcript comprising miR-17, 20a, 18a, 19a, 92-1 and 19b-1. It is activated by MYC. miR-19 alters the gene expression and signal transduction pathways in multiple hematopoietic cells, and it triggers leukemogenesis and lymphomagenesis. It is implicated in a wide variety of human solid tumors and hematological cancers. miR-21 is an oncogenic miRNA that reduces the expression of multiple tumor suppressors. It stimulates cancer cell invasion and is associated with a wide variety of human cancers including breast, ovarian, cervix, colon, lung, liver, brain, esophagus, prostate, pancreas, and thyroid cancers. Accordingly, in some embodiments of the methods described above, the target miRNA is selected from miR-155, miR-17-92, miR-19, miR-21, or miR-10b. In some embodiments, the disease or disorder is a cancer selected from breast cancer, ovarian cancer, cervical cancer, thyroid carcinoma, colon cancer, liver cancer, brain cancer, esophageal cancer, prostate cancer, lung cancer, leukemia, or lymph node cancer. In some embodiments, the cancer is a solid tumor.

Beyond oncology, miRNAs play roles in many other diseases including cardiovascular and metabolic diseases (Quiant and Olson, J. Clin. Invest. 123:11-18, 2013; Olson, Science Trans. Med. 6: 239ps3, 2014; Baffy, J. Clin. Med. 4:1977-1988, 2015).

Many mature miRNAs are relatively short in length and thus may lack sufficient folded, three-dimensional structure to be targeted by small molecules. However, it is believed that the levels of such miRNA could be reduced by small molecules that bind the primary transcript or the pre-miRNA to block the biogenesis of the mature miRNA. Accordingly, in some embodiments of the methods described above, the target miRNA is a primary transcript or pre-miRNA.

lncRNA are RNAs of over 200 nucleotides (nt) that do not encode proteins (see Rinn & Chang, Ann. Rev. Biochem. 2012, 81, 145-166; (for reviews, see Morris and Mattick, Nature Reviews Genetics 15:423-437, 2014; Mattick and Rinn, Nature Structural & Mol. Biol. 22:5-7, 2015; Iyer et al., Nature Genetics 47(:199-208, 2015)). They can affect the expression of the protein-encoding mRNAs at the level of transcription, splicing and mRNA decay. Considerable research has shown that lncRNA can regulate transcription by recruiting epigenetic regulators that increase or decrease transcription by altering chromatin structure (e.g., Holoch and Moazed, Nature Reviews Genetics 16:71-84, 2015). lncRNAs are associated with human diseases including cancer, inflammatory diseases, neurological diseases and cardiovascular disease (for instance, Presner and Chinnaiyan, Cancer Discovery 1:391-407, 2011; Johnson, Neurobiology of Disease 46:245-254, 2012; Gutscher and Diederichs, RNA Biology 9:703-719, 2012; Kumar et al., PLOS Genetics 9:e1003201, 2013; van de Vondervoort et al., Frontiers in Molecular Neuroscience, 2013; Li et al., Int. J. Mol. Sci. 14:18790-18808, 2013). The targeting of lncRNA could be done to up-regulate or down-regulate the expression of specific genes and proteins for therapeutic benefit (e.g., Wahlestedt, Nature Reviews Drug Discovery 12:433-446, 2013; Guil and Esteller, Nature Structural & Mol. Biol. 19:1068-1075, 2012). In general, lncRNA are expressed at a lower level relative to mRNAs. Many lncRNAs are physically associated with chromatin (Werner et al., Cell Reports 12, 1-10, 2015) and are transcribed in close proximity to protein-encoding genes. They often remain physically associated at their site of transcription and act locally, in cis, to regulate the expression of a neighboring mRNA. The mutation and dysregulation of lncRNA is associated with human diseases; therefore, there are a multitude of lncRNAs that could be therapeutic targets. Accordingly, in some embodiments of the methods described above, the target non-coding RNA is a lncRNA. In some embodiments, the lncRNA is associated with a cancer, inflammatory disease, neurological disease, or cardiovascular disease.

lncRNAs regulate the expression of protein-encoding genes, acting at multiple different levels to affect transcription, alternative splicing and mRNA decay. For example, lncRNA has been shown to bind to the epigenetic regulator PRC2 to promote its recruitment to genes whose transcription is then repressed via chromatin modification. lncRNA may form complex structures that mediate their association with various regulatory proteins. A small molecule that binds to these lncRNA structures could be used to modulate the expression of genes that are normally regulated by an individual lncRNA.

One exemplary target lncRNA is HOTAIR, an lncRNA expressed from the HoxC locus on human chromosome 12. Is expression level is low (˜100 RNA copies per cell). Unlike many lncRNAs, HOTAIR can act in trans to affect the expression of distant genes. It binds the epigenetic repressor PRC2 as well as the LSD1/CoREST/REST complex, another repressive epigenetic regulator (Tsai et al., Science 329, 689-693, 2010). HOTAIR is a highly structured RNA with over 50% of its nucleotides being involved in base pairing. It is frequently dysregulated (often up-regulated) in various types of cancer (Yao et al., Int. J. Mol. Sci. 15:18985-18999, 2014; Deng et al., PLOS One 9:e110059, 2014). Cancer patients with high expression levels of HOTAIR have a significantly poorer prognosis, compared with those with low expression levels. HOTAIR has been reported to be involved in the control of apoptosis, proliferation, metastasis, angiogenesis, DNA repair, chemoresistance and tumor cell metabolism. It is highly expressed in metastatic breast cancers. High levels of expression in primary breast tumors are a significant predictor of subsequent metastasis and death. HOTAIR also has been reported to be associated with esophageal squamous cell carcinoma, and it is a prognostic factor in colorectal cancer, cervical cancer, gastric cancer and endometrial carcinoma. Therefore, HOTAIR-binding small molecules are novel anti-cancer drug candidates. Accordingly, in some embodiments of the methods described above, the target non-coding RNA is HOTAIR. In some embodiments, the disease or disorder is breast cancer, esophageal squamous cell carcinoma, colorectal cancer, cervical cancer, gastric cancer, or endometrial carcinoma.

Another potential cancer target among lncRNA is MALAT-1 (metastasis-associated lung adenocarcinoma transcript 1), also known as NEAT2 (nuclear-enriched abundant transcript 2) (Gutschner et al., Cancer Res. 73:1180-1189, 2013; Brown et al., Nat. Structural & Mol. Biol. 21:633-640, 2014). It is a highly conserved 7 kb nuclear lncRNA that is localized in nuclear speckles. It is ubiquitously expressed in normal tissues, but is up-regulated in many cancers. MALAT-1 is a predictive marker for metastasis development in multiple cancers including lung cancer. It appears to function as a regulator of gene expression, potentially affecting transcription and/or splicing. MALAT-1 knockout mice have no phenotype, indicating that it has limited normal function. However, MALAT-1-deficient cancer cells are impaired in migration and form fewer tumors in a mouse xenograft tumor models. Antisense oligonucleotides (ASO) blocking MALAT-1 prevent metastasis formation after tumor implantation in mice. Some mouse xenograft tumor model data indicates that MALAT-1 knockdown by ASOs may inhibit both primary tumor growth and metastasis. Thus, a small molecule targeting MALAT-1 is expected to be effective in inhibiting tumor growth and metastasis. Accordingly, in some embodiments of the methods described above, the target non-coding RNA is MALAT-1. In some embodiments, the disease or disorder is a cancer in which MALAT-1 is upregulated, such as lung cancer.

In some embodiments, the present invention provides a method of treating a disease or disorder mediated by non-coding RNA (such as HOTAIR or MALAT-1), comprising the step of administering to a patient in need thereof a compound of the present invention. Such compounds are described in detail herein.

Targeting Toxic RNA (Repeat RNA)

Simple repeats in mRNA often are associated with human disease. These are often, but not exclusively, repeats of three nucleotides such as CAG (“triplet repeats”) (for reviews, see Gatchel and Zoghbi, Nature Reviews Genetics 6:743-755, 2005; Krzyzosiak et al., Nucleic Acids Res. 40:11-26, 2012; Budworth and McMurray, Methods Mol. Biol. 1010:3-17, 2013). Triplet repeats are abundant in the human genome, and they tend to undergo expansion over generations. Approximately 40 human diseases are associated with the expansion of repeat sequences. Diseases caused by triplet expansions are known as Triplet Repeat Expansion Diseases (TRED). Healthy individuals have a variable number of triplet repeats, but there is a threshold beyond which a higher repeat number causes disease. The threshold varies in different disorders. The triplet repeat can be unstable. As the gene is inherited, the number of repeats may increase, and the condition may be more severe or have an earlier onset from generation to generation. When an individual has a number of repeats in the normal range, it is not expected to expand when passed to the next generation. When the repeat number is in the premutation range (a normal, but unstable repeat number), then the repeats may or may not expand upon transmission to the next generation. Normal individuals who carry a premutation do not have the condition, but are at risk of having a child who has inherited a triplet repeat in the full mutation range and who will be affected. TREDs can be autosomal dominant, autosomal recessive or X-linked. The more common triplet repeat disorders are autosomal dominant.

The repeats can be in the coding or noncoding portions of the mRNA. In the case of repeats within noncoding regions, the repeats may lie in the 5′ UTR, introns, or 3′ UTR sequences. Some examples of diseases caused by repeat sequences within coding regions are shown in Table 1.

TABLE 1
Repeat Expansion Diseases in Which the Repeat
Resides in the Coding Regions of mRNA

| | | | Normal repeat | Disease repeat |
|---|---|---|---|---|
| Disease | Gene | Repeat | number | number |
| HD | HTT | CAG | 6-35 (SEQ ID | 36-250 (SEQ ID |
| | | | NO: 1) | NO: 8) |
| DRPLA | ATN1 | CAG | 6-35 (SEQ ID | 49-88 (SEQ ID |
| | | | NO: 1) | NO: 9) |
| SBMA | AR | CAG | 9-36 (SEQ ID | 38-62 (SEQ ID |
| | | | NO: 2) | NO: 10) |
| SCA1 | ATXN1 | CAG | 6-35 (SEQ ID | 49-88 (SEQ ID |
| | | | NO: 1) | NO: 9) |
| SCA2 | ATXN2 | CAG | 14-32 (SEQ ID | 33-77 (SEQ ID |
| | | | NO: 3) | NO: 11) |
| SCA3 | ATXN3 | CAG | 12-40 (SEQ ID | 55-86 (SEQ ID |
| | | | NO: 4) | NO: 12) |
| SCA6 | CACNA1A | CAG | 4-18 (SEQ ID | 21-30 (SEQ ID |
| | | | NO: 5) | NO: 13) |
| SCA7 | ATXN7 | CAG | 7-17 (SEQ ID | 38-120 (SEQ ID |
| | | | NO: 6) | NO: 14) |
| SCA17 | TBP | CAG | 25-42 (SEQ ID | 47-63 (SEQ ID |
| | | | NO: 7) | NO: 15) |

Some examples of diseases caused by repeat sequences within noncoding regions of mRNA are shown in Table 2.

TABLE 2
Repeat Expansion Diseases in Which the Repeat
Resides in the Noncoding Regions of mRNA

| | | | Repeat | Normal repeat | Disease repeat |
|---|---|---|---|---|---|
| Disease | Gene | Repeat | location | number | number |
| Fragile X | FMR1 | CGG | 5′ UTR | 6-53 (SEQ ID | ≥230 |
| | | | | NO: 16) | |
| DM1 | DMPK | CTG | 3′ UTR | 5-37 (SEQ ID | ≥50 |
| | | | | NO: 17) | |
| FRDA | FXN | GAA | Intron | 7-34 (SEQ ID | ≥100 |
| | | | | NO: 18) | |
| SCA8 | ATXN8 | CTG | Noncoding | 16-37 (SEQ ID | 110-250 (SEQ ID |
| | | | antisense | NO: 19) | NO: 22) |
| SCA10 | ATXN10 | ATTCT | Intron | 9-32 (SEQ ID | 800-4500 (SEQ ID |
| | | | | NO: 20) | NO: 23) |
| SCA12 | PPP2R2B | CAG | 5′ UTR | 7-28 (SEQ ID | 66-78 (SEQ ID |
| | | | | NO: 21) | NO: 24) |
| C9FTD/ALS | C9orf72 | GGGGCC | Intron | ~30 | 100 s |

The toxicity that results from the repeat sequence can be direct consequence of the action of the toxic RNA itself, or, in cases in which the repeat expansion is in the coding sequence, due to the toxicity of the RNA and/or the aberrant protein. The repeat expansion RNA can act by sequestering critical RNA-binding proteins (RBP) into foci. One example of a sequestered RBP is the Muscleblind family protein MBNL1. Sequestration of RBPs leads to defects in splicing as well as defects in nuclear-cytoplasmic transport of RNA and proteins. Sequestration of RBPs also can affect miRNA biogenesis. These perturbations in RNA biology can profoundly affect neuronal function and survival, leading to a variety of neurological diseases.

Repeat sequences in RNA form secondary and tertiary structures that bind RBPs and affect normal RNA biology. One specific example disease is myotonic dystrophy (DM1; dystrophia myotonica), a common inherited form of muscle disease characterized by muscle weakness and slow relaxation of the muscles after contraction (Machuca-Tzili et al., Muscle Nerve 32:1-18, 2005). It is caused by a CUG expansion in the 3′ UTR of the dystrophia myotonica protein kinase (DMPK) gene. This repeat-containing RNA causes the misregulation of alternative splicing of several developmentally regulated transcripts through effects on the splicing regulators MBNL1 and the CUG repeat binding protein (CELF1) (Wheeler et al., Science 325:336-339, 2009). Small molecules that bind the CUG repeat within the DMPK transcript would alter the RNA structure and prevent focus formation and alleviate the effects on these spicing regulators. Fragile X Syndrome (FXS), the most common inherited form of mental retardation, is the consequence of a CGG repeat expansion within the 5′ UTR of the FMR1 gene (Lozano et al., Intractable Rare Dis. Res. 3:134-146, 2014). FMRP is critical for the regulation of translation of many mRNAs and for protein trafficking, and it is an essential protein for synaptic development and neural plasticity. Thus, its deficiency leads to neuropathology. A small molecule targeting this CGG repeat RNA may alleviate the suppression of FMR1 mRNA and FMRP protein expression. Another TRED having a very high unmet medical need is Huntington's disease (HD). HD is a progressive neurological disorder with motor, cognitive, and psychiatric changes (Zuccato et al., Physiol Rev. 90:905-981, 2010). It is characterized as a poly-glutamine or polyQ disorder since the CAG repeat within the coding sequence of the HTT gene leads to a protein having a poly-glutamine repeat that appears to have detrimental effects on transcription, vesicle trafficking, mitochondrial function, and proteasome activity. However, the HTT CAG repeat RNA itself also demonstrates toxicity, including the sequestration of MBNL1 protein into nuclear inclusions. One other specific example is the GGGGCC repeat expansion in the C9orf72 (chromosome 9 open reading frame 72) gene that is prevalent in both familial frontotemporal dementia (FTD) and amyotrophic lateral sclerosis (ALS) (Ling et al., Neuron 79:416-438, 2013; Haeusler et al., Nature 507:195-200, 2014). The repeat RNA structures form nuclear foci that sequester critical RNA binding proteins. The GGGGCC repeat RNA also binds and sequesters RanGAPI to impair nucleocytoplasmic transport of RNA and proteins (Zhang et al., Nature 525:56-61, 2015). Selectively targeting any of these repeat expansion RNAs could add therapeutic benefit in these neurological diseases.

The present invention contemplates a method of treating a disease or disorder wherein aberrant RNAs themselves cause pathogenic effects, rather than acting through the agency of protein expression or regulation of protein expression. In some embodiments, the disease or disorder is mediated by repeat RNA, such as those described above or in Tables 1 and 2. In some embodiments, the disease or disorder is a repeat expansion disease in which the repeat resides in the coding regions of mRNA. In some embodiments, the disease or disorder is a repeat expansion disease in which the repeat resides in the noncoding regions of mRNA. In some embodiments, the disease or disorder is selected from Huntington's disease (HD), dentatorubral-pallidoluysian atrophy (DRPLA), spinal-bulbar muscular atrophy (SBMA), or a spinocerebellar ataxia (SCA) selected from SCA1, SCA2, SCA3, SCA6, SCA7, or SCA17. In some embodiments, the disease or disorder is selected from Fragile X Syndrome, myotonic dystrophy (DM1 or dystrophia myotonica), Friedreich's Ataxia (FRDA), a spinocerebellar ataxia (SCA) selected from SCA8, SCA10, or SCA12, or C9FTD (amyotrophic lateral sclerosis or ALS).

In some embodiments, the disease is amyotrophic lateral sclerosis (ALS), Huntington's disease (HD), frontotemporal dementia (FTD), myotonic dystrophy (DM1 or dystrophia myotonica), or Fragile X Syndrome.

In some embodiments, the present invention provides a method of treating a disease or disorder mediated by repeat RNA, comprising the step of administering to a patient in need thereof a compound of the present invention. Such compounds are described in detail herein.

Also provided is a method of producing a small molecule that modulates the activity of a target repeat expansion RNA to treat a disease or disorder, comprising the steps of: screening one or more disclosed compounds for binding to the target repeat expansion RNA; and analyzing the results by an RNA binding assay disclosed herein. In some embodiments, the repeat expansion RNA causes a disease or disorder selected from HD, DRPLA, SBMA, SCA1, SCA2, SCA3, SCA6, SCA7, or SCA17. In some embodiments, the disease or disorder is selected from Fragile X Syndrome, DM1, FRDA, SCA8, SCA10, SCA12, or C9FTD.

Other Target RNAs and Diseases/Conditions

An association is known to exist between a large number of additional RNAs and diseases or conditions, some of which are shown below in Table 3. Accordingly, in some embodiments of the methods described above, the target RNA is selected from those in Table 3. In some embodiments, the disease or disorder is selected from those in Table 3.

TABLE 3
Target RNAs and Associated Diseases/Conditions

| RNA Target | Indication |
|---|---|
| A20 | inflammatory diseases; liver failure; liver |
| | transplant |
| ABCA1 | coronary artery disease |
| ABCB11 | Primary Biliary Sclerosis |
| ABCB4 | Primary Biliary Sclerosis |
| ABCG5/8 | coronary artery disease |
| Adiponectin | diabetes; obesity; metabolic syndrome |
| AMPK | diabetes |
| ApoA1 | hypercholesterolemia |
| ApoA5 | hypercholesterolemia |
| ApoC3 | chylomicronemia syndrome |
| AR | prostate cancer |
| ARlnc-1 | prostate cancer |
| ATXN1 | spinocerebellar ataxia 1 |
| ATXN10 | spinocerebellar ataxia 10 |
| ATXN2 | spinocerebellar ataxia 2 |
| ATXN3 | spinocerebellar ataxia 3 |
| ATXN7 | spinocerebellar ataxia 7 |
| ATXN8 | spinocerebellar ataxia 8 |
| BACE1 | AD |
| BCL2 | cancer |
| BCR/ABL | CML |
| BDNF | Huntington's Disease |
| Beta-catenin | cancer |
| BRAF | cancer |
| BRCA1 | cancer |
| BRD4 | cancer |
| BTK | cancer |
| C9orf72 (ALS, FTD) | ALS, FTD |
| CACNA1A | spinocerebellar ataxia 6 |
| CD274 | tumor immunology |
| CD279 | tumor immunology |
| CD3zeta | inflammation and autoimmune diseases |
| CD40LG | inflammation |
| CFTR | Cystic Fibrosis |
| cKIT | GIST; mastocytoma |
| CNTF | macular degeneration |
| Complement Factor H | macular degeneration |
| CRACM1 | inflammatory diseases; autoimmune disease; |
| | organ transplant |
| CTLA4 | cancer; inflammatory diseases |
| DGAT2 | NASH |
| DIO2 | dyslipidemia |
| Dystrophin | Duchenne Muscular Dystrophy; Becker's |
| | Muscular Dystrophy |
| EGFR | cancer |
| EIF4E | cancer |
| EZH2 | cancer |
| Factor 7 | hemophilia |
| Factor 8 | hemophilia |
| Factor 9 | hemophilia |
| Fetal Hemoglobin | sickle cell anemia; beta-thalassemia |
| FLT3 | AML |
| FMR1 | Fragile X Syndrome |
| Foxp3 | inflammation & autoimmune diseases |
| Frataxin | Friedreich's Ataxia |
| HAMP/Hepcidin | thalassemia; hereditary hemochromatosis |
| HER2 | cancer |
| HIF-1a | cancer |
| HOTAIR | cancer |
| HTT | Huntington's Disease |
| IL-1 | rheumatoid arthritis |
| IL-17 | inflammatory & autoimmune diseases |
| IL-23 | inflammatory & autoimmune diseases |
| IL-6 | rheumatoid arthritis |
| Ipf1/Pdx1 | diabetes |
| KRAS | cancer |
| Laminin-1a | Merosin-deficient congenital muscular dystrophy |
| | MDCA1 |
| LARGE | Muscular Dystroglycanopathy Type B, 6 |
| LDLR | hypercholesterolemia |
| LINGO1 | neurodegeneration |
| MALAT1 | cancer |
| MAX | cancer |
| MBNL1 | Myotonic Dystrophy |
| MCL1 | cancer |
| MECP2 | Rett Syndrome |
| Mertk | Lupus |
| miR-103 | NASH |
| miR-107 | NASH |
| miR-10b | GBM |
| miR-155 | ALS and others |
| miR-21 | solid tumors |
| miR-221 | HCC |
| mTOR | cancer |
| MYC | cancer |
| Nanog | neurological diseases |
| NF1 | neurofibromatosis |
| Nrf2 | multiple sclerosis |
| PAH | phenylketonuria |
| PCSK6 | hypertension |
| PCSK9 | hypercholesterolemia |
| PD-1 | cancer; inflammation |
| PD-L1 | cancer; inflammation |
| PDK1/2 | Polycystic kidney disease |
| PGC1-a/FNDC5 | PGC1-a/FNDC5 |
| Progranulin | neurological diseases |
| PTB-1B | diabetes |
| PTEN | cancer |
| PTPN1 | Type II diabetes |
| r(AUUCU)^{exp} | SCA10 |
| r(CAG)^{exp} | Huntington's Disease |
| r(CCUG)^{exp} | DM2 |
| r(CGG)^{exp} | FXTAS |
| r(CUG)^{exp} | DM1 |
| r(GGGGCC)^{exp} | c9ALS (familial) |
| r(GGGGCC)^{exp} | c9FTD |
| Ras | Cancer |
| RORC | autoimmune disease |
| RTN4 | Neurodegeneration |
| RTN4R | Neurodegeneration |
| Sarcospan | Duchenne Muscular Dystrophy |
| Serca2a | congestive heart failure |
| SirT6 | Cancer |
| SMAD7 | IBD |
| SMN2 | Spinal Muscular Atrophy |
| SNCA | AD |
| SORT1 | coronary artery disease |
| SRBI | coronary artery disease |
| STAT3 | Cancer |
| STAT5 | Cancer |
| T-bet | Cancer |
| Thyroid Hormone | dyslipidemia; NASH; NAFLD |
| Receptor beta | |
| TIM-3 | inflammatory diseases; cancer |
| TNFa | inflammatory disease |
| TNFRSF11A | Osteoporosis |
| TNFSF11 | Osteoporosis |
| TRIB1 | coronary artery disease |
| TTR | Amyloidosis |
| TWIST1 | Cancer |
| Utrophin | Duchenne Muscular Dystrophy |
| Wnt | Cancer |

### 2. Compounds and Embodiments Thereof

It has now been found that compounds of this invention, and pharmaceutically acceptable compositions thereof, are effective as agents for use in drug discovery; as RNA modulators for treating, preventing, or ameliorating a disease or condition associated with a target RNA; and for use in methods of determining the location and/or structure of an active site or allosteric sites and/or the tertiary structure of a target RNA.

In one aspect, compounds of the present invention, and pharmaceutical compositions thereof, are useful in identifying a small molecule ligand that binds selectively to one or more binding sites (such as active or allosteric sites) on a target RNA to treat, prevent, or ameliorate a disease or condition associated with the target RNA.

In another aspect, compounds of the present invention, and pharmaceutical compositions thereof, are useful as therapeutic agents, for example by modulating a target RNA to treat, prevent, or ameliorate a disease or condition associated with the target RNA. For example, without wishing to be bound by theory, disclosed compounds may act as irreversible inhibitors of a target RNA by covalent binding of the modifying moiety to a 2′-OH of the target RNA that is proximal to the binding site of the small molecule ligand.

In another aspect, compounds of the present invention, and pharmaceutical compositions thereof, are useful in determining the location and/or structure of an active site or allosteric sites and/or the tertiary structure of a target RNA.

In some embodiments, the present invention provides a compound comprising:
(a) a small molecule ligand that binds selectively to one or more binding sites on a target RNA;
(b) a modifying moiety (or “warhead”) that forms a covalent bond to one or more 2′-OH of the target RNA;
(c) optionally, a click-ready group;
(d) optionally, a pull-down group; and
(e) a tethering group that covalently links the small molecule ligand and the modifying moiety and, optionally, the click-ready group.

Without wishing to be bound by any particular theory, it is believed that compounds of the present invention bind selectively to one or more active or allosteric sites on a target RNA, or other sites determined by binding interactions between the small molecule ligand and the structure of the target RNA; covalently modify one or more 2′-OH groups of the target RNA; and may subsequently be used to identify the active site or other binding sites by sequencing analysis of the distribution of 2′-OH modified nucleotides because the pattern of 2′-OH modification will be constrained by the length and conformation of the tether that connects the RNA ligand with the RNA warhead. The target RNA may be inside a cell, in a cell lysate, or in isolated form prior to contacting the compound. Screening of libraries of disclosed compounds will identify highly potent small-molecule modulators of the activity of the target RNA. It is understood that such small molecules identified by such screening may be used as modulators of a target RNA to treat, prevent, or ameliorate a disease or condition in a patient in need thereof.

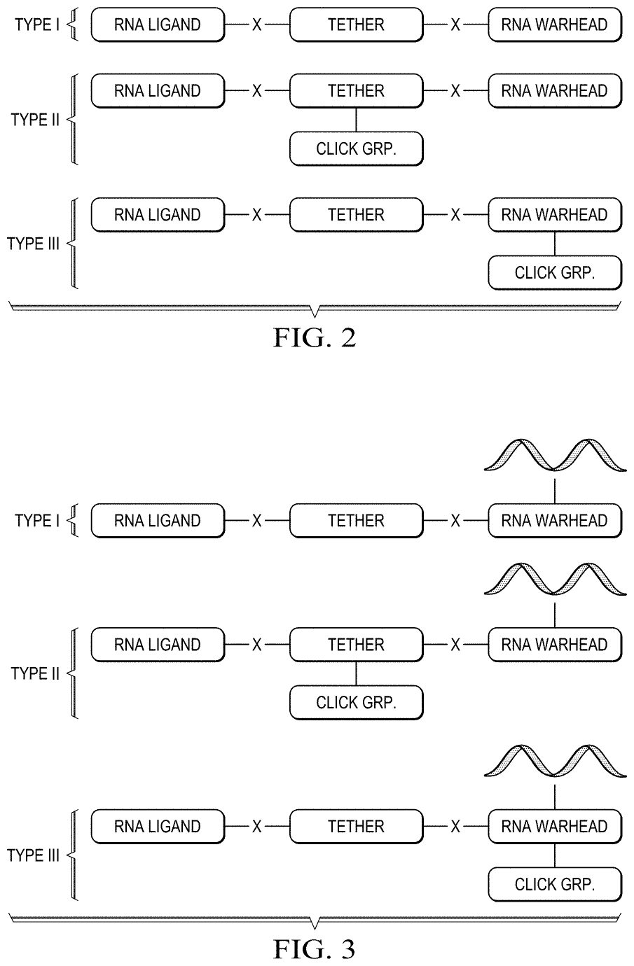

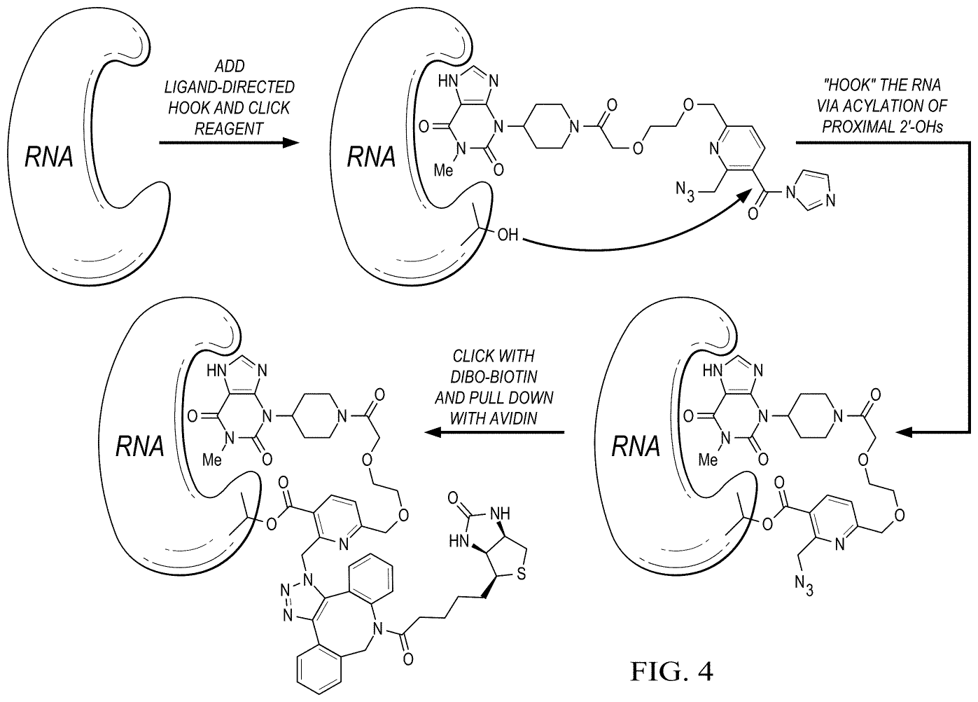

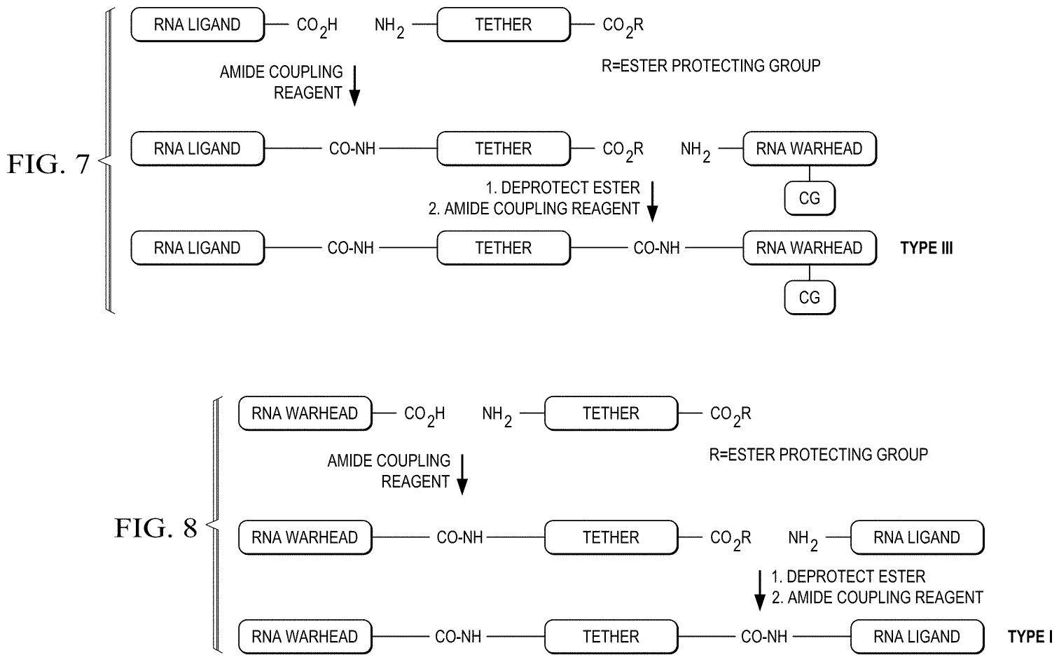

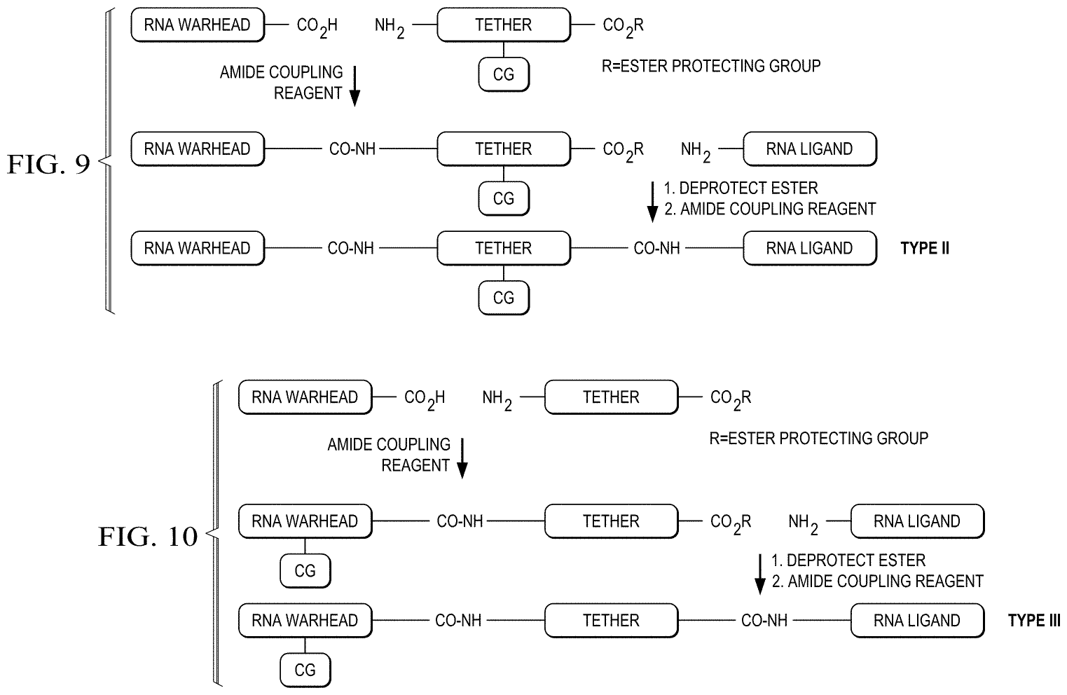

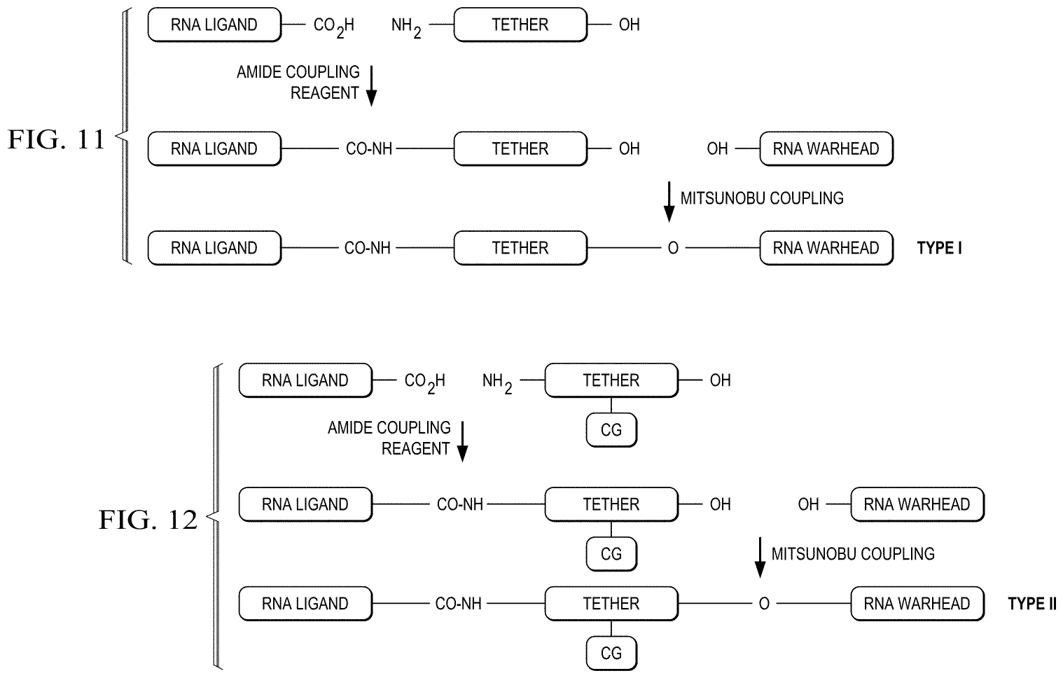

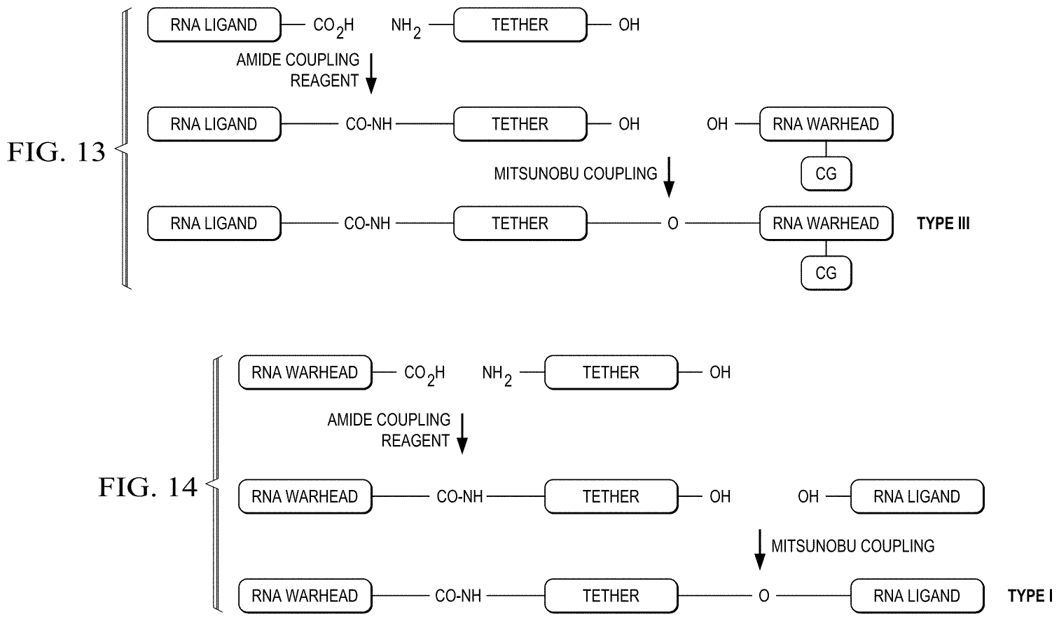

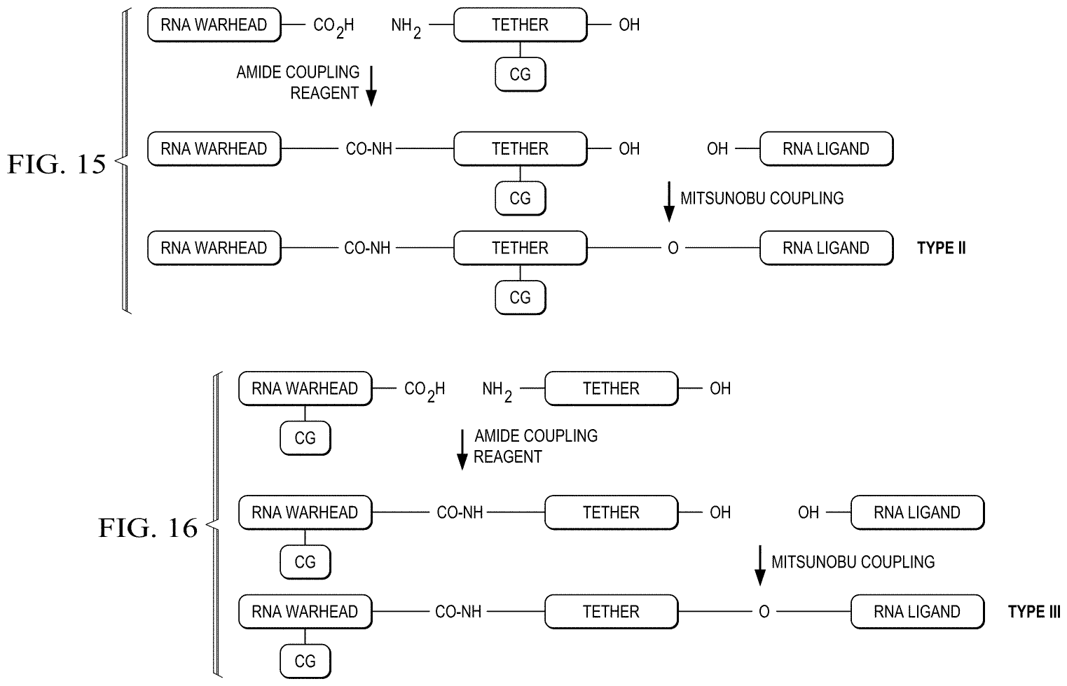

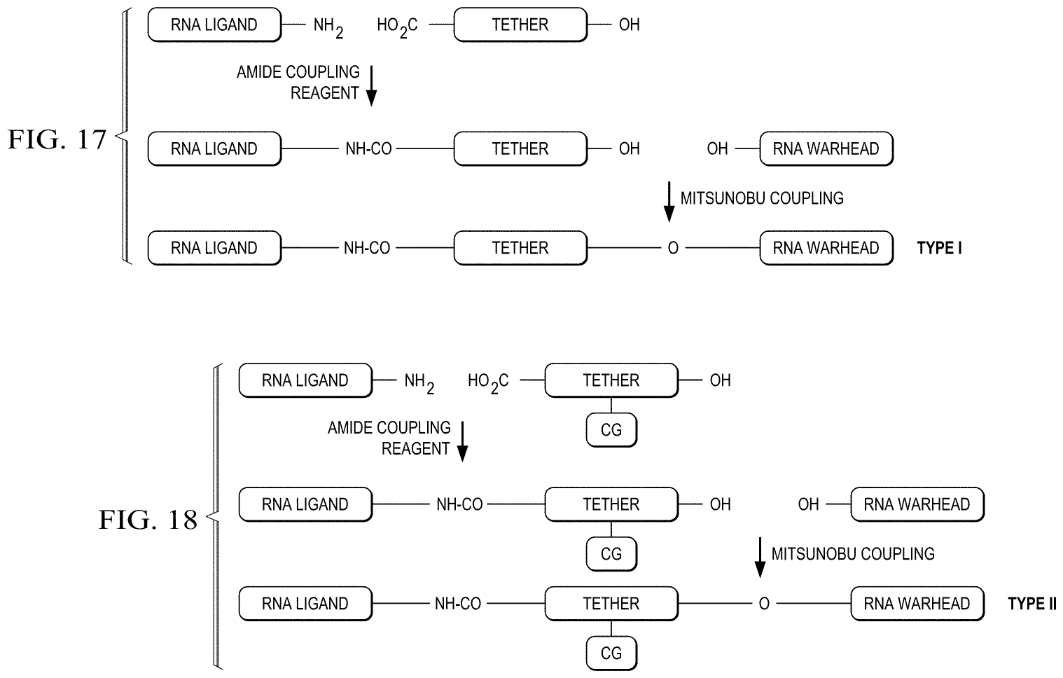

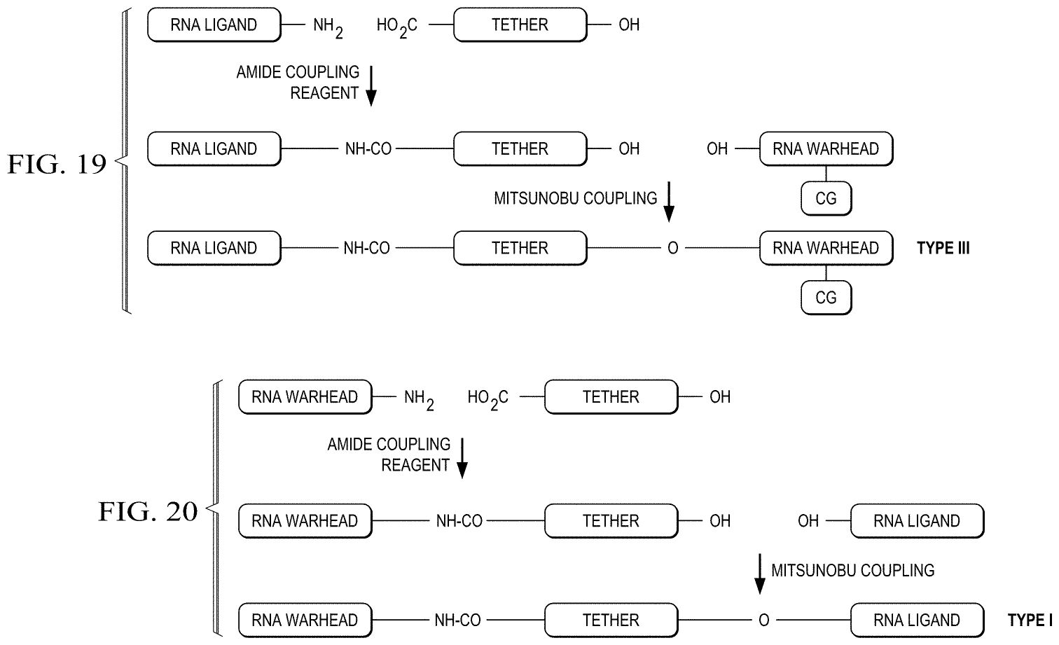

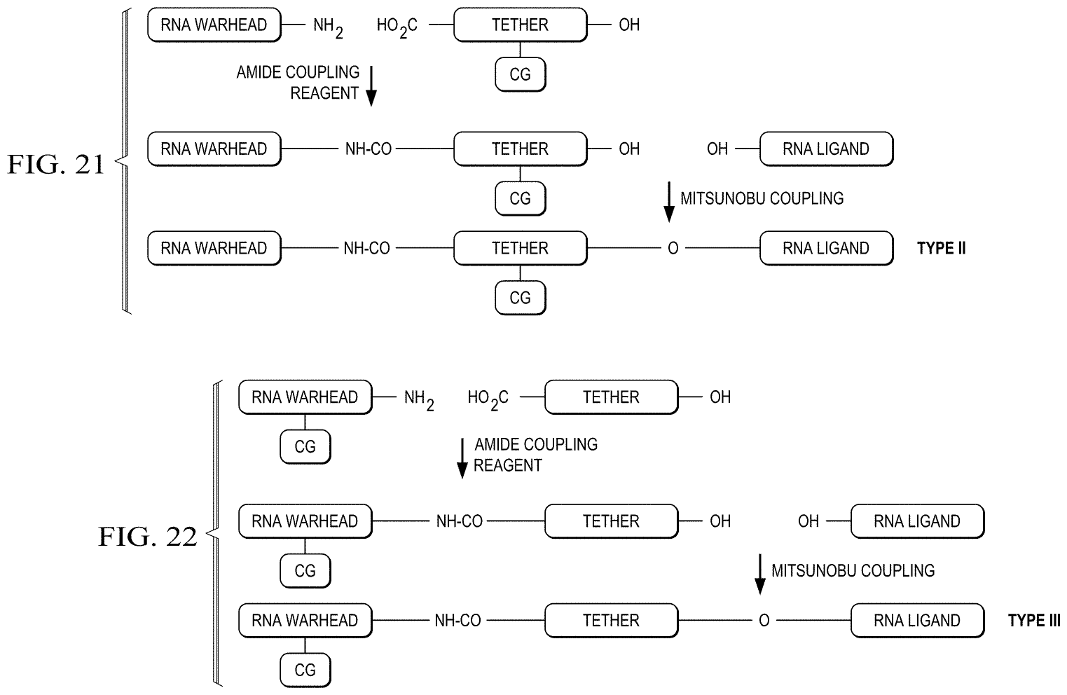

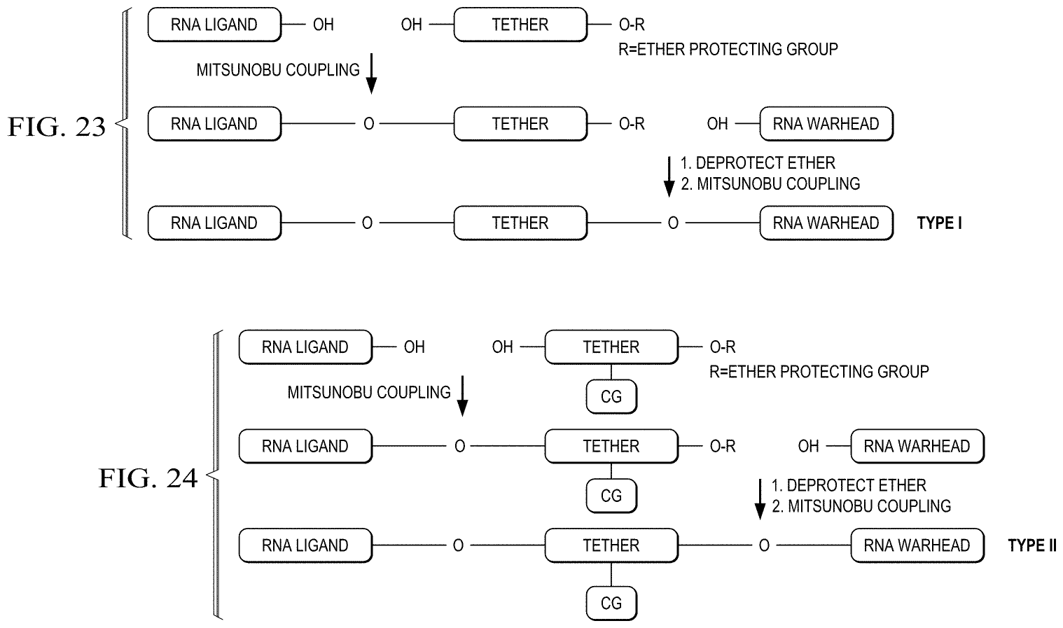

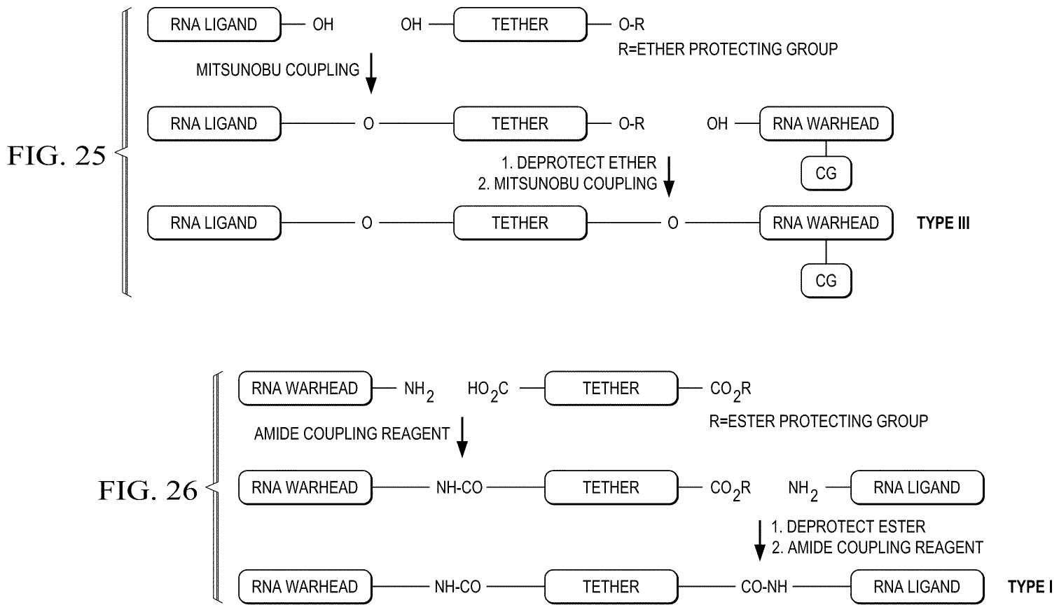

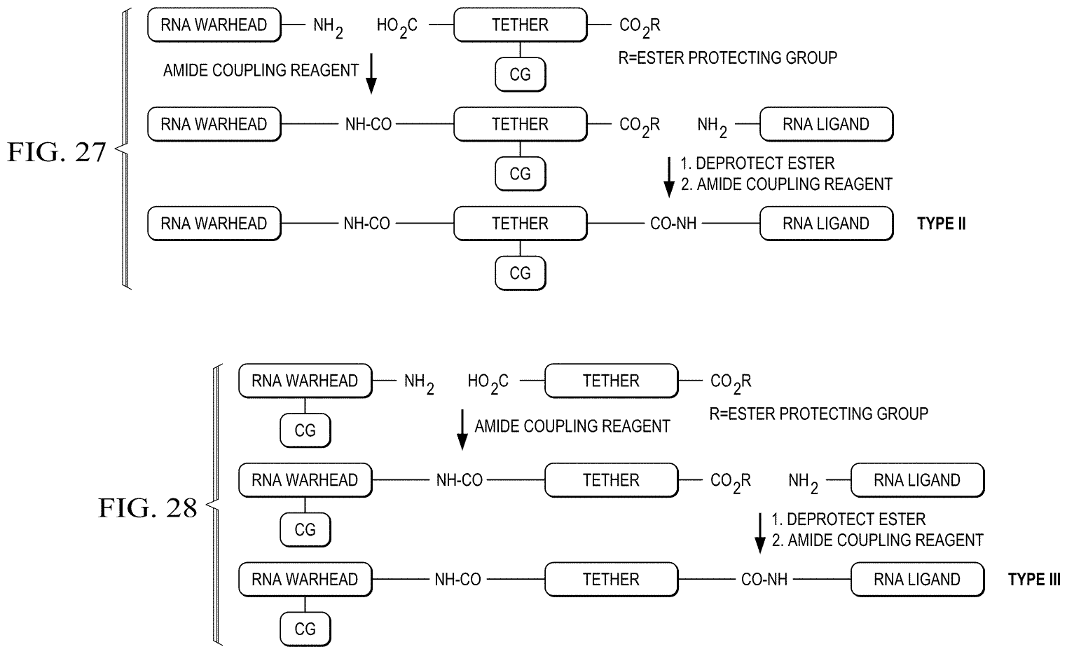

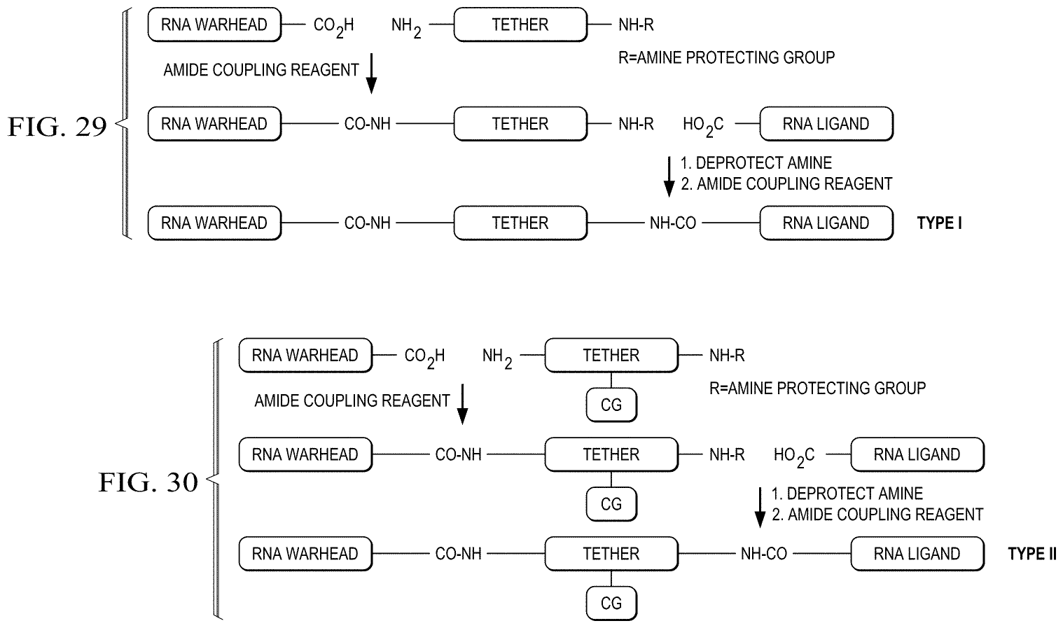

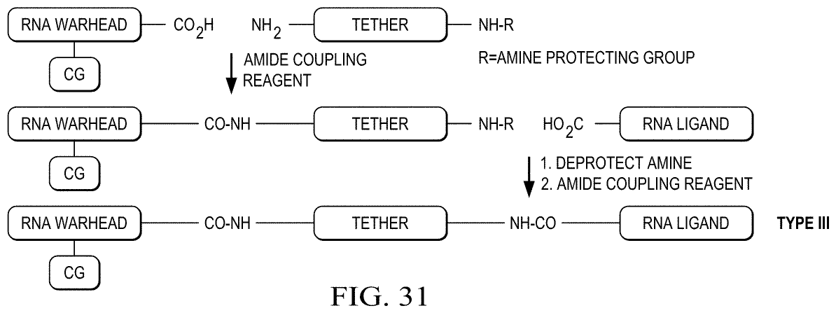

In certain embodiments, a provided compound falls into three groups as shown in FIG. 2 and FIGS. 5-31: Type I, Type II, and Type III.

Compounds of Type I have the general Formula I:

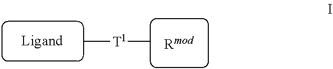

or a pharmaceutically acceptable salt thereof, wherein:
Ligand is a small molecule RNA binder;
T¹ is a bivalent tethering group; and
R^{mod} is a RNA-modifying moiety; wherein each variable is as defined below.

Compounds of Type II have the general Formula II:

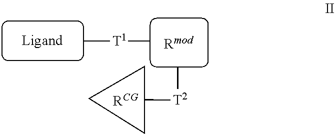

or a pharmaceutically acceptable salt thereof, wherein:
Ligand is a small molecule RNA binder;
each of T¹ and T² is independently a bivalent tethering group;
R^{mod} is a RNA-modifying moiety; and
and R^{CG} is a click-ready group; wherein each variable is as defined below.

Compounds of Type III have the general Formula III:

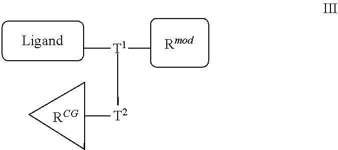

or a pharmaceutically acceptable salt thereof, wherein:
Ligand is a small molecule RNA binder;
T¹ is a trivalent tethering group;
T² is a bivalent tethering group;
R^{mod} is a RNA-modifying moiety; and
R^{CG} is a click-ready group; wherein each variable is as defined below.

In another aspect, the present invention provides a RNA conjugate comprising a target RNA and a compound of any of Formulae I, II, or III, wherein R^{mod} forms a covalent bond to the target RNA.

In some embodiments, the present invention provides a RNA conjugate of Formula IV:

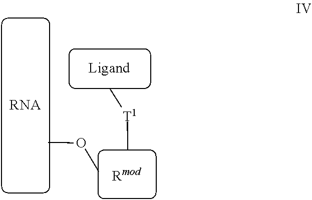

wherein Ligand is a small molecule that binds to a target RNA;
RNA represents the target RNA;
T¹ is a bivalent tethering group; and
R^{mod} is an RNA-modifying moiety;
wherein —O— between R^{mod} and RNA represents a covalent bond from the 2′ hydroxyl of the target RNA to R^{mod}; wherein each variable is as defined below.

In some embodiments, the present invention provides a RNA conjugate of Formula V:

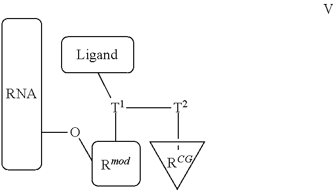

wherein Ligand is a small molecule that binds to a target RNA;
RNA represents the target RNA;
T¹ is a trivalent tethering group;
T² is a bivalent tethering group;
R^{mod} is an RNA-modifying moiety; and
R^{CG} is a click-ready group;
wherein —O— between R^{mod} and RNA represents a covalent bond from the 2′ hydroxyl of the target RNA to R^{mod}; wherein each variable is as defined below.

In some embodiments, the present invention provides a RNA conjugate of Formula VI:

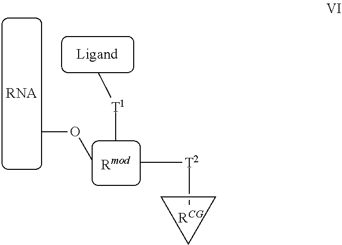

wherein Ligand is a small molecule that binds to a target RNA;
RNA represents the target RNA;
T¹ and T² are each independently a bivalent tethering group;
R^{mod} is an RNA-modifying moiety; and
R^{CG} is a click-ready group;
wherein —O— between R^{mod} and RNA represents a covalent bond from the 2′ hydroxyl of the target RNA to R^{mod}; wherein each variable is as defined below.

In another aspect, the present invention provides a conjugate comprising a target RNA, a compound of Formulae II or III, and a pull-down group, wherein R^{mod} forms a covalent bond to the target RNA.

In some embodiments, the present invention provides a RNA conjugate of Formula VII:

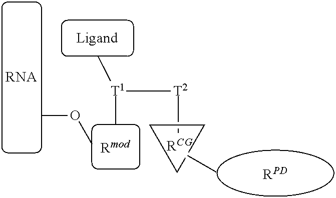

wherein Ligand is a small molecule that binds to a target RNA;
RNA represents the target RNA;
T¹ is a trivalent tethering group;
T² is a bivalent tethering group;
R^{mod} is an RNA-modifying moiety;
R^{CG} is a click-ready group; and
R^{PD} is a pull-down group;
wherein —O— between R^{mod} and RNA represents a covalent bond from the 2′ hydroxyl of the target RNA to R^{mod}; wherein each variable is as defined below. In some embodiments, R^{CG} is

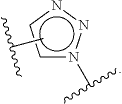

In some embodiments, the present invention provides a RNA conjugate of Formula VIII.

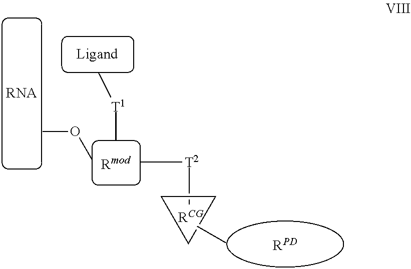

wherein Ligand is a small molecule that binds to a target RNA;
RNA represents the target RNA;
T¹ and T² are bivalent tethering groups;
R^{mod} is an RNA-modifying moiety; and
R^{PD} is a pull-down group; wherein —O— between R^{mod} and RNA represents a covalent bond from the 2′ hydroxyl of the target RNA to R^{mod}; wherein each variable is as defined below. In some embodiments, R^{CG} is

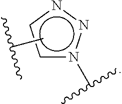

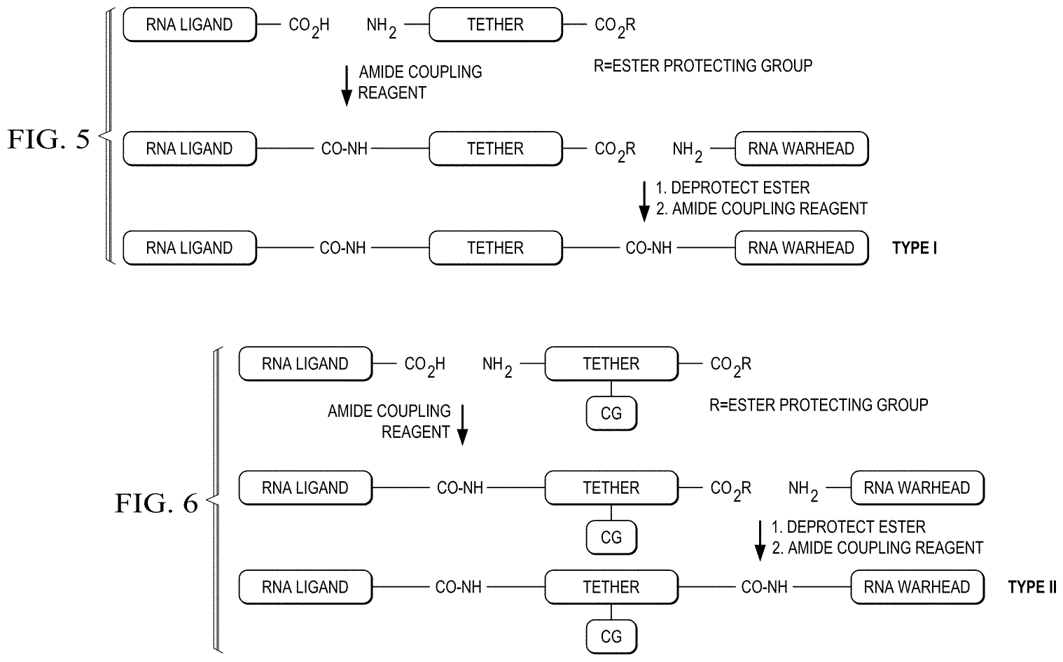

In some embodiments, the compound or conjugate is selected from those formulae shown in FIGS. 5-31, or a pharmaceutically acceptable salt, stereoisomer, or tautomer thereof.

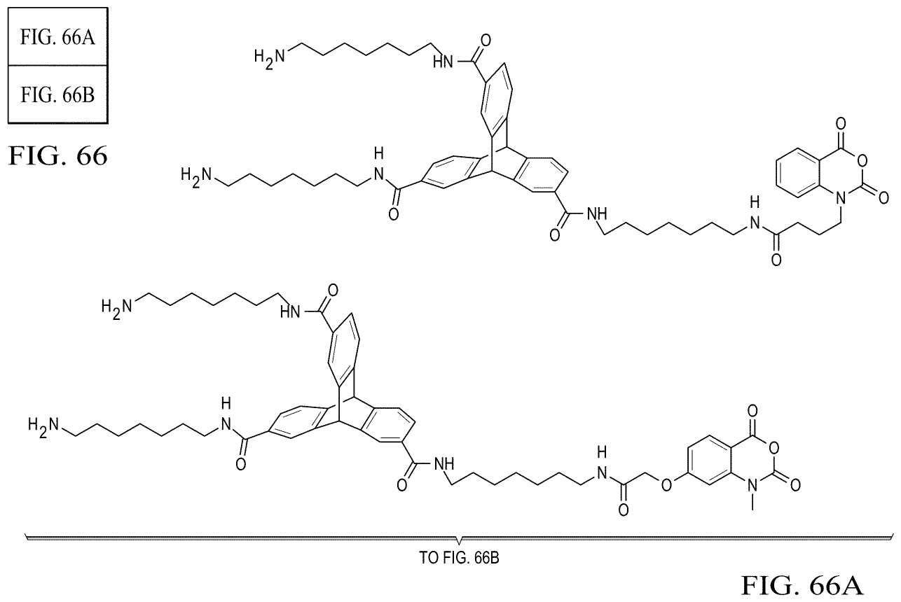

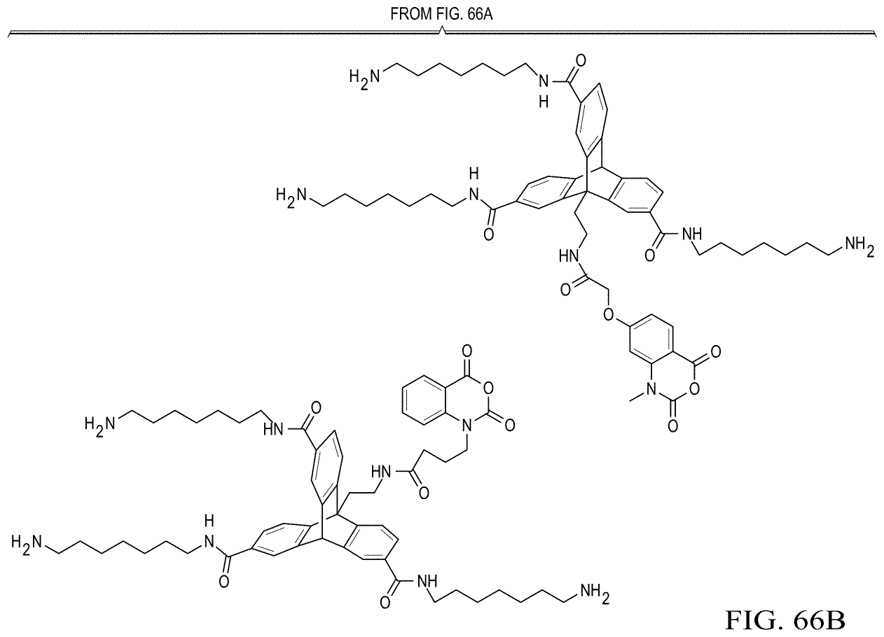

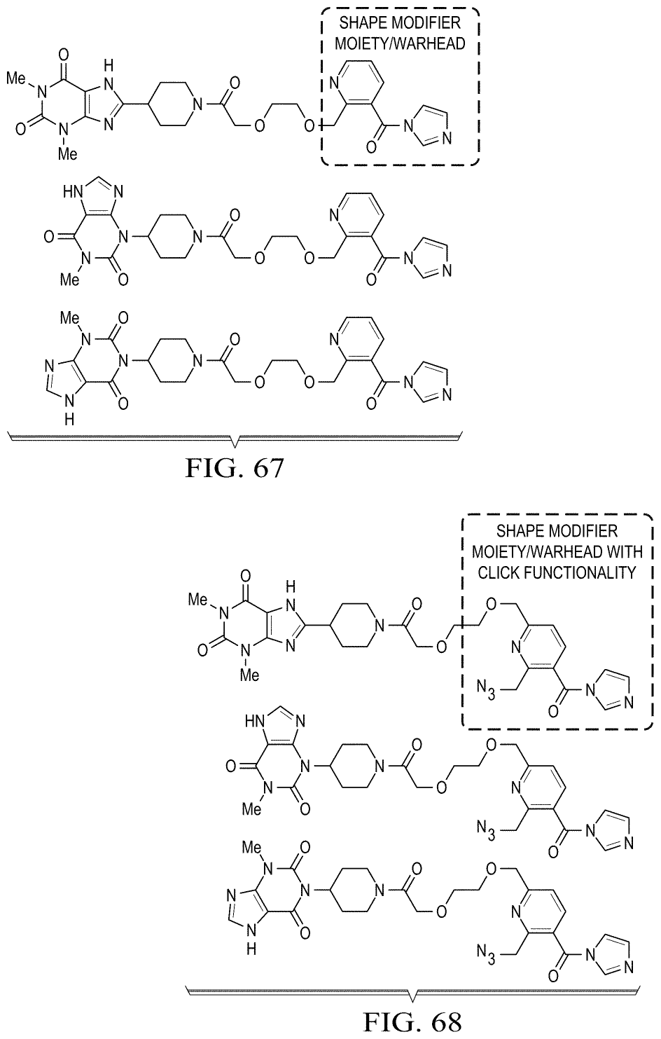

In some embodiments, the compound is selected from those shown in FIG. 66-68, 70-75, or 77-94, or a pharmaceutically acceptable salt, stereoisomer, or tautomer thereof.

Small Molecule RNA Ligands

The design and synthesis of novel, small-molecule ligands capable of binding RNA represents largely untapped therapeutic potential. Certain small-molecule ligands including macrolides (e.g., erythromycin, azithromycin), alkaloids (e.g., berberine, palmatine), aminoglycosides (e.g., paromomycin, neomycin B, kanamycin A), tetracyclines (e.g., doxycycline, oxytetracycline), theophyllines, ribocil, triptycenes, and oxazolidinones (e.g., linezolid, tedizolid) are known to bind to RNA, paving the way for the search for small molecules as RNA targeting drugs. Furthermore, it has now been found that certain compounds comprising a quinoline core, of which CPNQ is one, are capable of binding RNA. CPNQ has the following structure:

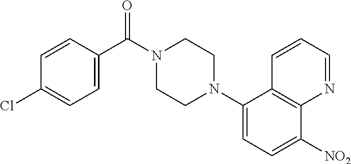 [SMILES: O=C(c1ccc(Cl)cc1)N1CCN(c2ccc([N+](=O)[O-])c3ncccc23)CC1]

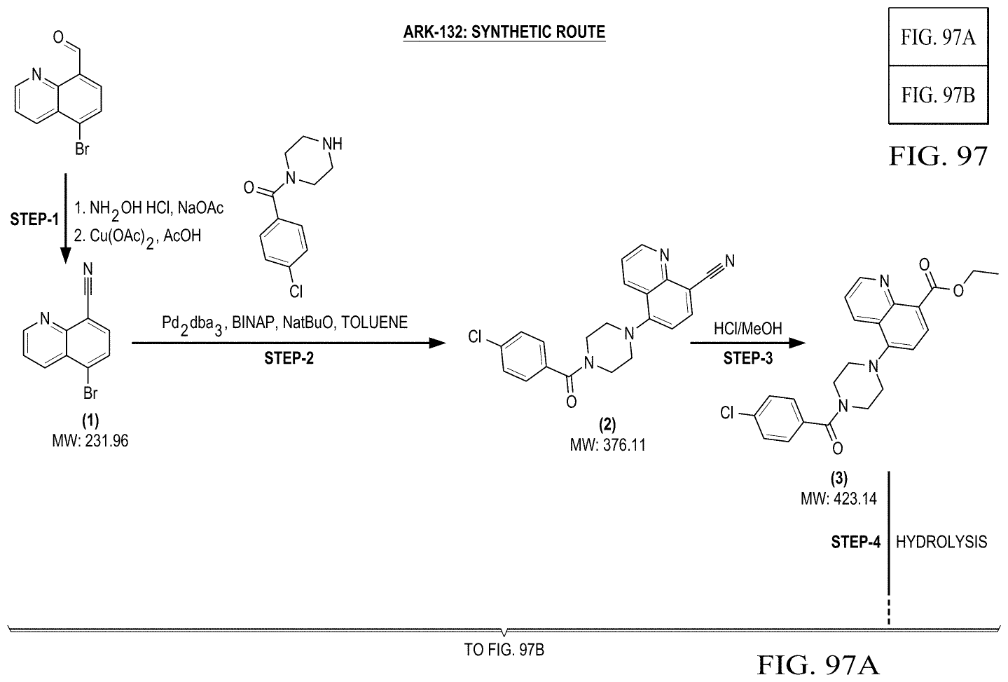

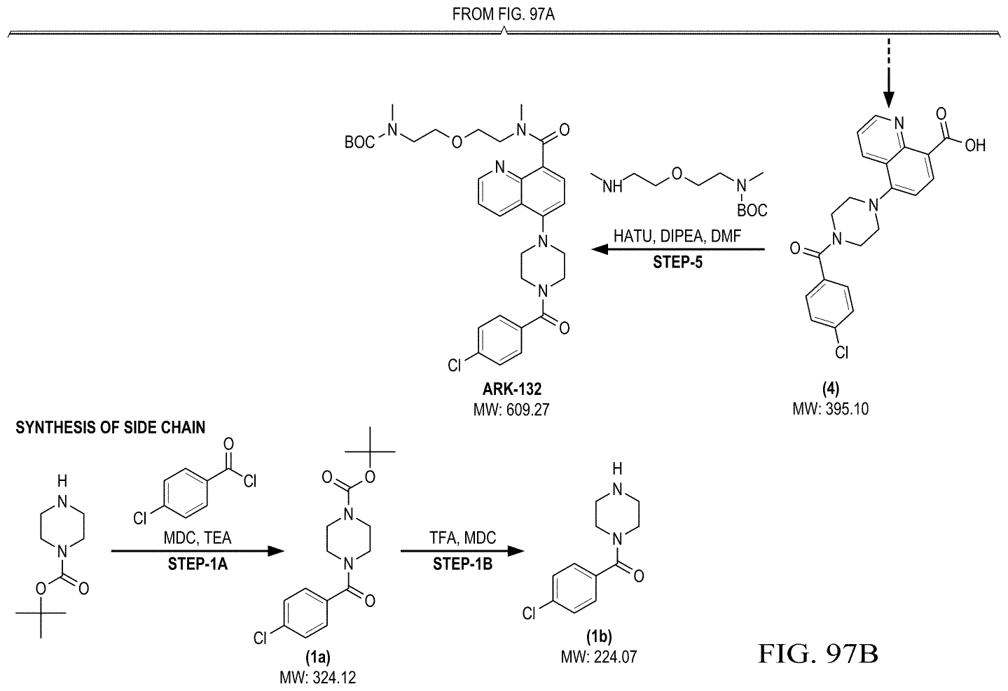

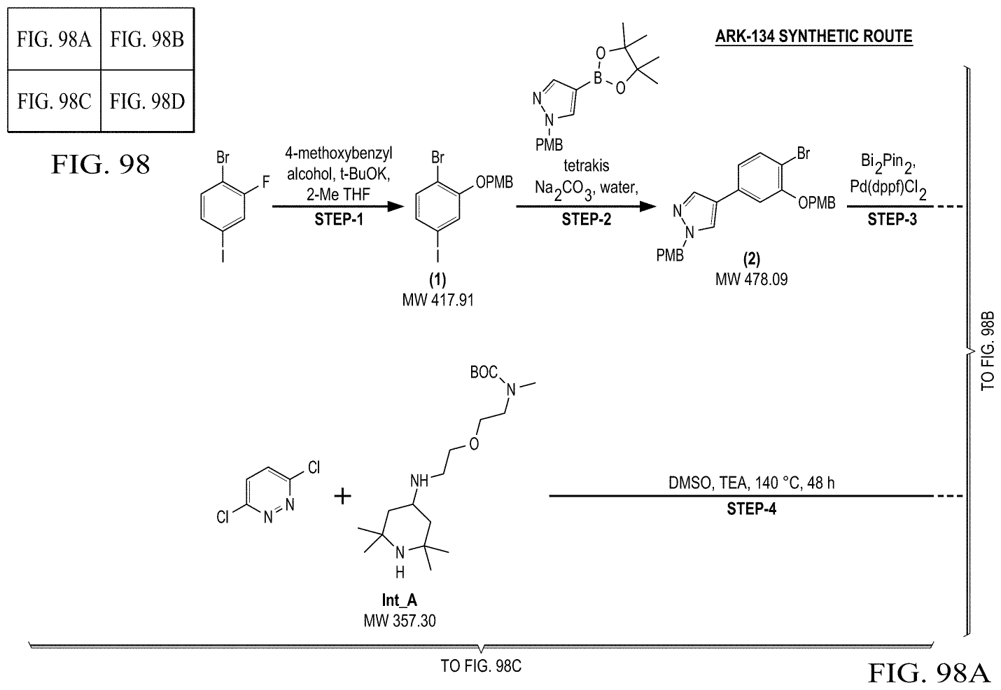

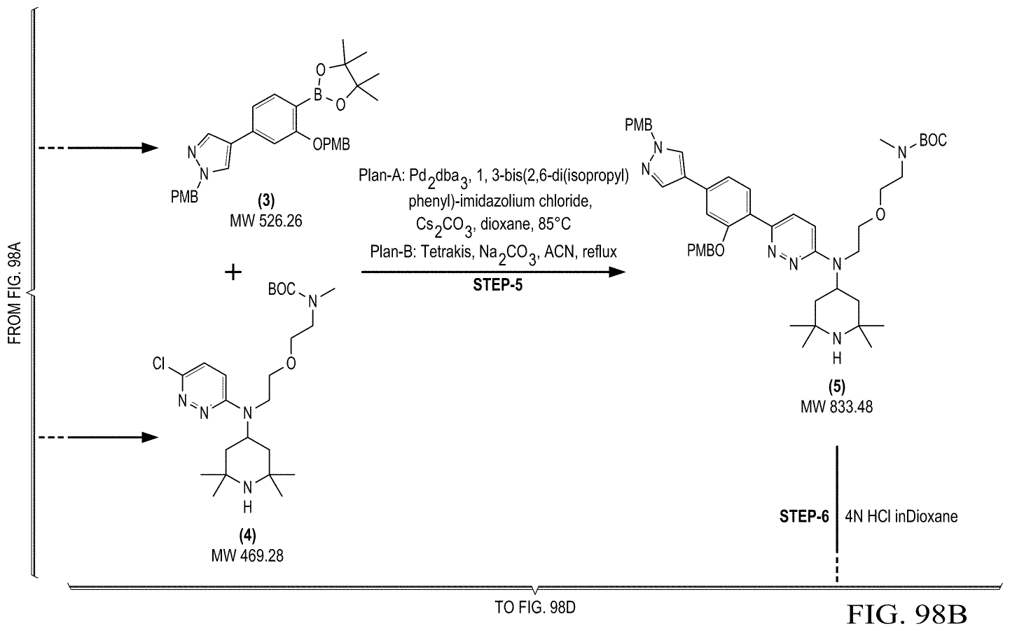

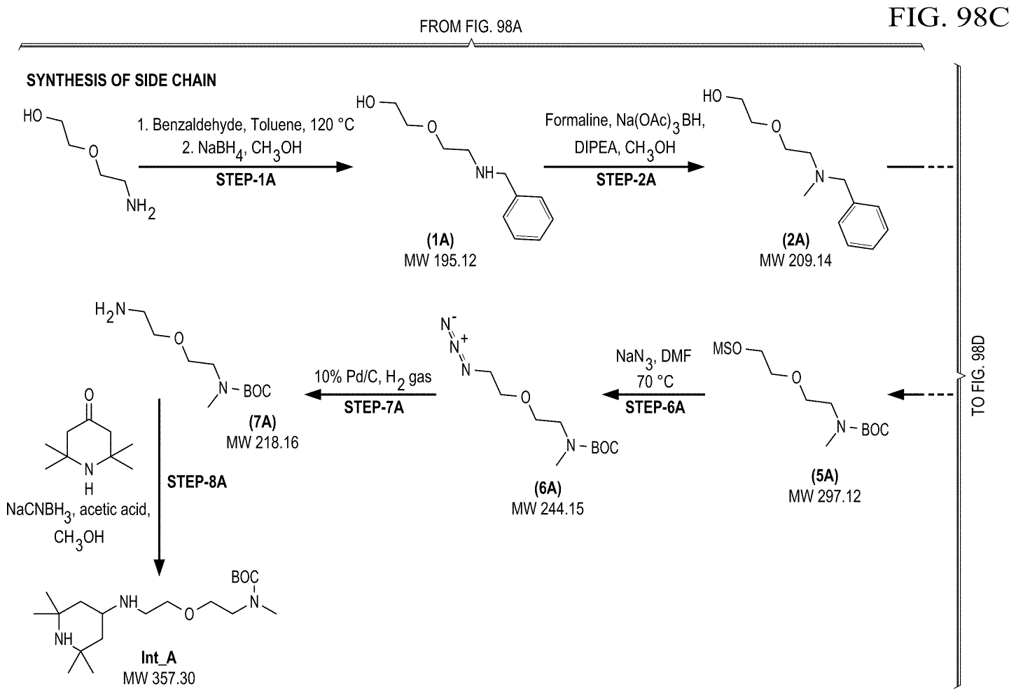

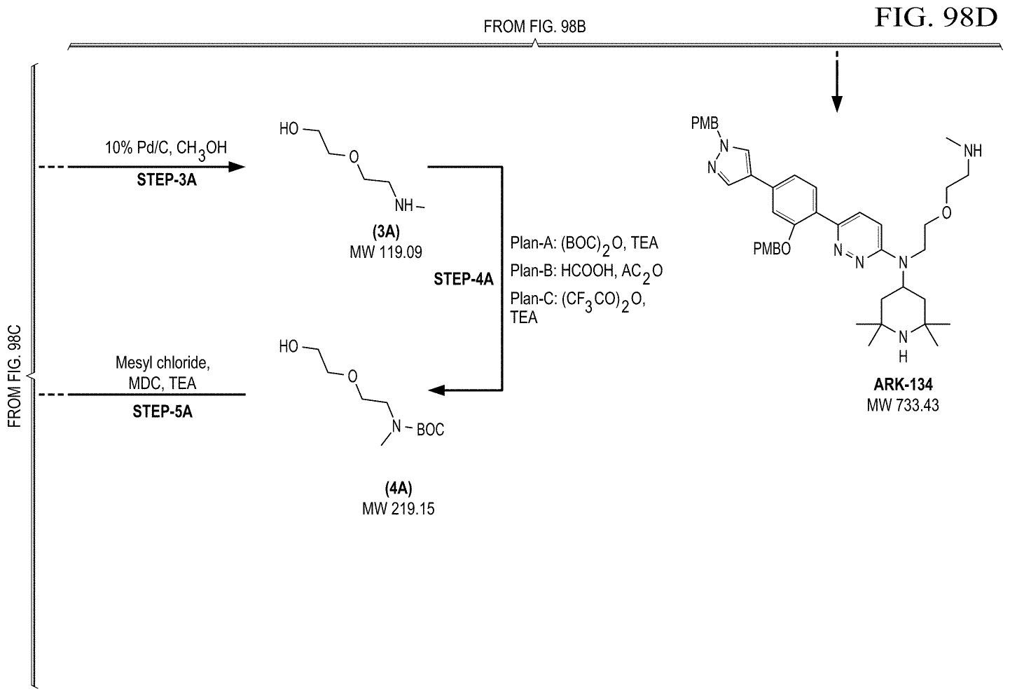

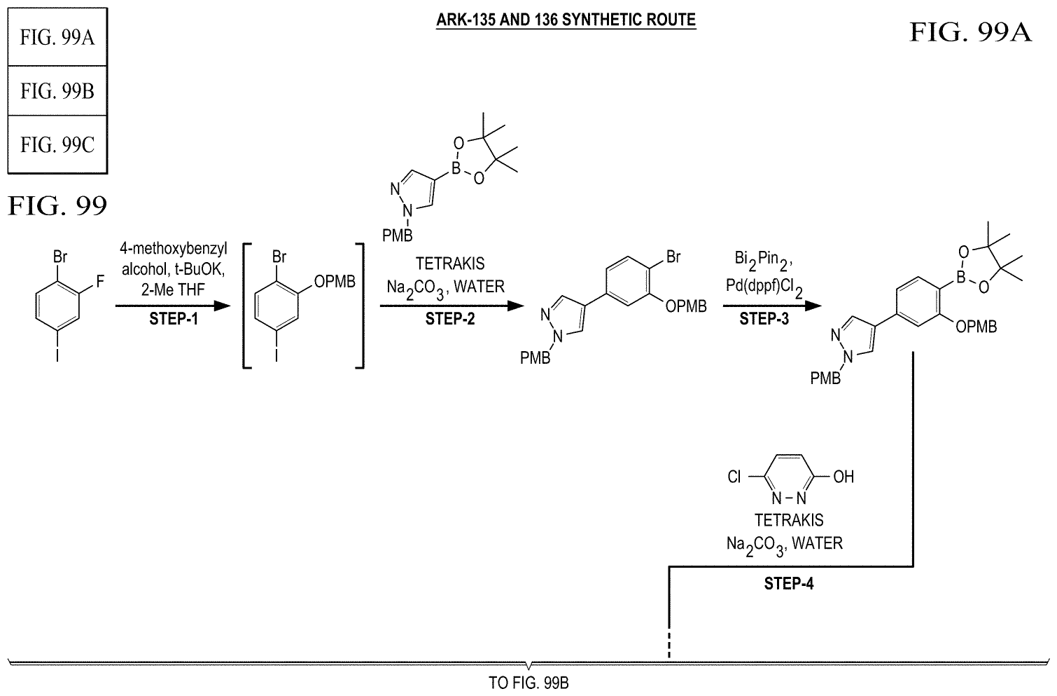

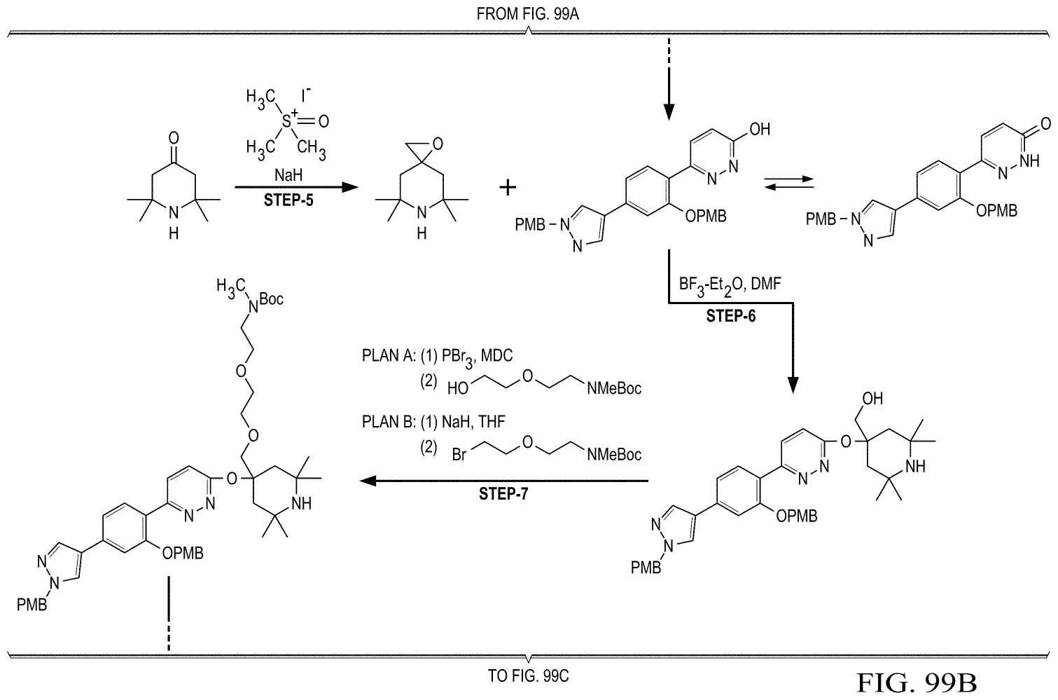

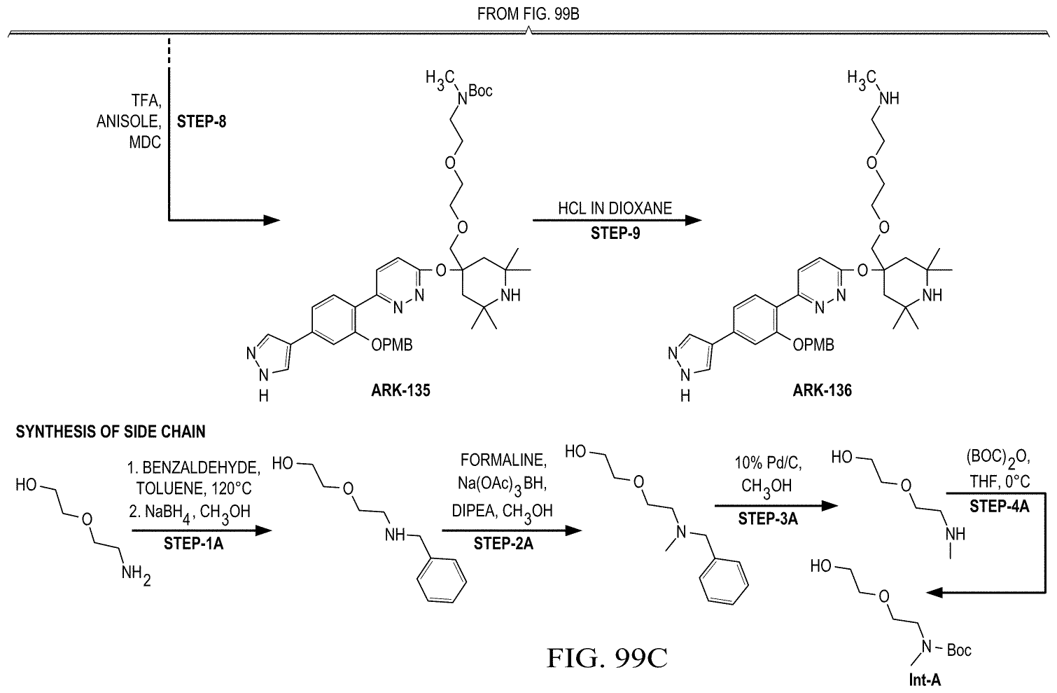

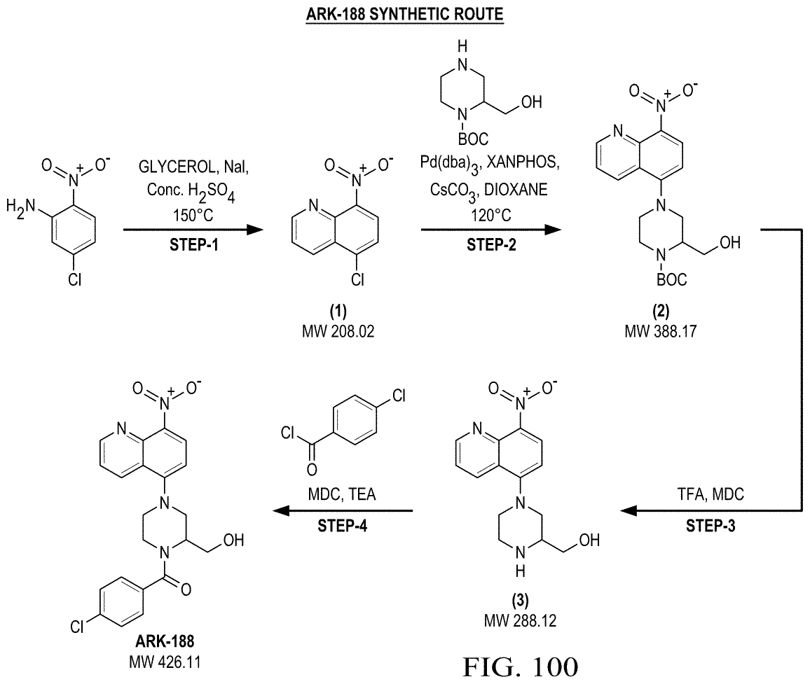

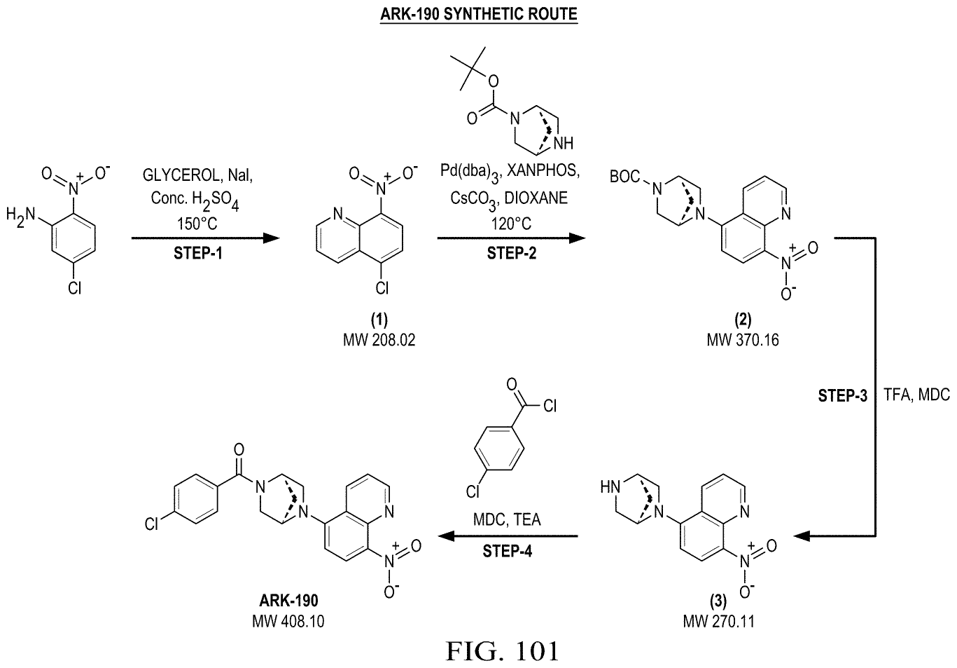

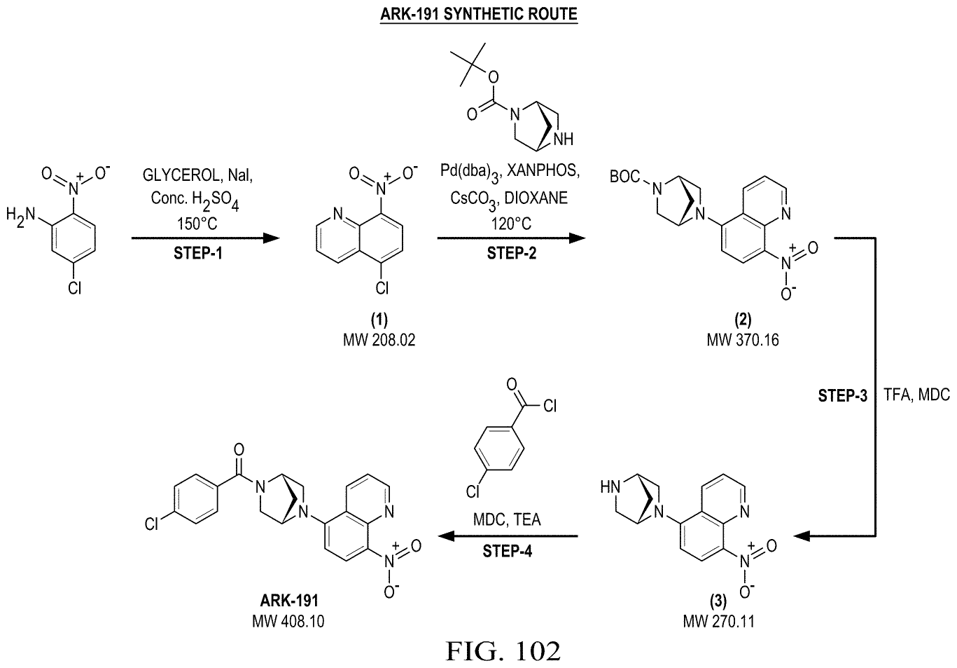

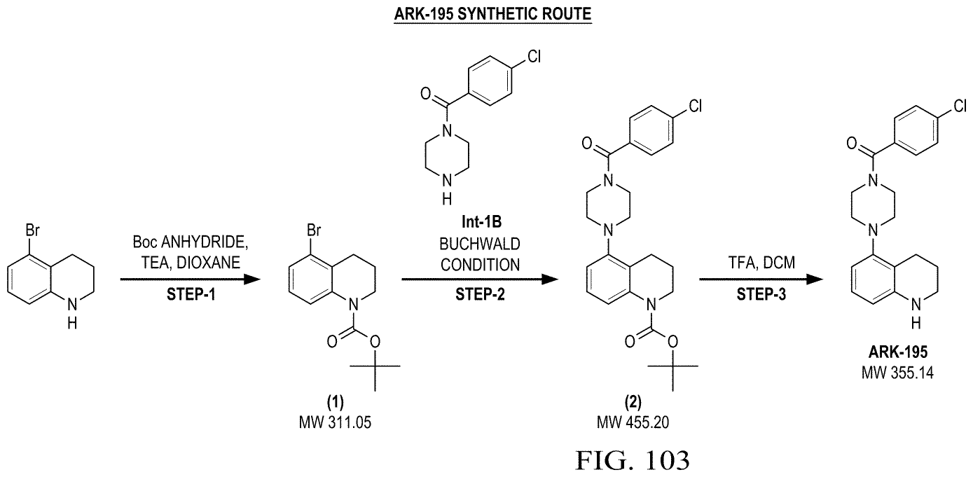

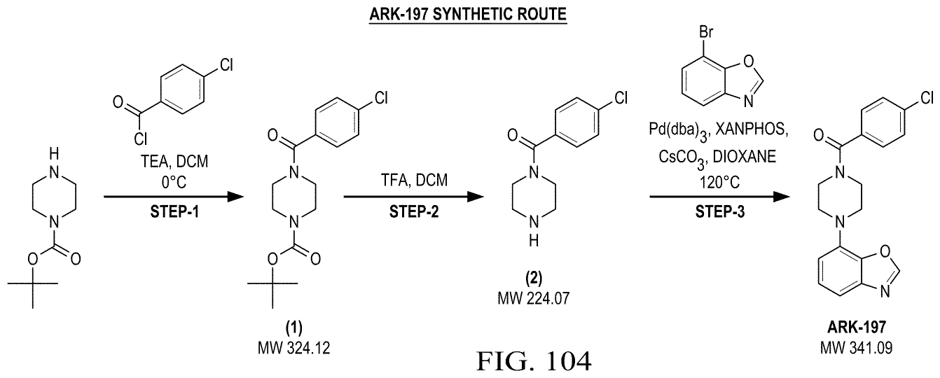

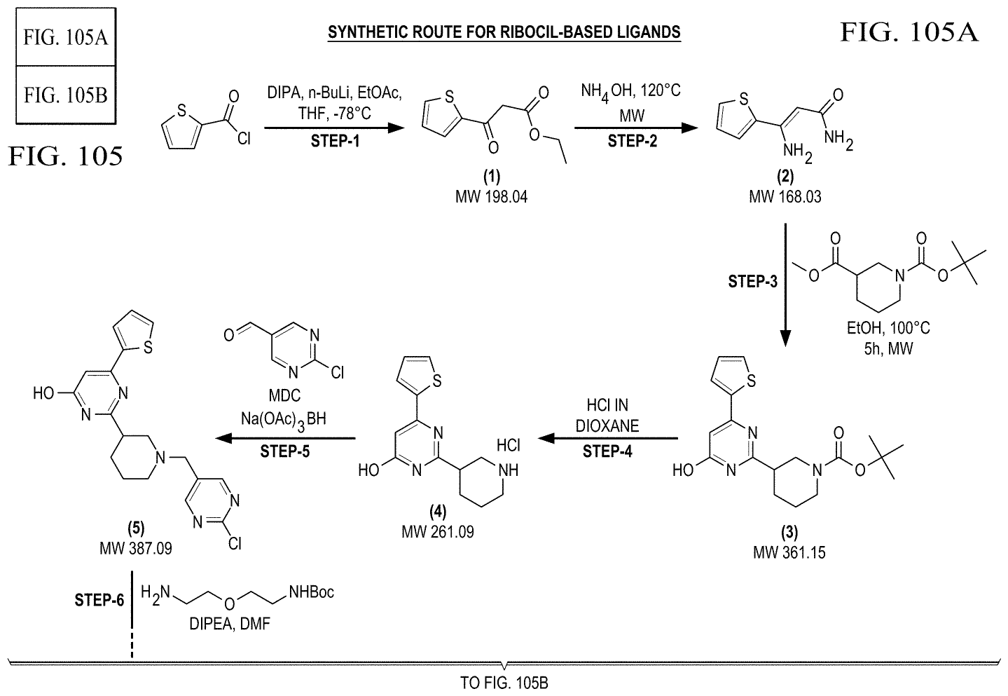

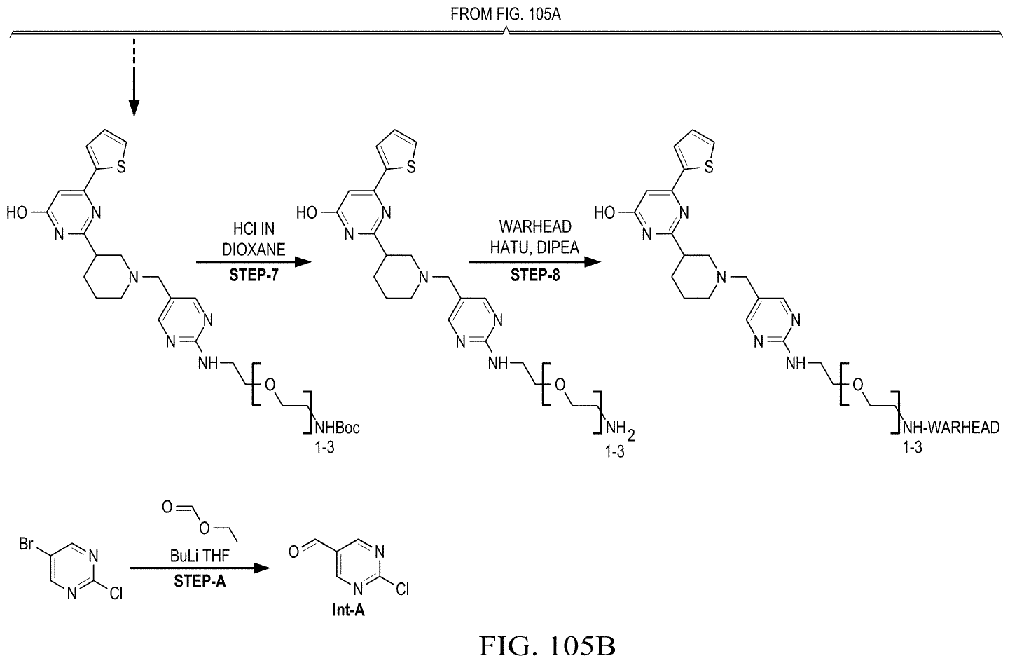

Accordingly, in some embodiments, the small molecule ligand is selected from CPNQ or a pharmaceutically acceptable salt thereof. In other embodiments, the ligand is selected from a quinoline compound related to CPNQ, such as those provided in any one of Tables 6 or 7, below, or in any one of FIGS. 97-105; or a pharmaceutically acceptable salt thereof.

In some embodiments, CPNQ or a quinoline related to CPNQ is modified at one or more available positions to replace a hydrogen with a tether (-T¹- and/or -T²-), click-ready group (—R^{CG}), or warhead (—R^{mod}), according to embodiments of each as described herein. For example, CPNQ or a quinoline related to CPNQ may have one of the following formulae:

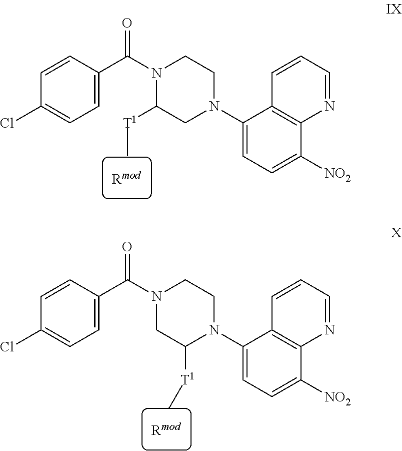

or a pharmaceutically acceptable salt thereof; wherein R^{mod} is optionally substituted with —R^{CG} or -T²-R^{CG}, and further optionally substituted with a pull-down group. The compound of formulae IX or X may further be optionally substituted with one or more optional substituents, as defined below, such as 1 or 2 optional substituents.

Organic dyes, amino acids, biological cofactors, metal complexes as well as peptides also show RNA binding ability. It is possible to modulate RNAs such as riboswitches, RNA molecules with expanded nucleotide repeats, and viral RNA elements.

The term “small molecule that binds a target RNA,” “small molecule RNA binder,” “affinity moiety,” or “ligand moiety,” as used herein, includes all compounds generally classified as small molecules that are capable of binding to a target RNA with sufficient affinity and specificity for use in a disclosed method, or to treat, prevent, or ameliorate a disease associated with the target RNA. Small molecules that bind RNA for use in the present invention may bind to one or more secondary or tertiary structure elements of a target RNA. These sites include RNA triplexes, hairpins, bulge loops, pseudoknots, internal loops, and other higher-order RNA structural motifs described or referred to herein.

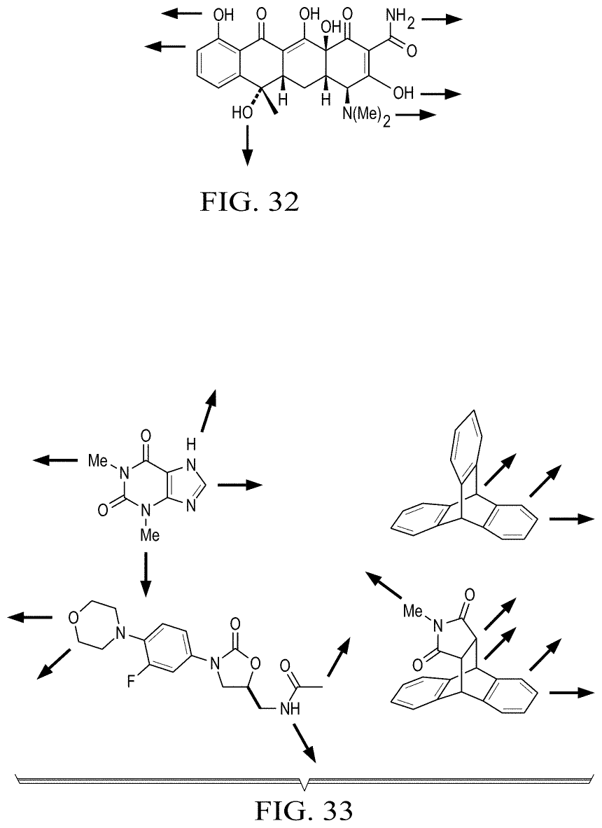

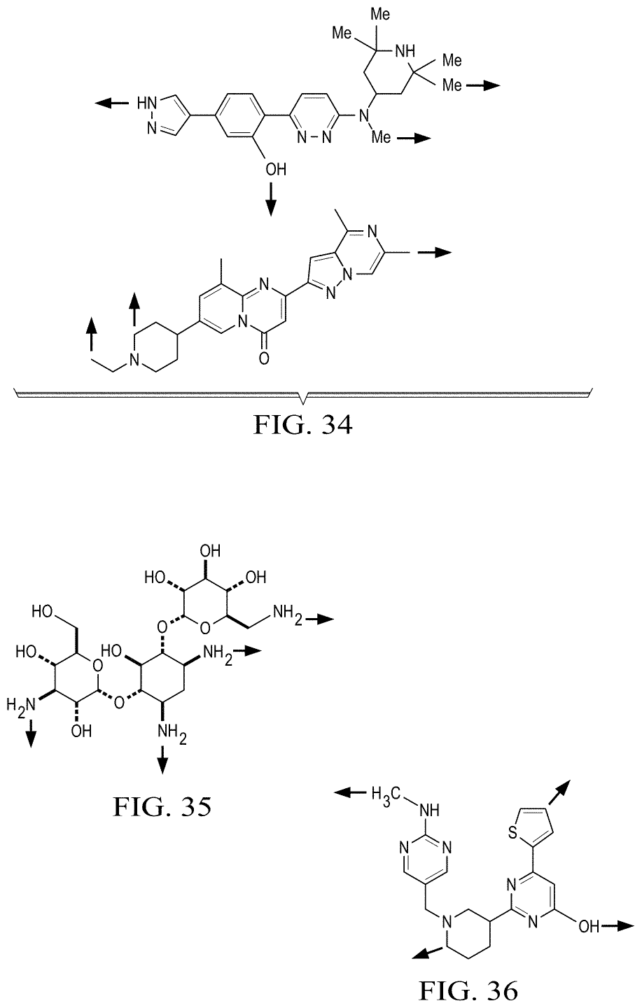

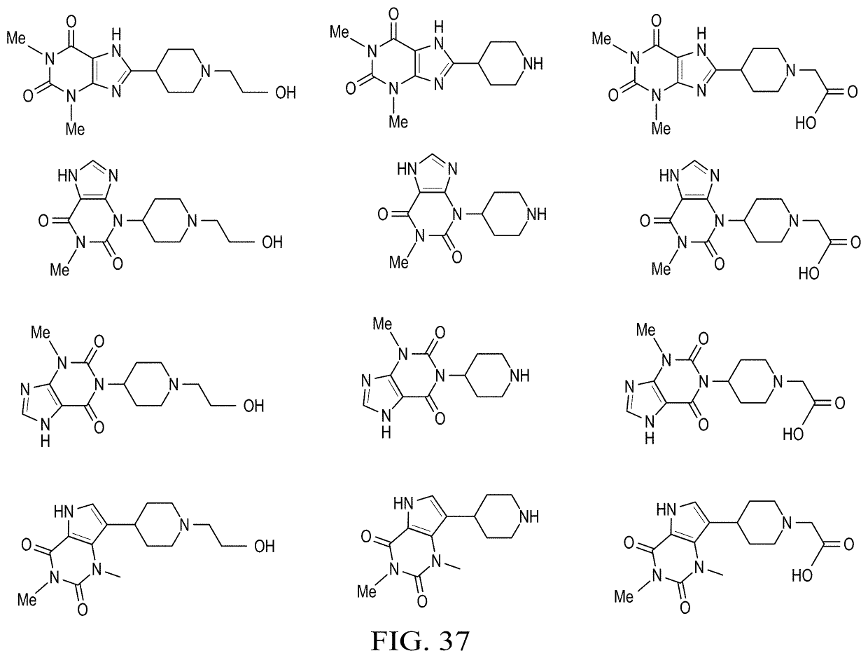

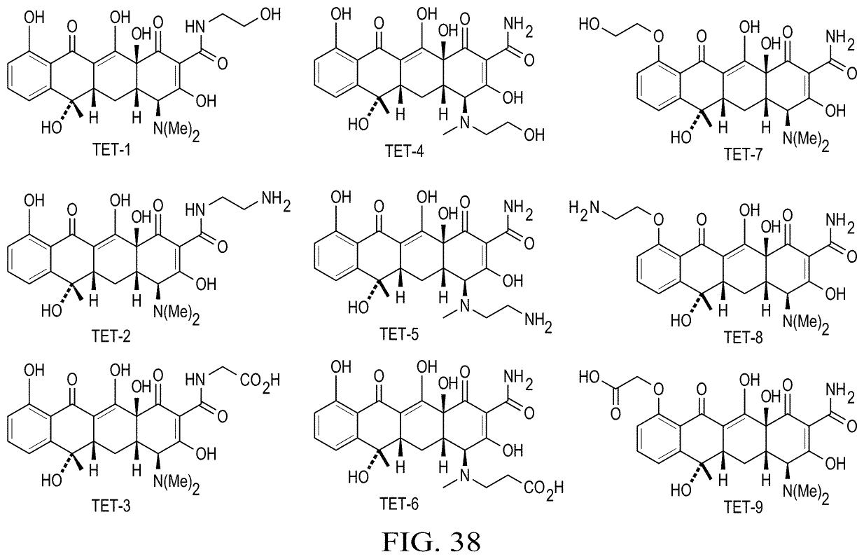

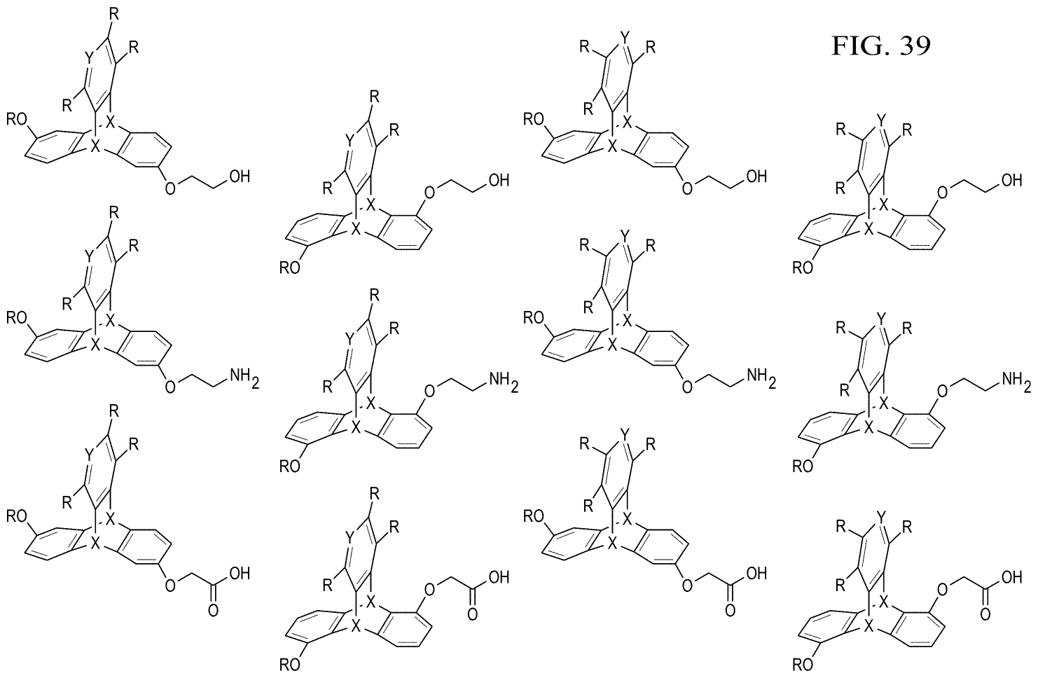

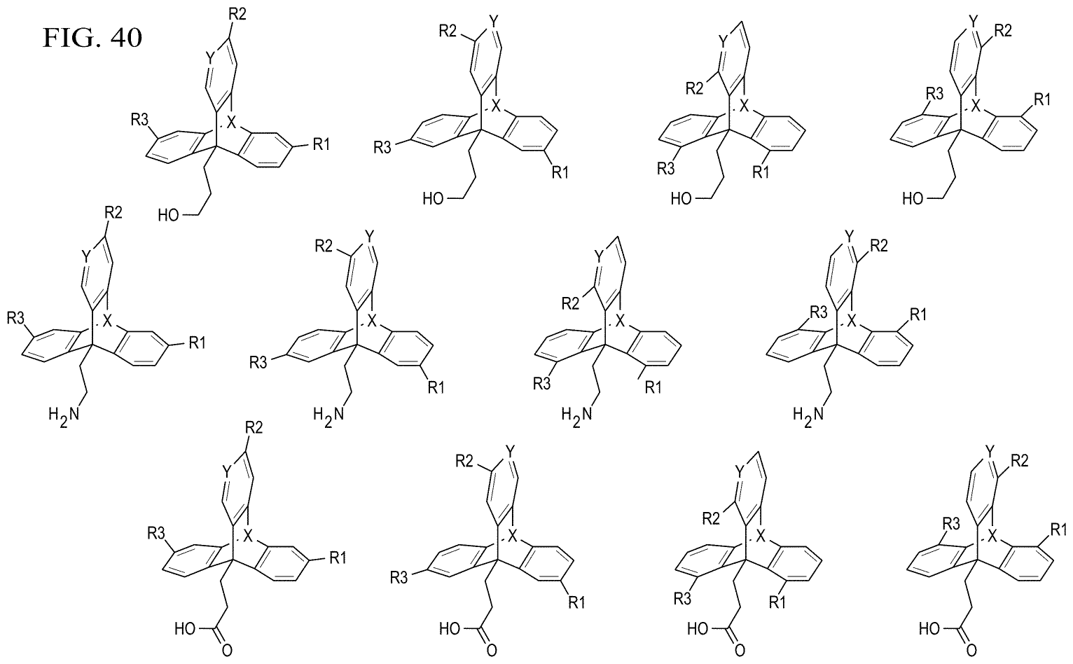

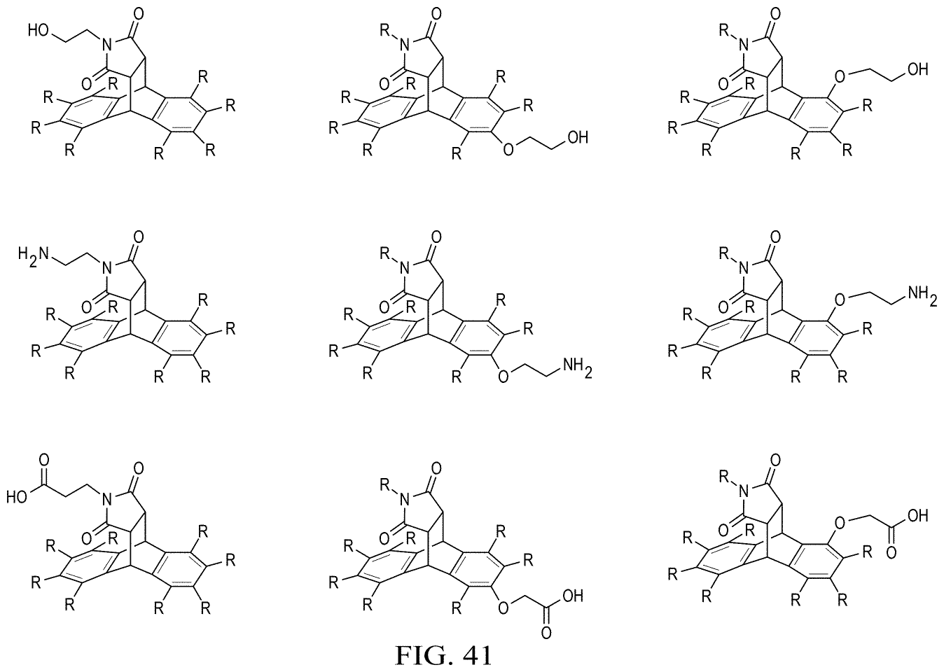

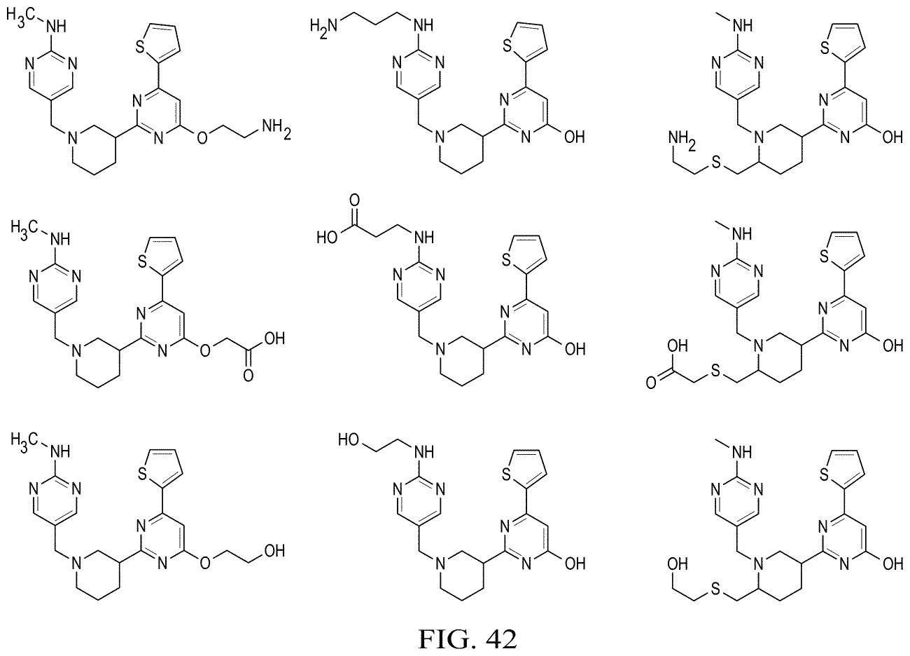

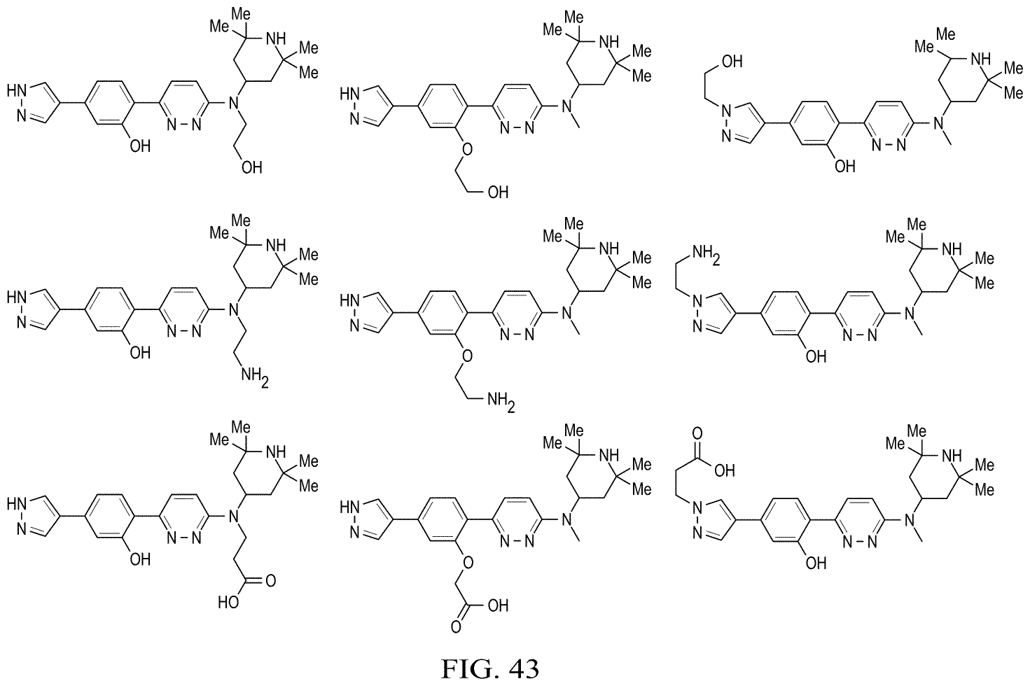

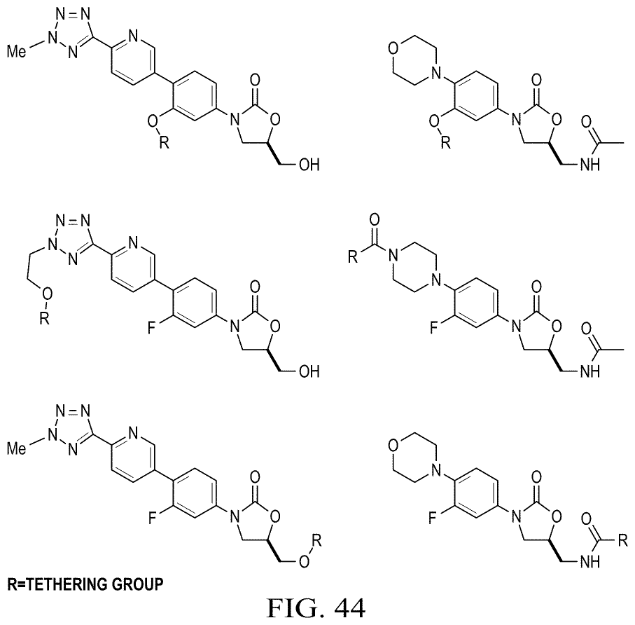

Accordingly, in some embodiments, the small molecule that binds to a target RNA (e.g., Ligand in Formulae I-VIII above) is selected from a macrolide, alkaloid, aminoglycoside, a member of the tetracycline family, an oxazolidinone, a SMN2 ligand (e.g., those shown in FIG. 34), ribocil or an analogue thereof, an anthracene, a triptycene, theophylline or an analogue thereof, or CPNQ or an analogue thereof. In some embodiments, the small molecule that binds to a target RNA is selected from paromomycin, a neomycin (such as neomycin B), a kanamycin (such as kanamycin A), linezolid, tedizolid, pleuromutilin, ribocil, NVS-SM1, anthracene, triptycene, or CPNQ or an analogue thereof; wherein each small molecule may be optionally substituted with one or more “optional substituents” as defined below, such as 1, 2, 3, or 4, for example 1 or 2, optional substituents. In some embodiments, the small molecule is selected from those shown in FIGS. 32-36, or a pharmaceutically acceptable salt, stereoisomer, or tautomer thereof. In some embodiments, the small molecule is selected from those shown in FIGS. 37-44, or a pharmaceutically acceptable salt, stereoisomer, or tautomer thereof. In some embodiments, the small molecule is selected from those shown in FIGS. 97-105, or a pharmaceutically acceptable salt, stereoisomer, or tautomer thereof. In some embodiments, the small molecule is selected from those shown in Table 6 or 7, or a pharmaceutically acceptable salt, stereoisomer, or tautomer thereof.

In some embodiments, the Ligand binds to a junction, stem-loop, or bulge in a target RNA. In some embodiments, Ligand binds to a nucleic acid three-way junction (3WJ). In some embodiments, the 3WJ is a trans 3WJ between two RNA molecules. In some embodiments, the 3WJ is a trans 3WJ between a miRNA and mRNA.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

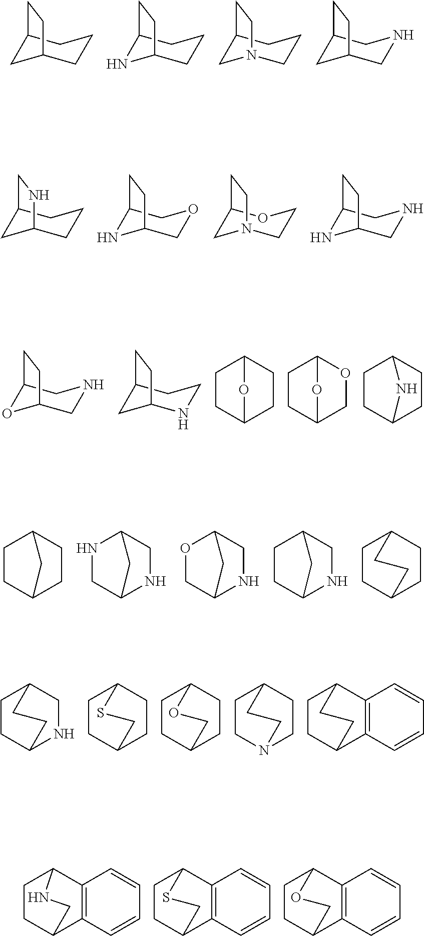

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

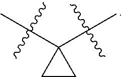 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent (“optional substituent”) at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; —C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SR—, SC(S)SRº; —(CH₂)₀₋₄SC(O)Rº; —(CH₂)₀₋₄C(O)NRº₂; —C(S)NRº₂; —C(S)SRº; —SC(S)SRº, —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —P(O)₂Rº; —P(O)Rº₂; —OP(O)Rº₂; —OP(O)(ORº)₂; SiRº₃; —(C₁₋₄ straight or branched alkylene)O—N(Rº)₂; or —(C₁₋₄ straight or branched alkylene)C(O)O—N(Rº)₂, wherein each Rº may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on Rº (or the ring formed by taking two independent occurrences of Rº together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of Rº include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms.

As used herein, the term “inhibitor” is defined as a compound that binds to and/or modulates or inhibits a target RNA with measurable affinity. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

The terms “measurable affinity” and “measurably inhibit,” as used herein, mean a measurable change in a downstream biological effect between a sample comprising a compound of the present invention, or composition thereof, and a target RNA, and an equivalent sample comprising the target RNA, in the absence of said compound, or composition thereof.

The term “RNA” (ribonucleic acid) as used herein, means naturally-occurring or synthetic oligoribonucleotides independent of source (e.g., the RNA may be produced by a human, animal, plant, virus, or bacterium, or may be synthetic in origin), biological context (e.g., the RNA may be in the nucleus, circulating in the blood, in vitro, cell lysate, or isolated or pure form), or physical form (e.g., the RNA may be in single-, double-, or triple-stranded form (including RNA-DNA hybrids), may include epigenetic modifications, native post-transcriptional modifications, artificial modifications (e.g., obtained by chemical or in vitro modification), or other modifications, may be bound to, e.g., metal ions, small molecules, protein chaperones, or cofactors, or may be in a denatured, partially denatured, or folded state including any native or unnatural secondary or tertiary structure such as junctions (e.g., cis or trans three-way junctions (3WJ)), quadruplexes, hairpins, triplexes, hairpins, bulge loops, pseudoknots, and internal loops, etc., and any transient forms or structures adopted by the RNA). In some embodiments, the RNA is 100 or more nucleotides in length. In some embodiments, the RNA is 250 or more nucleotides in length. In some embodiments, the RNA is 350, 450, 500, 600, 750, or 1,000, 2,000, 3,000, 4,000, 5,000, 7,500, 10,000, 15,000, 25,000, 50,000, or more nucleotides in length. In some embodiments, the RNA is between 250 and 1,000 nucleotides in length. In some embodiments, the RNA is a pre-RNA, pre-miRNA, or pretranscript. In some embodiments, the RNA is a non-coding RNA (ncRNA), messenger RNA (mRNA), micro-RNA (miRNA), a ribozyme, riboswitch, lncRNA, lincRNA, snoRNA, snRNA, scaRNA, piRNA, ceRNA, pseudo-gene, viral RNA, or bacterial RNA. The term “target RNA,” as used herein, means any type of RNA having a secondary or tertiary structure capable of binding a small molecule ligand described herein. The target RNA may be inside a cell, in a cell lysate, or in isolated form prior to contacting the compound.

Covalent Modifier Moieties

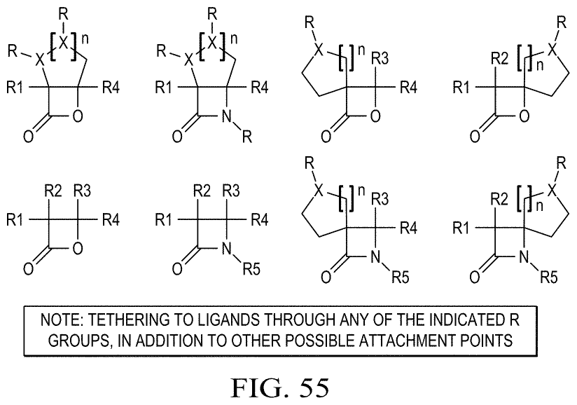

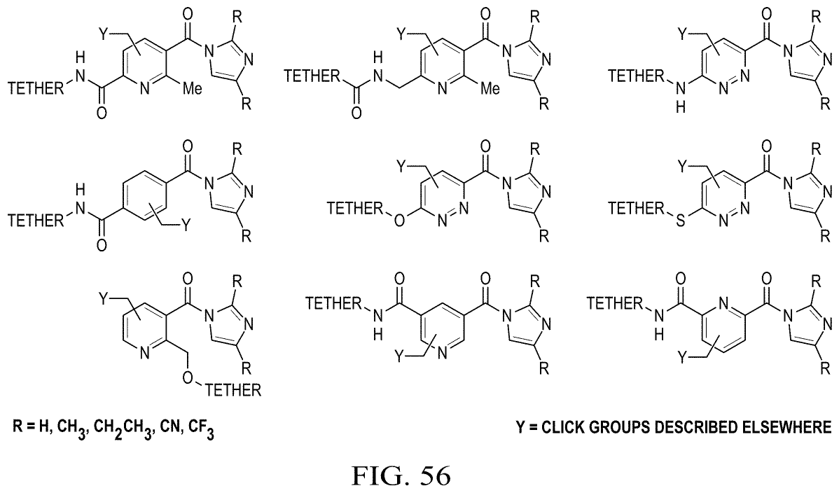

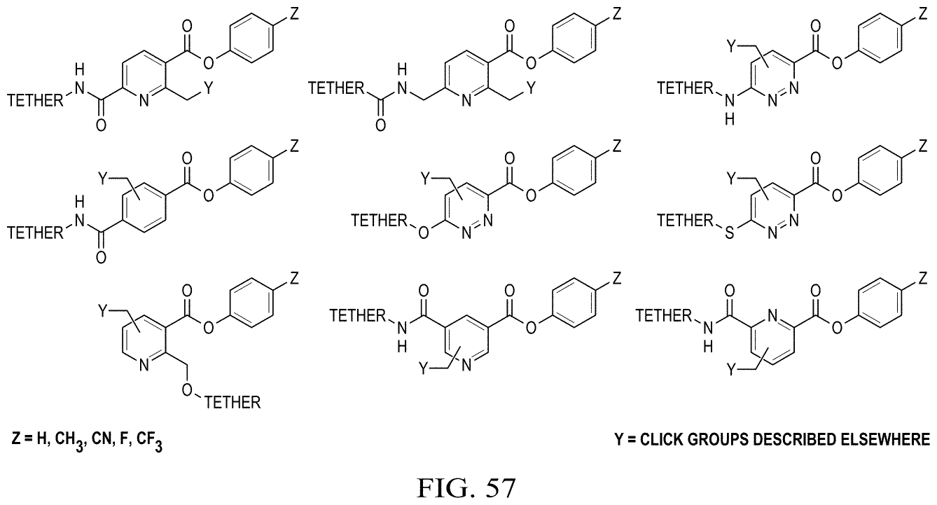

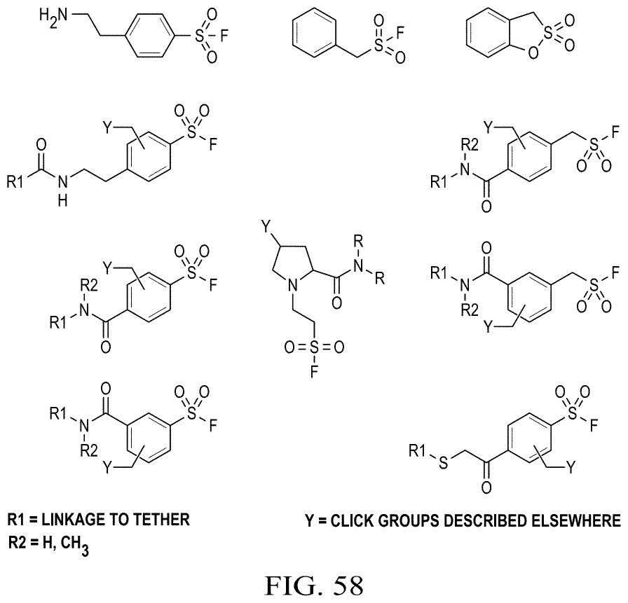

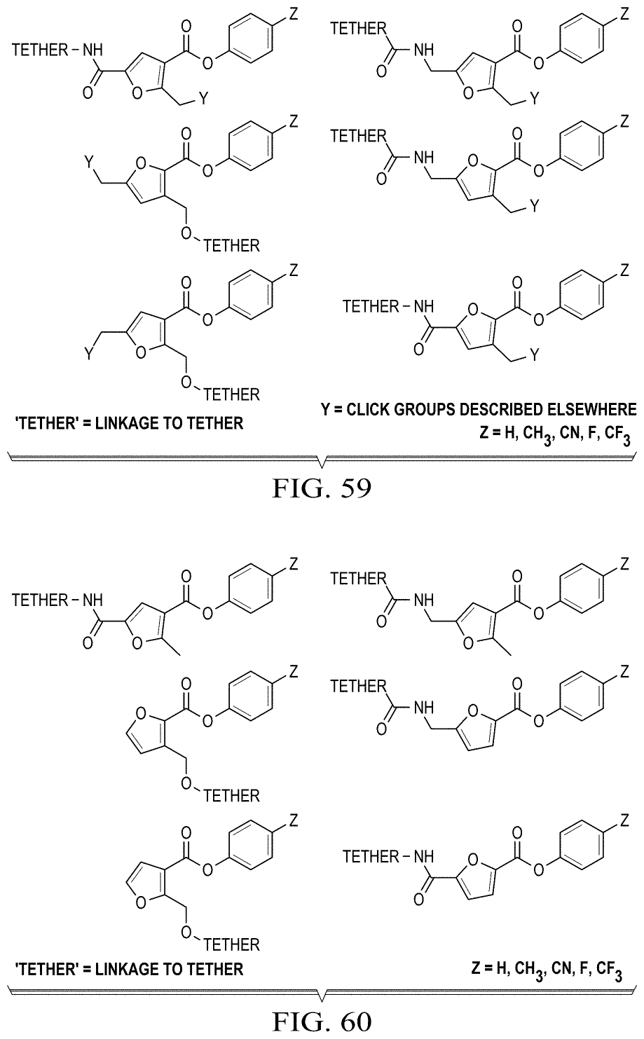

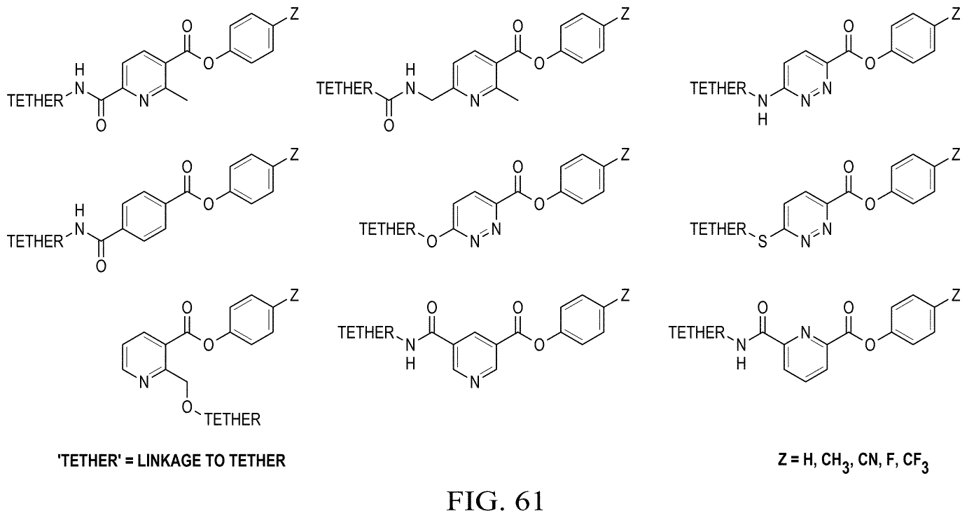

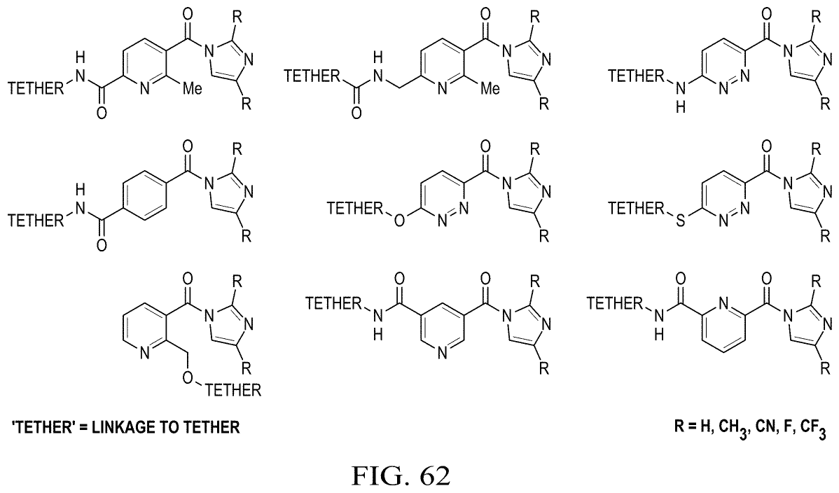

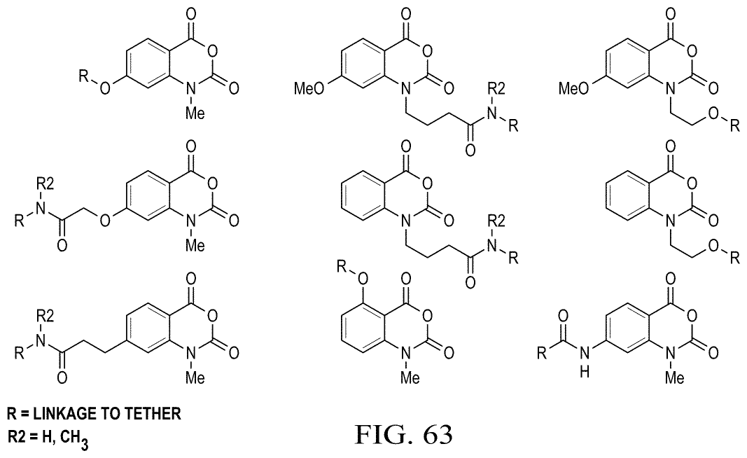

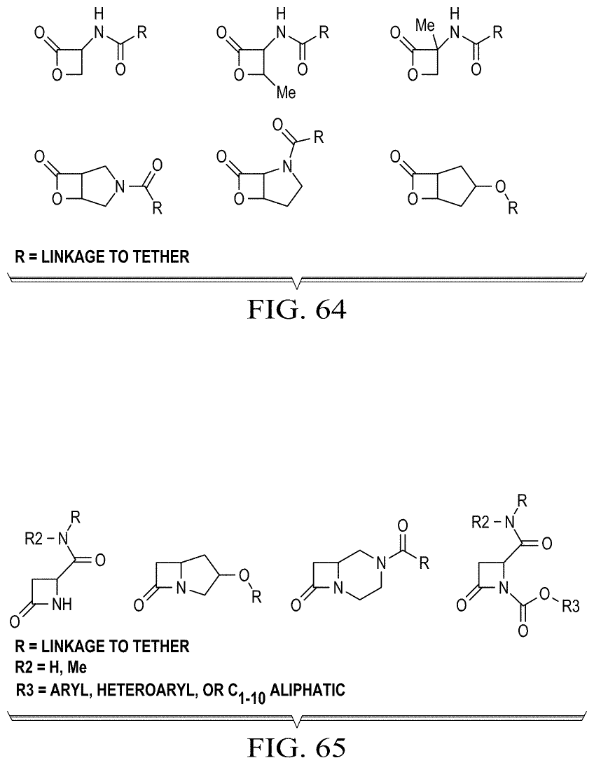

A variety of covalent modifier moieties (i.e. R^{mod} shown in, e.g., Formulae I-X above) may be used in the present invention. In some embodiments, the covalent modifier is aryl-C(O)—X, heteroaryl-C(O)—X, aryl-SO₂—X, or heteroaryl-SO₂—X, wherein X is an appropriate leaving group such as a halide or N-heteroaryl, e.g. imidazolyl. In some embodiments, the covalent modifier moiety is one of those shown in FIGS. 54-65.

The term “covalent modifier moiety” or “warhead” as used herein, means any small molecule group that includes a reactive functionality capable of selectively forming a covalent bond with an unconstrained nucleotide of a RNA to produce a 2′-modified RNA. In some embodiments, the covalent modifier moiety is an aromatic or heteroaromatic group bound to a reactive functionality. In some embodiments, the reactive functionality is selected from sulfonyl halides, arenecarbonyl imidazoles, active esters, epoxides, oxiranes, oxidizing agents, aldehydes, alkyl halides, benzyl halides, isocyanates, or other groups such as those described by Hermanson, Bioconjugate Techniques, Second Edition, Academic Press, 2008. In some embodiments, the reactive functionality is an active ester. The active ester may react with an unconstrained 2′-hydroxyl group (or one that is otherwise more reactive than neighboring 2′-hydroxyl groups) of an RNA to produce a 2′-covalently modified RNA. In some embodiments, the active ester is an acyl imidazole. In some embodiments, the reactive functionality is selected from an aryl ester, a heteroaryl ester, a sulfonyl halide, a lactone, a lactam, an α,β-unsaturated ketone, an aldehyde, an alkyl halide, or a benzyl halide. In some embodiments, the reactive functionality is selected from an aryl ester, a heteroaryl ester, a sulfonyl fluoride, or a lactam.

In some embodiments, the covalent modifier moiety is 1-methyl-7-nitroisatoic anhydride (1M7), benzoyl cyanide (BzCN), 2-methylnicotinic acid imidazolide (NAI), or 2-methyl-3-furoic acid imidazolide (FAI).

Further examples of covalent modifier moieties suitable for use in the present invention are described in WO 2015/054247, US 2014/0154673, and U.S. Pat. No. 8,313,424, each of which is hereby incorporated by reference.

Tethering Group

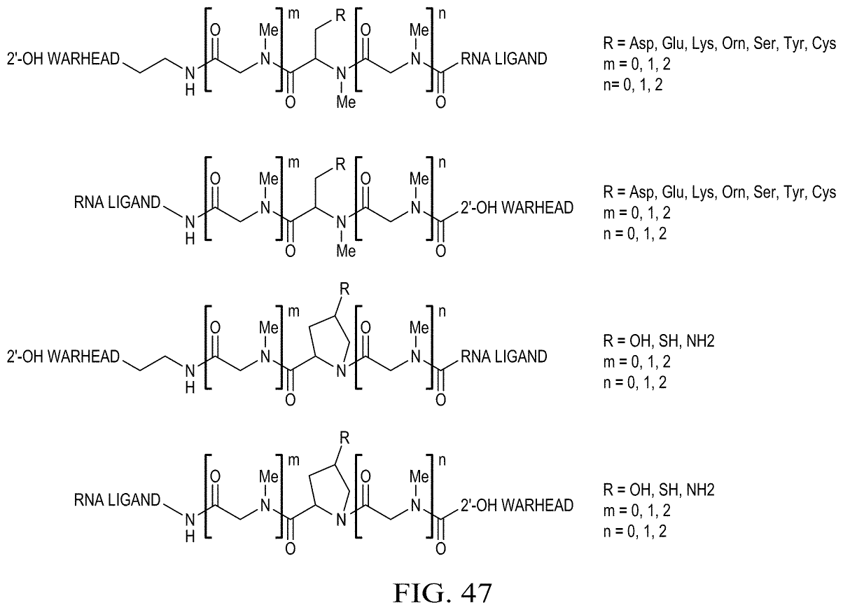

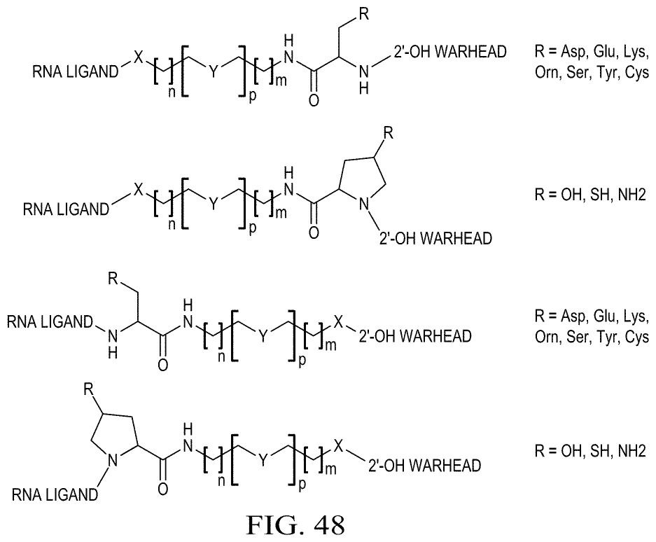

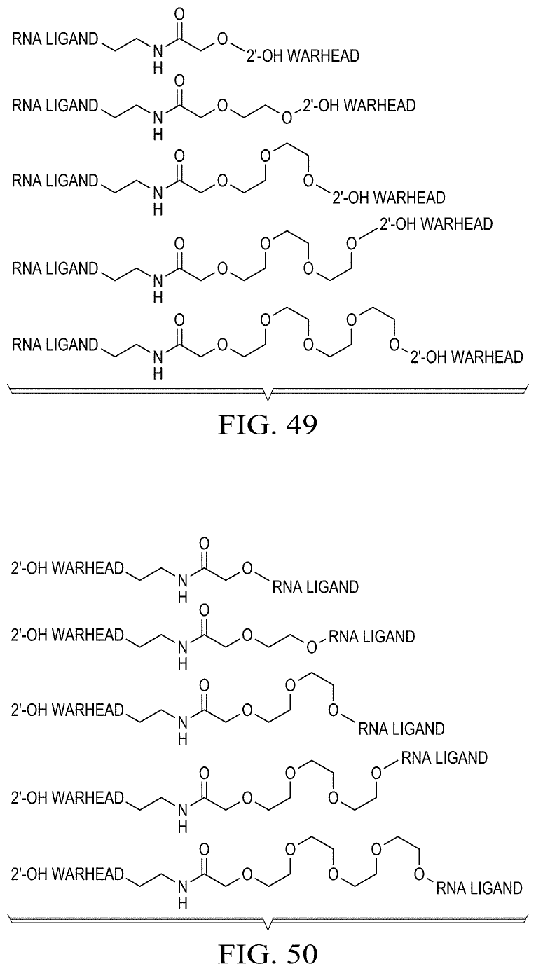

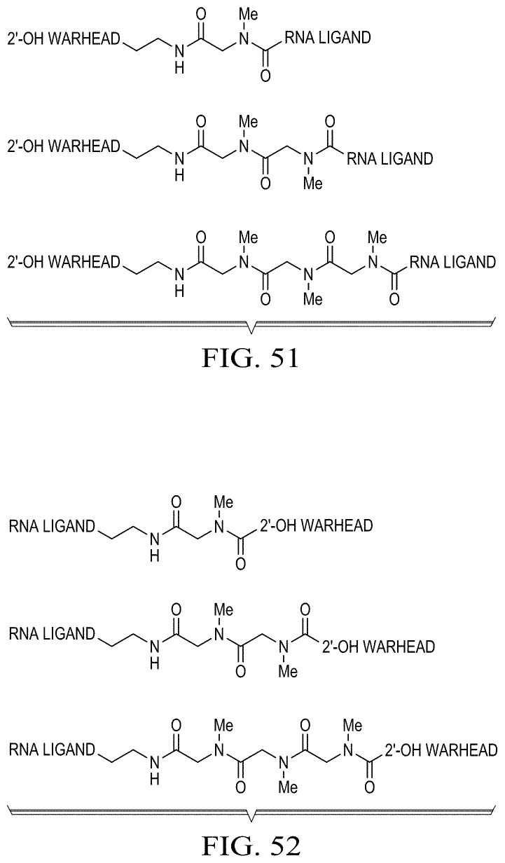

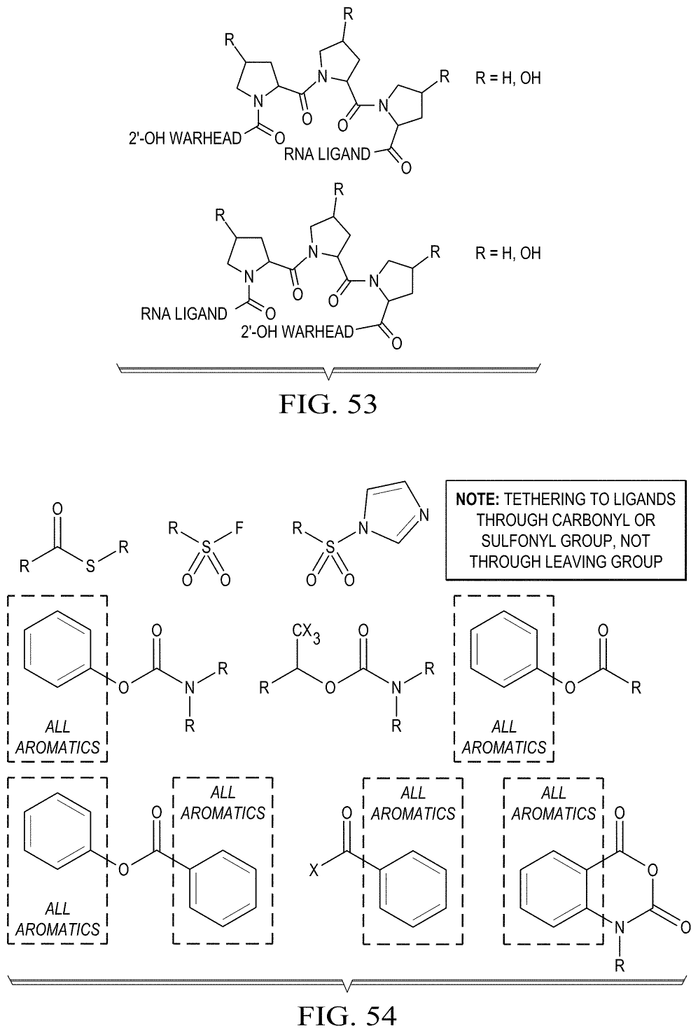

The present invention contemplates the use of a wide variety of bivalent or trivalent tethering groups (tethers; e.g., variables T¹ and T² as shown in, e.g., Formulae I-X above) to provide optimal binding and reactivity toward 2′-OH groups proximal to the binding site of a target RNA. In some embodiments, T¹ and T² are selected from those shown in FIGS. 46-53. For example, in some embodiments, T¹ and/or T² is a polyethylene glycol (PEG) group of, e.g., 1-10 ethylene glycol subunits. In some embodiments, T¹ and/or T² is an optionally substituted C₁₋₁₂ aliphatic group or a peptide comprising 1-8 amino acids.

In some embodiments, the physical properties such as the length, rigidity, hydrophobicity, and/or other properties of the tether are selected to optimize the pattern of proximity-induced covalent bond formation between the 2′-OH of a target RNA and the modifying moiety (warhead). In some embodiments, the physical properties of the tether (such as those above) are selected so that, upon binding of the compound to the active or allosteric sites of a target RNA, the modifying moiety selectively reacts with one or more 2′-OH groups of the target RNA proximal to the active site or allosteric sites.

Click-Ready Groups

A variety of bioorthogonal reaction partners (e.g., R^{CG} in Formulae I-X above) may be used in the present invention to couple a compound described herein with a pull-down moiety. The term “bioorthogonal chemistry” or “bioorthogonal reaction,” as used herein, refers to any chemical reaction that can take place in living systems without interfering with native biochemical processes. Accordingly, a “bioorthogonal reaction partner” is a chemical moiety capable of undergoing a bioorthogonal reaction with an appropriate reaction partner to couple a compound described herein to a pull-down moiety. In some embodiments, a bioorthogonal reaction partner is covalently attached to the chemical modifying moiety or the tethering group. In some embodiments, the bioorthogonal reaction partner is selected from a click-ready group or a group capable of undergoing a nitrone/cyclooctyne reaction, oxime/hydrazone formation, a tetrazine ligation, an isocyanide-based click reaction, or a quadricyclane ligation.

In some embodiments, the bioorthogonal reaction partner is a click-ready group. The term “click-ready group” refers to a chemical moiety capable of undergoing a click reaction, such as an azide or alkyne.

Click reactions tend to involve high-energy (“spring-loaded”) reagents with well-defined reaction coordinates, that give rise to selective bond-forming events of wide scope. Examples include nucleophilic trapping of strained-ring electrophiles (epoxide, aziridines, aziridinium ions, episulfonium ions), certain carbonyl reactivity (e.g., the reaction between aldehydes and hydrazines or hydroxylamines), and several cycloaddition reactions. The azide-alkyne 1,3-dipolar cycloaddition and the Diels-Alder cycloaddition are two such reactions.

Such click reactions (i.e., dipolar cycloadditions) are associated with a high activation energy and therefore require heat or a catalyst. Indeed, use of a copper catalyst is routinely employed in click reactions. However, in certain instances where click chemistry is particularly useful (e.g., in bioconjugation reactions), the presence of copper can be detrimental (See Wolbers, F. et al.; Electrophoresis 2006, 27, 5073). Accordingly, methods of performing dipolar cycloaddition reactions were developed without the use of metal catalysis. Such “metal free” click reactions utilize activated moieties in order to facilitate cycloaddition. Therefore, the present invention provides click-ready groups suitable for metal-free click chemistry.

Certain metal-free click moieties are known in the literature. Examples include 4-dibenzocyclooctynol (DIBO) (from Ning et al; Angew Chem Int Ed, 2008, 47, 2253); gem-difluorinated cyclooctynes (DIFO or DFO) (from Codelli, et al.; J. Am. Chem. Soc. 2008, 130, 11486-11493.); biarylazacyclooctynone (BARAC) (from Jewett et al.; J. Am. Chem. Soc. 2010, 132, 3688.); or bicyclononyne (BCN) (From Dommerholt, et al.; Angew Chem Int Ed, 2010, 49, 9422-9425).

As used herein, the phrase “a moiety suitable for metal-free click chemistry” refers to a functional group capable of dipolar cycloaddition without use of a metal catalyst. Such moieties include an activated alkyne (such as a strained cyclooctyne), an oxime (such as a nitrile oxide precursor), or oxanorbornadiene, for coupling to an azide to form a cycloaddition product (e.g., triazole or isoxazole).

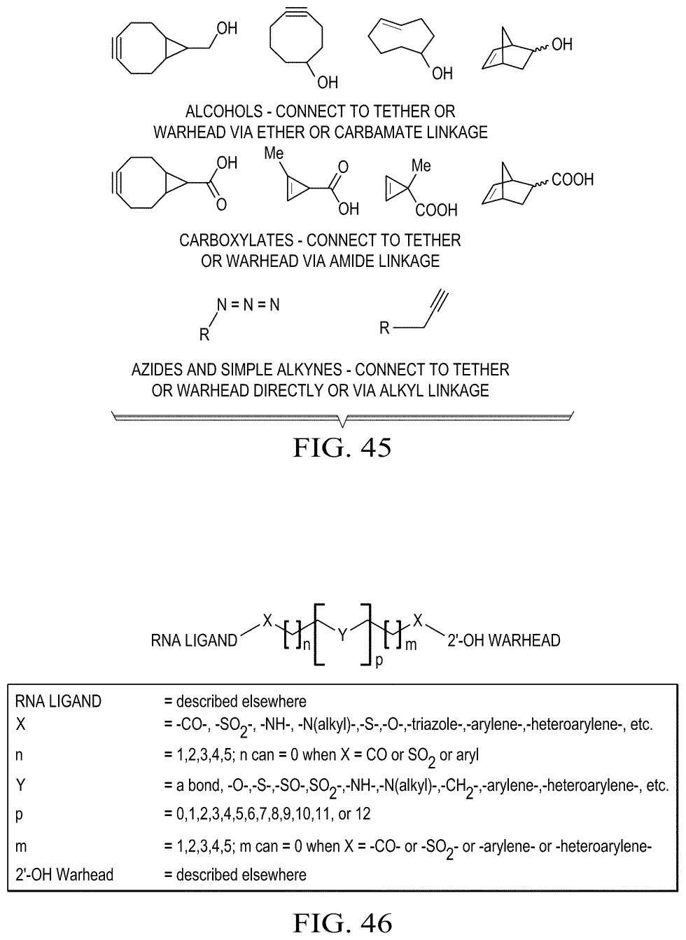

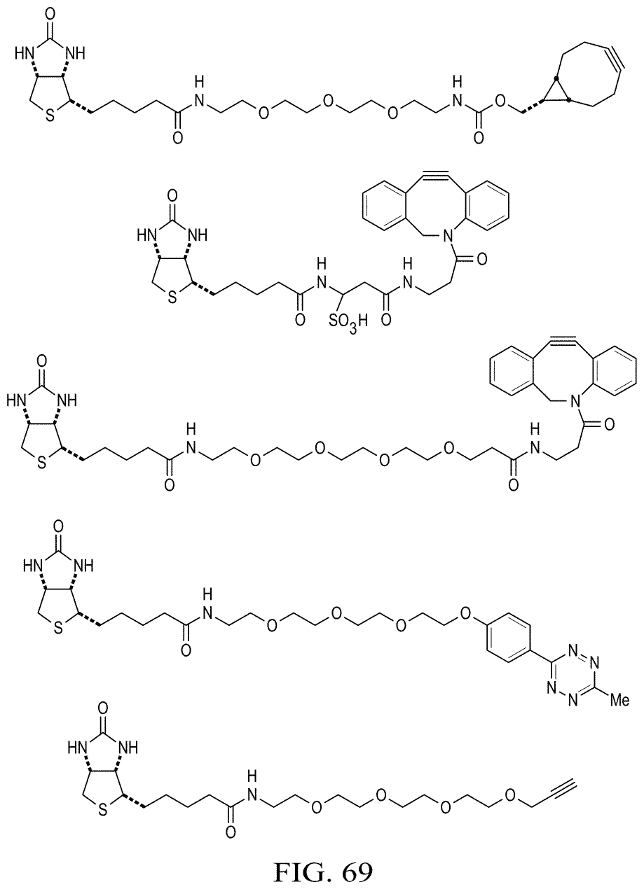

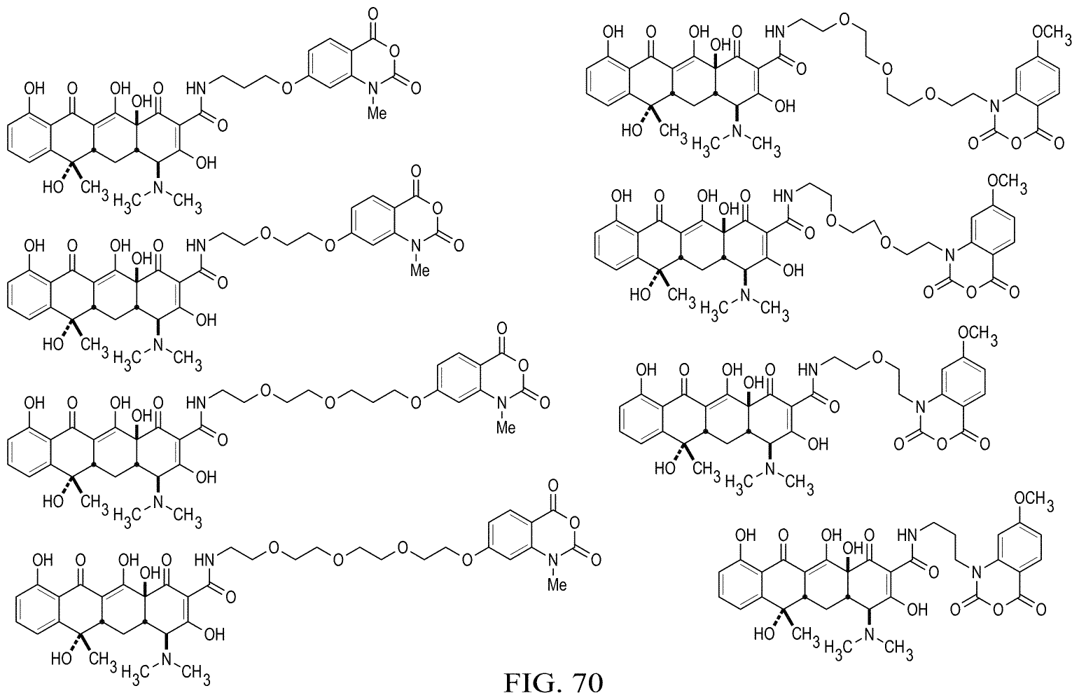

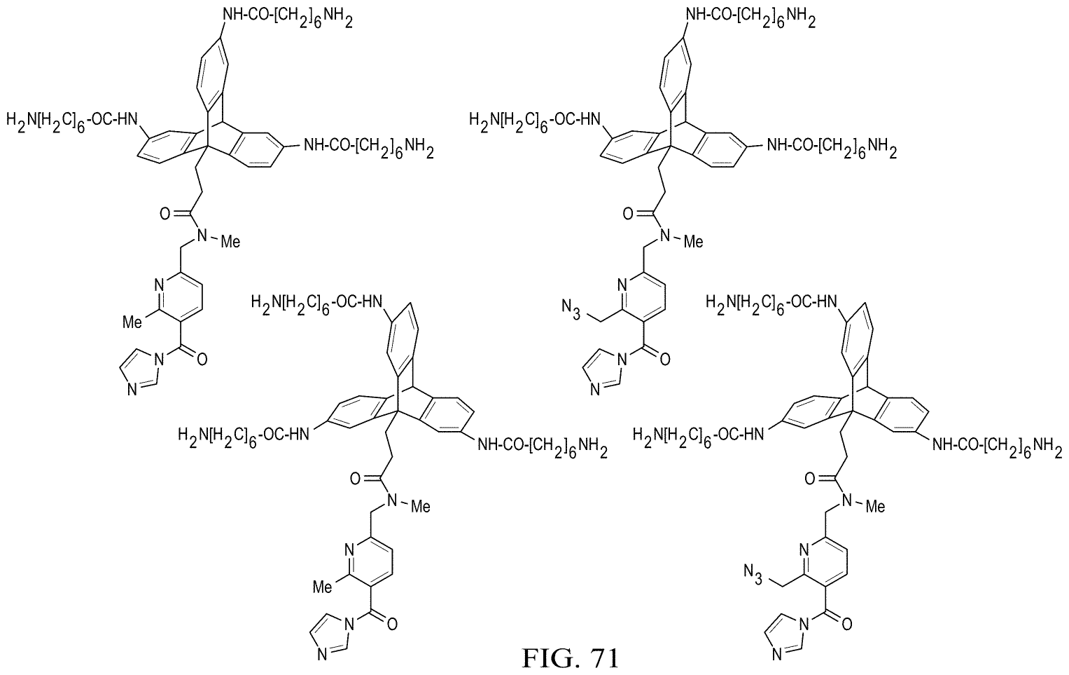

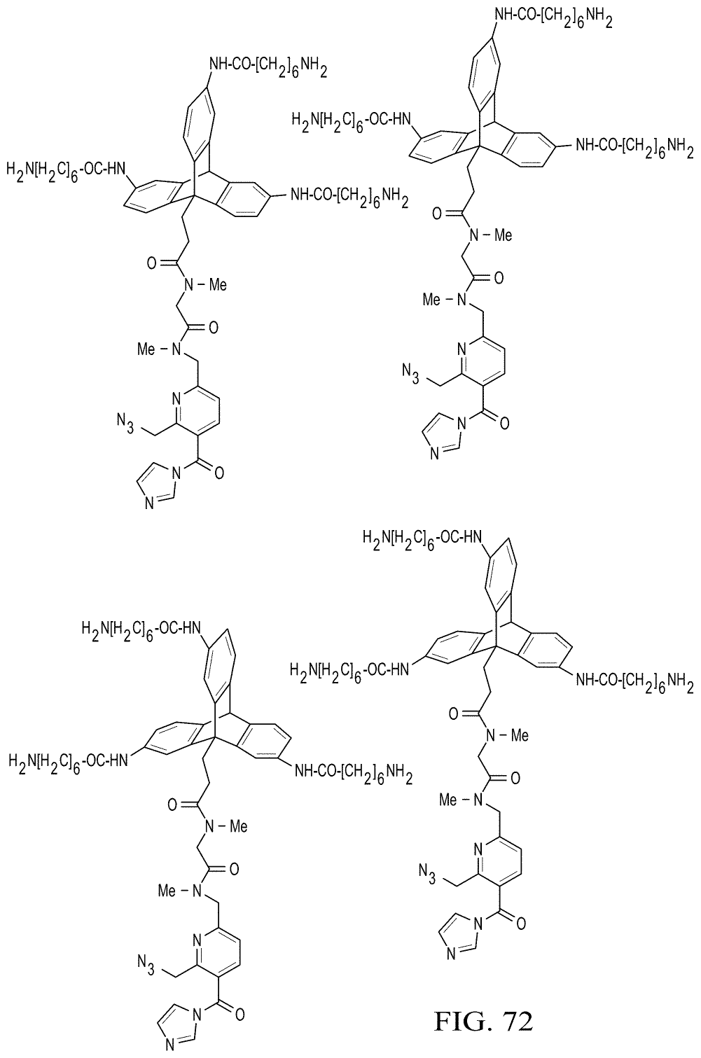

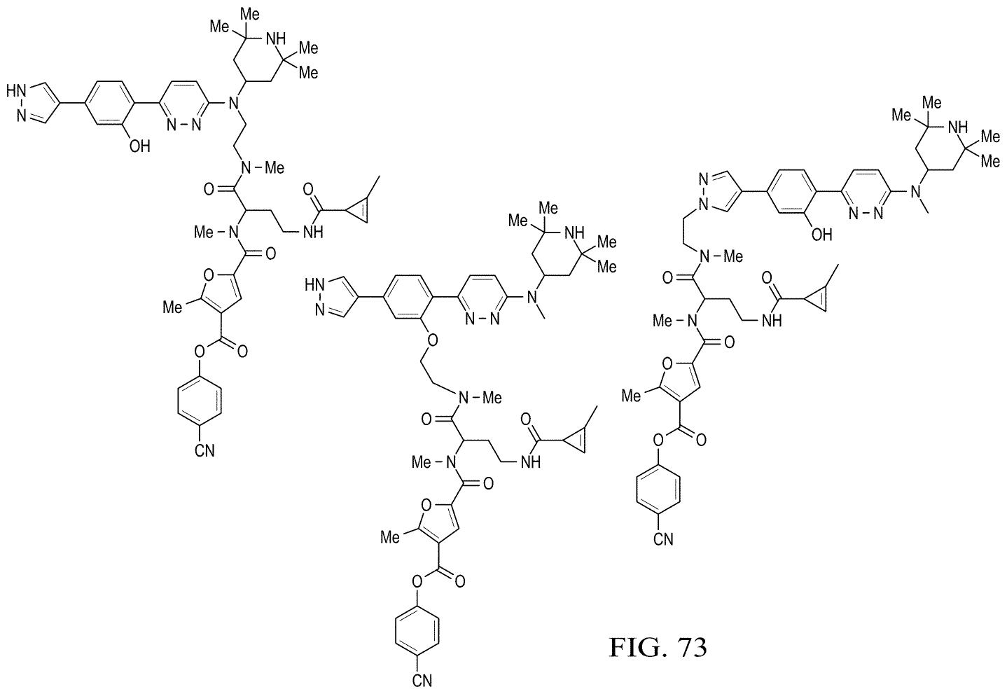

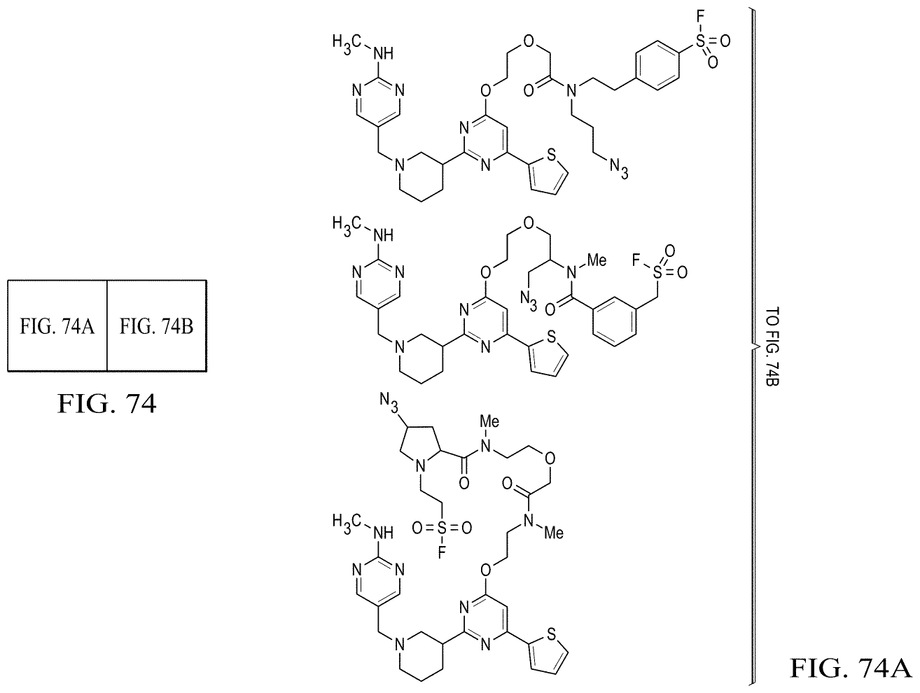

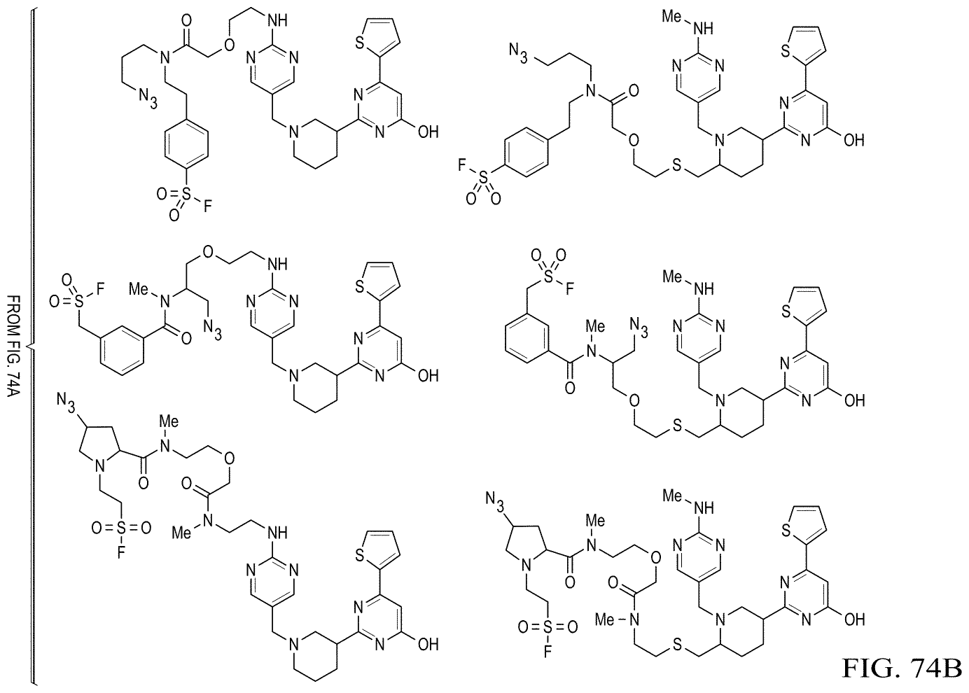

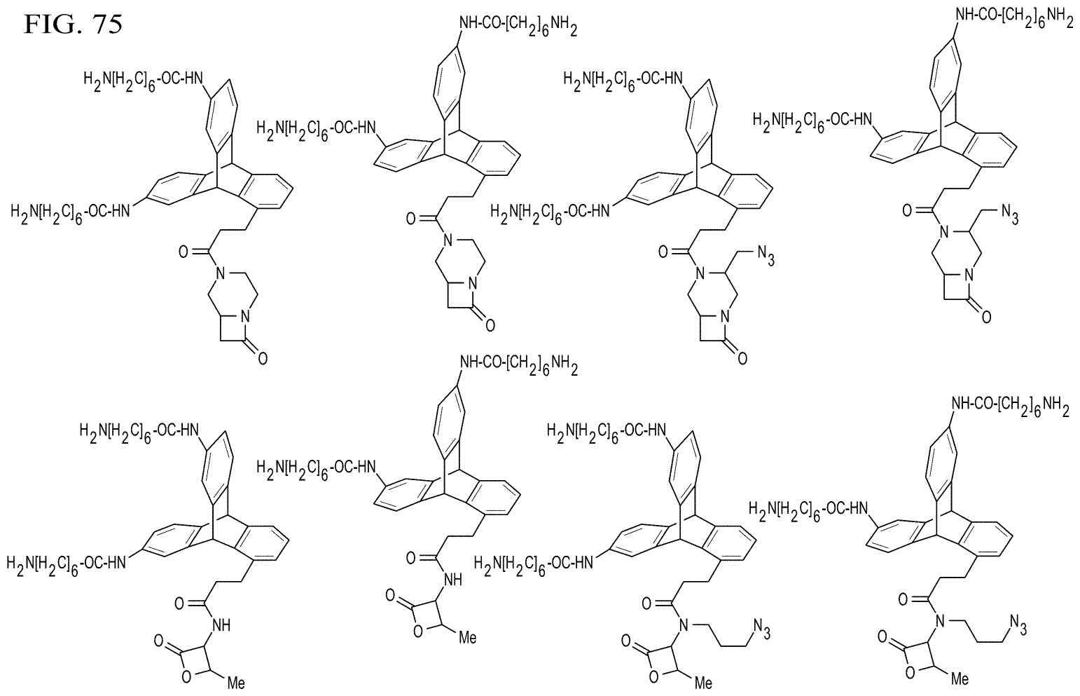

In some embodiments, the click-ready group is selected from those shown in FIG. 45 or 69.

Pull-Down Groups

A number of pull-down groups (R^{PD} in, for example, Formulae I-X above) may be used in the present invention. In some embodiments, pull-down groups contain a bioorthogonal reaction partner that reacts with a click-ready group to attach the pull-down group to the rest of the compound, as well as appropriate group allowing for selective isolation or detection of the pulled-down compound. For example, use of avidin or streptavidin in a pull-down group would allow isolation of only those RNAs that had been ‘hooked’, as explained in further detail below. In some embodiments, the pull-down group is selected from those shown in FIG. 69.

Another method for focused pull-down is to employ standard methods of pulling down RNAs of interest using DNA micro-arrays displaying sequences complementary to the sequences of RNAs of interest. This will allow selective isolation of RNAs of interest, which can be assayed via sequencing to determine whether any hook constructs are attached.

### 3. General Methods of Providing the Present Compounds

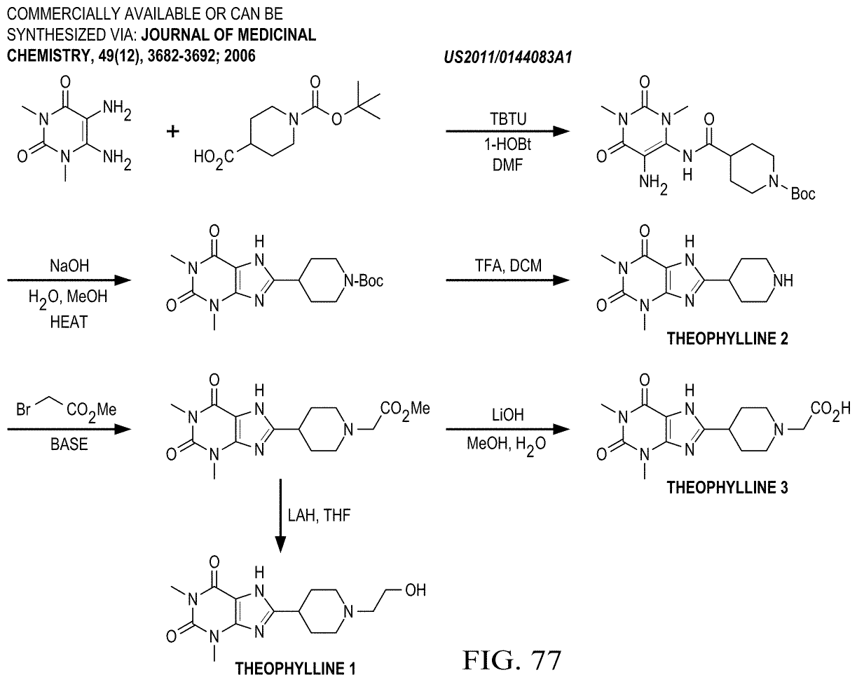

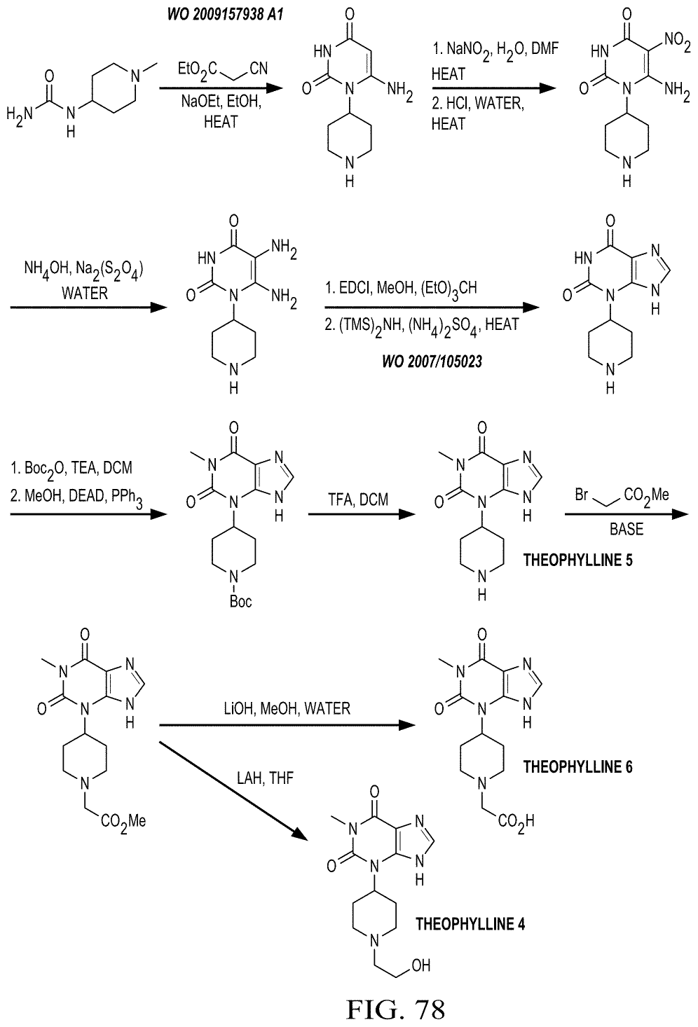

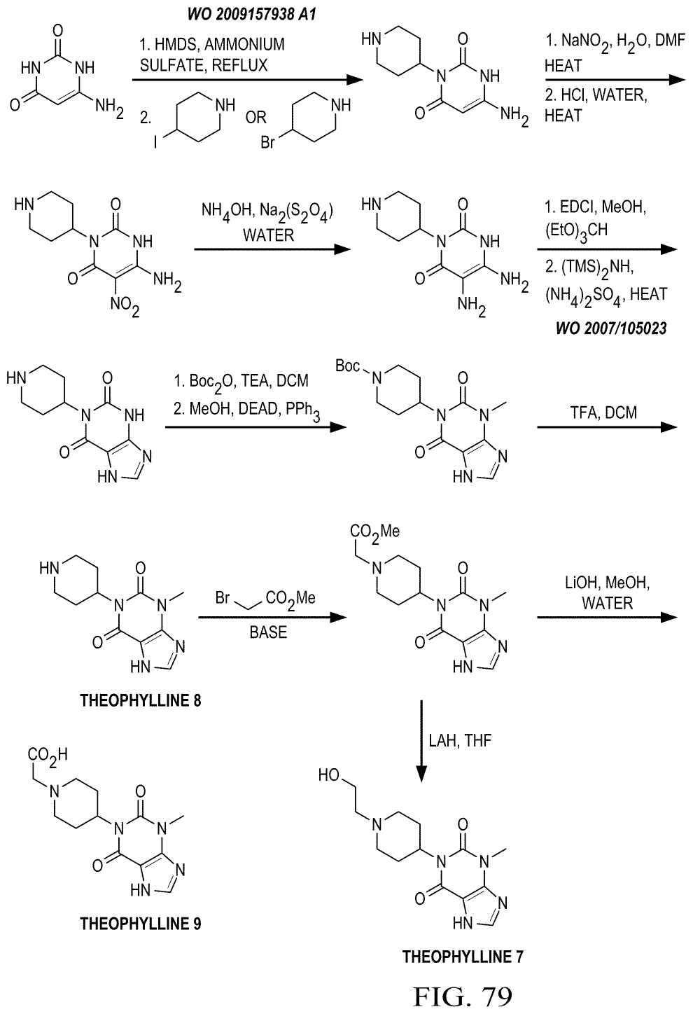

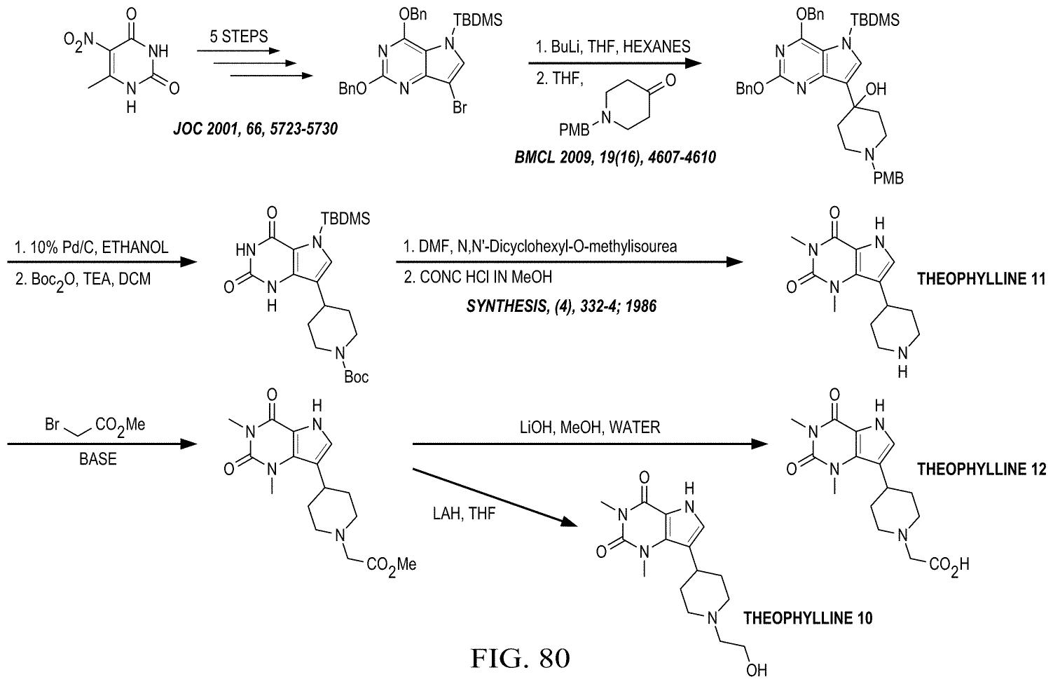

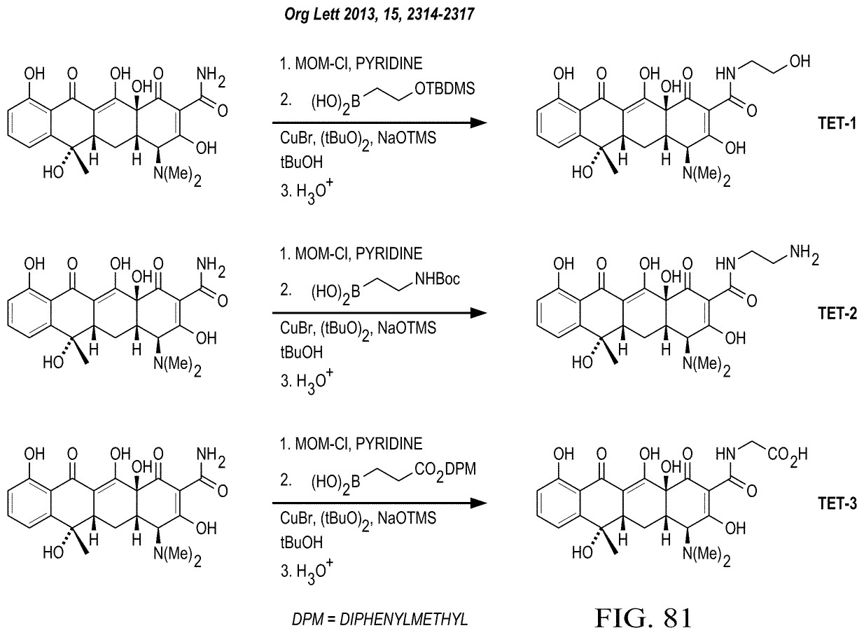

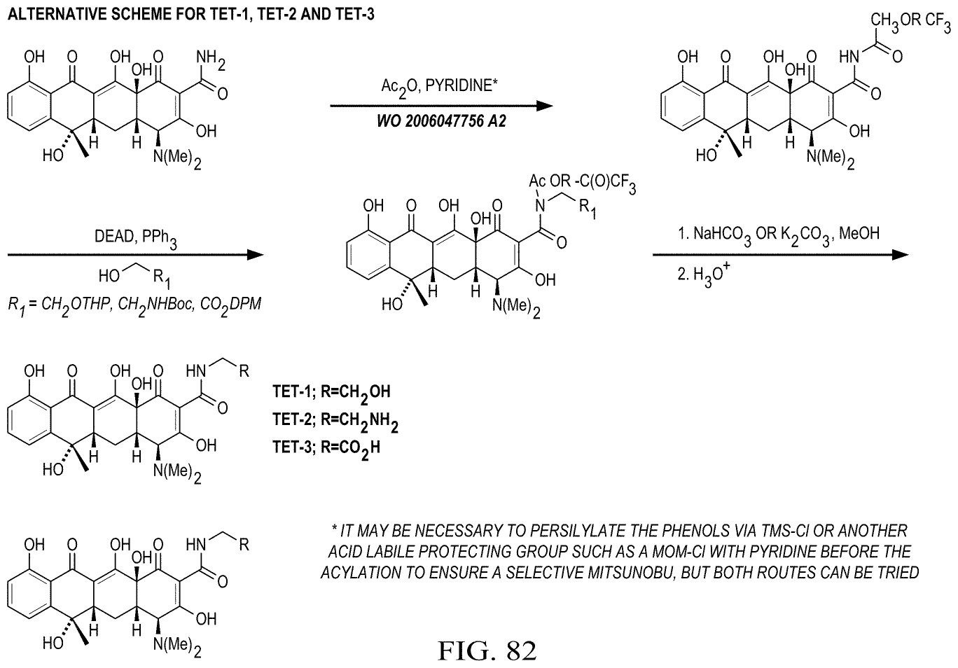

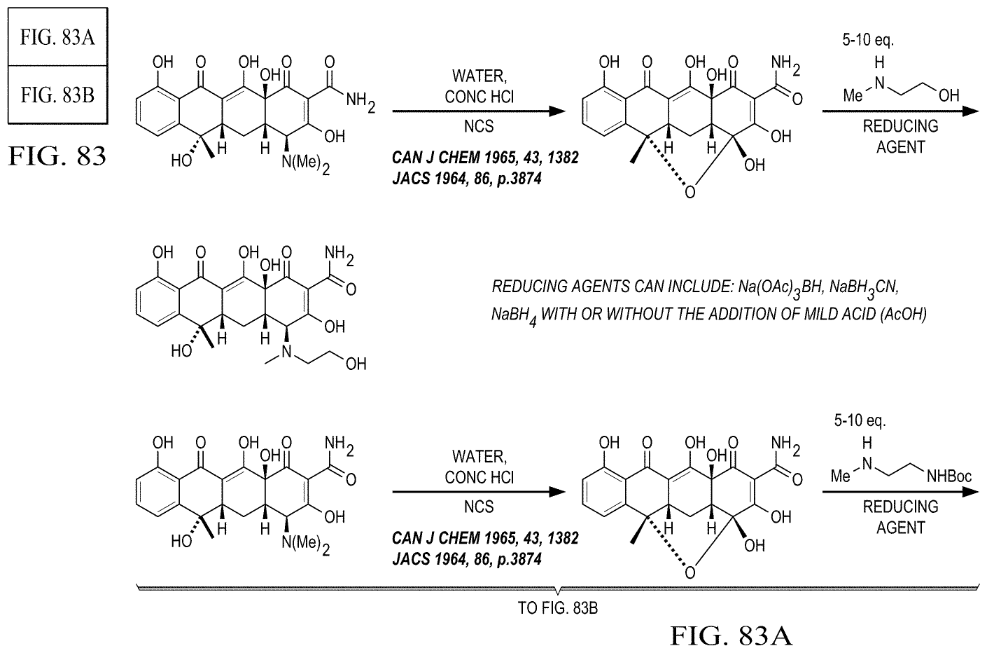

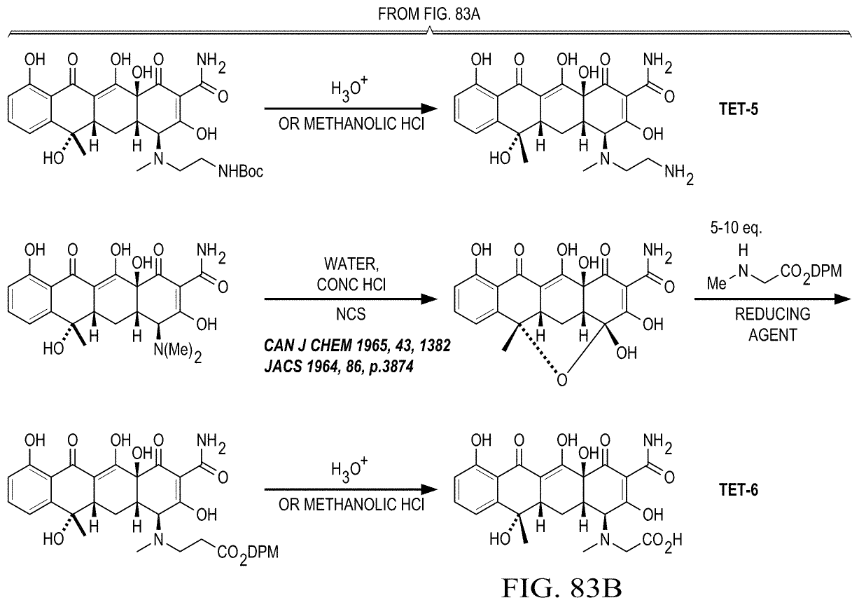

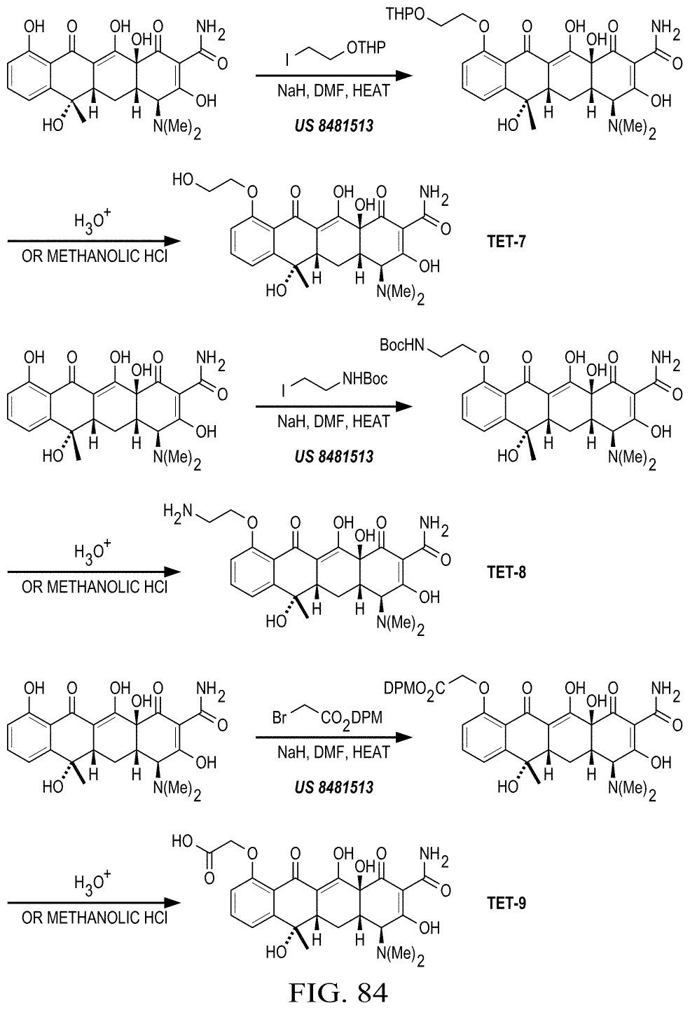

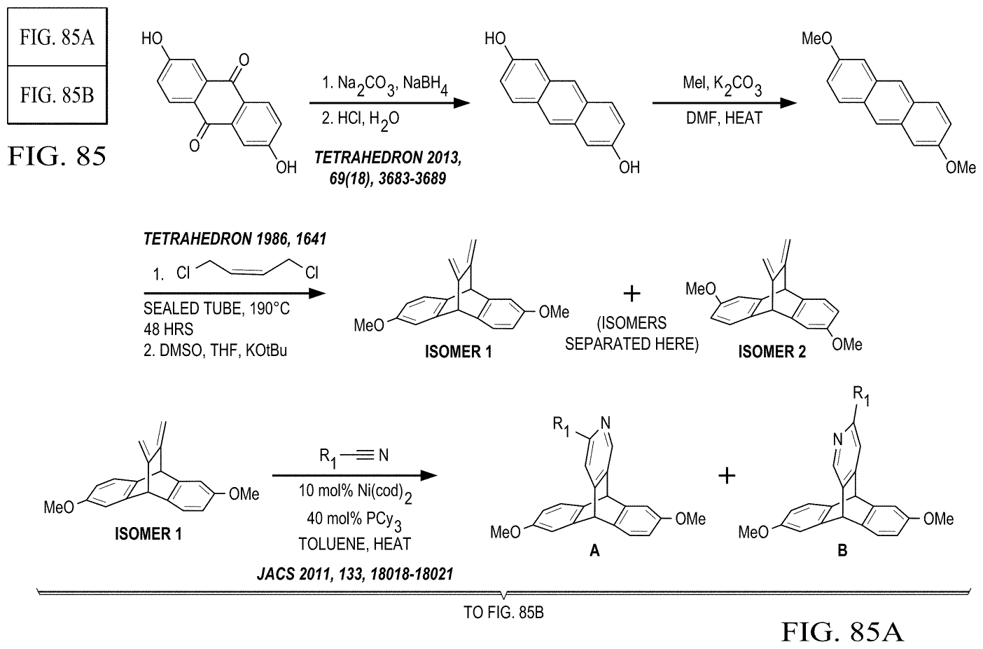

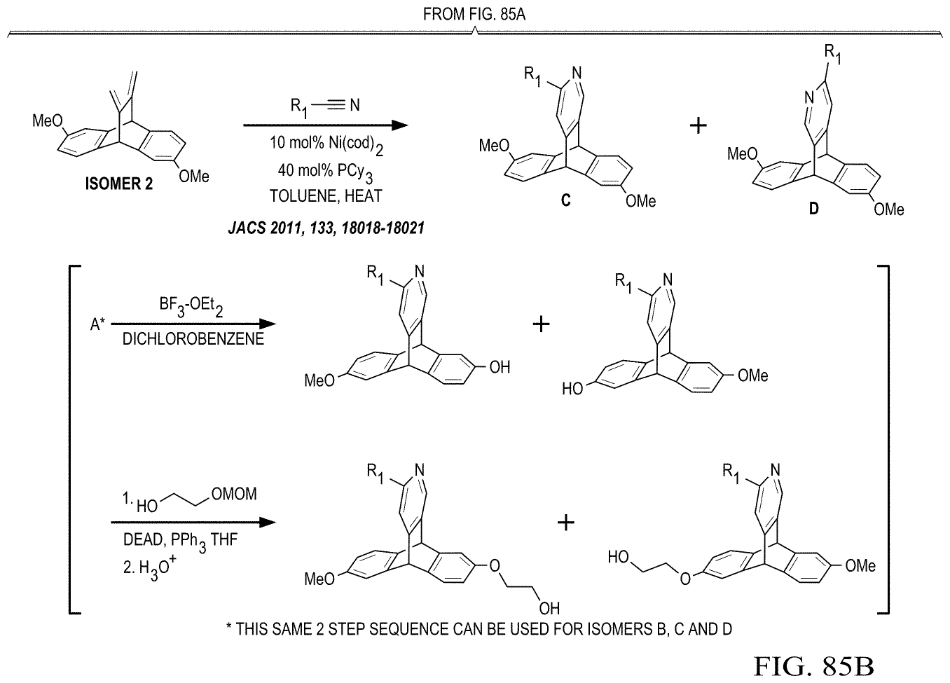

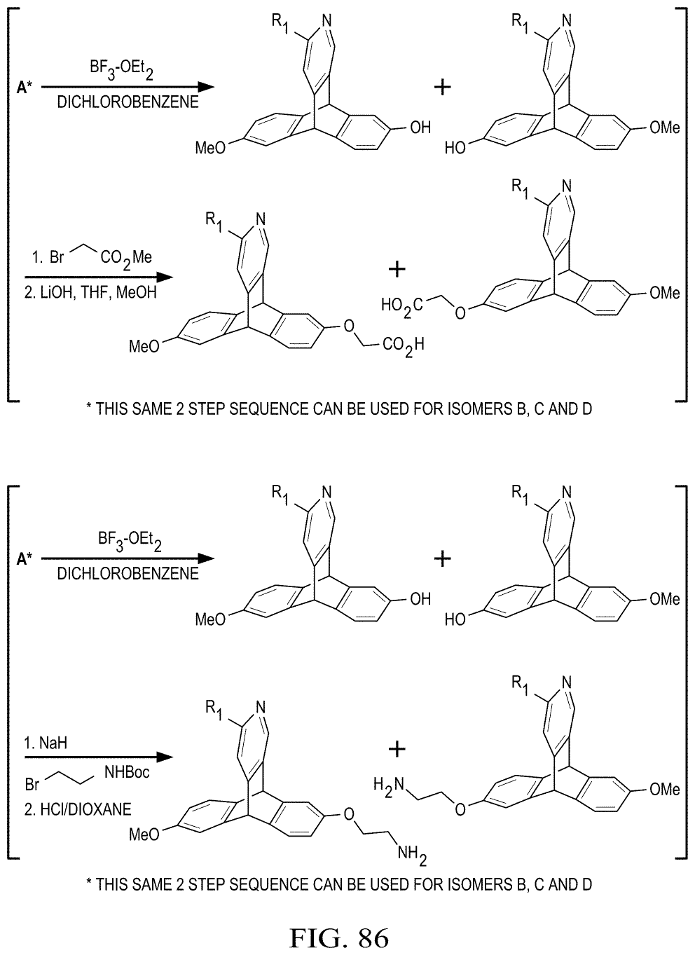

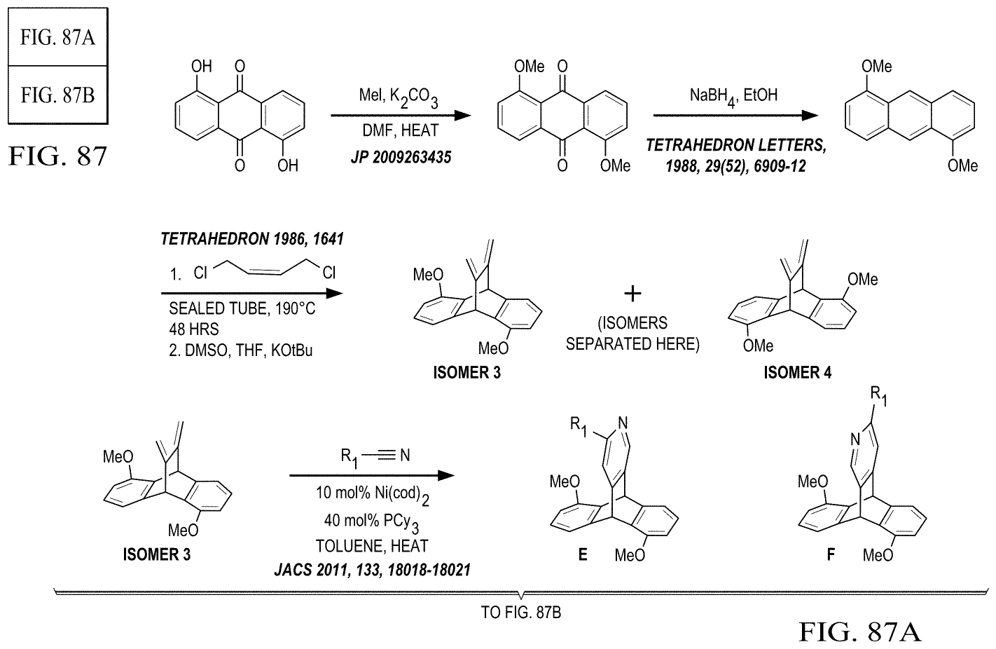

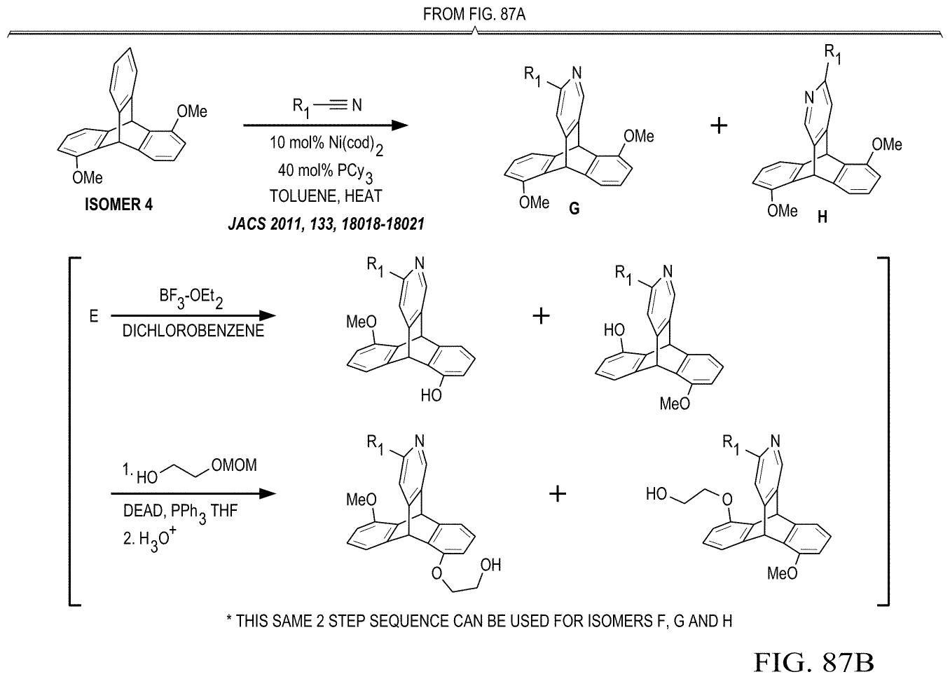

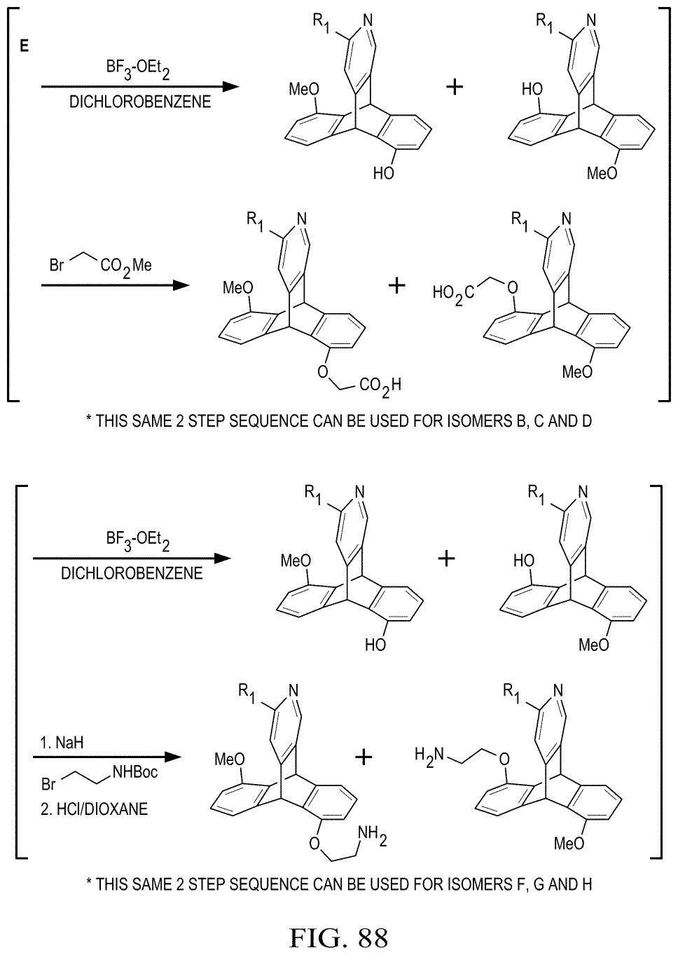

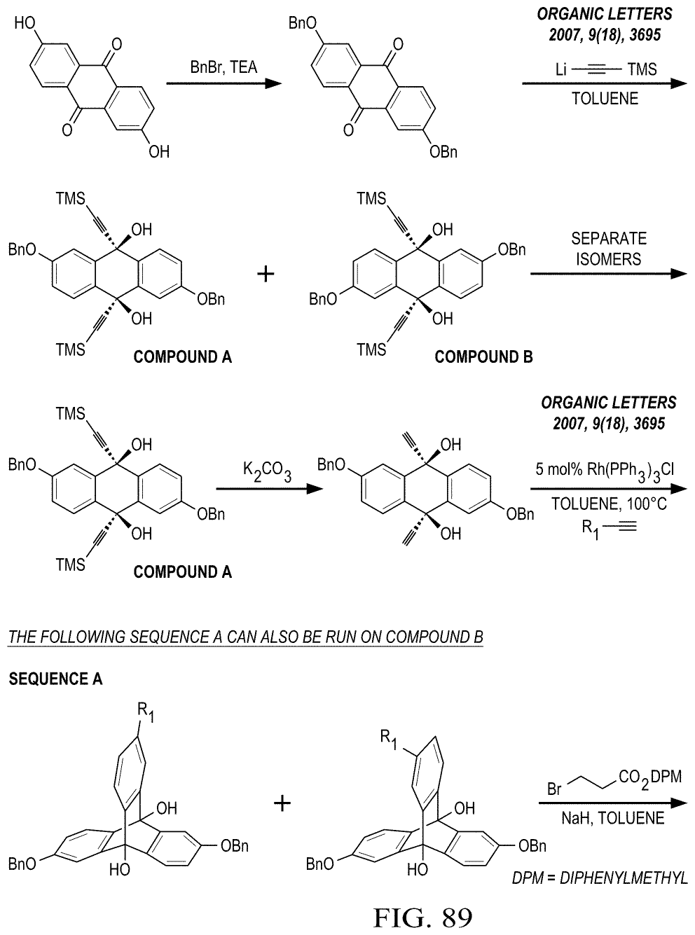

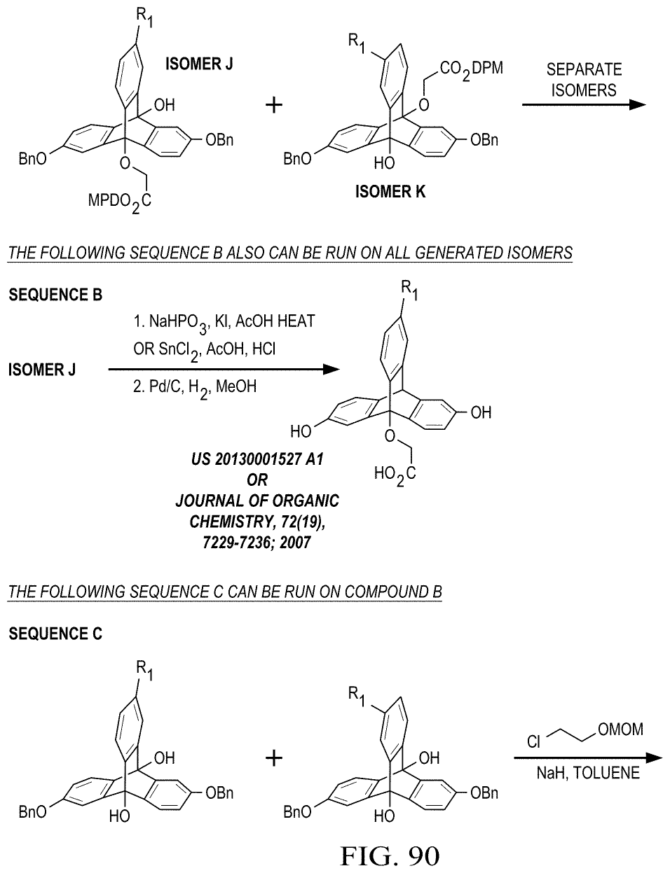

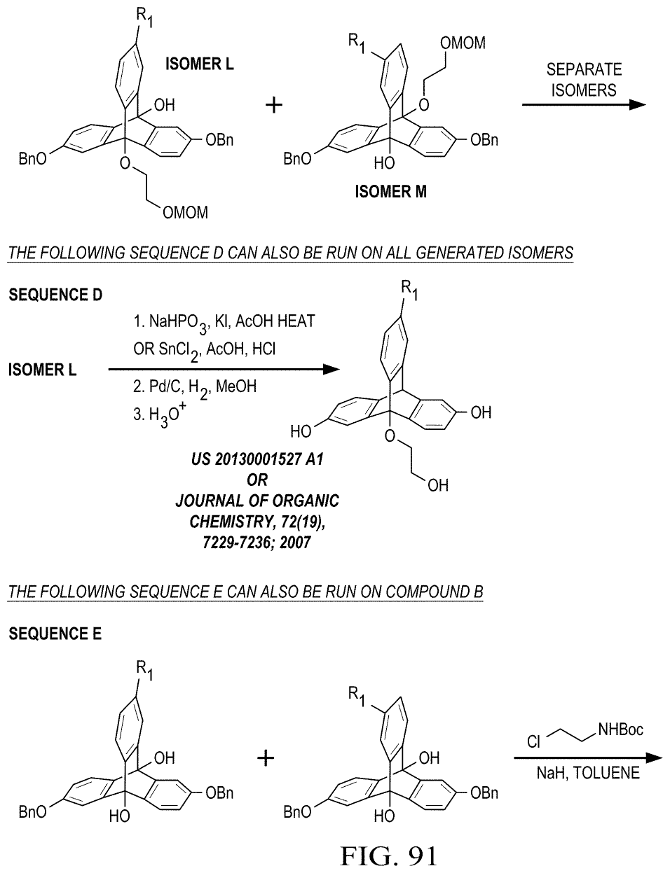

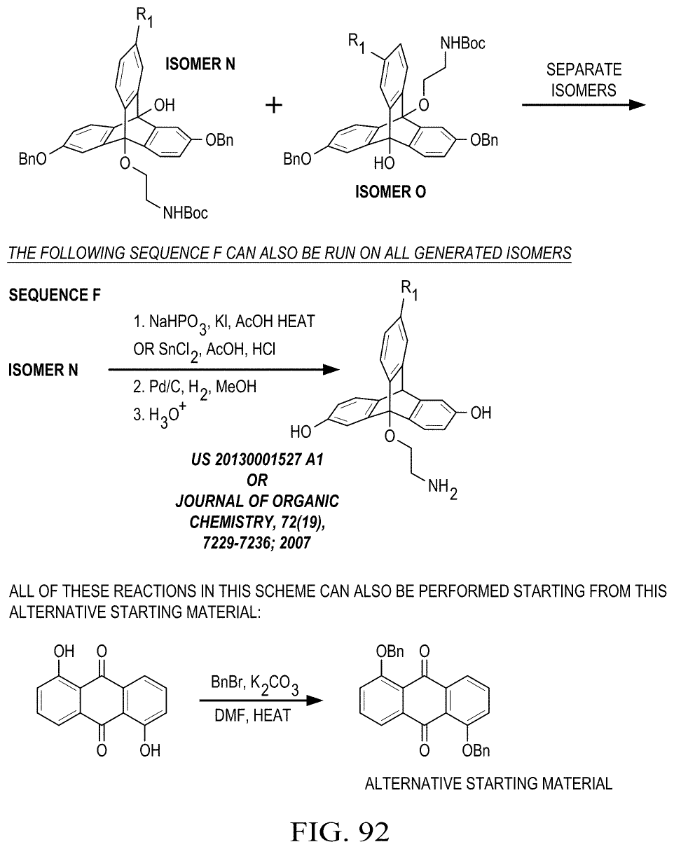

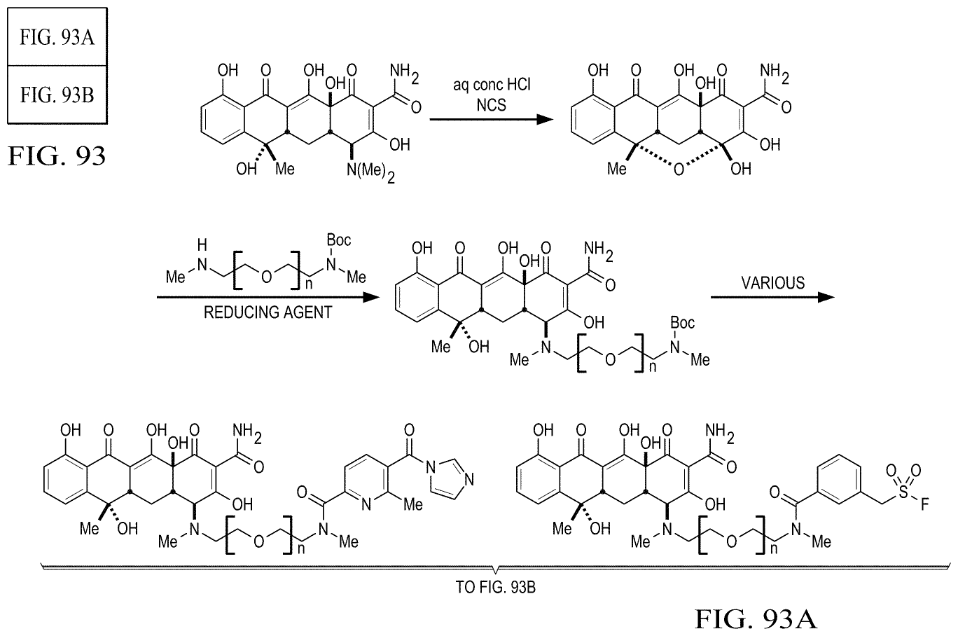

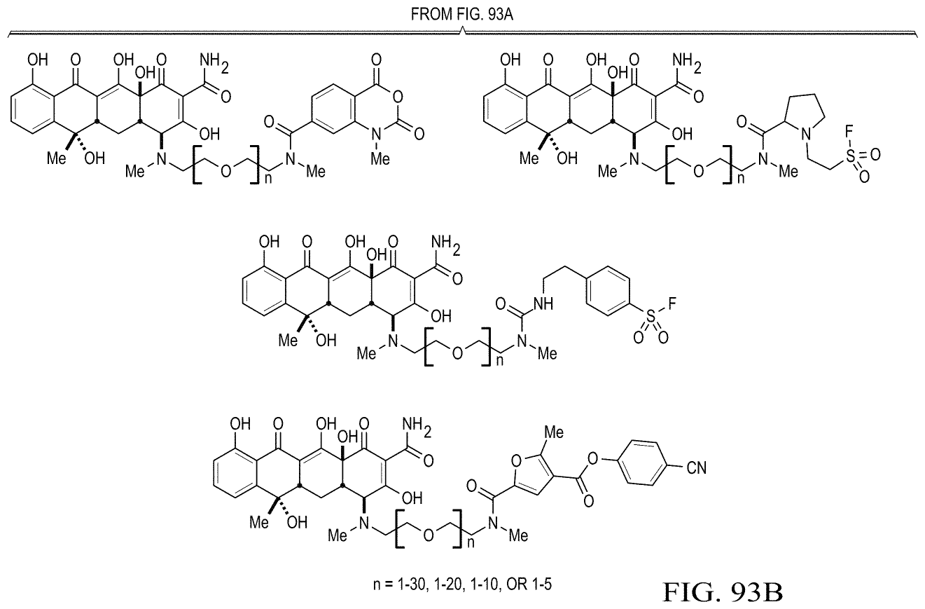

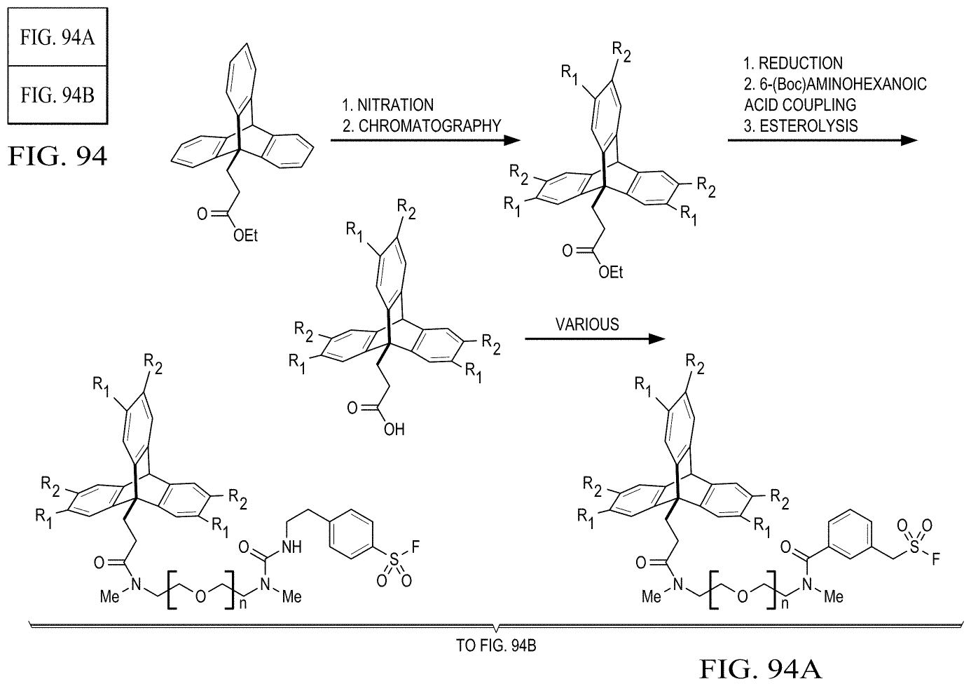

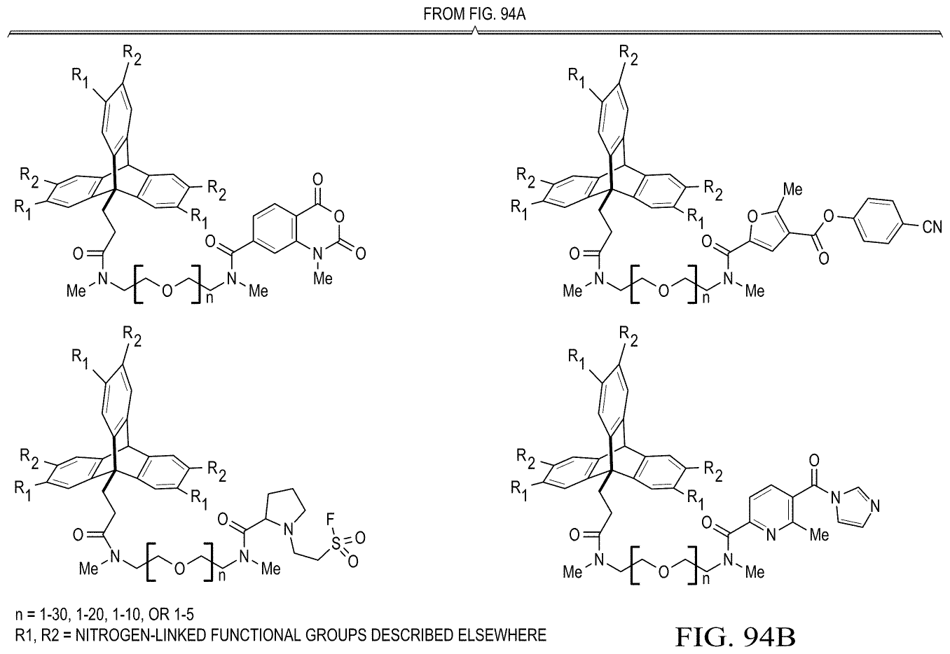

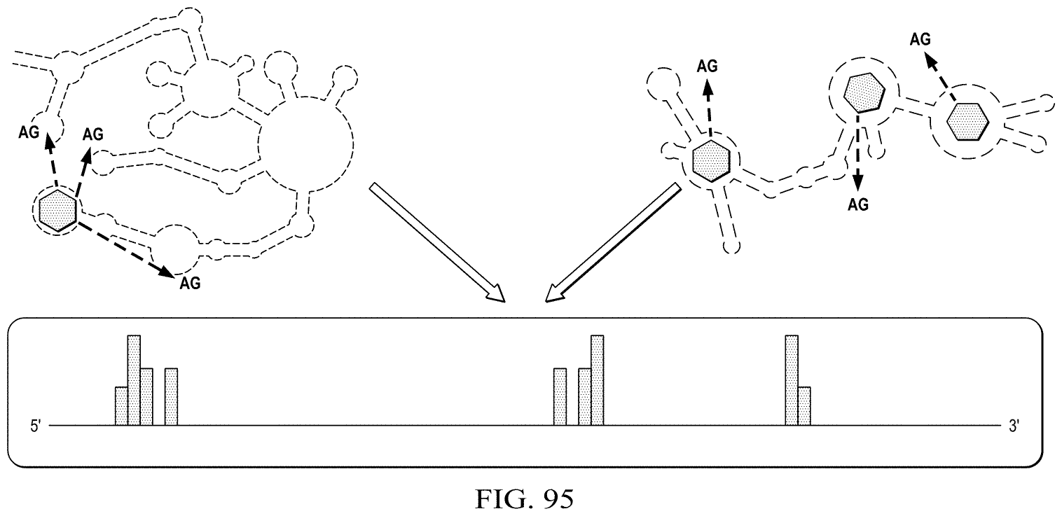

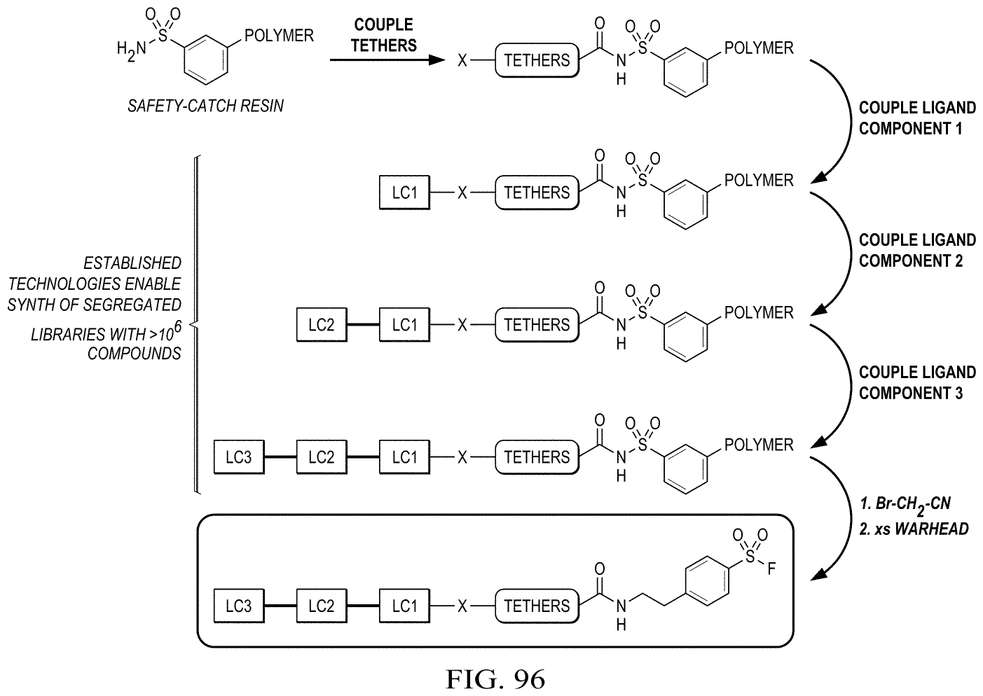

The compounds of this invention may be prepared or isolated in general by synthetic and/or semi-synthetic methods known to those skilled in the art for analogous compounds and by methods described in detail in the Examples and Figures, herein. For example, various compounds of the present invention may be synthesized by reference to FIG. 5-31 or 77-94 or 96.

In the schemes and chemical reactions depicted in the detailed description, Examples, and Figures, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “leaving group” (LG) includes, but is not limited to, halogens (e.g. fluoride, chloride, bromide, iodide), sulfonates (e.g. mesylate, tosylate, benzenesulfonate, brosylate, nosylate, triflate), diazonium, and the like.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3d edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification and Figures.

### 4. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit or modulate a target RNA, or a mutant thereof, in a biological sample or in a patient. In certain embodiments, the amount of compound in compositions of this invention is such that is effective to measurably inhibit or modulate a target RNA, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the modulation of a target RNA to retreat an RNA-mediated disease or condition.

The activity of a compound utilized in this invention to modulate a target RNA may be assayed in vitro, in vivo or in a cell line. In vitro assays include assays that determine modulation of the target RNA. Alternate in vitro assays quantitate the ability of the compound to bind to the target RNA. Detailed conditions for assaying a compound utilized in this invention to modulate a target RNA are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are modulators of a target RNA and are therefore useful for treating one or more disorders associated with or affected by (e.g., downstream of) the target RNA. Thus, in certain embodiments, the present invention provides a method for treating an RNA-mediated disorder comprising the step of administering to a patient in need thereof a compound of the present invention, or pharmaceutically acceptable composition thereof.

As used herein, the terms “RNA-mediated” disorders, diseases, and/or conditions as used herein means any disease or other deleterious condition in which RNA, such as an overexpressed, underexpressed, mutant, misfolded, pathogenic, or oncogenic RNA, is known to play a role. Accordingly, another embodiment of the present invention relates to treating or lessening the severity of one or more diseases in which RNA, such as an overexpressed, underexpressed, mutant, misfolded, pathogenic, or oncogenic RNA, is known to play a role.

In some embodiments, the present invention provides a method for treating one or more disorders, diseases, and/or conditions wherein the disorder, disease, or condition includes, but is not limited to, a cellular proliferative disorder.

### Cellular Proliferative Disorders

The present invention features methods and compositions for the diagnosis and prognosis of cellular proliferative disorders (e.g., cancer) and the treatment of these disorders by modulating a target RNA. Cellular proliferative disorders described herein include, e.g., cancer, obesity, and proliferation-dependent diseases. Such disorders may be diagnosed using methods known in the art.

### Cancer

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). In some embodiments, the cancer is melanoma or breast cancer.

Cancers includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the present invention provides a method for treating a tumor in a patient in need thereof, comprising administering to the patient any of the compounds, salts or pharmaceutical compositions described herein. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments the tumor comprises small cell lung cancer (SCLC). In some embodiments the tumor comprises non-small cell lung cancer (NSCLC).

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

### Other Proliferative Diseases

Other proliferative diseases include, e.g., obesity, benign prostatic hyperplasia, psoriasis, abnormal keratinization, lymphoproliferative disorders (e.g., a disorder in which there is abnormal proliferation of cells of the lymphatic system), chronic rheumatoid arthritis, arteriosclerosis, restenosis, and diabetic retinopathy. Proliferative diseases that are hereby incorporated by reference include those described in U.S. Pat. Nos. 5,639,600 and 7,087,648.

### Inflammatory Disorders and Diseases

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods of this invention is an disease of the skin. In some embodiments, the inflammatory disease of the skin is selected from contact dermatitits, atompic dermatitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic jubenile idiopathic arthritis (SJIA), Cryopyrin Associated Periodic Syndrome (CAPS), and osteoarthritis.

In some embodiments the inflammatory disease which can be treated according to the methods of this invention is a TH17 mediated disease. In some embodiments the TH17 mediated disease is selected from Systemic lupus erythematosus, Multiple sclerosis, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

### Metabolic Disease

In some embodiments the invention provides a method of treating a metabolic disease. In some embodiments the metabolic disease is selected from Type 1 diabetes, Type 2 diabetes, metabolic syndrome or obesity.

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of modulating the activity of a target RNA in a biological sample comprising the step of contacting said biological sample with a compound of this invention, or a composition comprising said compound.

According to another embodiment, the invention relates to a method of modulating the activity of a target RNA in a biological sample comprising the step of contacting said biological sample with a compound of this invention, or a composition comprising said compound. In certain embodiments, the invention relates to a method of irreversibly inhibiting the activity of a target RNA in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Another embodiment of the present invention relates to a method of modulating the activity of a target RNA in a patient comprising the step of administering to said patient a compound of the present invention, or a composition comprising said compound.

According to another embodiment, the invention relates to a method of inhibiting the activity of a target RNA in a patient comprising the step of administering to said patient a compound of the present invention, or a composition comprising said compound. According to certain embodiments, the invention relates to a method of irreversibly inhibiting the activity of a target RNA in a patient comprising the step of administering to said patient a compound of the present invention, or a composition comprising said compound. In other embodiments, the present invention provides a method for treating a disorder mediated by a target RNA in a patient in need thereof, comprising the step of administering to said patient a compound according to the present invention or pharmaceutically acceptable composition thereof. Such disorders are described in detail herein.

### EXEMPLIFICATION

As depicted in the Examples below, in certain exemplary embodiments, compounds are prepared according to the following general procedures and used in biological assays and other procedures described generally herein. It will be appreciated that, although the general methods depict the synthesis of certain compounds of the present invention, the following general methods, and other methods known to one of ordinary skill in the art, can be applied to all compounds and subclasses and species of each of these compounds, as described herein. Similarly, assays and other analyses can be adapted according to the knowledge of one of ordinary skill in the art.

### Example 1: Procedure for SHAPE-MaP to Locate and Quantify Sites of Modifications in RNA

As discussed above, a variety of RNA molecules play important regulatory roles in cells. RNA secondary and tertiary structures are critical for these regulatory activities. Various tools are available for determining RNA structure. One of the most effective methods is SHAPE (selective 2′-hydroxyl acylation and primer extension). This methodology takes advantage of the characteristic that the ribose group in all RNAs has a 2′-hydroxyl whose reactivity is affected by local nucleotide flexibility and accessability to solvent. This 2′-hydroxyl is reactive in regions of the RNA that are single-stranded and flexible, but is unreactive at nucleotides that are base-paired. In other words, SHAPE reactivity is inversely proportional to the probability that a nucleotide is base paired within an RNA secondary structure. Reagents that chemically modify the RNA at this 2′-hydroxyl can be used as probes to discern RNA structure. SHAPE reagents are small-molecules such as 1-methyl-7-nitroisatoic anhydride (1M7) and benzoyl cyanide (BzCN) that react with the 2′-hydroxyl group of flexible nucleotides to form a 2′-O-adduct. Besides 1M7, other acylation electrophiles such as 2-methylnicotinic acid imidazolide (NAI) and 2-methyl-3-furoic acid imidazolide (FAI) could be utilized. The sites at which this chemical modification takes place can be detected by either primer extension or by protection from exoribonuclease digestion. SHAPE-MaP (SHAPE mutational profiling) takes advantage of the ability of reverse transcriptase to read through RNA chemical modifications and incorporate nucleotides that are not complementary to the original template RNA. Through this mis-incorporation, the sites of 2′-OH modification by the SHAPE reagent are recorded and detected by deep sequencing of the cDNA. The secondary structure of the RNA can be elucidated by determining the SHAPE reactivity values at each RNA nucleotide position relative to controls such as denatured RNA.

Since specific RNA molecules play critical regulatory roles in healthy and diseased human cells, small molecules that selectively bind distinct RNA structures could modulate these biological and pathophysiological processes, and could be promising novel therapeutic candidates. In addition to the use of SHAPE-MaP to determine RNA structures, a modified version of SHAPE-MaP could be employed to (a) identify small molecule compounds that bind RNA and (b) to determine the site of interaction of these compounds on the target RNA. The central feature of the present invention is the tethering of a small molecule or a library of small molecules to the SHAPE reagent. In the case of acylating SHAPE reagents, the tether links the acylation event with the ligand binding event. The acylation pattern on the RNA will be decisively altered because the activity of the acylation agent will be constrained to riboses proximal to ligand binding pockets on the RNA. Thus, one can infer the existence and the location of ligand binding pockets from the altered SHAPE-MaP acylation pattern, as revealed in the sequencing data.

SHAPE-MaP analysis provides a reliable pathway to the three-dimensional structure of folded RNAs. The essence of SHAPE-MaP is: (1) Low-level benzoylation of solvent-exposed 2′-OH groups found along the entire spine of RNA. The success of this reaction relies on the relative acidity of the 2′-OH of a ribose (pKa 13) relative to other, less reactive alcohols.

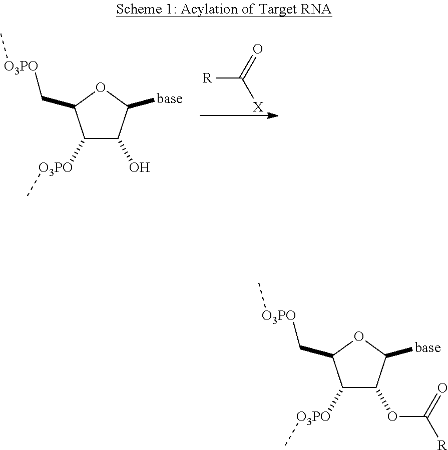

(2) Denaturation of these covalently modified RNAs followed by enzyme-mediated formation of a corresponding cDNA or cDNA library. (3) A key finding is that when the cDNA or cDNA library is formed, RNA riboses that are benzoylated in the target RNA induce random incorporation of bases in the complementary cDNA strand. Put differently, there is “read through,” but 2′-O-benzoyl riboses induce “mutation” in the cDNA. (4) Upon sequencing of the resulting cDNA, sites with random mutation reflect sites on the original folded that were exposed to solvent. When these inferences about which portions of the folded RNA are solvent-exposed are then imposed as constraints on the computational models for predicting RNA structure, a high-accuracy model of the 3D structure of the RNA can be developed.

Further details of the SHAPE method including alternate reagents, conditions, and data analysis are described in WO 2015/054247, US 2014/0154673, U.S. Pat. Nos. 7,745,614, and 8,313,424, each of which is hereby incorporated by reference.

### Example 2: Modification of SHAPE-MaP to Identify Small Molecule RNA Ligands (the Hook the Worm and Hook and Click (PEARL-Seq) Method)

Historical efforts to identify small-molecule ligands that bind to RNA have focused on base-pairing or on canonical structural motifs in duplex RNA: intercalation between bases and/or groove binding. But these motifs do not support selective binding of small molecules to specific RNAs. However, RNA folds into an enormous variety of complex tertiary structures that present pockets conducive to small molecule binding—small molecules that are complementary to the shape and electrostatics presented by those pockets. Insofar as the details of shape and electrostatics reflect the underlying sequence of the RNA, small molecules can achieve selectivity, much as they do when binding protein pockets.

Indeed, there are now several reports of drug-like small molecules that bind to RNA, many of them FDA-approved (see Table 4 below).

### Small-Molecule Ligands by Class

Though a range of small-molecule chemotypes has been demonstrated to bind to folded RNA (Guan & Disney, ACS Chem. Biol. 2012 7, 73-86), hereby incorporated by reference, there are limited reports of high-throughput screening of large libraries (>10⁵ compounds) to identify RNA-binding ligands. Accordingly there are also few reports of small molecules synthetically optimized for RNA binding. The present invention paves the path to a remedy for these deficiencies. Below is a table summarizing the broad chemotypes which have demonstrable RNA binding and will serve as the starting point to optimize and validate our screening method, which will in turn enable the systematic screening of essentially all known chemotypes against RNA structures of therapeutic interest.

TABLE 4
RNA-binding Small Molecules

| Small Molecule | Status | RNA Target | Reference |
|---|---|---|---|
| Linezolid | FDA-approved | Bacterial | Leach et al. Mol. |
| | antibiotic | ribosomal | Cell 2007, 26, 393- |
| | | RNA | 402 |
| Tedizolid | FDA-approved | Bacterial | Leach et al. Mol. |
| | antibiotic | ribosomal | Cell 2007, 26, 393- |
| | | RNA | 402 |
| Tetracycline | FDA-approved | Bacterial | Brodersen et al. Cell |
| | antibiotic | 30S ribosomal | 2000, 103, 1143- |
| | | RNA | 1154 |
| Aminoglycosides | FDA-approved | Bacterial | Fourmy et al. |
| | antibiotics | 16S ribosomal | Science 1996, 274, |
| | | RNA | 1367-1371 |
| Theophylline | FDA-approved | Aptameric | Jenison et al. |
| | for COPD and | RNA | Science 1994, 263, |
| | asthma | | 1425-1429 |

These discoveries revealed a molecular mechanism of action that was not anticipated. The intentional design of small molecules that bind to folded RNA has been pursued only rarely because of substantial technical challenges, with one notable example being the design of triptycene-based ligands able to bind selectively to RNA three-way junctions (Barros et al., Angew. Chem. Int. Ed 2014, 53, 13746-13750. Triptycene-based ligands will thus provide another chemotype with RNA binding ability to serve as another starting point in the described screening methods. Technical challenges in investigating small molecule binding to RNA include the instability of many RNAs in solution, substantial differences between the native structure in the cell versus unadorned RNA in solution, and frequent difficulties in recovering the original (presumably biologically relevant) fold after denaturation. In addition, in contrast to protein targets, the specific molecular “partners” of the target RNA in the cell and the sub-site on the RNA are often unknown. Finally, the methods often employed in determining the structure of other biomolecules (e.g., DNA, proteins), such as X-ray crystallography, NMR, and cryo-EM, are not reliable paths to precise structure determination for RNA in commercially relevant timeframes. All of these challenges conspire to make RNA a difficult target to screen against libraries of small molecules.

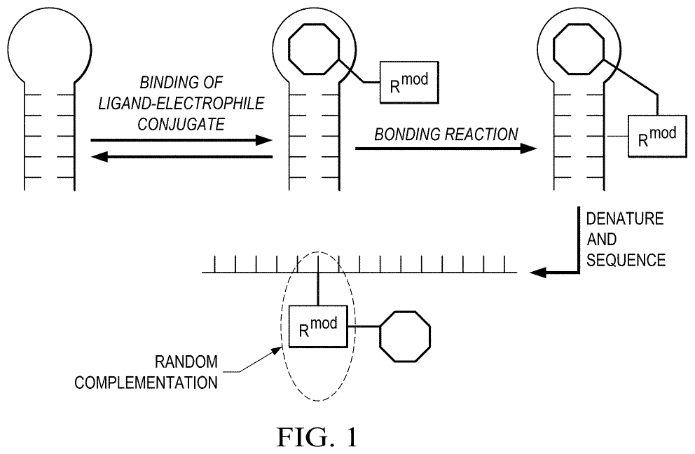

A element of the present method to discover small-molecule RNA modulators is to exploit the ubiquity of the 2′-OH nucleophile on the target RNA for a different purpose than in SHAPE-MaP (see FIG. 1). By tethering, for example, an acylating or sulfonylating agent (aka ‘warhead’) to an RNA-binding ligand, this will impose a novel bias to the sites of 2′-OH covalent modification: specifically, the tethering will strongly favor acylation of nucleotide riboses proximal to the ligand binding site. Proximity will not be limited to riboses near in sequence because the RNA will be folded. Optimization of the warheads and tethers will render the acylation process highly selective by minimizing ‘background’ acylation that is not accelerated by ligand-mediated pre-association with binding pockets on the folded RNA. Insofar as we can carry out the acylation event in cells, we bypass any residual concerns about poor fidelity of RNA structure in free solution relative to RNA structure inside cells. From these data we can infer a wide range of information critical to drug discovery and optimization:
The existence of a pocket on an RNA complementary to a small molecule.
The subsite on the target RNA that binds to the identified small molecule.
Constraints that inform the 3D structure of the folded RNA proximal to the binding pocket.
The identity of other RNAs that a given small molecule also binds to.

In addition to the above method, it is also possible to incorporate functional groups that enable various pull-down methods that limit the breadth of sequencing. We can incorporate so-called ‘click’ groups on the warhead or the tether. These click groups enable facile incorporation—after the RNA ligand-mediated acylation—of biotin which in turn allows streptavidin or avidin-mediated isolation of only those RNAs that were targeted by the hook construct. This will accelerate the overall discovery process and limit the amount of sequencing required.

Another method for focused screening of a single RNA or class of RNAs in cells is to employ standard methods of pulling down RNAs of interest using DNA micro-arrays displaying sequences complementary to the sequences of RNAs of interest. This will allow selective isolation of RNAs of interest, which can be assayed via sequencing to determine whether any hook constructs are attached. Focused screening against a single RNA in cells can also be achieved by sequencing the target via specific primer extension techniques, thus bypassing the need to isolate the RNA of interest.

An additional advantage to performing small-molecule lead identification against RNA targets inside cells is that there are many post-transcriptional modifications that will impact the precise shape of the three-dimensional fold as well as the concave surface of the small molecule binding pocket. Insofar as these post-transcriptional modifications are difficult to identify at all, difficult to assess in the pathological cell, and even more difficult to recapitulate chemically or enzymatically outside of the cell, there are substantial advantages in being able to address the RNA targets in their native environment. Below is a table of some of the principal post-transcriptional modifications that contribute to the complexity of RNA as a target for screening:

TABLE 5

| Post-transcriptional Modification | Enzyme Mediating Modification |
|---|---|
| Adenine −> inosine | adenosine deaminase acting on RNA |
| | (ADAR) |
| Guanine −> 7-methylguanine | RNA (guanine-7-)methyltransferase |
| 5-methylcytosine −> 5- | Ten-eleven translocation (Tet) enzymes |
| hydroxymethylcytosine | |
| Adenine −> 6-methyladenine | m⁶A methyltransferase complex |
| Cytosine −> 5-methylcytosine | NSUN2 and TRDMT1 |

Covalent Affinity Transcriptomics

The method:
1. Small-molecule ligands are selected for screening with a view to assessing their potential for binding to RNA, either in solution or in cells. The number of molecules could be small (1-10) or large (>1,000,000). Implementation of this technology on a robotic liquid-handling platform would make it possible to screen >10,000 molecule in a single screening campaign.
2. The selected ligands would all be tethered to warheads capable of selective (which is to say, proximity-induced) formation of covalent bonds with the 2′-OH of riboses on RNA. The reactions that are the focus of this work are acylations and sulfonylations.

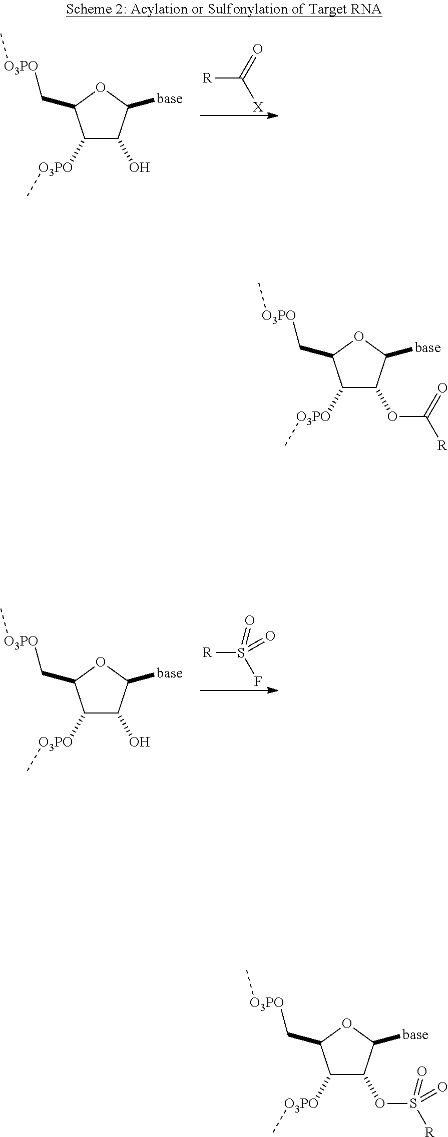

3. The constructs can optionally contain functional groups capable of participating in ‘click reactions’ that enable bio-orthogonal, bio-compatible covalent linkage with additional reagents, most importantly biotin.
4. The ligand-tether-warhead or ligand-tether-warhead-click constructs (‘hooks’ or ‘click-ready hooks’, respectively) are exposed to isolated RNA, synthetic RNA, or RNA in cells for one minute to an hour, as needed, to allow covalent modification to proceed to completion.
5. Isolated or synthetic RNAs are washed to remove excess ‘hook’. For RNAs in cells, the cells are lysed and the RNA-containing fraction isolated.
6. Depending on which constructs are employed, the overall process now branches into at least three possible paths:
7. All the RNA can be sequenced. The conditions that yield the cDNA from the RNA use a reverse transcriptase that “reads through” the acylated or sulfonylated nucleotide but with random base incorporation opposite that site. Bases in the sequence that exhibit random incorporation (or ‘mutation’) reveal where acylation or sulfonylation took place on the original RNA. When a ‘hook’ is used, those acylations or sulfonylations will take place at nucleotides that are proximal—in three dimensions—to the pockets that bind the ligand portion of the ‘hook’. Put differently, mutations in the sequence are the ‘signal’ that indicates where on the target RNA a given ligand bound.
8. Alternatively, using well-known techniques, only those RNAs that are of interest can be isolated and only those sequenced. While this path has the disadvantage that it will not detect association of the ligand with secondary targets, it has the advantage of curtailing the amount of sequencing data that needs to be generated and analyzed. Focused screening against a single RNA in cells can also be achieved by sequencing the target via specific primer extension techniques, thus bypassing the need to isolate the RNA of interest.
9. The third path is available when the ‘hook’ also bears clickable functional groups. On this path, the RNAs isolated after ‘hooking’ are subjected to a click reaction using well-known techniques to create the click product. Typical click reactions are azide/alkyne cycloadditions (either Cu-catalyzed or non-Cu-catalyzed) or Diels-Alder cycloadditions, though other chemistries also answer to the description of ‘hook’. In most applications the click reaction would be used to attach a biotin to all RNAs that are ‘hooked’. Subsequent pull-down with avidin or streptavidin would afford only those RNAs that had been ‘hooked’. This pathway enjoys both advantages: all the RNAs that are ‘hooked’ by a given ligand would be subject to sequencing and without having to sequence the entire transcriptome. For screening large numbers of ligands, the efficiency conferred by the click step is substantial.

### Example 3: SHAPE-MaP Procedure for Use with Hook and Click Compounds (Alternately Referred to Herein as PEARL-Seq)

SHAPE experiments use 2′-hydroxyl-selective reagents that react to form covalent 2′-O-adducts at flexible RNA nucleotides. SHAPE can be performed using purified RNA or intact cells. The SHAPE-MaP approach exploits conditions that cause reverse transcriptase to misread SHAPE-modified nucleotides and incorporate a nucleotide non-complementary to the original sequence into the newly synthesized cDNA. The positions and relative frequencies of SHAPE adducts are recorded as mutations in the cDNA primary sequence. In a SHAPE-MaP experiment, the RNA is treated with a SHAPE reagent or treated with solvent only, and the RNA is modified. RNA from each experimental condition is reverse-transcribed, and the resulting cDNAs are then sequenced. Reactive positions are identified by subtracting data for the treated sample from data obtained for the untreated sample and by normalizing to data for a denatured (unfolded) control RNA.

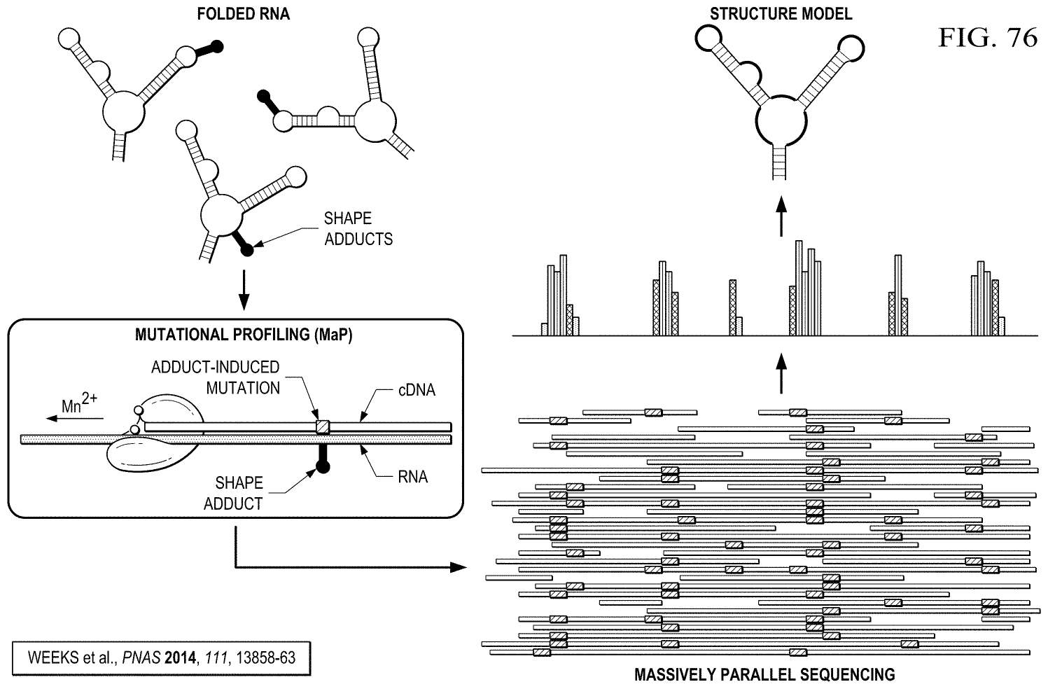

The process is shown in FIG. 76 (Figure taken from Weeks et al., PNAS 2014, 111, 13858-63; see also Siegfried et al., Nature Methods 2014; 11:959-965, each of which is hereby incorporated by reference).

SHAPE-MaP can be performed and analyzed according to detailed published methods (Martin et al., RNA 2012; 18:77-87; McGuinness et al., J. Am. Chem. Soc. 2012; 134:6617-6624; Siegfried et al., Nature Methods 2014; 11:959-965; Lavender et al., PLoS Comput. Biol. 2015; 11(5)e1004230; McGuinness et al., Proc. Natl. Acad. Sci. USA 2015; 112:2425-2430). The SHAPE-MaP sequence data can be analyzed using ShapeFinder (Vasa et al., RNA 2008; 14:1979-1990) or ShapeMapper (Siegfried et al., Nature Methods 2014; 11:959-965) or other software. Each of the foregoing publications is hereby incorporated by reference.

SHAPE-MaP can be performed on synthetic RNA or RNA isolated from any prokaryotic or eukaryotic cell. In addition, SHAPE-MaP can be performed on intact cells, including human cells.

SHAPE-MaP on Pure RNA

In the case of SHAPE-MaP experiments performed with pure RNA, the RNA to be analyzed can be generated in a variety of different ways. RNA can be chemically synthesized as oligonucleotides. Typically, synthetic oligonucleotides are short, having lengths of roughly 20 to 100 nucleotides (nt). However, oligonucleotides as long as approximately 200 nt can be chemically synthesized. For RNAs above 200 nt, including very long transcripts, the RNA can be produced using the T7 in vitro transcription system that is well-known in the field and for which kits are commercially available from a variety of sources (e.g., Epicentre; Madison, Wis.; New England Biolabs, Beverly, Mass.) and the RNA can be cleaned up using a variety of kits (e.g., MegaClear kit; Ambion/ThermoFisher Scientific).

RNA is denatured and then renatured to fold the RNA. Alternatively, one can gently extract RNA from cells (Chillon et al., Methods Enzymol. 2015; 558:3-37) under conditions that maintain the native RNA structure and then perform SHAPE-MaP on this RNA ex vivo. If denatured-and-renatured RNA is used, the RNA is denatured at 95° C. for 2 minutes, snap-cooled on ice for 2 minutes, and then refolded at 37° C. for 30 minutes in 100 mM HEPES (pH 8.0), 100 mM NaCl, and 10 mM MgCl₂.

Various SHAPE reagents are available. In this example, the SHAPE reagent is 1-methyl-7-nitroisatoic anhydride (1M7). 100 to 1000 ng of RNA is used in the SHAPE reaction. The RNA is incubated with 10 mM 1M7 at 37° C. for 3 minutes. Control reactions that lack the SHAPE reagent and contain DMSO rather than 1M7 are performed in parallel. To account for sequence-specific biases in adduct detection, RNAs are modified using 1M7 under strongly denaturing conditions in 50 mM HEPES (pH 8.0), 4 mM EDTA and 50% formamide at 95° C. After modification, RNAs can be purified using either RNA affinity columns (RNeasy Mini Kit; Qiagen) or G-50 spin columns (GE Healthcare).

The treated RNA then undergoes reverse transcription (RT) using primers specific for the target RNA in order to construct a cDNA library by traditional methods. Specifically, enzyme conditions are selected to produce minimal adduct-induced reverse transcription stops and maximal full-length cDNA products. Of the divalent metal ions tested, manganese most effectively promotes enzyme read-through at the sites of bulky 2′-O-adducts. 6 mM Mn²⁺ is used in the RT reaction (0.7 mM premixed dNTPs, 50 mM Tris-HCl (pH 8.0), 75 mM KCl, 6 mM MnCl₂ and 14 mM DTT). The preferred reverse transcriptase enzyme is the Moloney murine leukemia virus reverse transcriptase (Superscript II, Invitrogen). The RT reaction runs for 3 hours, or longer. The reaction product is cleaned up using a G-50 spin column. Double-stranded DNA libraries for massively parallel sequencing are generated using NEBNext sample preparation modules for Illumina sequencing. Second-strand synthesis (NEB E6111) of the cDNA library is performed using 100 ng input DNA, and the library is purified using a PureLink Micro PCR cleanup kit (Invitrogen K310250). End repair of the double-stranded DNA libraries is performed using the NEBNext End Repair Module (NEB E6050). Reaction volumes are adjusted to 100 μl, subjected to a cleanup step (Agencourt AMPure XP beads A63880, 1.6:1 beads-to-sample ratio), d(A)-tailed (NEB E6053) and ligated with Illumina-compatible forked adapters (TruSeq) with a quick ligation module (NEB M2200). Emulsion PCR44 (30 cycles) using Q5 hot-start, high-fidelity polymerase (NEB M0493) is performed to maintain library sample diversity. Resulting libraries are quantified (Qubit fluorimeter; Life Technologies), verified using a Bioanalyzer (Agilent), pooled and subjected to sequencing using the Illumina MiSeq or HiSeq sequencing platform. The SHAPE-MaP sequence data can be analyzed using the ShapeMapper data analysis pipeline as described in Siegfried et al., Nature Methods 2014; 11:959-965.

SHAPE-MaP in Cells

SHAPE-MaP reagents such as 1M7 can be directly added to cells. Individual RNAs can be sequenced following RT-PCR using primers that are specific for the target RNA. Or, a multitude of RNAs can be analyzed by deep sequencing (RNA-seq) of the total SHAPE-MaP transcriptome. Extracted RNA can be analyzed without pull-down or modified RNA can be isolated by pull down of biotin modified RNA by use of streptavidin beads or a streptavidin column.

Besides 1M7, other acylation electrophiles such as 2-methylnicotinic acid imidazolide (NAI) and 2-methyl-3-furoic acid imidazolide (FAI) could be utilized. In this cellular example, NAI is used.

A variety of bacterial, yeast or mammalian cells could be used. Preferably, the cell will be human. Established human cell lines such as HeLa or 293 could be employed. Alternatively, patient-derived cells such as fibroblasts could be used if it is desired that the RNA to be analyzed be in the setting of a disease genotype. In the case of hereditary neurological or musculoskeletal diseases (the TREDs are such examples), patient-derived iPS cells that are differentiated to neurons or muscle cells could be employed. It is also possible to lyse or otherwise rupture the cells just prior to contacting the cells with a compound.

Mammalian cells are grown in the recommended culture medium (typically D-MEM culture medium supplemented with 10% fetal bovine serum, 0.1 mM MEM NonEssential Amino Acids (NEAA), 2 mM L-glutamine, and 1% Penicillin-Streptomyocin). Cells are washed 3× with phosphate-buffered saline (PBS), then scraped and spun down at 700 rpm for 5 minutes at 25° C. Cells (˜3-6×10⁷) are resuspended in PBS and either DMSO (negative control; 10% final concentration) or NAI in DMSO added to the desired final concentration, typically 200 mM. Cell suspensions are placed at 37° C. and reacted for various times. Reactions are then spun down and decanted. To the pelleted cells, 1 mL of Trizol LS (Ambion) is added, followed by 200 ul of chloroform. RNA is precipitated following the Trizol LS manufacturer's instructions. Pellets are washed twice with 70% ethanol and resuspended in 10 ul RNase-free water. Reverse transcription, cRNA library construction, sequencing and data analysis would be performed as described above.

In some cases, RNAs that have reacted with the small molecules can be enriched by pulling down the RNA using a tool such as the streptavidin-biotin system. The strong streptavidin-biotin bond can be used to attach various biomolecules to one another or onto a solid support. Streptavidin can be used for the purification of macromolecules that are tagged by conjugation to biotin. Biotin can be incorporated into the RNA binding small molecule-tether-reactive warhead via click chemistry. In cell-based SHAPE-MaP experiments, the cells are treated with the compound(s) above, RNA is extracted from cells, and the RNA that has reacted is isolated by passing the total RNA over a streptavidin column (can be obtained from Sigma-Aldrich or ThermoFisher Scientific) or through the use of streptavidin magnetic beads (can be obtained from GenScript, EMD Millipore or ThermoFisher Scientific) according to manufacturer's instructions.

### Example 4: Covalent Affinity Transcriptomics

Review of the Basic Concepts

An important feature of the present invention is the tether. The tether links the acylation event to the ligand binding event, thus decisively altering the acylation pattern, which is observed as ‘mutations’ in the sequencing, because only riboses proximal to ligand binding pockets will be acylated. From this we infer the existence of small-molecule binding sites on the targeted RNA as well as the location of those ligand binding sites across the transcriptome. Those RNA ligand/tether/warhead constructs (‘hooks’) that also bear a click functional group can be pulled down clicking to a clickable biotin and then complexing with streptavidin on beads. This click/pull-down protocol enables sequencing of only those RNAs that have been covalently modified by a ‘hook’. SHAPE-MaP & RING-MaP protocols carried out separately on the targeted RNAs enable the building of structural models of targeted RNAs as a framework that will enhance the interpretation of “covalent affinity transcriptomics” sequence data.

Success is measured by bioactivities of free ligands in cells.

Experiments (Compounds and RNA Targets) to Develop the Platform

### Building the Library

The libraries that enable Covalent Affinity Transcriptomics will contain small molecules (“RNA ligands”) tethered to electrophilic warheads that selectively form covalent bonds irreversibly with the 2′-hydroxyl of riboses in the target RNA. The library's diversity encompasses variation in RNA ligand structure, tether structure, and warhead structure.

The RNA ligands are designed based on hypotheses about the structural determinant of RNA affinity and then synthesized and attached to the tether and warhead. As an example, the triptycene series of ligands is designed to bind to three-way junctions (3WJ) in RNA. Alternatively, the RNA ligands are selected from commercially available sources based on their similarity to known RNA ligands or complementarity to RNA binding pockets, purchased, and subjected to further synthesis to attach to the tether and warhead. Examples include but are not limited to: tetracycline antibiotics, aminoglycoside antibiotics, theophylline and similar structures (e.g., xanthines), and ribocil and similar structures, linezolid and similar structures. In a third and complementary approach, libraries of RNA ligands are prepared using combinatorial chemistry techniques. Specifically, the tethers of choice are affixed to polymers that support organic synthesis, and through a series of synthetic chemistry steps, compounds are made in a one-bead-one-compound format. These steps lead to the incorporation in the final RNA ligand a wide range of fragments and reactants connected by a wide range of functional groups. Those compounds are released and the final off-bead step is attachment of the RNA warhead.

As a key element of the library's functional outcome, for each RNA ligand and RNA warhead, a number of structurally diverse tethers are incorporated in order to optimize tether length, tether flexibility, and the ability to tolerate additional functionality (in particular, click functional groups). Specific tethers that are explored include oligoethylene glycols containing one to six ethylene units, oligopeptides that are highly flexible (e.g., oligoglycines or oligo-N-methylglycines containing one to six amino acids) or more rigid (e.g., oligoprolines or oligo-4-hydroxyprolines containing one to six amino acids). Incorporation of click functional groups into the oligoethylene glycol tethers requires insertion of an amino acid, bearing a clickable functional group, at either the RNA ligand or the RNA warhead end of the tether. Incorporation of click functional groups into the oligopeptides tethers simply requires replacing any one of the amino acid residues with an amino acid bearing the clickable functional group.

The RNA warheads will be selected initially based on those specific warheads and related functional groups already demonstrated to acylate RNA at the 2′-OH group on riboses. Such warheads include the isatoic anhydrides, acyl imidazoles, aryl esters (e.g., aspirin) and sulfonyl fluorides. Additional warheads will be identified by (1) synthetic modifications to the aforementioned warheads to establish the structure/activity relationship for RNA warheads as well as (2) screening commercially available electrophiles for their ability to acylate ribose 2′-OH groups. Examples of the latter include beta-lactam antibiotics and related structures, beta-lactones, and electron-poor carbamates known to covalently modify catalytic serines in serine hydrolases.

Click functional groups are selected from the standard ‘toolkit’ of published click reagents and reactants. The present work focuses on azides, alkynes (both terminal and strained), dienes, tetrazines, and dienophiles. When incorporated into the tether segment (mentioned above), it would typically be on the sidechain of an incorporated amino acid. When incorporated into the RNA warhead, more careful and compact design is required with concomitant bespoke synthesis of that enhanced RNA warhead.

### Building the Platform—Type I Hooks

With the ‘hooks’ in hand, the first step is to demonstrate that RNA warheads tethered to RNA ligands yield ribose modifications that reflect tether-constrained proximity to the binding site. This set of results are the basis for further optimization proximity-induced and affinity-induced ribose 2′-OH covalent modification in known RNA/ligand pairs. The binding site and binding mode of tetracycline to both 30S ribosomal RNA [Brodersen et al. Cell 2000, 103, 1143-1154] and to an evolved aptamer [Ferré-D'Amaré et al., Chem & Bio 2008] have been determined by x-ray crystallography. Tetracycline tethered to RNA warheads are studied initially against these two RNAs to demonstrate proximity-induced ribose modification in those RNAs. Triptycene ligands have been demonstrated [Barros & Chenoweth, Angew. Chem. 2014] to bind into shape-complementary cavities in RNA three-way junctions. Triptycene tethered to RNA warheads enables probing of proximal modification in three-way junctions. Both systems (tetracycline and triptycene) are well-controlled based on precedent and structure, enabling similarly well-controlled optimization of tether length and tether rigidity and RNA warhead SAR. These two systems, tetracycline and triptycene, also enable the optimization of sequencing methods in the context of new RNA warheads.

Having demonstrated proximity-induced ribose modification patterns in the isolated model RNAs, the same RNAs are expressed in cells and the optimal ‘hooks’ exposed to those cells, demonstrating the ability of the ‘hooks’ enter the cells, bind the target RNA, and covalently modify it in a pattern substantially the same as in the non-cellular conditions. Initially the sequencing focuses only on the RNA target of interest by using a target-specific primer sequence for the PCR. However, broad PCR and deep sequencing in the same experiment yields a survey of all the RNA in the cell that is also bound by the tetracycline hook or triptycene hook. These data reflect on both the inherent selectivity of the chosen RNA ligands and on the ability to assess selectivity across the transcriptome using the sequencing methods.

Because the ultimate goal is to identify RNA ligands that can be liberated from the ‘hook’ and exhibit cellular biology of interest, the first step is a series of competition experiments: (1) In initial cell-free hook experiments, when free (untethered) RNA ligand is added to the solution, it should compete with its cognate ‘hooks’ for occupancy of the small molecule binding pocket and suppress proximity-induced ribose modification. (2) Similarly, in the cell experiments, the addition of free (untethered) RNA ligand will produce the same competition, though across all the RNAs targeted by the ligand and the cognate ‘hook’.

### Building the Platform—Type II & III Hooks

Having demonstrated proximity-induced covalent modification of ribose 2′-OH, both biochemically and in cells, the same experiments are carried out with “Type II” or “Type III”, which incorporate clickable functionality into the tether or the RNA warhead, respectively. These ‘hooks’ are examined to demonstrate that they recapitulate the results described above, and that the added clickable functional group(s) do no compromise their function as RNA ‘hooks’. After exposure of Type II and Type III ‘hooks’ to RNAs, either biochemically or in cells, the resulting hook/RNA adducts are exposed to the complementary, commercially available click agents that bear biotin. In the first exemplifications, the clickable functional groups on the ‘hooks’ will be azides and the clickable biotins will be strained cyclooctynes that enable copper-free cycloadditions. It is important to monitor the extent of click reaction to ensure that the click reaction reaches completion. In those cases where the experiments are carried out in cells, the cells can be lysed either before or after the click reaction.

The resulting clicked adducts are then exposed to streptavidin on beads and the beads pulled down. After washing away cellular debris and non-adducted RNA, the pulled down RNA can be denatured and sequenced.

### Compounds and Conditions to Pursue a Target of Interest

The molecular etiology of both familial amyotrophic lateral sclerosis (ALS) and frontotemporal dementia (FTD) can be traced to the accumulation, over a series of generations, of the (GGGGCC) hexanucleotide repeat in c9orf72. Selective interdiction of this aberrant RNA in the brain has compelling therapeutic potential. This RNA is an initial and clinically high-value target well suited to the ‘hook’ library technology.

The library described above will be exposed to the c9orf72 hexanucleotide repeat RNA structure in two settings: (1) varying lengths of synthetic RNA in solutions and (2) in diseased cells from patients that express this RNA. These exposures are one ‘hook’ per well. Initial work does not require the clickable ‘hooks’ as sequencing is carried out using target-specific primers. Clickable ‘hooks’ are used as a secondary screen to assess transcriptome-wide selectivity for agents that are determined to bind to the hexanucleotide repeat.

Insofar as there is little to no precedent for molecules that bind to the c9orf72 hexanucleotide repeats, tackling this RNA target requires the breadth of the ‘hook’ library ligand diversity. Furthermore, insofar as the conformation of the target may be strongly influenced by the microenvironment of the cells (e.g., RNA-binding proteins), tackling this RNA target also requires the ability to screen small molecules in cells. Of particular interest will be whether molecules are identified that bind to unique sites on the target or whether the periodicity of the target is retained in its folded form, yielding a periodic series of binding pockets.

Finally, for those ‘hooks’ that yield proximity-induced modification of the c9orf72 RNA target, the RNA ligand segments are resynthesized or re-isolated without tethering to the ‘hook’ constructs and tested for biological activity consistent with binding to the endogenous c9orf72 RNA target.

The same protocol will be brought to bear on several high-value initial targets: UORFs in the 5′-UTR of MYC and other pre-mRNAs, introns in pre-mRNAs, the primary transcript leading to miR-155 (pri-pre-miR-155), and the lncRNAs MALAT-1 and HOTAIR.

### The Omniplex Experiment

It is interesting to note that it is possible to carry out the cell-based screens with a broad and diverse ‘hook’ library in a completely unbiased fashion. In such a case, with sufficient sequencing resources, what is enabled is comprehensive, transcriptome-wide target identification. Thus, in some embodiments of the invention, (1) a library of clickable ‘hooks’ is screened against a cell, (2) in each well all RNA that is ‘hooked’ by the RNA warheads is pulled down and sequenced, and (3) the resulting sequence data can be analyzed to find all targets addressed by all the ligands in the ‘hook’ library used.

### Example 5: Synthesis of Warhead Type 1A

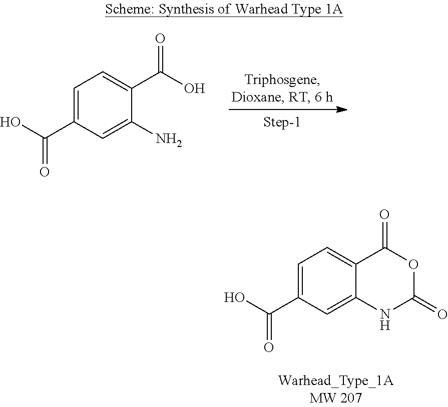 [SMILES: Nc1cc(C(=O)O)ccc1C(=O)O ; O=C(O)c1ccc2c(=O)oc(=O)[nH]c2c1]

### 2,4-dioxo-1,4-dihydro-2H-benzo[d][1,3]oxazine-7-carboxylic acid, Warhead Type 1A

To a solution of 2-aminoterephthalic acid (2.0 g, 11.05 mmol) in 1,4-Dioxane (160 mL) was added triphosgene (3.28 g, 11.05 mmol) at room temperature. The resulting reaction mixture was stirred for 6 h at room temperature. The reaction mixture was poured in DM water (400 mL) and extracted with ethyl acetate (3×150 mL). The organic layers were combined, washed with brine and concentrated under reduced pressure to afford Warhead_Type_1A (2.2 g, 96.2%) as an off white solid. ¹H NMR (400 MHz, DMSO-d₆) δ 13.67 ppm (1H, broad), 11.89 ppm (1H, broad), 8.03-8.01 ppm (1H, d), 7.73-7.68 ppm (2H, m). MS (ESI-MS): m/z calcd for C₉H₅NO₅ [MH]⁻ 206.02, found 206.17.

### Example 6: Synthesis of Warhead Type 1B

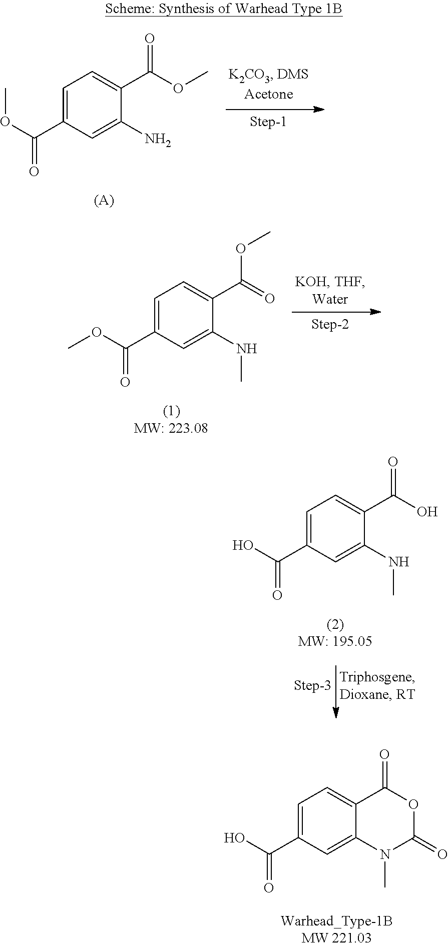

### 1,4-dimethyl 2-(methylamino)benzene-1,4-dicarboxylate (1)

To a solution of dimethyl 2-aminobenzene-1,4-dicarboxylate (10.0 g, 0.05 mol) in acetone (150 mL) was sequentially added potassium carbonate (19.8 g, 0.143 mol) and dimethylsulphate (18.1 g, 0.143 mol) at room temperature. The resulting reaction mixture was stirred at 60° C. for 24 h. The reaction mixture was slowly cooled to room temperature and diluted with water (200 mL). The resulted mixture was then extracted with ethyl acetate (4×750 mL). The organic layers were combined, washed with brine and concentrated under reduced pressure to get crude 1 as a brown solid. The crude mixture was purified by column chromatography on silica gel (7% EtOAc/hexanes) to yield 1 (4.5 g, 42%) as a pale yellow solid. MS (ESI-MS): m/z calcd for C₁₁H₁₃NO₄ [MH]⁺ 224.08, found 224.2.

### 2-(methylamino)benzene-1,4-dicarboxylic Acid (2)

To a solution of dimethyl 2-(methylamino)benzene-1,4-dicarboxylate (1) (4.5 g, 0.02 mol) in THE (100 mL) and water (50 mL) was added potassium hydroxide (3.4 g, 0.06 mol) at room temperature. The resulting reaction mixture was stirred at 70° C. for 4 h. The reaction mixture was cooled to room temperature, diluted with water (200 mL) and acidified using potassium bisulfate. The resulted mixture was then extracted with ethyl acetate (4×75 mL). The organic layers were combined, washed with brine and concentrated under reduced pressure to get crude 2 (3.0 g, 76.33%) as a buff white solid. The crude mixture was used in next step without further purification. ¹H NMR (400 MHz, DMSO-d₆) δ 13.14 ppm (1H, s), 7.87-7.85 ppm (1H, d, J=8.0 Hz), 7.21-7.21 ppm (1H, d, J=1.6 Hz), 7.10-7.07 (1H, dd, J=8.0), 2.87 (1H, s). MS (ESI-MS): m/z calcd for C₉H₉NO₄ [MH]⁺ 196.05, found 196.21.

### 1-methyl-2,4-dioxo-2,4-dihydro-1H-3,1-benzoxazine-7-carboxylic Acid, Warhead Type 1B

To a suspension of 2-(methylamino) benzene-1,4-dicarboxylic acid (2) (3.0 g, 0.015 mol) in tetrahydrofuran (90 mL) was added triphosgene (2.28 g, 0.076 mol) at room temperature. The resulting reaction mixture was stirred at 30° C. for 30 min. The reaction mixture was cooled to room temperature, diluted with water (50 mL) and extracted with ethyl acetate (3×100 mL). The organic layers were combined, washed with brine and concentrated under reduced pressure to get crude Warhead Type 1B as a yellow solid. The crude mixture was purified by trituration using diethyl ether to yield Warhead Type 1B (3.1 g, 91.17%) as yellow solid. ¹H NMR (400 MHz, DMSO-d₆) δ 13.78 ppm (1H, s), 8.12-8.09 (1H, d, J=8.4), 7.82-7.80 (2H, m), 3.51 (3H, S). MS (ESI-MS): m/z calcd for C₁₀H₇NO₅ [MH]⁻ 220.03, found 220.07.

Additional warheads similar to this type include N-methylisatoic anhydride, 1-methyl-6-nitroisatoic anhydride, and 1-methyl-7-nitroisatoic anhydride. These are commercially available.

### Example 7: Synthesis of Warhead Type 2

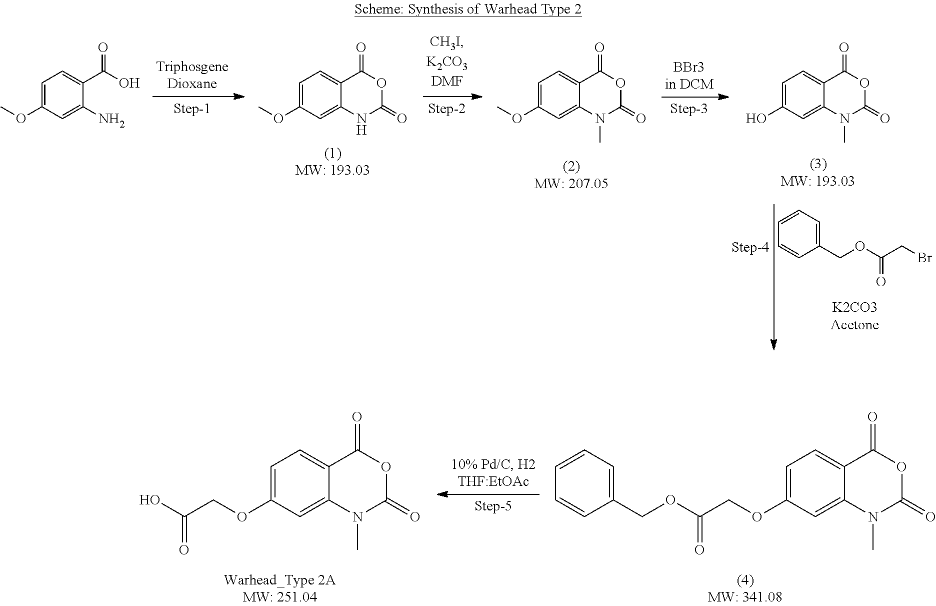

### 7-methoxy-2H-benzo[d][1,3]oxazine-2,4(1H)-dione (1)

To a solution of 2-amino-4-methoxybenzoic acid (20 g, 119.73 mmol) in 1,4-dioxane (400 mL) was added triphosgene (17.8 g, 59.86 mmol) at room temperature. The resulting reaction mixture was stirred at room temperature for 6 h. The reaction mixture was poured in DM water (1 L) and extracted with ethyl acetate (3×350 mL). The organic layers were combined, washed with brine and concentrated under reduced pressure to afford 1 (20.5 g, 88%) as off white solid. ¹H NMR (400 MHz, DMSO-d₆) δ 11.66 ppm (1H, broad), 7.85-7.83 ppm (1H, d, J=8.8 Hz), 6.85-6.83 ppm (1H, dd, J=2.4, 6.4 Hz), 6.59-6.58 ppm (1H, d, J=2.4 Hz), 3.86 ppm (3H, s). MS (ESI-MS): m/z calcd for C₉H₇NO₄ [MH]⁻ 192.04, found 192.16.

### 7-methoxy-1-methyl-2H-benzo[d][1,3]oxazine-2,4(1H)-dione (2)

To a solution of 7-methoxy-2H-benzo[d][1,3]oxazine-2,4(1H)-dione (1) (20.5 g, 106.2 mmol) in N,N-dimethyl formamide (200 mL) was added K₂CO₃ (14.65 g, 106.2 mmol) at room temperature and the resulting reaction mixture was stirred for 10 min. To this, methyl iodide (18.08 g, 127.44 mmol) was added drop wise at room temperature. The reaction mixture was poured into DM water (1 L) and extracted with ethyl acetate (3×350 mL). The organic layers were combined, washed with brine and concentrated under reduced pressure to get crude 2. The crude was purified by triturating with hexane to yield 2 (17.9 g, 93.23%) as off white solid. The product was used in the next step without further purification. ¹H NMR (400 MHz, DMSO-d₆) δ 7.95-7.93 ppm (1H, d, J=8.4 Hz), 6.94-6.91 ppm (1H, dd, J=2.4, 6.4 Hz), 6.86-6.85 ppm (1H, d, J=2 Hz), 3.94 ppm (3H, s), 3.46 ppm (3H, s). MS (ESI-MS): m/z calcd for C₁₀H₉NO₄ [MH]⁺ 208.05, found 208.2.

### 7-hydroxy-1-methyl-2H-benzo[d][1,3]oxazine-2,4(1H)-dione (3)

To a solution of 7-methoxy-1-methyl-2H-benzo[d][1,3]oxazine-2,4(1H)-dione (2) (10 g, 48.30 mmol) in dichloromethane (500 mL) at 0° C., BBr₃ (1 M solution in dichloromethane) (72.44 mL, 72.44 mmol) was added dropwise. The resulting reaction mixture was stirred at 0° C. for 1 h and slowly brought to room temperature and further stirred for 24 h. The reaction mixture was diluted with n-Hexane (500 mL) and the residues obtained were filtered. The collected solid was washed with n-Hexane (3×50 mL) and dried under reduced pressure. The solid was further suspended in water (1 L) and extracted with dichloromethane (5×350 mL). The organic layers were combined, washed with brine and concentrated under reduced pressure to get 3 (7.9 g, 84.74%) as a brown solid. ¹H NMR (400 MHz, MeOD) δ 7.96-7.94 ppm (1H, d, J=8.8 Hz), 6.78-6.75 ppm (1H, dd, J=2, 6.4 Hz), 6.69-6.69 ppm (1H, d, J=2.4 Hz), 3.52 ppm (3H, s). MS (ESI-MS): m/z calcd for C₉H₇NO₄ [MH]⁻ 192.04, found 191.96.

### Benzyl 2-((1-methyl-2,4-dioxo-1,4-dihydro-2H-benzo[d][1,3]oxazin-7-yl)oxy)acetate (4)

To a solution of 7-hydroxy-1-methyl-2H-benzo[d][1,3]oxazine-2,4(1H)-dione (3) (7.9 g, 40.93 mmol) in acetone (800 mL) was added K₂CO₃ (14.12 g, 102.315 mmol) and the reaction mixture was stirred for 20 min at room temperature. To this, benzyl-2-bromoacetate (11.251 g, 49.111 mmol) was added dropwise at room temperature and the resulting reaction mixture was further stirred for 5 h. The reaction mixture was filtered and residues collected were washed with acetone (3×20 mL). The filtrate was concentrated under reduced pressure to afford a solid mass. The solid mass was dissolved in ethyl acetate (1 L) and washed with water (3×300 mL). The organic layers were combined, washed with brine and concentrated under reduced pressure to get crude 4. The crude mixture was purified by column chromatography on silica gel (20% EtOAc/n-Hexane) to yield pure 4 (0.39 g, 62.9%) as a yellow oil. ¹H NMR (400 MHz, DMSO-d₆) δ 7.94-7.92 ppm (1H, d, J=8.4 Hz), 7.38-7.35 ppm (5H, m), 6.95-6.92 ppm (1H, dd, J=2, 6.8 Hz), 6.87-6.87 ppm (1H, d, J=2 Hz), 5.23 ppm (2H, s), 5.14 ppm (2H, s), 3.40 ppm (3H, s). MS (ESI-MS): m/z calcd for C₁₈H₁₅NO₆ [MH]⁺ 342.09, found 342.28.

### 2-((1-methyl-2,4-dioxo-1,4-dihydro-2H-benzo[d][1,3]oxazin-7-yl)oxy)acetic Acid, Warhead_type_2

To a suspension of 10% Pd/C (dry basis) (1.25 g, 5% w/v) in a 1:1 mixture of THF: EtOAc (400 mL) was added a solution of Benzyl 2-((1-methyl-2,4-dioxo-1,4-dihydro-2H-benzo[d][1,3]oxazin-7-yl)oxy)acetate (4) (6.5 g, 19.057 mmol) at room temperature. H₂ gas was purged into the reaction mixture for 3 h at room temperature. The reaction mixture was filtered through a celite bed and the collected filtrate was concentrated under reduced pressure to afford crude Warhead_type_2. The crude mixture was purified by triturating with n-Hexane (3×20 mL) to yield Warhead_type_2 (0.39 g, 62.9%) as an off white solid. ¹H NMR (400 MHz, DMSO-d₆) δ 13.25 ppm (1H, br s), 7.95-7.92 ppm (1H, d, J=8.4 Hz), 6.92-6.88 ppm (2H, m), 4.94 ppm (2H, s), 3.44 ppm (3H, s). MS (ESI-MS): m/z calcd C₁₁H₉NO₆ [MH]⁺ 252.04, found 252.47.

### Example 8: Synthesis of ARK-1 (Ark000007)

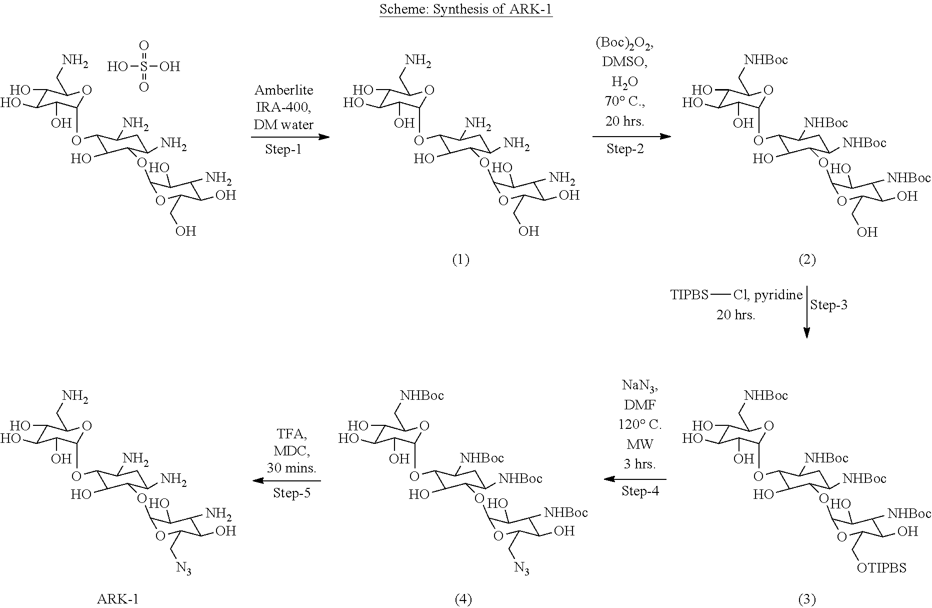

Kanamycin a Free Base, 1.

In 250 mL beaker, kanamycin A monosulfate (5.0 g, 8.582 mmol) was dissolved in water (100 mL) and the resulting aqueous solution was passed through Amberlite® IRA-400 —OH form ion exchange resin. The free base was eluted using DM water and the fractions collected were lyophilized to obtain free base 1 (3.8 g, 91%) as a white solid which was used without further purification. MS (ESI-MS): m/z calcd for C₁₈H₃₆N₄O₁₁ [MH]⁺ 485.23, found 485.26.

### 1,3,6′,3″-tetra-N-(tert-butoxycarbonyl) kanamycin A, 2

To a stirred solution of Kanamycin A free base (1) (3.7 g, 7.641 mmol) in DMSO (140 mL) and water (40 L) (180 mL) was added Boc anhydride (20 g, 91.692 mmol) at room temperature and the resulting reaction mixture was heated at 70° C. for 20 h. After cooling to room temperature, an aqueous solution of NH₄OH (30 mL) was added to the resulting reaction mixture, resulting in a precipitate. The precipitate was collected through filtration, washed with water (2×350 mL) and dried under reduced pressure to afford pure 2 (5.7 g, 84%) as a white solid. ¹H NMR (400 MHz, DMSO-d₆) δ 6.92 ppm (1H, s), 6.62 ppm (1H, s), 6.53-6.51 ppm (1H, d, J=6.8 Hz), 6.38 ppm (1H, s), 5.40 ppm (1H, broad s), 5.27 ppm (1H, broad s), 4.71 ppm (1H, broad s), 4.22 ppm (1H, broad s), 3.80-3.25 ppm (15H, broad m), 3.07 ppm (1H, broad s), 1.82-1.75 ppm (1H, broad s), 1.37 ppm (36H, broad s); MS (ESI-MS): m/z calcd for C₃₈H₆₈N₄O₁₉ [MH]⁺ 885.44, found 907.7 (M+Na adduct).

### 6″-(2,4,6-Triisopropylbenzenesulfonyl)-1,3,6′,3″-tetra-N-(tert-butoxycarbonyl) kanamycin A, 3

To a stirred solution of 1,3,6′,3″-Tetra-N-(tert-butoxycarbonyl) kanamycin A (2) (2 g, 2.261 mmol) in pyridine (35 mL) was added a solution of 2,4,6-triisopropylbenzenesulfonyl chloride (4.11 g, 13.567 mmol) in pyridine (4 mL) at room temperature. The resulting reaction mixture was stirred at room temperature for 20 h. After this, the reaction mixture was added methanol (30 mL) and further stirred for 30 min. The reaction mixture was then poured into a cooled 10% HCl solution (400 mL) and extracted with ethyl acetate (4×200 mL). The organic layers were combined, washed with brine, dried using anhydrous Na₂SO₄ and concentrated under reduced pressure to get crude 3 as a yellow solid. The crude mixture was purified by column chromatography on silica gel (2% MeOH/chloroform) to get pure 3 (0.5 g, 73%) as a light yellow solid. MS (ESI-MS): m/z calcd for C₅₃H₉₀N₄O₂₁S [MH]⁺ 1151.58, found 908.6 (M-TIPBS fragment+Na adduct).

### 6″-Azido-1,3,6′,3″-tetra-N-(tert-butoxycarbonyl)kanamycin A, 4

A 35 mL pressure vial was charged with 6″-(2,4,6-Triisopropylbenzenesulfonyl)-1,3,6′,3″-tetra-N-(tert-butoxycarbonyl) kanamycin A (3) (0.5 g, 0.434 mmol), NaN₃ (0.565 g, 8.691 mmol), DMF (15 mL) at room temperature. The resulting reaction mixture was irradiated under microwave at 120° C. for 3 h. After cooling to room temperature, the reaction mixture was quenched with cold water (150 mL) and extracted with ethyl acetate (3×50 mL). The organic layers were combined, washed with brine, dried using anhydrous Na₂SO₄ and concentrated under reduced pressure to get crude 4 as brown oil. The crude mixture was purified by preparative HPLC using the following method to get pure 4 (0.11 g, 27%) as a light yellow solid. ¹H NMR (400 MHz, CD₃OD) δ 5.11-5.02 ppm (2H, t, J=9.6 Hz), 4.37-4.35 ppm (1H, d), 3.73-3.36 ppm (15H, m), 3.23-3.18 ppm (1H, t, J=9.2 Hz), 2.07-2.04 ppm (1H, d, J=13.2 Hz), 1.47-1.45 ppm (36H, br s). MS (ESI-MS): m/z calcd for C₃₈H₆₇N₇O₁₈ [MH]⁺ 910.45, found 932.67 (M+Na adduct).

Method of Preparative HPLC:

(A) 10 mM ammonium bicarbonate in H₂O (HPLC grade) and (B) MeCN:IPA (90:10) (HPLC grade), using X-BRIDGE C18, 250*19 mm, 5 Un with a flow rate of 19.0 mL/min and with the following gradient:

| Time | % A | % B |
|---|---|---|
| 0.01 | 60.0 | 40.0 |
| 17.00 | 35.0 | 65.0 |
| 17.01 | 0.0 | 100.0 |
| 21.00 | 0.0 | 100.0 |
| 21.01 | 60.0 | 40.0 |
| 22.00 | 60.0 | 40.0 |

### 6″-Azido-kanamycin A trifluoroacetate salt, ARK-1-TFA Salt

6″-Azido-1,3,6′,3″-tetra-N-(tert-butoxycarbonyl)kanamycin A, (4) (0.11 g, 0.121 mmol) was dissolved in 1:1 mixture of DCM:TFA (3.2 mL) and the resulting solution was stirred at room temperature for 30 min. The reaction mixture was concentrated under reduced pressure and triturated using diethyl ether to get pure ARK-1-TFA SALT (0.12 g, 102%) as a light yellow solid. ¹H NMR (400 MHz, D₂O) δ 5.39-5.38 ppm (1H, d, J=3.6 Hz), 4.95-4.94 ppm (1H, d, J=3.2 Hz), 3.796-3.71 ppm (5H, m), 3.64-3.31 ppm (11H, m), 3.07-3.01 ppm (1H, q, J=14.4, 9.2 Hz), 2.40-2.37 ppm (1H, m), 1.77-1.74 ppm (1H, q, J=12.8 Hz), 1.09-1.02 ppm (1H, m). MS (ESI-MS): m/z calcd for C₁₈H₃₅N₇O₁₀+3TFA [MH]⁺ 509.24, found 510.4. HPLC retention time: 7.103 min.

### 6″-Azido-kanamycin A hydrochloride salt, ARK-1-HCl SALT (Ark000007)

6″-Azido-kanamycin A trifluoroacetate salt, ARK-1-TFA SALT (0.12 g, 0.124 mmol) was dissolved in water (40 mL) and the resulting aqueous solution was passed through Amberlite® IRA-400 —OH form ion exchange resin. The free base was eluted using DM water and the fractions collected were lyophilized to obtain ARK-1 as a free base. The free base was dissolved in 0.01 N HCl (4 mL) and the resulting solution was lyophilized to obtain pure ARK-1-HCl-SALT (0.06 g, 77%) as a yellow solid. ¹H NMR (400 MHz, D₂O) δ 5.41-5.40 ppm (1H, d, J=2.4 Hz), 4.96 ppm (1H, br s), 3.90-3.76 ppm (5H, m), 3.62-3.60 ppm (2H, d, J=8.8 Hz), 3.55-3.19 ppm (10H, m), 3.07-3.01 ppm (1H, m), 2.41-2.38 ppm (1H, d, J=12), 1.82-1.73 ppm (1H, q, J=12.8 Hz). MS (ESI-MS): m/z calcd for C₁₈H₃₅N₇O₁₀.3 HCl [MH]⁺ 510.24, found 510.2. HPLC retention time: 14.897 min.

### Example 9: Synthesis of ARK-7 (Ark0000013)

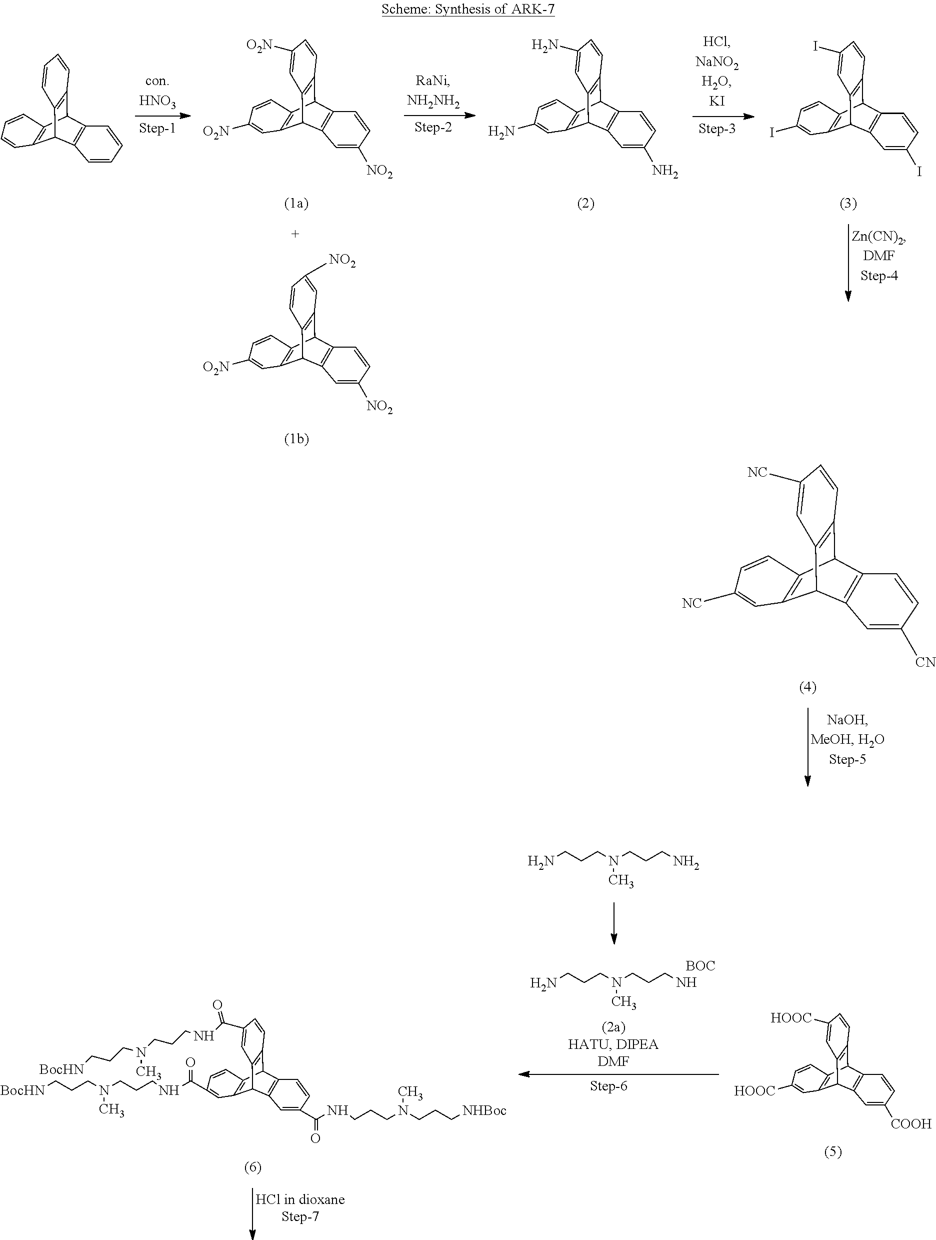

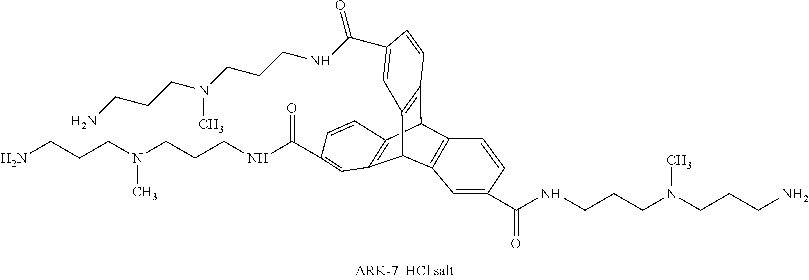 [SMILES: CN(CCCN)CCCNC(=O)c1ccc2c(c1)C1c3cc(C(=O)NCCCN(C)CCCN)ccc3C2c2ccc(C(=O)NCCCN(C)CCCN)cc21]

### 2,7,15-trinitro-9,10-dihydro-9,10-[1,2]benzenoanthracene, 1a

Concentrated HNO₃ (400 mL) was added dropwise to triptycene (10 g, 39.3 mmol) at room temperature and the resulting reaction mixture was heated at 80° C. for 16 h. The resulting brown solution was allowed to cool to room temperature, poured into ice cold water (3000 mL) and stirred for 30 min. The obtained precipitates were collected, washed with cold water, and then dried in air to get the crude mixture of 1a and 1b. The crude mixture was purified by flash column chromatography on silica gel (20% EtOAc/hexanes) to afford pure product 1a (2.23 g, 14.10%) as a white solid. 1a mp: >300° C. ¹H NMR (400 MHz, CDCl₃) δ 8.37-8.36 ppm (3H, d, J=2 Hz), 8.08-8.06 ppm (3H, dd, J=8 Hz, J=2 Hz), 7.66-7.64 ppm (3H, d, J=8.4 Hz), 5.87 ppm (1H, S), 5.84 ppm (1H, s), ¹³C NMR (400 MHz, DMSO-d₆) 150.24, 145.91, 145.76, 126.10, 122.60, 119.93, 52.18, 51.48; MS (ESI-MS): m/z calcd for C₂₀H₂₁N₃O₆ [MH]⁺ 390.06, No mass response observed.

1b mp: 178-180° C. ¹H NMR (400 MHz, CDCl₃) δ 8.36-8.35 ppm (3H, m), 8.09-8.06 ppm (3H, m), 7.69-7.65 ppm (3H, m), 5.86 ppm (1H, s), 5.85 ppm (1H, s)¹³C NMR 150.93, 150.57, 145.72, 145.33, 144.92, 125.97, 122.54, 119.93, 55.33, 51.98, 51.74.

### 9,10-dihydro-9,10-[1,2]benzenoanthracene-2,7,15-triamine, 2

To a solution of 2,7,15-trinitro-9,10-dihydro-9,10-[1,2]benzenoanthracene (1a) (2.23 g, 5.73 mmol) in THE (100 mL) was added Raney Nickel (1.0 g) and the resulted reaction mixture was cooled to 0° C. Hydrazine hydrate (4 mL) was added to the resulting mixture at 0° C. The reaction mixture was stirred at 60° C. for 1 h. The resulting reaction mixture was allowed to cool to room temperature and filtered through celite eluting with THF. The filtrate was concentrated under reduced pressure to afford crude 2 (1.5 g, 88.23%) as a brown solid which was used without further purification. ¹H NMR (400 MHz, CDCl₃) δ 7.09-7.07 ppm (3H, d, J=7.6 Hz), 6.75-6.75 ppm (3H, d, J=2 Hz), 6.29-6.27 ppm (3H, dd, J=7.6 Hz, J=2 Hz), 5.10 ppm (1H, S), 5.02 ppm (1H, s), 3.51-3.35 ppm (6H, broad s). MS (ESI-MS): m/z calcd for C₂₀H₁₇N₃[MH]⁺ 300.14, found 300.4.

### 2,7,15-triiodo-9,10-dihydro-9,10-[1,2]benzenoanthracene, 3

In 100 mL round bottom flask, 9,10-dihydro-9,10-[1,2]benzenoanthracene-2,7,15-triamine (2) (0.9 g, 3.01 mmol) was dissolved in concentrated hydrochloric acid (7.5 mL) and water (15 mL) and the resulting solution was cooled to 0° C. To this, a solution of sodium nitrite (0.72 g, 10.5 mmol) in water (7.5 mL) was added dropwise over 10 min and the resulting reaction mixture was stirred for 20 min at 0° C. After this, a solution of potassium iodide (3.74 g, 22.58 mmol) in water (10 mL) was added drop wise to the reaction mixture at 0° C. and further stirred for 5 min. The reaction mixture was then slowly warmed to room temperature and heated at 80° C. for 2 h. After cooling to room temperature, the reaction mixture was diluted with water (50 mL) and extracted with dichloromethane (3×25 mL). The organic layers were combined, washed with saturated sodium bisulfate (3×30 mL), dried using anhydrous Na₂SO₄ and concentrated under reduced pressure to get crude 3 as a brown semisolid. The crude mixture was purified by flash column chromatography on silica gel (5% EtOAc/hexanes) to get pure product 3 (0.57 g, 30.0%) as yellow solid. ¹H NMR (400 MHz, CDCl₃) δ 7.74-7.73 ppm (3H, d, J=1.6 Hz), 7.39-7.36 ppm (3H, dd, J=7.6 Hz, J=1.6 Hz), 7.66-7.64 ppm (3H, d, J=7.6 Hz), 5.31 ppm (1H, S), 5.26 (1H, s).

### 9,10-dihydro-9,10-[1,2]benzenoanthracene-2,7,15-tricarbonitrile, 4

To a solution of 2,7,15-triiodo-9,10-dihydro-9,10-[1,2]benzenoanthracene (3) (0.55 g, 0.87 mmol) in DMF (5 mL) was added zinc cyanide (0.33 g, 2.79 mmol) and the resulting reaction mixture was degassed with nitrogen gas for 20 min. To this, tetrakis (0.10 g, 0.1 mmol) was added and the resulting reaction mixture was stirred at 140° C. for 16 h. After cooling to room temperature, the reaction mixture was filtered through celite, quenched with cold water (20 mL) and extracted with dichloromethane (3×30 mL). The organic layers were combined, washed with brine, dried using anhydrous Na₂SO₄ and concentrated under reduced pressure to get crude 4 as a brown semisolid. The crude mixture was purified by flash column chromatography on silica gel (25% EtOAc/hexanes) to get pure product 4 (0.2 g, 70.0%) as light yellow solid. ¹H NMR (400 MHz, CDCl₃) δ 7.74-7.74 ppm (3H, d, J=1.2 Hz), 7.39-7.36 ppm (3H, dd, J=7.6 Hz, J=1.6 Hz), 7.66-7.64 ppm (3H, d, J=7.6 Hz), 5.31 ppm (1H, S), 5.26 (1H, s).

### 9,10-dihydro-9,10-[1,2]benzenoanthracene-2,7,15-tricarboxylic Acid, 5

To a solution of 9,10-dihydro-9,10-[1,2]benzenoanthracene-2,7,15-tricarbonitrile (4) (0.40 g, 1.22 mmol) in MeOH (5 mL) was added 15% aqueous NaOH solution (5 mL, 18.24 mmol) at room temperature and the resulting reaction mixture was stirred at 60° C. for 16 h. After cooling to room temperature, excess of MeOH was removed under reduced pressure and the resulting mixture was poured in ice-cold water (50 mL). The pH of this aqueous solution was adjusted to ˜2 using 1 N HCl and the residues obtained were collected through filtration to get crude 5 (0.30 g, 65.3%) as a white solid which was used without further purification. ¹H NMR (400 MHz, MeOD) δ 8.12 ppm (3H, d, J=1.2 Hz), 7.79-7.77 ppm (3H, dd, J=7.6 Hz, J=1.6 Hz), 7.58-7.56 ppm (3H, d, J=4 Hz), 5.832 ppm (2H, S); MS (ESI-MS): m/z calcd for C₁₂H₂₆O₆ [MH]-385.07, found 385.1.

### Tert-butyl (3-((3-aminopropyl)(methyl)amino)propyl)carbamate, 2a

To a solution of N¹-(3-aminopropyl)-N¹-methylpropane-1,3-diamine (5 g, 38.48 mmol) in THF (10 mL) at 0° C. was added Boc anhydride (1.50 g, 6.89 mmol) dropwise over a period of 20 min and the resulting reaction mixture was stirred at room temperature for 16 h. THE was removed under reduced pressure and the resulting mixture was poured in water (50 mL). The aqueous mixture was extracted with ethyl acetate (3×30 mL). The organic layers were combined, washed with water, dried using anhydrous Na₂SO₄ and concentrated under reduced pressure to get pure 2a (1.3 g, 15.4%) as a colorless oil. ¹H NMR (400 MHz, d₆-DMSO) δ 6.80-6.79 ppm (1H, d, J=4 Hz), 3.17 (3H, broad s) 2.94-2.89 ppm (2H, dd, J=12.4, 6 Hz), 2.51 ppm (2H, broad s), 2.28-2.21 ppm (4H, m), 2.08-2.07 (2H, d, J=4 Hz), 1.50-1.44 ppm (4H, m), 1.37 (9H, s); MS (ESI-MS): m/z calcd for C₁₂H₂₆N₂O₂ [MH]⁺ 246.21, No mass response observed.

### N²,N⁷,N¹⁵-tris(3-((3-tert-butylcarbonylaminopropyl)(methyl)amino)propyl)-9,10-dihydro-9,10-[1,2]benzenoanthracene-2,7,15-tricarboxamide, 6

To a solution of tert-butyl (3-((3-aminopropyl)(methyl)amino)propyl)carbamate (2a) (0.71 g, 2.91 mmol) in DMF (3 mL) was added 9,10-dihydro-9,10-[1,2]benzenoanthracene-2,7,15-tricarboxylic acid (0.35 g, 0.91 mmol), HATU (1.1 g, 2.91 mmol), DIPEA (1.0 mL, 5.82 mmol) and the resulting reaction mixture was stirred at room temperature for 2 h. The reaction mixture was poured in water (50 mL) and extracted with dichloromethane (3×25 mL). The organic layers were combined, washed with brine, dried using anhydrous Na₂SO₄ and concentrated under reduced pressure to get crude 6 as brown oil. The crude mixture was purified by preparative HPLC using the following method to afford pure product 6 (0.2 g, 20.7%) as light yellow solid. ¹H NMR (400 MHz, d₆-DMSO) δ 8.40-8.37 (3H, t, J=5.2 Hz), 7.93 (3H, s) 7.55-7.49 ppm (6H, dd, J=16, 7.6 Hz), 6.78 ppm (3H, broad s), 5.87 ppm (2H, broad s), 3.23-3.21 ppm (6H, m), 2.93-2.90 (6H, m), 2.30-2.22 (12H, m), 1.61-1.58 (6H, m), 1.50-1.46 (6H, m), 1.31 (27H, s). MS (ESI-MS): m/z calcd for C₅₉H₈₉N₉O₉ [MH]⁺ 1068.68, found 1068.9.

Method of Preparative HPLC:

(A) 10 mM NH₄HCO₃ in water (B) MeCN:MeOH:IPA (65:25:10), using WATERS X-BRIDGE C18 250 mm*19 mm, 5.0 μM with the flow rate of 15.0 mL/min and with the following gradient:

| Time | % A | % B |
|---|---|---|
| 0.01 | 75.0 | 25.0 |
| 23.00 | 30.0 | 70.0 |
| 23.01 | 0.0 | 100.0 |
| 24.00 | 0.0 | 100.0 |
| 24.01 | 75.0 | 25.0 |
| 25.00 | 75.0 | 25.0 |

### N²,N⁷,N¹⁵-tris(3-((3-aminopropyl)(methyl)amino)propyl)-9,10-dihydro-9,10-[1,2]benzenoanthracene-2,7,15-tricarboxamide, ARK-7

To a solution of N²,N⁷,N¹⁵-tris(3-((3-tert-butylcarbonylaminopropyl)(methyl)amino)propyl)-9,10-dihydro-9,10-[1,2]benzenoanthracene-2,7,15-tricarboxamide (6) (0.2 g) in 1,4-dioxane (5 mL) was added 4 M HCl in dioxane (1 mL) at room temperature and the resulting reaction mixture was stirred for 2 hours. The mixture was concentrated under reduced pressure to get pure hydrochloride salt of ARK-7 (0.072 g, 50.3%) as a light yellow solid. ¹H NMR (400 MHz, D₂O) δ 7.70 ppm (3H, s), 7.42-7.40 ppm (3H, d, J=7.6 Hz), 7.34-7.32 ppm (3H, d, J 8 Hz), 5.73 ppm (1H, s), 5.71 (1H, s), 3.34-3.30 ppm (6H, t), 3.23-3.03 ppm (12H, m), 2.97-2.93 ppm (6H, t), 2.76 ppm (9H, s), 2.06-1.92 ppm (12H, m), MS (ESI-MS): m/z calcd for C₄₄H₆₅N₉O₃ [MH]⁺ 768.52, found 768.7. HPLC retention time: 4.277 min.

### Example 10: Synthesis of ARK-8 (Ark0000014)

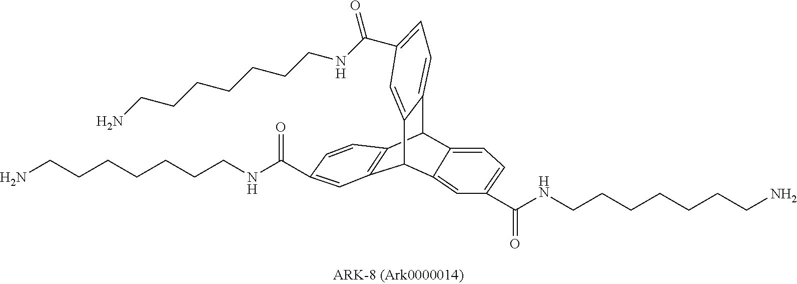 [SMILES: NCCCCCCCNC(=O)c1ccc2c(c1)C1c3cc(C(=O)NCCCCCCCN)ccc3C2c2ccc(C(=O)NCCCCCCCN)cc21]

ARK-8 was synthesized following the method for ARK-7 above to provide intermediate 5. This was then coupled with intermediate 2a below and converted to ARK-8 as described below.

### Tert-butyl (7-aminoheptyl)carbamate, 2a

To a solution of heptane-1,7-diamine (5 g, 38.46 mmol) in THF (10 mL) at 0° C. was added Boc anhydride (1.68 g, 7.69 mmol) dropwise over a period of 20 min and the resulting reaction mixture was stirred at room temperature for 16 h. THE was removed under reduced pressure and the resulting mixture was poured into water (50 mL). The aqueous mixture was extracted with ethyl acetate (3×25 mL). The organic layers were combined, washed with water, dried using anhydrous Na₂SO₄ and concentrated under reduced pressure to get pure 2a (1 g, 11.3%) as a colourless oil. ¹H NMR (400 MHz, CDCl₃) δ 6.80-6.77 (1H, t, J=5.2 Hz), 2.91-2.85 (2H, dd, J=13.2, 6.8 Hz) 2.55-2.44 ppm (2H, m), 1.36 ppm (11H, s), 1.31 ppm (4H, s), 1.23 (6H, s), MS (ESI-MS): m/z calcd for C₁₂H₂₆N₂O₂ [MH]⁺ 231.20, found 231.5.

### N²,N⁷,N¹⁵-tris(7-tert-butylcarbonylaminoheptyl)-9,10-dihydro-9,10-[1,2]benzenoanthracene-2,7,15-tricarboxamide, 6

To a solution of Tert-butyl (7-aminoheptyl)carbamate (2a) (0.51 g, 2.24 mmol) in DMF (3 mL) was added 9,10-dihydro-9,10-[1,2]benzenoanthracene-2,7,15-tricarboxylic acid (0.27 g, 0.70 mmol), HATU (0.85, 2.24 mmol), DIPEA (0.77 mL, 4.47 mmol) and the resulting reaction mixture was stirred at room temperature for 2 h. The reaction mixture was poured into water (50 mL) and extracted with dichloromethane (3×25 mL). The organic layers were combined, washed with brine, dried using anhydrous Na₂SO₄ and concentrated under reduced pressure to get crude 6 as a brown semisolid. The crude mixture was purified by flash column chromatography on silica gel (0.5% MeOH/chloroform) to afford pure product 6 (0.65 g, 91.5%) as light yellow solid. ¹H NMR (400 MHz, DMSO) δ 8.34-8.32 (3H, d, J=8.8 Hz), 7.93 (3H, s) 7.53 ppm (6H, s), 6.75 ppm (3H, broad s), 5.87 ppm (1H, s), 5.76 ppm (1H, s), 3.20-3.14 (6H, d, J=24 Hz), 2.29 (6H, s), 1.37 (27H, s), 1.25-1.24 (30H, m), MS (ESI-MS): m/z calcd for C₅₉H₈₆N₆O₉ [MH]⁺ 1023.65, found 1045.5 (M+23).

### N²,N⁷,N¹⁵-tris(7-aminoheptyl)-9,10-dihydro-9,10-[1,2]benzenoanthracene-2,7,15-tricarboxamide, ARK-8

To a solution of N²,N⁷,N¹⁵-tris(7-tert-butylcarbonylaminoheptyl)-9,10-dihydro-9,10-[1,2]benzenoanthracene-2,7,15-tricarboxamide (6) (0.7 g) in 1,4-dioxane (5 mL) was added 4 M HCl in dioxane (3 mL) at room temperature and the resulting reaction mixture was stirred for 2 hours. The mixture was concentrated under reduced pressure to get crude hydrochloride salt of ARK-8 as a yellow solid. The crude mixture was purified by preparative HPLC using following method to afford pure ARK-8_HCl salt (0.2 g, 40.5%) as a white solid. ¹H NMR (400 MHz, D₂O) δ 7.62 ppm (3H, broad s), 7.13 ppm (3H, broad s), 7.01 ppm (3H, broad s), 5.53 ppm (1H, S), 5.2 (1H, s), 2.92 ppm (6H, broad s), 2.60 ppm (6H, broad s), 1.22 ppm (6H, broad s), 1.07 ppm (6H, broad s), 0.76 ppm (6H, broad s), MS (ESI-MS): m/z calcd for C₄₄H₆₂N₆O₃ [MH]⁺ 724.0, found 723.6. HPLC retention time: 4.947 min.

Method of Preparative HPLC:

(A) 0.05% HCl in water (B) MeCN:MeOH:IPA (65:25:10) (HPLC GR), using X SELECT FLUORO PHENYL COLUMN 250*19 mm, 5.0 μM with the flow rate of 22.0 mL/min and with the following gradient:

| Time | % A | % B |
|---|---|---|
| 0.01 | 93.0 | 7.0 |
| 15.00 | 85.0 | 15.0 |
| 15.50 | 0.0 | 100.0 |
| 18.50 | 0.0 | 100.0 |
| 18.60 | 93.0 | 7.0 |
| 20.00 | 93.0 | 7.0 |

### Example 11: Synthesis of ARK-9 (Ark000015), ARK-10 (Ark000016), ARK-11 (Ark000017), and ARK-12 (Ark000018)

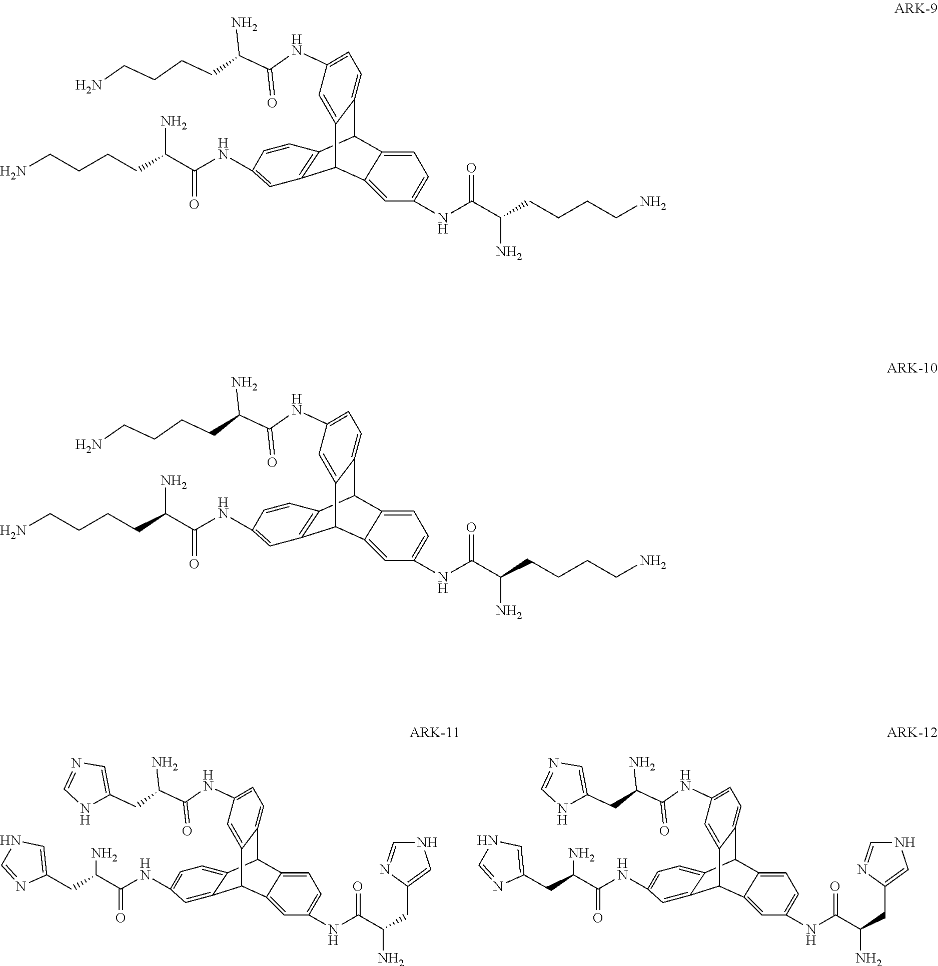 [SMILES: NCCCC[C@H](N)C(=O)Nc1ccc2c(c1)C1c3cc(NC(=O)[C@@H](N)CCCCN)ccc3C2c2ccc(NC(=O)[C@@H](N)CCCCN)cc21 ; NCCCC[C@@H](N)C(=O)Nc1ccc2c(c1)C1c3cc(NC(=O)[C@H](N)CCCCN)ccc3C2c2ccc(NC(=O)[C@H](N)CCCCN)cc21 ; N[C@@H](Cc1c[nH]cn1)C(=O)Nc1ccc2c(c1)C1c3cc(NC(=O)[C@@H](N)Cc4c[nH]cn4)ccc3C2c2ccc(NC(=O)[C@@H](N)Cc3cnc[nH]3)cc21 ; N[C@H](Cc1c[nH]cn1)C(=O)Nc1ccc2c(c1)C1c3cc(NC(=O)[C@H](N)Cc4c[nH]cn4)ccc3C2c2ccc(NC(=O)[C@H](N)Cc3cnc[nH]3)cc21]

ARK-9 was prepared analogously to ARK-7 above through compound 2. Compound 2 was then coupled with Boc-L-Lys(Boc)-OH as described below and then deprotected to provide ARK-9 (ARK-10 (Ark000016) was provided analogously by substituting Boc-D-Lys(Boc)-OH). In a similar way, ARK-11 (Ark000017) and ARK-12 (Ark000018) were provided by coupling with protected L or D-His amino acids.

### Hexa-tert-butyl ((5S,5′S,5″S)-((9,10-dihydro-9,10-[1,2]benzenoanthracene-2,7,15 triyl)tris(azanediyl))tris(6-oxohexane-6,1,5-triyl))hexacarbamate, 3

To a solution of 9,10-dihydro-9,10-[1,2]benzenoanthracene-2,7,15-triamine (2) (0.1 g, 0.3344 mmol) in DMF (1 mL) were added Boc-L-Lys(Boc)-OH (0.37 g, 1.07 mmol), HATU (0.406, 1.07 mmol) and DIPEA (0.258 g, 2.006 mmol) at room temperature. The reaction mixture was stirred at room temperature for 60 min. The resulting reaction mixture was poured into ice-cold water. The obtained solid precipitate was collected by filtration and dried under reduced pressure to afford crude 3 (0.38 g, 88.57%) as a white solid which was used without further purification. MS (ESI-MS): m/z calcd for C₆₈H₁₀₁N₉O₁₅ [MH]⁺ 1283.74, found 1185.0 (M−100).

### (2S,2′S,2″S)—N,N′,N″-(9,10-dihydro-9,10-[1,2]benzenoanthracene-2,7,15-triyl)tris(2,6-diaminohexanamide), ARK-9

The crude product hexa-tert-butyl ((5S,5′S,5″S)-((9,10-dihydro-9,10-[1,2]benzenoanthracene-2,7,15 triyl)tris(azanediyl))tris(6-oxohexane-6,1,5-triyl))hexacarbamate (3) (0.3 g, 0.234 mmol) obtained from previous step was suspended in 4 M HCl in dioxane and stirred at room temperature for 2 h. The resulting reaction mixture was concentrated under reduced pressure to afford crude ARK-9 hydrochloride salt as a white solid. The crude product was purified by preparative HPLC using the method shown below to afford pure salt of ARK-9 (0.19 g, 46.91%) as a white solid. The pure salt of ARK-9 was dissolved in DM water (4 mL) and passed through Amberlite® IRA-400 —OH form ion exchange resin. The free base was eluted using DM water and the fractions collected were lyophilized to obtain free base (0.15 g) as a white solid. The free base (0.05 g) was treated with aqueous 1 N HCl (3 mL) and lyophilized the material to generate hydrochloride salt of ARK-9 (0.05 g, 83.33%) as a white solid. ¹H NMR (400 MHz, D₂O) δ 7.56-7.55 ppm (3H, d, J=1.6 Hz), 7.41-7.39 ppm (3H, d, J=8.0 Hz), 7.01-6.99 ppm (H, dd, J=8 Hz, J=1.6 Hz), 5.62 ppm (1H, S), 5.59 ppm (1H, s), 4.01-3.98 ppm (3H, t), 2.88-2.84 ppm (6H, t), 1.90-1.86 ppm (6H, m), 1.61-1.57 ppm (6H, 3), 1.40-1.36 ppm (6H, m), MS (ESI-MS): m/z calcd for C₂₂H₂₇N₅O₂ [MH]⁺ 684.4, found 684.7. HPLC retention time: 5.092 min.

Method of Preparative HPLC:

(A) 0.1% TFA in water and (B) MeCN:MeOH:IPA (65:25:10) (HPLC grade), using X SELECT FLUORO PHENYL COLUMN 250×19 mm, 5.0 μm with the flow rate of 12.0 mL/min and with the following gradient:

| Time | % A | % B |
|---|---|---|
| 0.01 | 100.0 | 0.0 |
| 5.00 | 100.0 | 0.0 |
| 15.00 | 90.0 | 10.0 |
| 15.01 | 50.0 | 50.0 |
| 18.00 | 50.0 | 50.0 |
| 18.01 | 0.0 | 0.0 |
| 19.00 | 0.0 | 0.0 |

Synthesis of ARK-10 (Ark000016):

Hexa-tert-butyl ((5R,5′R,5″R)-((9,10-dihydro-9,10-[1,2]benzenoanthracene-2,7,15-triyl)tris(azanediyl))tris(6-oxohexane-6,1,5-triyl))hexacarbamate, 3.

To a solution of 9,10-dihydro-9,10-[1,2]benzenoanthracene-2,7,15-triamine (2) (0.3 g, 1.00 mmol) in DMF (5 mL) was added Boc-D-Lys(Boc)-OH (1.1 g, 3.210 mmol), HATU (1.2 g, 3.210 mmol) and DIPEA (0.774 g, 6.00 mmol) at room temperature. The reaction mixture was stirred at room temperature for 60 min. The resulting reaction mixture was poured into ice-cold water. The obtained solid precipitates were collected by filtration and dried under reduced pressure to afford crude 3. The crude mixture was purified by preparative HPLC using following method to afford pure 3 (0.25 g, 19.53%) as a white solid. MS (ESI-MS): m/z calcd for C₆₈H₁₀₁N₉O₁₅ [MH]⁺ 1283.74, found 1185.0 (M−100; de-protection of one Boc group).

Method of Preparative HPLC:

(A) 10 mM ammonium bicarbonate in water (HPLC grade) and (B) ACN:MeOH:IPA (65:25:10) (HPLC GR), using X BRIDGE 250 mm*30 mm*5 μm with a flow rate of 28.0 mL/min and with the following gradient:

| Time | % A | % B |
|---|---|---|
| 0.01 | 25.0 | 75.0 |
| 19.00 | 21.0 | 79.0 |
| 19.01 | 0.0 | 100.0 |
| 20.00 | 0.0 | 100.0 |
| 20.01 | 25.0 | 75.0 |
| 21.00 | 25.0 | 75.0 |

### (2R,2′R,2″R)—N,N′,N″-(9,10-dihydro-9,10-[1,2]benzenoanthracene-2,7,15-triyl)tris(2,6-diaminohexanamide), ARK-10

The crude product hexa-tert-butyl ((5R,5′R,5″R)-((9,10-dihydro-9,10-[1,2]benzenoanthracene-2,7,15-triyl)tris(azanediyl))tris(6-oxohexane-6,1,5-triyl))hexacarbamate (3) (0.25 g, 0.1947 mmol) obtained from previous step was suspended in 4 M HCl in dioxane and stirred at room temperature for 2 hours. The resulting reaction mixture was concentrated under reduced pressure to afford crude ARK-10 hydrochloride salt as a white solid. The crude product was purified by preparative HPLC using the method shown below to afford pure salt of ARK-10 (0.14 g, 26.41%) as a white solid. The pure salt of ARK-10 was dissolved in DM water (4 mL) and passed through Amberlite® IRA-400 —OH form ion exchange resin. The free base was eluted using DM water and the fractions collected were lyophilized to obtain free base (0.07 g) as a white solid. The free base (0.07 g) was treated with aqueous 1 N HCl (3 mL) and lyophilized to generate hydrochloride salt of ARK-10 (0.085 g, 92.39%) as a light brown solid. ¹H NMR (400 MHz, D₂O) δ 7.54-7.53 ppm (3H, d, J=2 Hz), 7.38-7.36 ppm (3H, d, J=8.0 Hz), 6.99-6.97 ppm (3H, dd, J=8 Hz, J=2 Hz), 5.60 ppm (1H, S), 5.56 (1H, s), 3.99-3.96 ppm (3H, t), 2.86-2.82 ppm (6H, t), 1.89-1.82 ppm (6H, m), 1.61-1.53 ppm (6H, m), 1.40-1.34 ppm (6H, m). MS (ESI-MS): m/z calcd for C₂₂H₂₇N₅O₂ [MH]⁺ 684.4, found 684.6. HPLC retention time: 6.393 min.

Method of Preparative HPLC:

(A) 0.1% TFA in water (HPLC grade) and (B) MeCN:MeOH:IPA (65:25:10) (HPLC GR), using X SELECT PFP C18, 250*19 mm, Sum with the flow rate of 15.0 mL/min and with the following gradient:

| Time | % A | % B |
|---|---|---|
| 0.01 | 100.0 | 0.0 |
| 3.00 | 100.0 | 0.0 |
| 16.00 | 97 | 3 |
| 16.01 | 20 | 80 |
| 18.00 | 20 | 80 |
| 18.01 | 100.0 | 0.0 |

### Synthesis of ARK-11 and ARK-12

### Tri-tert-butyl ((2S,2′S,2″S)-((9,10-dihydro-9,10-[1,2]benzenoanthracene-2,7,15-triyl) tris (azanediyl))tris (3-(1H-imidazol-4-yl)-1-oxopropane-1,2-diyl))tricarbamate

To a stirred solution of 9,10-dihydro-9,10-[1,2]benzenoanthracene-2,7,15-triamine (2) (0.3 g, 1.0 mmol) in DMF (6 mL) was added Boc-L-Histidine (0.82 g, 3.2 mmol), HATU (1.22 g, 3.2 mmol), and DIPEA (0.8 g, 6.2 mmol) at room temperature. The resulting reaction mixture was stirred overnight at room temperature. The reaction mixture was poured in ice-cold water and residues obtained were collected through filtration, dried under reduced pressure to get crude 3 (0.65 g, 65%) as light brown solid which was directly used in the next step without purification. MS (ESI-MS): m/z calcd for C₅₃H₆₂N₁₂O₉ [MH]⁺ 1011.15, found 1011.9.

### (2S,2′S,2″S)—N,N′,N″-(9,10-dihydro-9,10-[1,2]benzenoanthracene-2,7,15-triyl)tris(2-amino-3-(1H-imidazol-4-yl)propanamide) hydrochloride, ARK-11_HCl Salt

To a stirred solution of tri-tert-butyl ((2S,2′S,2″S)-((9,10-dihydro-9,10-[1,2]benzenoanthracene-2,7,15-triyl) tris (azanediyl))tris (3-(1H-imidazol-4-yl)-1-oxopropane-1,2-diyl))tricarbamate (3) (0.65 g, 0.643 mmol) in dichloromethane (8 mL) was added 4N HCl in Dioxane (5 mL) at 0° C. The resulting reaction mixture was stirred at room temperature for 3 h. The reaction mixture was concentrated under reduced pressure to get crude ARK-11. The crude mixture was purified by preparative HPLC using following method to afford pure product ARK-11_TFA salt (0.32 g, 64.42%) as colorless viscous oil. The ARK-11_TFA salt was dissolved in methanol (10 mL). To this, polymer bound tetraalkylammonium carbonate and the resulting mixture was stirred at room temperature for 30 min. The mixture was filtered through celite and the resulting filtrate was concentrated under reduced pressure to get ARK-11_Free base. The free base was dissolved in 0.01 N HCl (10 mL) and resulting solution was lyophilized to obtain pure ARK-11_HCl salt (0.16 g, 61.06%) as white solid. ¹H NMR (400 MHz, D₂O) δ 8.56 ppm (3H, s), 7.51 ppm (3H, s), 7.39-7.31 ppm (6H, m), 6.93-6.91 ppm (3H, s), 5.61-5.58 ppm (2H, s), 4.26 ppm (3H, s), 3.36-3.34 ppm (6H, m), 3.21 ppm (2H, s); MS (ESI-MS): m/z calcd for C₃₈H₃₈N₁₂O₃ [MH]⁺ 710.8, found 712.2. HPLC retention time: 5.770 min.

Method for Preparative HPLC:

(A) 0.1% TFA in water (HPLC grade) and (B) 10% IPA in acetonitrile (HPLC grade), using WATERS X-BRIDGE C18, 250 mm*30 mm*5 μm with the flow rate of 35.0 mL/min and with the following gradient

| Time | % A | % B |
|---|---|---|
| 0.01 | 90.0 | 10.0 |
| 3.00 | 90.0 | 10.0 |
| 21.00 | 87.0 | 13.0 |
| 21.01 | 5.0 | 95.0 |
| 22.00 | 5.0 | 95.0 |
| 22.01 | 90.0 | 10.0 |
| 23.00 | 90.0 | 10.0 |

### Tri-tert-butyl ((2R,2′R,2″R)-((9,10-dihydro-9,10-[1,2]benzenoanthracene-2,7,15-triyl) tris (azanediyl)) tris (3-(1H-imidazol-4-yl)-1-oxopropane-1,2-diyl))tricarbamate

To a stirred solution of 9,10-dihydro-9,10-[1,2]benzenoanthracene-2,7,15-triamine (2) (0.25 g, 0.84 mmol) in DMF (6 mL) was added Boc-D-Histidine (0.68 g, 2.67 mmol), HATU (1.01 g, 2.67 mmol), DIPEA (0.69 g, 5.35 mmol) at room temperature. The resulting reaction mixture was stirred over night at room temperature. The reaction mixture was poured in ice-cold water and residues obtained were collected through filtration, dried under reduced pressure to get crude 3 (0.75 g, 88.9%) as white solid which was directly used in the next step without purification. MS (ESI-MS): m/z calcd for C₅₃H₆₂N₁₂O₉ [MH]⁺ 1011.48, found 1011.6.

### (2R,2′R,2″R)—N,N′,N″-(9,10-dihydro-9,10-[1,2]benzenoanthracene-2,7,15-triyl)tris(2-amino-3-(1H-imidazol-4-yl)propanamide) hydrochloride, ARK-12_HCl Salt

To a stirred solution of tri-tert-butyl ((2S,2′S,2″S)-((9,10-dihydro-9,10-[1,2]benzenoanthracene-2,7,15-triyl) tris (azanediyl))tris (3-(1H-imidazol-4-yl)-1-oxopropane-1,2-diyl))tricarbamate (3) (0.75 g, 0.742 mmol) in dichloromethane (8 mL) was added 4N HCl in Dioxane (5 mL) at 0° C. The resulting reaction mixture was stirred at room temperature for 3 h. The reaction mixture was concentrated under reduced pressure to get crude ARK-12. The crude mixture was purified by preparative HPLC using following method to afford pure product ARK-12_TFA salt (0.70 g, 72.53%) as white solid. The pure salt of ARK-12 was dissolved in DM water (4 mL) and passed through Amberlite® IRA-400 —OH form ion exchange resin. The free base was eluted using DM water and the fractions collected were lyophilized to get free base (0.06 g) as a white solid. The free base (0.06 g) was dissolved in aqueous 1 N HCl solution (3 mL) and lyophilized the material to generate hydrochloride salt of ARK-12 (0.07 g, 10.16%) as a white solid. ¹H NMR (400 MHz, D₂O) δ 8.54 ppm (3H, s), 7.50 ppm (3H, s), 7.37-7.35 ppm (3H, d, J=8 Hz), 7.28 ppm (3H, S), 6.90-6.88 ppm (3H, dd, J=7.6 Hz), 5.59 ppm (1H, s), 5.56 ppm (1H, s), 4.25-4.22 ppm (3H, t, J=7.2 Hz), 3.33-3.31 ppm (6H, d, J=7.2 Hz). MS (ESI-MS): m/z calcd for C₃₈H₃₈N₁₂O₃[MH]⁺ 711.32, found 684.6. HPLC retention time: 6.347 min.

Method for Preparative HPLC:

0.1% TFA in water (HPLC grade) and (B) 10% IPA in acetonitrile (HPLC grade), using WATERS X-BRIDGE C18, 250 mm*30 mm*5 μm with the flow rate of 35.0 mL/min and with the following gradient:

| Time | % A | % B |
|---|---|---|
| 0.01 | 90.0 | 10.0 |
| 3.00 | 90.0 | 10.0 |
| 21.00 | 87.0 | 13.0 |
| 21.01 | 5.0 | 95.0 |
| 22.00 | 5.0 | 95.0 |
| 22.01 | 90.0 | 10.0 |
| 23.00 | 90.0 | 10.0 |

### Example 12: Synthesis of ARK-77 and ARK-77A (Ark000033 and Ark000034)

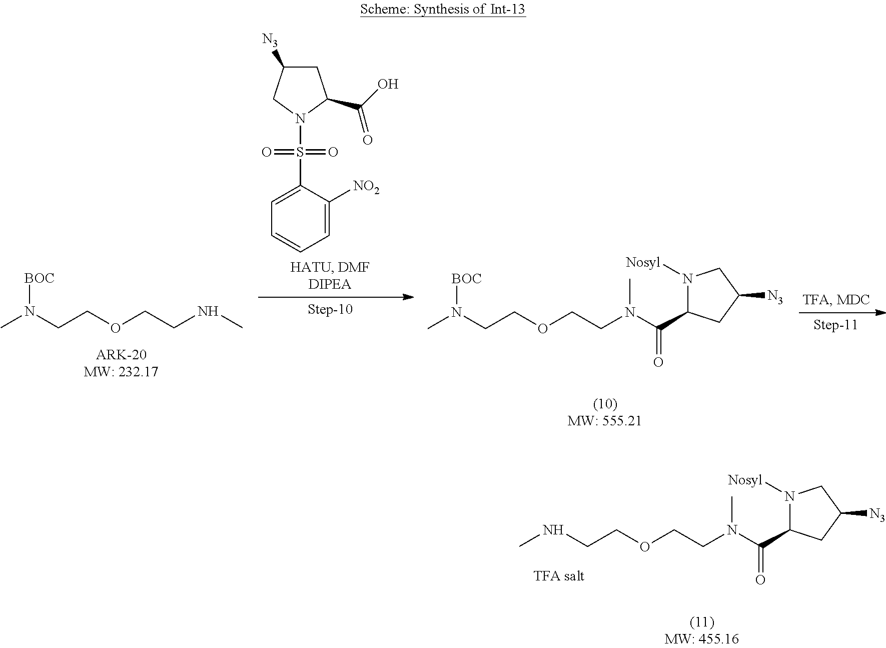

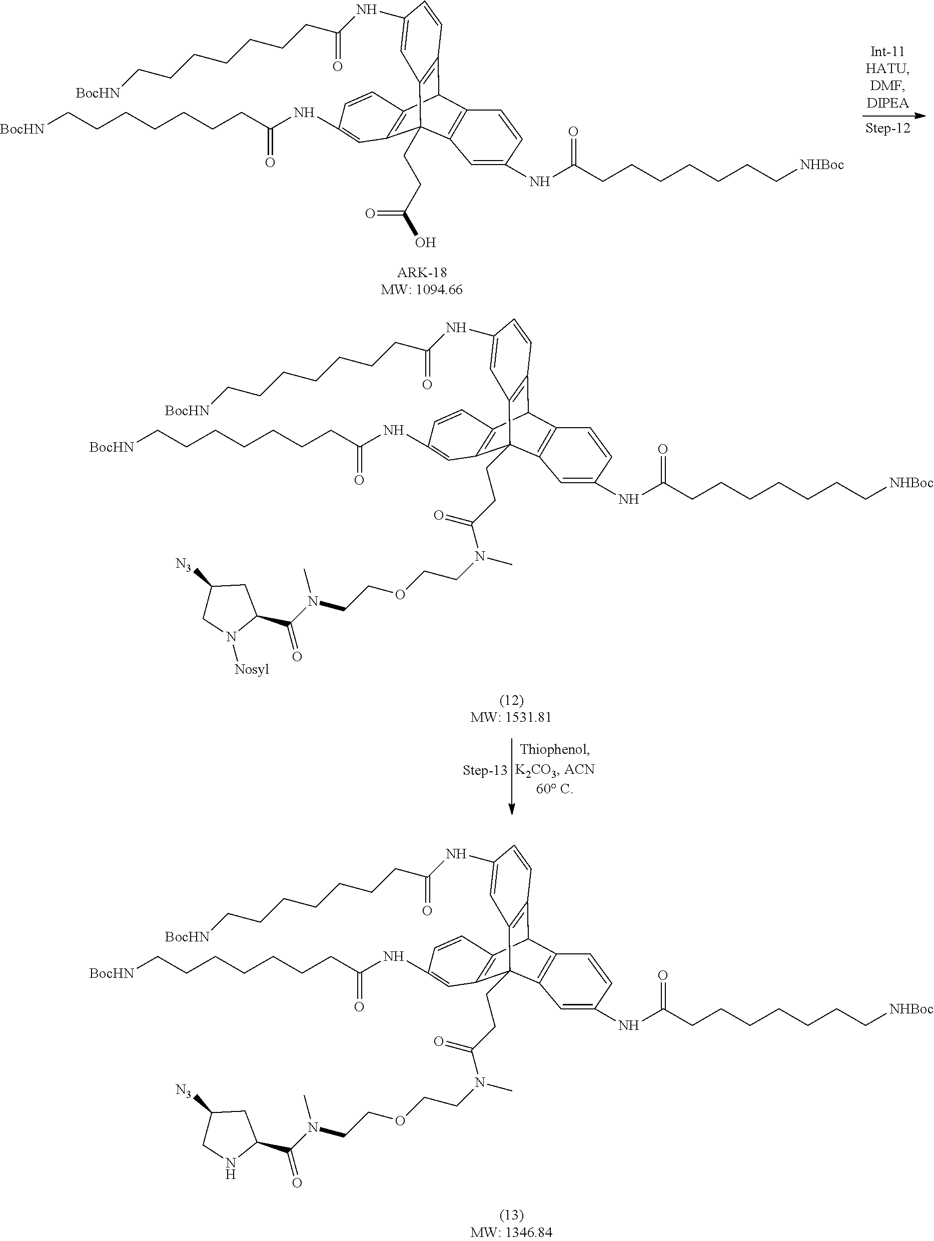

### Tert-butyl (2-(2-((2S,4S)-4-azido-N-methyl-1-((2-nitrophenyl)sulfonyl)pyrrolidine-2-carboxamido) ethoxy)ethyl)(methyl)carbamate, 10

To a solution of ARK-20 (2.0 g, 8.614 mmol) in N,N-dimethylformamide (40 mL) were sequentially added (2S,4S)-4-azido-1-((2-nitrophenyl)sulfonyl)pyrrolidine-2-carboxylic acid (2.34 g, 6.89 mmol), HATU (2.62 g, 6.89 mmol) and N,N-diisopropylethylamine (3.33 g, 25.84 mmol) at room temperature. The resulting reaction mixture was stirred for 1 h at room temperature. The reaction mixture was poured in ice-cold water and extracted with ethyl acetate (3×100 mL). The organic layers were combined, washed with brine and concentrated under reduced pressure to get crude 10 (3.5 g, 91.6%) as brown semisolid. The crude mixture was used in next step without further purification. MS (ESI-MS): m/z calcd for C₂₂H₃₃N₇O₈S [MH]⁺ 556.21, found 573.43 (M+18, water adduct).

### (2S,4S)-4-azido-N-methyl-N-(2-(2-(methylamino)ethoxy)ethyl)-1-((2-nitrophenyl)sulfonyl) pyrrolidine-2-carboxamide_TFA Salt, 11

To a solution of tert-butyl (2-(2-((2S,4S)-4-azido-N-methyl-1-((2-nitrophenyl)sulfonyl)pyrrolidine-2-carboxamido) ethoxy)ethyl)(methyl)carbamate (10) (3.5 g, 6.30 mmol) in dichloro methane (30 mL) was added trifluoro acetic acid (3.15 mL, 31.52 mmol) at room temperature. The resulted reaction mixture was stirred at room temperature for 2 h. The reaction mixture was filtered through celite bed and filtrate thus collected was concentrated under reduced pressure to get crude 11 (4.3 g, quantitative yield) as a brown oil which was used in next step without further purification. MS (ESI-MS): m/z calcd for C₁₇H₂₅N₇O₆S.TFA [MH]⁺ 456.16, found 456.32.

### Tri-tert-butyl (((9-(3-((2-(2-((2S,4S)-4-azido-N-methyl-1-((2-nitrophenyl)sulfonyl)pyrrolidine-2-carboxamido)ethoxy)ethyl)(methyl)amino)-3-oxopropyl)-9,10-dihydro-9,10[1,2]benzenoanthracene-2,7,15-triyl)tris(azanediyl))tris(8-oxooctane-8,1-diyl))tricarbamate, 12

To a solution of (2S,4S)-4-azido-N-methyl-N-(2-(2-(methylamino)ethoxy)ethyl)-1-((2-nitrophenyl)sulfonyl) pyrrolidine-2-carboxamide_TFA Salt (11) (1.25 g, 2.19 mmol) in N,N-dimethylformamide (30 mL) were sequentially added 3-(2,7,15-tris(8-((tert-butoxycarbonyl)amino)octanamido)-9,10-[1,2]benzenoanthracen-9(10H)-yl)propanoic acid (ARK-18) (2.0 g, 1.83 mmol), HATU (0.833 g, 2.192 mmol) and N,N-diisopropylethylamine (0.942 g, 7.31 mmol) at room temperature. The resulting reaction mixture was stirred for 1 h at room temperature. The reaction mixture was poured in ice-cold water and extracted with ethyl acetate (3×100 mL). The organic layers were combined, washed with brine and concentrated under reduced pressure to get crude 12. The crude mixture was purified by column chromatography on silica gel (3.2% methanol/chloroform) to yield 12 (2.3 g, 82.17%) as a dark yellow solid. MS (ESI-MS): m/z calcd for C₇₉H₁₁₃N₁₃O₁₆S [MH]⁺ 1532.81, found 1433.19 (M−100, one Boc group fell off).

### Tri-tert-butyl (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate, 13

To a solution of tri-tert-butyl (((9-(3-((2-(2-((2S,4S)-4-azido-N-methyl-1-((2-nitrophenyl)sulfonyl) pyrrolidine-2-carboxamido)ethoxy)ethyl)(methyl)amino)-3-oxopropyl)-9,10-dihydro-9,10[1,2] benzenoanthracene-2,7,15-triyl)tris(azanediyl))tris(8-oxooctane-8,1-diyl))tricarbamate (12) (2.2 g, 1.44 mmol) in acetonitrile (30 mL) were sequentially added potassium carbonate (0.99 g, 7.18 mmol) and thiophenol (0.44 mL, 4.31 mmol) at room temperature. The resulted reaction mixture was stirred at 80° C. for 2 h. The reaction mixture was filtered through celite bed and the collected filtrate was concentrated under reduced pressure to get crude 13 as yellow oil. The crude mixture was subjected to reverse phase chromatography to yield 13 (1.1 g, 56.88%) as a light yellow solid. The yellow solid was further subjected to preparative HPLC (method mentioned below) purification followed by lyophilization to yield pure 13 (0.41 g, 52.17%) as a white amorphous powder. MS (ESI-MS): m/z calcd for C₇₃H₁₁₀N₁₂O₁₂ [MH]⁺ 1347.84, found 1349.28.

Method for Preparative HPLC:

(A) 10 mM NH₄HCO₃ IN WATER (HPLC GRADE) and (B) 100% Acetonitrile (HPLC GRADE) in water (HPLC GRADE), using X-BRIDGE C18, 250 mm*30 mm*5 μm with the following flow rate and gradient:

| Time | Flow rate | % A | % B |
|---|---|---|---|
| 0.01 | 22.0 | 30.0 | 70.0 |
| 21.00 | 22.0 | 28.0 | 72.0 |
| 21.01 | 30.0 | 0.0 | 100 |
| 27.00 | 30.0 | 0.0 | 100 |
| 27.01 | 22.0 | 30.0 | 70.0 |
| 28.00 | 22.0 | 30.0 | 70.0 |

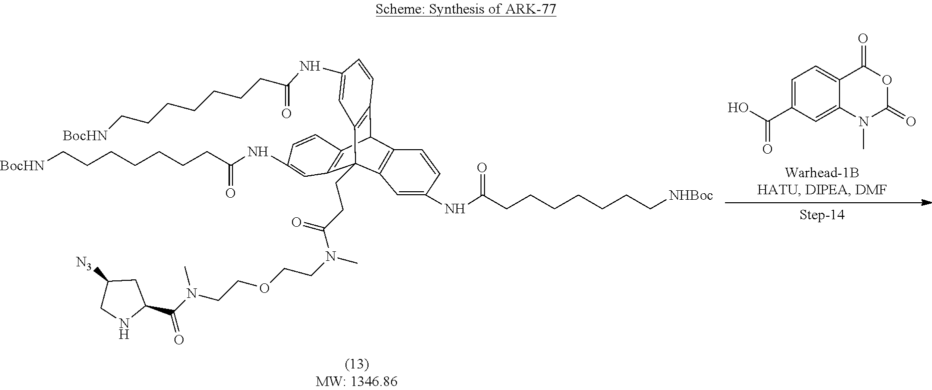

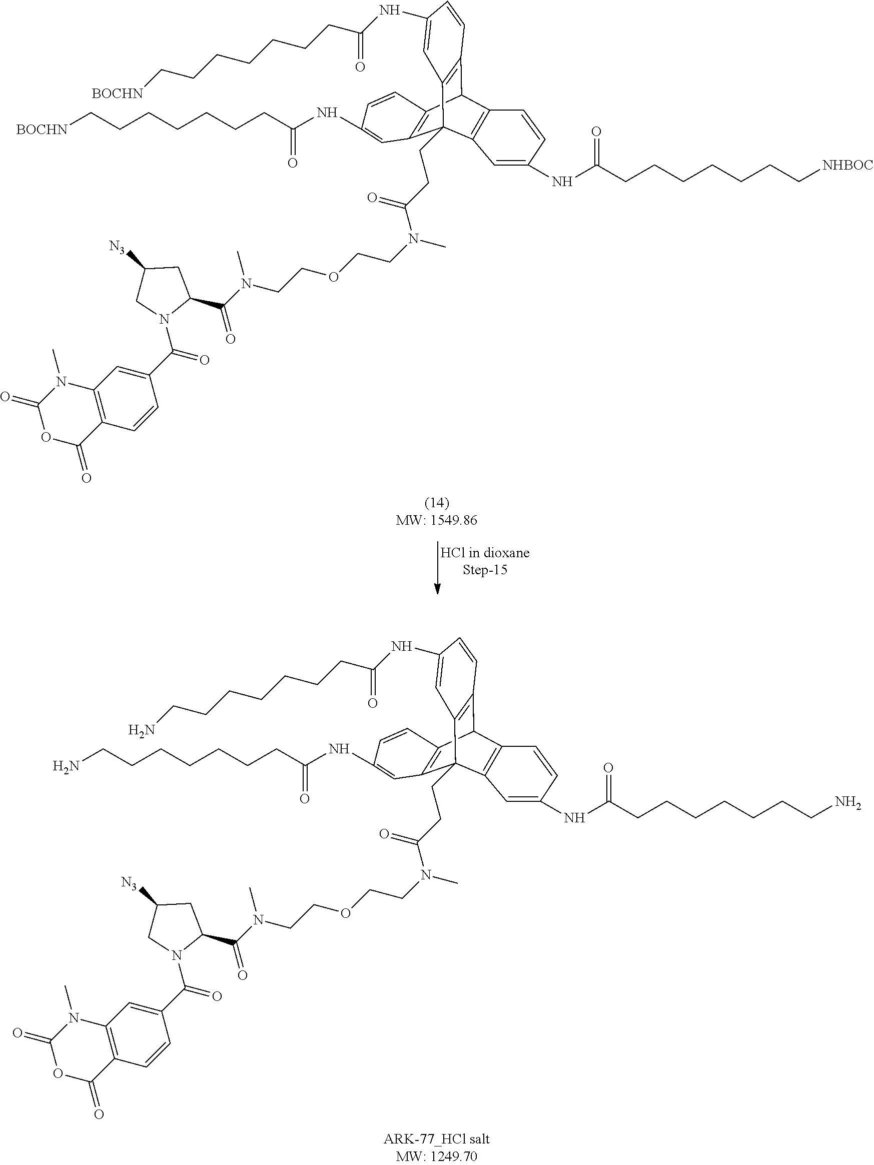

### Tri-tert-butyl (((9-(3-((2-(2-((2S,4S)-4-azido-N-methyl-1-(1-methyl-2,4-dioxo-1,4-dihydro-2H-benzo[d][1,3]oxazine-7-carbonyl)pyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate (13) (0.2 g, 0.148 mmol) in N,N-dimethylformamide (8 mL) were sequentially added 1-methyl-2,4-dioxo-2,4-dihydro-1H-3,1-benzoxazine-7-carboxylic acid (Warhead_type_1B) (0.039 g, 0.178 mmol) and HATU (0.068 g, 0.178 mmol) at room temperature. The reaction mixture was stirred for 5 minutes. To this, N,N-diisopropylethylamine (0.038 g, 0.297 mmol) was added dropwise and the resulted reaction mixture was further stirred for 30 minutes at room temperature. The reaction mixture was diluted by ethyl acetate (100 mL) and washed with ice-cold water (3×30 mL). The organic layers were combined, washed with brine and concentrated under reduced pressure at 25° C. to get crude 14. The crude mixture was purified by preparative HPLC (method mentioned below) followed by lyophilization to yield 14 (0.12 g, 52.17%) as a white amorphous powder. MS (ESI-MS): m/z calcd Cs₃H₁₁₅N₁₃O₁₆ [MH]⁺ 1550.86, found 1452.42 (M−100, one Boc group fell off).

Method for Preparative HPLC:

(A) 100% Acetonitrile (HPLC GRADE) and (B) 100% Tetrahydrofuran (HPLC GRADE), using SUNFIRE SILICA, 150 mm*19 mm*5 μm with the flow rate of 19.0 mL/min and with the following gradient:

| Time | % A | % B |
|---|---|---|
| 0.01 | 98.0 | 2.0 |
| 20.00 | 98.0 | 2.0 |

### N,N′,N″-(9-(3-((2-(2-((2S,4S)-4-azido-N-methyl-1-(1-methyl-2,4-dioxo-1,4-dihydro-2H-benzo[d][1,3]oxazine-7-carbonyl)pyrrolidine-2-carboxamido)ethoxy)ethyl)(methyl)amino)-3-oxopropyl)-9,10-dihydro-9,10-[1,2]benzenoanthracene-2,7,15-triyl)tris(8-aminooctanamide), ARK-77_HCl Salt

To a solution of tri-tert-butyl (((9-(3-((2-(2-((2S,4S)-4-azido-N-methyl-1-(1-methyl-2,4-dioxo-1,4-dihydro-2H-benzo[d][1,3]oxazine-7-carbonyl)pyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate (14) (0.079 g, 0.051 mmol) in 1,4-dioxane (3.0 mL) was added 4 M HCl in dioxane solution (1.5 mL) at room temperature and the resulting reaction mixture was stirred for 30 minutes under nitrogen atmosphere. During this, solid residue started to precipitate out. The suspension was further stirred for 30 minutes and finally allowed to stand at room temperature. The solid residues started to deposit on bottom of the flask. The solvent was decanted and the residues left were triturated with acetonitrile (3×3 mL). Finally the solid was dried under reduced pressure at 25° C. to get pure ARK-77_HCl_Salt (0.054 g, 69.28%) as a white amorphous powder. ¹H NMR (400 MHz, DMSO-d6) δ 9.91 ppm (3H, broad), 8.09-8.03 ppm (1H, m), 7.90 ppm (8H, broad), 7.67 ppm (3H, broad), 7.37-7.33 ppm (2H, m), 7.29-7.27 ppm (3H, m), 7.23 ppm (3H, m), 5.38 ppm (1H, s), 5.01 ppm (1H, m), 4.86-4.79 ppm (1H, m), 4.31-4.23 ppm (1H, m), 4.09 ppm (1H, m), 3.79-3.64 ppm (4H, m), 3.48 ppm (14H, m), 3.44-3.40 ppm (4H, m), 3.18 ppm (1H, s), 3.08-3.01 ppm (6H, m), 2.77-2.66 ppm (7H, m), 2.25 ppm (6H, broad s), 1.53 ppm (12H, broad s), 1.27 ppm (18H, broad s). MS (ESI-MS): m/z calcd for C₆₈H₉₁N₁₃O₁₀ [MH]⁺ 1250.70, found 1251.48.

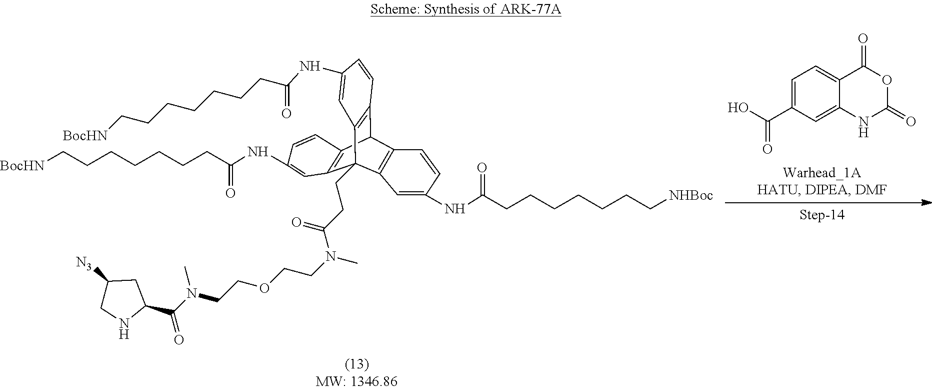

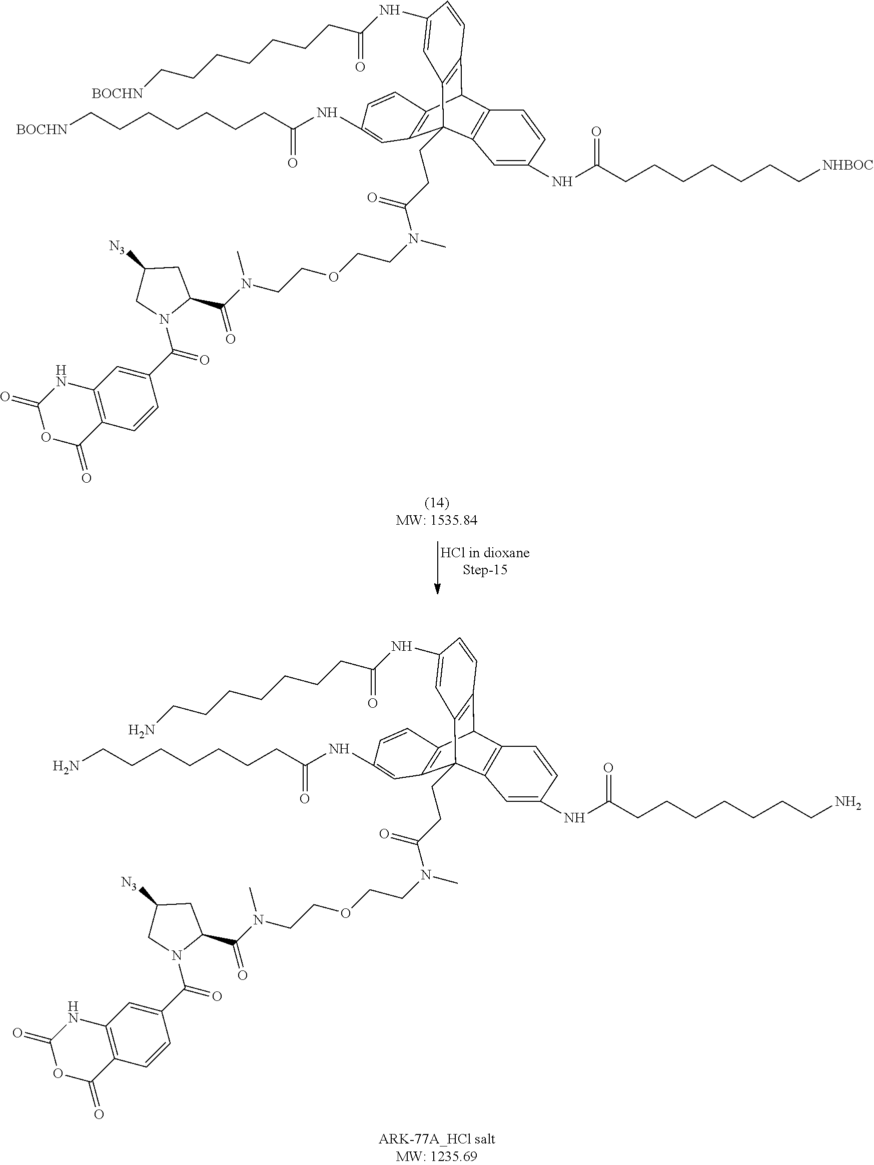

### Tri-tert-butyl (((9-(3-((2-(2-((2S,4S)-4-azido-1-(2,4-dioxo-1,4-dihydro-2H-benzo[d][1,3]oxazine-7-carbonyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate (13) (0.156 g, 0.116 mmol) in N,N-dimethylformamide (6 mL) were sequentially added 2,4-dioxo-1,4-dihydro-2H-benzo[d][1,3]oxazine-7-carboxylic acid (Warhead_type_1A) (0.029 g, 0.139 mmol) and HATU (0.053 g, 0.139 mmol) at room temperature. The reaction mixture was stirred for 5 minutes. To this, N,N-diisopropylethylamine (0.03 g, 0.232 mmol) was added drop wise and the resulted reaction mixture was further stirred for 30 minutes at room temperature. The reaction mixture was diluted by ethyl acetate (100 mL) and washed with ice-cold water (3×30 mL). The organic layers were combined, washed with brine and concentrated under reduced pressure at 25° C. to get crude 14. The crude mixture was purified by preparative HPLC using following method to yield pure 14 (0.093 g, 52.17%) as a white amorphous powder. The prep-fraction was concentrated by reduced pressure at 25° C. under nitrogen atmosphere. MS (ESI-MS): m/z calcd C₈₂H₁₁₃N₁₃O₁₆ [MH]⁺ 1536.84, found 1437.41 (M−100, one Boc group fell off).

Method for Preparative HPLC:

(A) 100% Acetonitrile (HPLC GRADE) and (B) 100% Tetrahydrofuran (HPLC GRADE), using SUNFIRE SILICA, 150 mm*19 mm*5 μm with the following flow rate and following gradient:

| Time | Flow rate | % A | % B |
|---|---|---|---|
| 0.01 | 17.0 | 100.0 | 0.0 |
| 5.0 | 17.0 | 100.0 | 0.0 |
| 19.00 | 17.0 | 98.0 | 2.0 |
| 19.01 | 19.0 | 100.0 | 0.0 |
| 20.00 | 19.0 | 100.0 | 0.0 |
| 20.01 | 17.0 | 100.0 | 0.0 |
| 21.00 | 17.0 | 100.0 | 0.0 |

### N,N′,N″-(9-(3-((2-(2-((2S,4S)-4-azido-1-(2,4-dioxo-1,4-dihydro-2H-benzo[d][1,3]oxazine-7-carbonyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(8-aminooctanamide), ARK-77A_HCl Salt

To a solution of tri-tert-butyl (((9-(3-((2-(2-((2S,4S)-4-azido-1-(2,4-dioxo-1,4-dihydro-2H-benzo[d][1,3]oxazine-7-carbonyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate (14) (0.06 g, 0.039 mmol) in 1,4-Dioxane (Dry) (3 ml) was added 4 M HCl in dioxane (1.2 mL) at room temperature and the resulting reaction mixture was stirred for 30 minutes under nitrogen atmosphere. The solid material was stable at the bottom of the flask and the solvent was decanted under inert atmosphere, then the solid material was triturating with acetonitrile (HPLC Grade) (3×3 mL). The remaining solid was concentrated by reduced pressure at 25° C. under nitrogen atmosphere to afford pure ARK-77A_HCl_Salt (0.054 g, 69.28%) as a white amorphous powder. ¹H NMR (400 MHz, DMSO-d₆) δ 12.04-11.95 ppm (1H, d), 9.91 ppm (3H, broad), 7.98-7.96 ppm (1H, m), 7.89 ppm (7H, broad), 7.71-7.67 ppm (3H, broad), 7.29-7.27 ppm (4H, d), 7.23 ppm (3H, broad), 5.38 ppm (1H, s), 5.03-5.01 ppm (1H, m), 4.86-4.79 ppm (1H, m), 4.30-4.23 ppm (1H, m), 4.07 ppm (1H, m), 3.76 ppm (1H, m), 3.35-3.44 ppm (2H, m), 3.17 ppm (1H, s), 3.08-3.04 ppm (5H, m), 2.99-2.84 ppm (1H, m), 2.79-2.68 ppm (7H, m), 2.25-2.23 ppm (6H, t), 1.53 ppm (12H, broad), 1.27 ppm (18H, broad). MS (ESI-MS): m/z calcd for C₆₇H₈₉N₁₃O₁₀ [MH]⁺ 1236.69, found 1238.46.

### Example 13: Synthesis of ARK-78 and ARK-78A (Ark000035 and Ark000037)

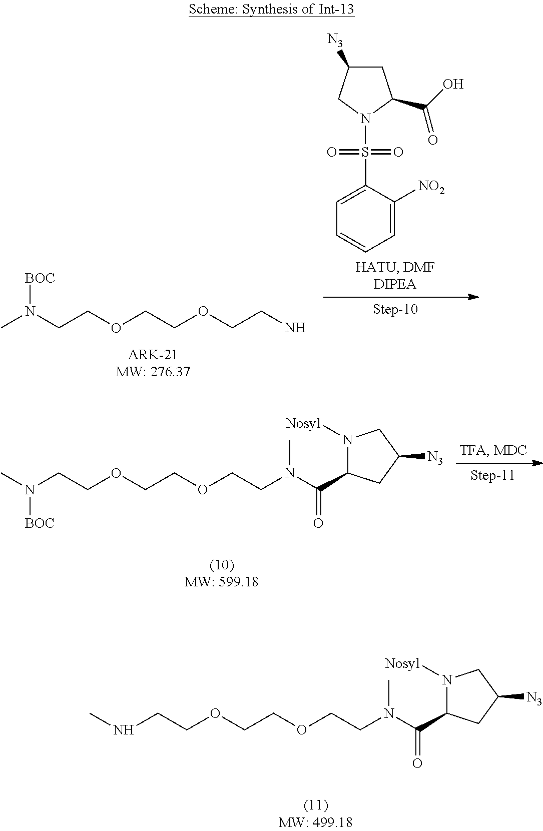

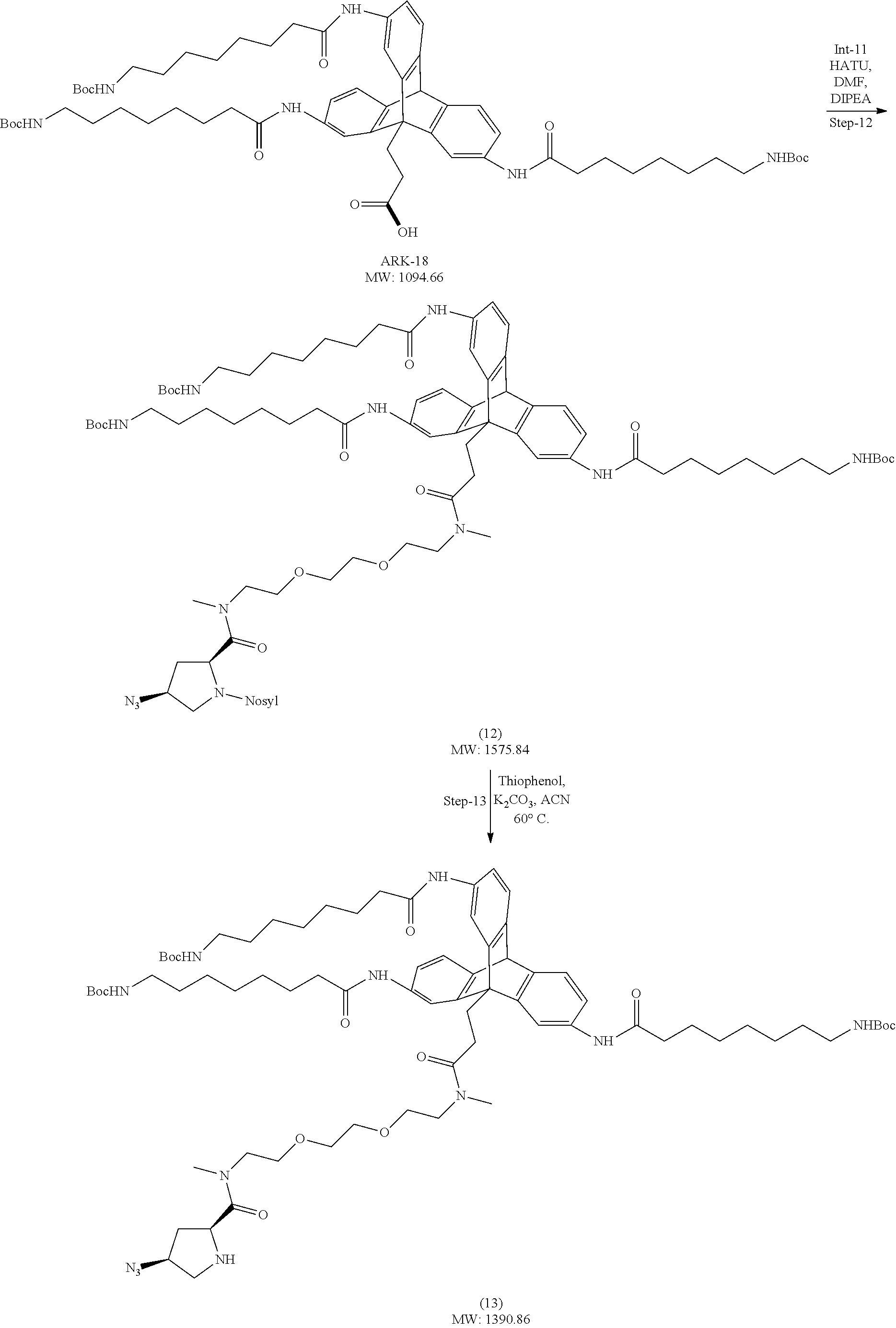

### Tert-butyl (2-(2-(2-((2S,4S)-4-azido-N-methyl-1-((2-nitrophenyl)sulfonyl)pyrrolidine-2-carboxamido)ethoxy)ethoxy)ethyl)(methyl)carbamate, 10

To a solution of ARK-21 (2.4 g, 8.68 mmol) in N,N-dimethylformamide (30 mL) were sequentially added (2S,4S)-4-azido-1-((2-nitrophenyl)sulfonyl)pyrrolidine-2-carboxylic acid (2.96 g, 8.68 mmol), HATU (3.96 g, 10.42 mmol) and N,N-diisopropylethylamine (3.36 g, 26.05 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 10 (4.0 g, 76.9%) as yellow viscous liquid. The crude mixture was used in next step without further purification. MS (ESI-MS): m/z calcd for C₂₄H₃₇N₇O₉S [MH]⁺ 600.18, found 617.5 (M+18).

### (2S,4S)-4-azido-N-methyl-N-(2-(2-(2-(methylamino)ethoxy)ethoxy)ethyl)-1-((2 nitrophenyl)sulfonyl)pyrrolidine-2-carboxamide_TFA Salt, 11

To a solution of tri-tert-butyl ((2R,2′R,2″R)-((9,10-dihydro-9,10-[1,2]benzenoanthracene-2,7,15-triyl)tris(azanediyl))tris(3-(1H-imidazol-4-yl)-1-oxopropane-1,2-diyl))tricarbamate (10) (4.0 g, 6.67 mmol) in dichloro methane (20 mL) was added trifluoro acetic acid (2.58 mL, 33.38 mmol) at room temperature. The resulted reaction mixture was stirred at room temperature for 2 h. The reaction mixture was filtered through celite bed and filtrate thus collected was concentrated under reduced pressure to get crude 11 (7.5 g, Quantitative yield) as a brown oil which was used in next step without further purification. MS (ESI-MS): m/z calcd for C₁₉H₂₉N₇O₇S [MH]⁺ 500.18, found 500.31.

### Tri-tert-butyl (((9-(1-((2S,4S)-4-azido-1-((2-nitrophenyl)sulfonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 12

To a solution of (2S,4S)-4-azido-N-methyl-N-(2-(2-(2-(methylamino)ethoxy)ethoxy)ethyl)-1-((2-nitrophenyl)sulfonyl)pyrrolidine-2-carboxamide_TFA Salt (11) (2.69 g, 4.38 mmol) in N,N-dimethylformamide (40 mL) were sequentially added 3-(2,7,15-tris(8-((tert-butoxycarbonyl)amino)octanamido)-9,10-[1,2]benzenoanthracen-9(10H)-yl)propanoic acid (ARK-18) (4.0 g, 3.65 mmol), HATU (1.67 g, 4.38 mmol) and N,N-diisopropylethylamine (1.41 g, 10.96 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 12. The crude mixture was purified by column chromatography on silica gel (4.3% methanol/chloroform) to yield 12 (4.7 g, 81.6%) as a dark yellow solid. MS (ESI-MS): m/z calcd for C₈₁H₁₁₇N₁₃O₁₇S [MH]⁺ 1576.84, found 1578.4.

### Tri-tert-butyl (((9-(1-((2S,4S)-4-azidopyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 13

To a solution of tri-tert-butyl (((9-(1-((2S,4S)-4-azido-1-((2-nitrophenyl)sulfonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (12) (4.7 g, 2.98 mmol) in acetonitrile (50 mL) were sequentially added potassium carbonate (2.06 g, 14.91 mmol) and thiophenol (0.92 mL, 8.95 mmol) at room temperature. The resulted reaction mixture was stirred at 80° C. for 2 h. The reaction mixture was filtered through celite bed and the collected filtrate was concentrated under reduced pressure to get crude 13 as yellow oil. The crude mixture was subjected to reverse phase chromatography to yield 13 (1.9 g, 45.8%) as a light yellow solid. The yellow solid was further subjected to preparative HPLC (method mentioned below) purification followed by lyophilization to yield pure 13 (0.34 g, 8.2%) as a white amorphous powder. MS (ESI-MS): m/z calcd for C₅₃H₆₂N₁₂O₉ [MH]⁺ 1391.86, found 1392.3.

Method for Preparative HPLC:

(A) 10 mM NH₄HCO₃ in water (HPLC grade) and (B) 100% acetonitrile (HPLC grade) in water (HPLC grade), using X-BRIDGE C18, 250 mm*30 mm*5 μm with the flow rate of 30.0 mL/min and with the following gradient:

| Time | % A | % B |
|---|---|---|
| 0.01 | 32.0 | 68.0 |
| 25.00 | 26.0 | 74.0 |
| 25.01 | 0.0 | 100 |
| 26.00 | 0.0 | 100 |
| 26.01 | 32.0 | 68.0 |
| 27.00 | 32.0 | 68.0 |

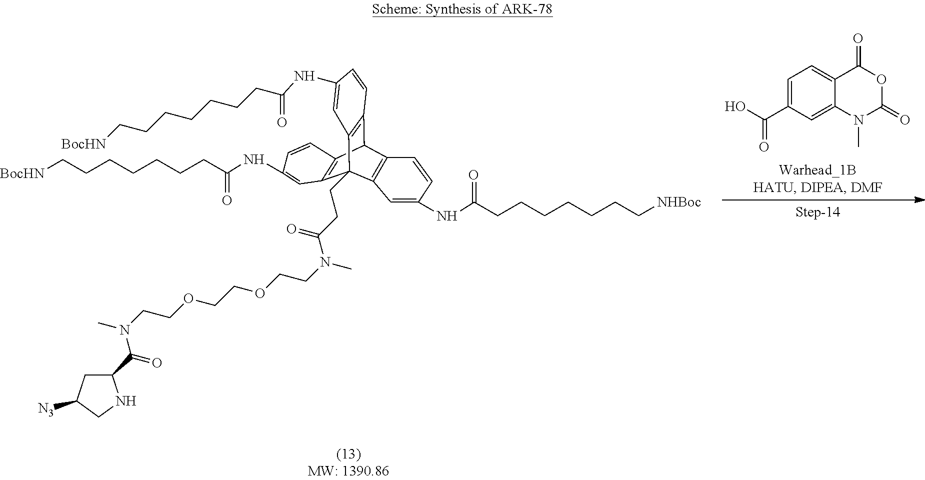

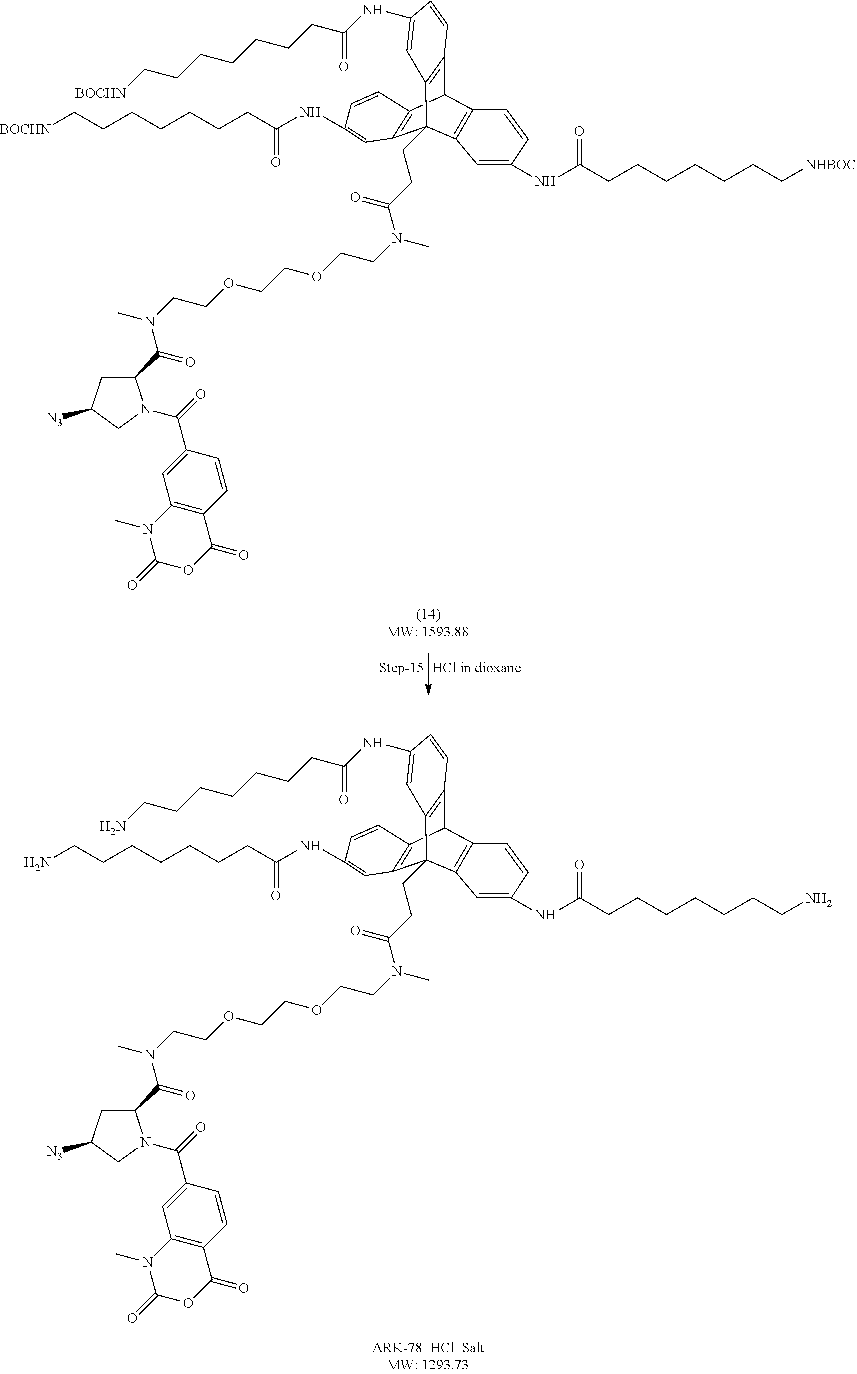

### Tri-tert-butyl (((9-(1-((2S,4S)-4-azido-1-(1-methyl-2,4-dioxo-1,4-dihydro-2H-benzo[d][1,3]oxazine-7-carbonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl tri-tert-butyl (((9-(1-((2S,4S)-4-azidopyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl)) tris(8-oxooctane-8,1-diyl))tricarbamate (13) (0.14 g, 0.1 mmol) in N,N-dimethylformamide (5 mL) were sequentially added 1-methyl-2,4-dioxo-2,4-dihydro-1H-3,1-benzoxazine-7-carboxylic acid (Warhead_type_1B) (0.027 g, 0.12 mmol) and HATU (0.046 g, 0.12 mmol) at room temperature. The reaction mixture was stirred for 5 minutes. To this, N,N-diisopropylethylamine (0.026 g, 0.201 mmol) was added dropwise and the resulted reaction mixture was further stirred for 30 minutes at room temperature. The reaction mixture was diluted by ethyl acetate (100 mL) and washed with ice-cold water (3×30 mL). The organic layers were combined, washed with brine and concentrated under reduced pressure at 25° C. to get crude 14 (0.1 g, 62.5%) as a light yellow solid which was used in the next step without further purification. MS (ESI-MS): m/z calcd C₉₅H₁₁₉N₁₃O₁₇ [MH]⁺ 1594.88, found 1496.61 (M−100).

### N,N′,N″-(9-(1-((2S,4S)-4-azido-1-(1-methyl-2,4-dioxo-1,4-dihydro-2H-benzo[d][1,3]oxazine-7-carbonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(8-aminooctanamide), ARK-78_HCl Salt

To a solution of tri-tert-butyl (((9-(1-((2S,4S)-4-azido-1-(1-methyl-2,4-dioxo-1,4-dihydro-2H-benzo[d][1,3]oxazine-7-carbonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (14) (0.067 g, 0.042 mmol) in 1,4-dioxane (3.0 mL) was added 4 M HCl in dioxane solution (1.5 mL) at room temperature and the resulted reaction mixture was stirred for 30 minutes under nitrogen atmosphere. During this, solid residue started to precipitate out. The suspension was further stirred for 30 minutes and finally allowed to stand at room temperature. The solid residues started to deposit on bottom of the flask. The solvent was decanted and the residues left were triturated with acetonitrile (3×3 mL). Finally the solid was dried under reduced pressure at 25° C. to get pure ARK-78_HCl_Salt (0.045 g, 76.3%) as a white amorphous powder. ¹H NMR (400 MHz, DMSO-d6) δ 9.91 ppm (3H, broad s), 8.11-7.97 ppm (1H, m), 7.89 ppm (8H, broad s), 7.66 ppm (3H, broad s), 7.37-7.34 ppm (2H, broad s), 7.29-7.22 ppm (6H, m), 5.39 ppm (1H, s), 4.97 ppm (1H, m), 4.82 ppm (1H, m), 4.28 ppm (2H, m), 4.03 ppm (1H, m), 3.74 ppm (1H, m), 3.64 ppm (3H, broad s), 3.57 ppm (12H, broad s), 3.50-3.47 ppm (5H, m), 3.15-3.03 ppm (7H, m), 2.90-2.85 ppm (2H, d), 2.75-2.72 ppm (7H, m), 2.25-2.23 ppm (6H, broad s), 1.54 ppm (12H, broad s), 1.27 ppm (17H, broad s). MS (ESI-MS): m z calcd for C₇₀H₉₅N₁₃O₁₁ [MH]⁺ 1294.73, found 1295.41.

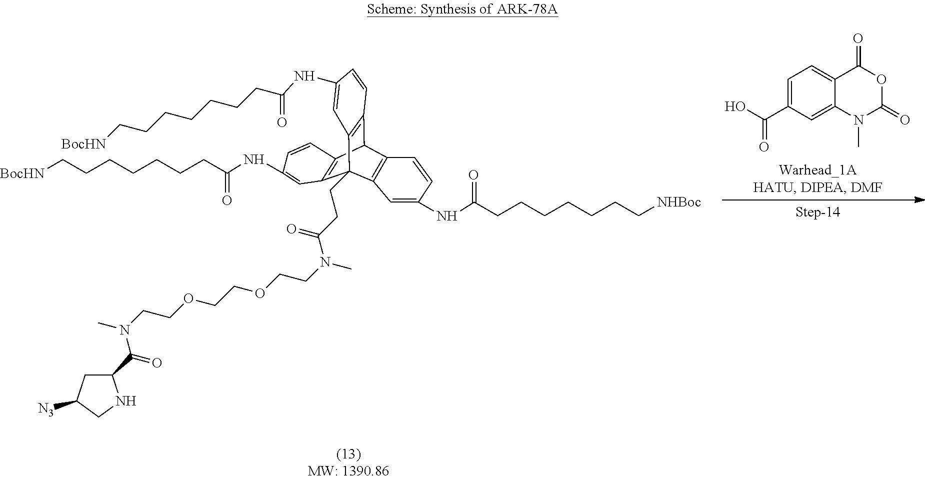

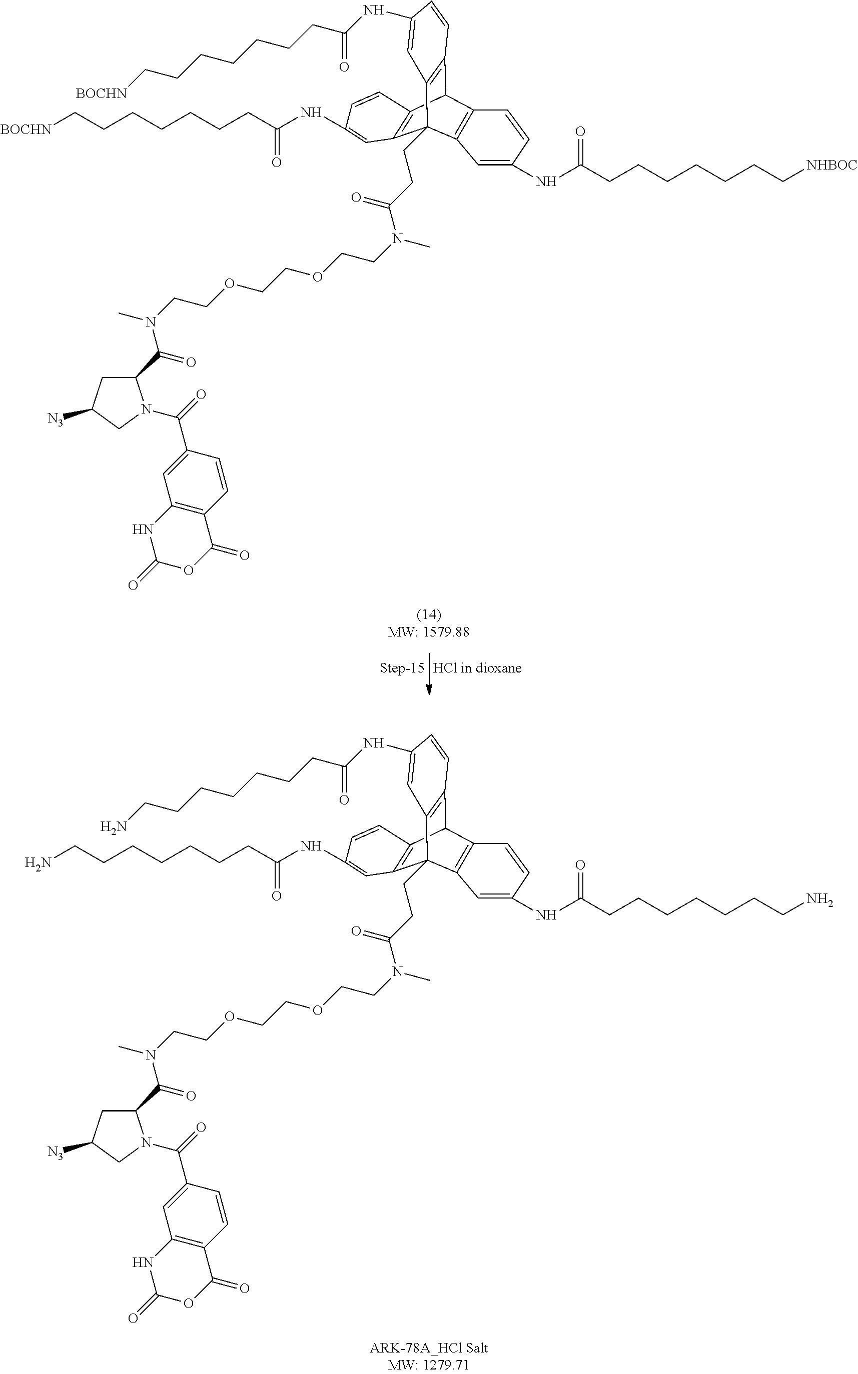

### Tri-tert-butyl (((9-(1-((2S,4S)-4-azido-1-(2,4-dioxo-1,4-dihydro-2H-benzo[d][1,3]oxazine-7-carbonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl)) tris(8-oxooctane-8,1-diyl))tricarbamate (13) (0.075 g, 0.05 mmol) in N,N-dimethylformamide (4 mL) were sequentially added 2,4-dioxo-1,4-dihydro-2H-benzo[d][1,3]oxazine-7-carboxylic acid (Warhead_type_1A) (0.013 g, 0.065 mmol) and HATU (0.024 g, 0.065 mmol) at room temperature. The reaction mixture was stirred for 5 minutes. To this, N,N-diisopropylethylamine (0.014 g, 0.108 mmol) was added drop wise and the resulted reaction mixture was further stirred for 30 minutes at room temperature. The reaction mixture was diluted by ethyl acetate (100 mL) and washed with ice-cold water (3×30 mL). The organic layers were combined, washed with brine and concentrated under reduced pressure at 25° C. to get crude 14. The crude mixture was purified by preparative HPLC using following method to yield pure 14 (0.04 g, 52%) as a white amorphous powder. The prep-fraction was concentrated by reduced pressure at 25° C. under nitrogen atmosphere. MS (ESI-MS): m/z calcd for C₈₄H₁₁₇N₁₃O₁₇ [MH]⁺ 1580.88, found 1481.75 (M−100).

Method for Preparative HPLC:

(A) 100% Acetonitrile (HPLC GRADE) and (B) 100% Tetrahydrofuran (HPLC GRADE), using SUNFIRE SILICA, 150 mm*19 mm*5 μm with the flow rate of 16.0 mL/min and with the following gradient:

| Time | % A | % B |
|---|---|---|
| 0.01 | 98.0 | 2.0 |
| 20.00 | 98.0 | 2.0 |

### N,N′,N″-(9-(1-((2S,4S)-4-azido-1-(2,4-dioxo-1,4-dihydro-2H-benzo[d][1,3]oxazine-7-carbonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(8-aminooctanamide), ARK-78A_HCl Salt

To a solution of tri-tert-butyl (((9-(1-((2S,4S)-4-azido-1-(2,4-dioxo-1,4-dihydro-2H-benzo[d][1,3]oxazine-7-carbonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (14) (0.04 g, 0.025 mmol) in 1,4-Dioxane (AR Grade) (2 mL) was added 4 M HCl in dioxane (1 mL) at room temperature and the resulting reaction mixture was stirred for 30 minutes under nitrogen atmosphere. The solid material stable at the bottom of the flask, the solvent was decanted under inert atmosphere, the solid material was triturating with acetonitrile (HPLC Grade) (3×3 mL). The remaining solid was concentrated by reduced pressure at 25° C. under nitrogen atmosphere to afford pure ARK-78A_HCl_Salt (0.032 g, 91.43%) as a white amorphous powder. ¹H NMR (400 MHz, DMSO-d6) δ 11.99-11.95 ppm (1H, t), 9.91-9.90 ppm (3H, d), 8.01-7.94 ppm (1H, m), 7.87 ppm (8H, broad s), 7.66 ppm (3H, broad s), 7.32-7.22 ppm (7H, m), 7.16-7.11 ppm (1H, m), 5.39 ppm (1H, s), 4.99-4.95 ppm (1H, t), 4.83-4.82 ppm (1H, m), 4.29-4.22 ppm (1H, m), 4.15-3.98 ppm (1H, m), 3.76-3.71 ppm (1H, m), 3.64-3.61 ppm (4H, m), 3.52 ppm (2H, broad s), 3.34-3.32 ppm (2H, m), 3.15 ppm (2H, m), 3.10-3.03 ppm (7H, m), 2.89-2.86 ppm (1H, d), 2.76-2.72 ppm (7H, m), 2.26-2.23 ppm (6H, t), 1.53 ppm (12H, broad s), 1.27 ppm (17H, broad s). MS (ESI-MS): m/z calcd for C₆₉H₉₃N₁₃O₁₁ [MH]⁺. 1280.71, found 1281.50.

### Example 14: Synthesis of ARK-79 and ARK-79A (Ark000036 and Ark000038)

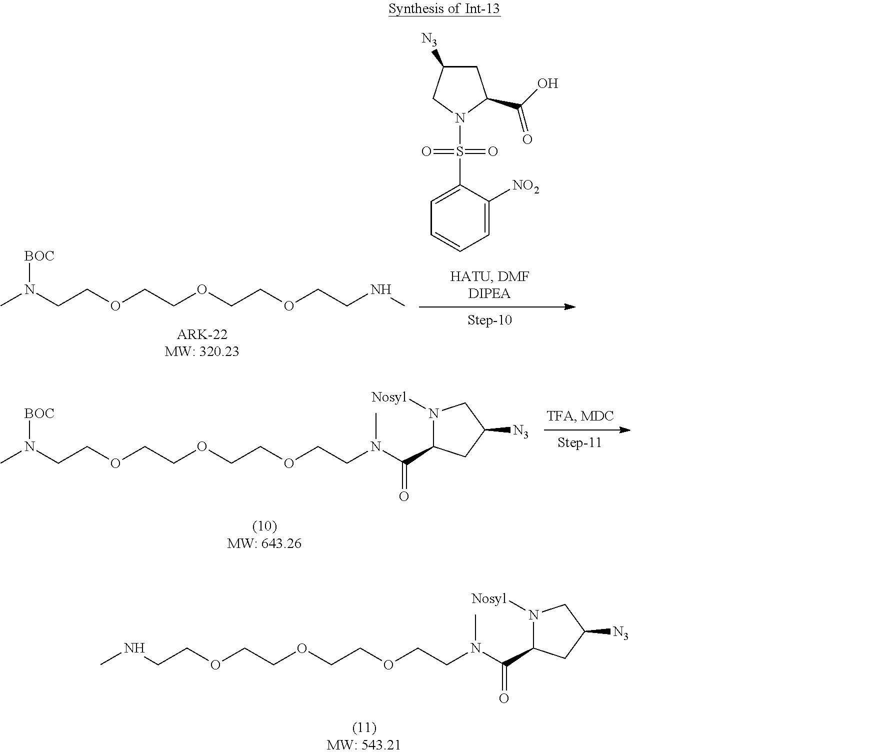

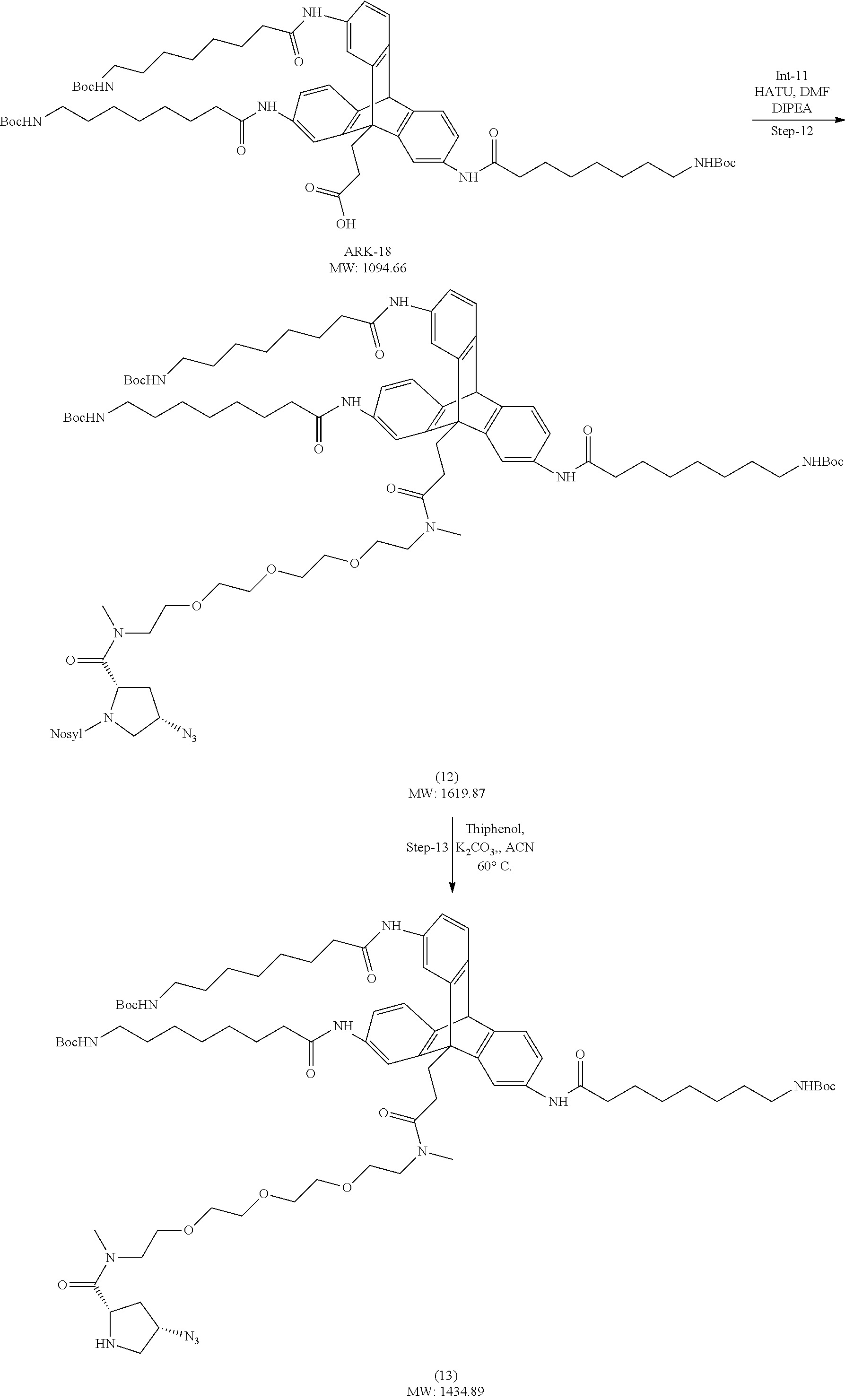

Tert-butyl (1-((2S,4S)-4-azido-1-((2-nitrophenyl)sulfonyl)pyrrolidin-2-yl)-2-methyl-1-oxo-5,8,11-trioxa-2-azatridecan-13-yl)(methyl)carbamate, 10.

To a solution of ARK-22 (3.1 g, 9.68 mmol) in N,N-dimethylformamide (40 mL) were sequentially added (2S,4S)-4-azido-1-((2-nitrophenyl)sulfonyl)pyrrolidine-2-carboxylic acid (3.96 g, 11.62 mmol), HATU (4.414 g, 11.62 mmol) and N,N-diisopropylethylamine (2.5 g, 19.36 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 10 (4 g, 64.2%) as yellow solid. The crude mixture was used in next step without further purification. MS (ESI-MS): m/z calcd for C₂₆H₄₁N₇O₁₀S [MH]⁺ 644.26, found 544.36 (M+18).

### (2S,4S)-4-azido-N-methyl-1-((2-nitrophenyl)sulfonyl)-N-(5,8,11-trioxa-2-azatridecan-13-yl) pyrrolidine-2-carboxamide_TFA Salt, 11

To a solution of tert-butyl (1-((2S,4S)-4-azido-1-((2-nitrophenyl)sulfonyl)pyrrolidin-2-yl)-2-methyl-1-oxo-5,8,11-trioxa-2-azatridecan-13-yl)(methyl) carbamate (10) (3 g, 4.66 mmol) in dichloro methane (20 mL) was added trifluoro acetic acid (1.8 mL, 23.32 mmol) at room temperature. The resulted reaction mixture was stirred at room temperature for 2 h. The reaction mixture was filtered through celite bed and filtrate thus collected was concentrated under reduced pressure to get crude 11 (3.1 g, quantitative yield) as a dark yellow oil which was used without further purification. MS (ESI-MS): m/z calcd for C₂₁H₃₃N₇O₈S.TFA [MH]⁺ 544.21, found 544.47.

### Tri-tert-butyl (((9-(1-((2S,4S)-4-azido-1-((2-nitrophenyl)sulfonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate, 12

To a solution of (2S,4S)-4-azido-N-methyl-1-((2-nitrophenyl)sulfonyl)-N-(5,8,11-trioxa-2-azatridecan-13-yl) pyrrolidine-2-carboxamide_TFA Salt (11) (2.88 g, 4.38 mmol) in N,N-dimethylformamide (40 mL) were sequentially added 3-(2,7,15-tris(8-((tert-butoxycarbonyl)amino)octanamido)-9,10-[1,2]benzenoanthracen-9(10H)-yl)propanoic acid (ARK-18) (4.0 g, 3.65 mmol), HATU (1.67 g, 4.38 mmol) and N,N-diisopropylethylamine (2.36 g, 18.27 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 12. The crude mixture was purified by column chromatography on silica gel (5.4% methanol/chloroform) to yield 12 (5.9 g, 99.7%) as a dark yellow solid. MS (ESI-MS): m/z calcd for C₉₃H₁₂₁N₁₃O₁₈S [MH]⁺ 1620.87, found 1522.31 (M−100; one Boc group fell off).

### Tri-tert-butyl (((9-(1-((2S,4S)-4-azidopyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris (8-oxooctane-8,1-diyl))tricarbamate, 13

To a solution of tri-tert-butyl (((9-(1-((2S,4S)-4-azido-1-((2-nitrophenyl)sulfonyl)pyrrolidin-2-yl)-2,14-dimethyl-1,15-dioxo-5,8,11-trioxa-2,14-diazaheptadecan-17-yl)-9,10-dihydro-9,10-[1,2] benzenoanthracene-2,7,15-triyl)tris(azanediyl))tris(8-oxooctane-8,1-diyl))tricarbamate (12) (5.9 g, 3.64 mmol) in acetonitrile (60 mL) were sequentially added potassium carbonate (2.51 g, 18.21 mmol) and thiophenol (1.11 mL, 10.93 mmol) at room temperature. The resulted reaction mixture was stirred at 80° C. for 2 h. The reaction mixture was filtered through celite bed and the collected filtrate was concentrated under reduced pressure to get crude 13 as yellow oil. The crude mixture was subjected to reverse phase chromatography to yield 13 (1.9 g, 36.3%) as a light yellow solid. The yellow solid was further subjected to preparative HPLC (method mentioned below) purification followed by lyophilization to yield pure 13 (0.51 g, 9.8%) as a white amorphous powder. MS (ESI-MS): m/z calcd for C₇₇H₁₁₈N₁₂O₁₄ [MH]⁺ 1435.89, found 1437.41.

Method for Preparative HPLC:

(A) 100% Acetonitrile (HPLC GRADE) IN WATER (HPLC GRADE) and (B) 10 mM NH₄HCO₃ IN WATER (HPLC GRADE), using GRACE DENIL C18, 250 mm*25 mm*5 μm with the flow rate of 22.0 mL/min and with the following gradient:

| Time | % A | % B |
|---|---|---|
| 0.01 | 50.0 | 50.0 |
| 3.00 | 25.0 | 75.0 |
| 25.00 | 22.0 | 78.0 |
| 25.01 | 0.0 | 100 |
| 26.00 | 0.0 | 100 |
| 26.01 | 50.0 | 50.0 |
| 27.00 | 50.0 | 50.0 |

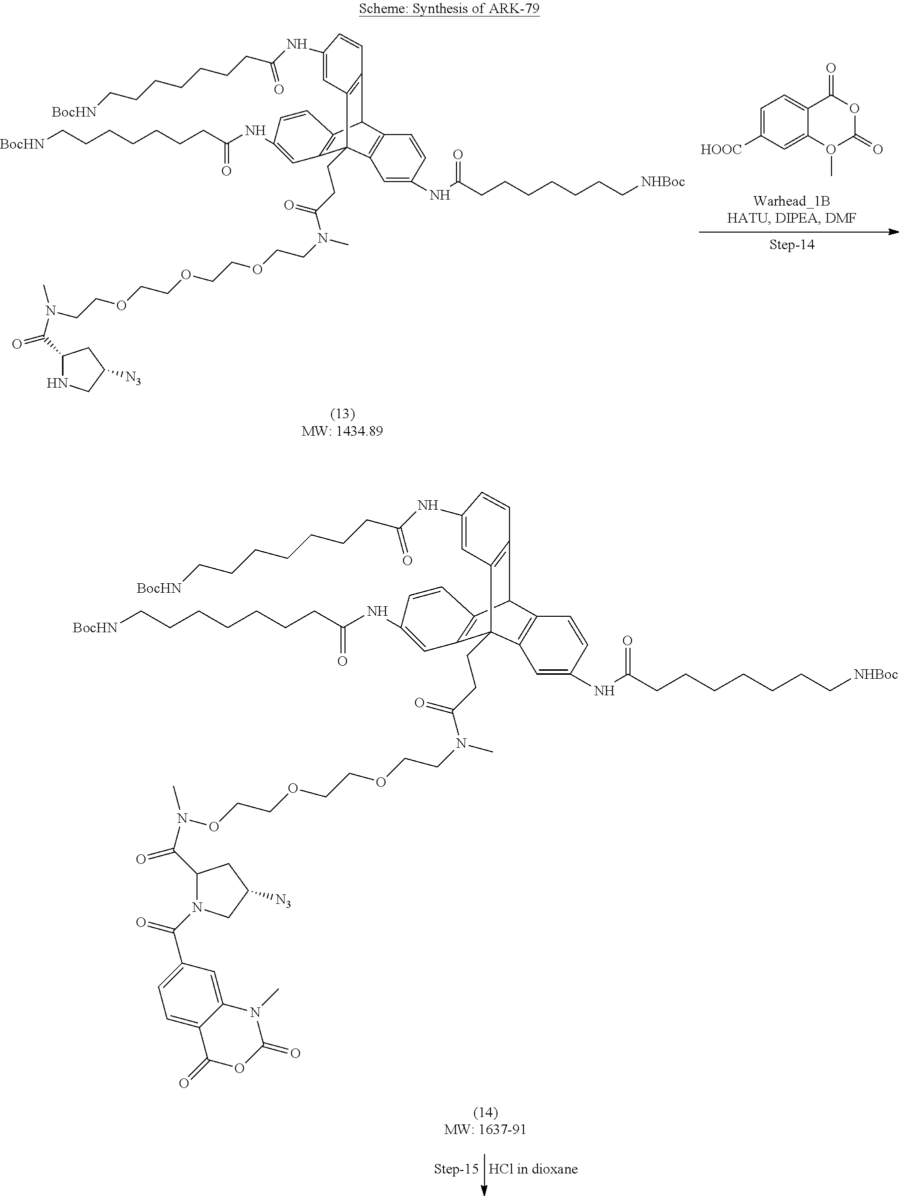

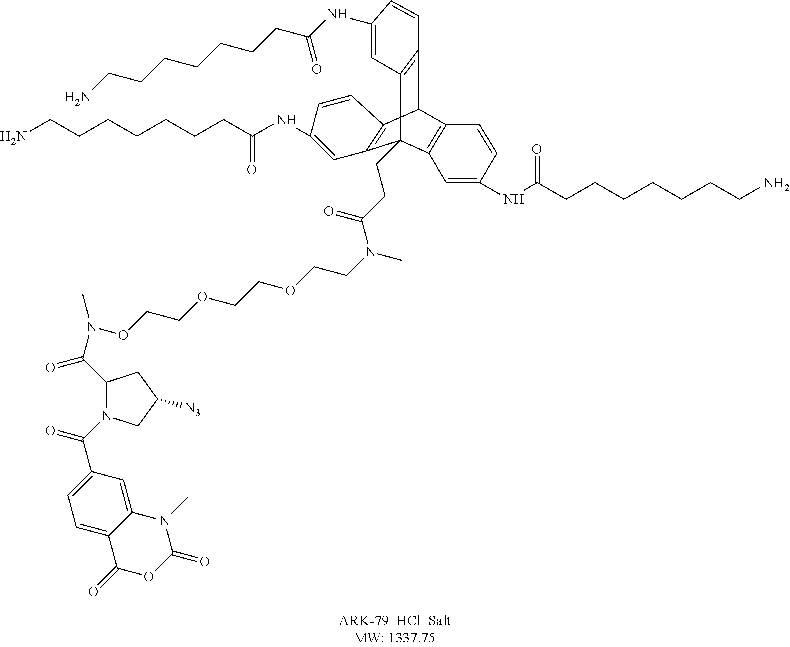 [SMILES: CN(CCOCCOCCON(C)C(=O)C1C[C@H](N=[N+]=[N-])CN1C(=O)c1ccc2c(=O)oc(=O)n(C)c2c1)C(=O)CCC12c3cc(NC(=O)CCCCCCCN)ccc3C(c3ccc(NC(=O)CCCCCCCN)cc31)c1ccc(NC(=O)CCCCCCCN)cc12]

### Tri-tert-butyl (((9-(1-((2S,4S)-4-azido-1-(1-methyl-2,4-dioxo-1,4-dihydro-2H-benzo[d][1,3]oxazine-7-carbonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris (8-oxooctane-8,1-diyl))tricarbamate (13) (0.1 g, 0.07 mmol) in N,N-dimethylformamide (4 mL) were sequentially added 1-methyl-2,4-dioxo-2,4-dihydro-1H-3,1-benzoxazine-7-carboxylic acid (Warhead_type_1B) (0.039 g, 0.18 mmol) and HATU (0.018 g, 0.084 mmol) at room temperature. The reaction mixture was stirred for 5 minutes. To this, N,N-diisopropylethylamine (0.018 g, 0.14 mmol) was added drop wise and the resulted reaction mixture was further stirred for 30 minutes at room temperature. The reaction mixture was diluted by ethyl acetate (100 mL) and washed with ice-cold water (3×30 mL). The organic layers were combined, washed with brine and concentrated under reduced pressure at 25° C. to get crude 14. The crude mixture was purified by preparative HPLC (method mentioned below) followed by lyophilization to yield 14 (0.053 g, 46.5%) as a white amorphous powder. MS (ESI-MS): m/z calcd C₈₇H₁₂₃N₁₃O₁₉ [MH]⁺ 1638.91, found 1540.40 (M−100).

Method for Preparative HPLC:

(A) 100% Acetonitrile (HPLC GRADE) and (B) 100% Tetrahydrofuran (HPLC GRADE), using SUNFIRE SILICA, 250 mm*19 mm*5 μm with the flow rate of 15.0 mL/min and with the following gradient:

| Time | % A | % B |
|---|---|---|
| 0.01 | 95.0 | 5.0 |
| 20.00 | 95.0 | 5.0 |

### N,N′,N″-(9-(1-((2S,4S)-4-azido-1-(1-methyl-2,4-dioxo-1,4-dihydro-2H-benzo[d][1,3]oxazine-7-carbonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(8-aminooctanamide), ARK-79_HCl Salt

To a solution of tri-tert-butyl (((9-(1-((2S,4S)-4-azido-1-(1-methyl-2,4-dioxo-1,4-dihydro-2H-benzo[d][1,3]oxazine-7-carbonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate (14) (0.035 g, 0.021 mmol) in 1,4-dioxane (3.0 mL) was added 4 M HCl in dioxane solution (1 mL) at room temperature and the resulted reaction mixture was stirred for 30 minutes under nitrogen atmosphere. During this, solid residue started to precipitate out. The suspension was further stirred for 30 minutes and finally allowed to stand at room temperature. The solid residues started to deposit on bottom of the flask. The solvent was decanted and the residues left were triturated with acetonitrile (3×3 mL). Finally the solid was dried under reduced pressure at 25° C. to get pure ARK-79_HCl_Salt (0.025 g, 80.6%) as a white amorphous powder. ¹H NMR (400 MHz, DMSO-d₆) δ 9.89 ppm (3H, broad s), 8.10-8.08 ppm (1H, m), 7.89 ppm (9H, broad s), 7.66 ppm (3H, broad s), 7.38-7.37 ppm (1H, d), 7.33-7.22 ppm (6H, m), 5.38 ppm (1H, s), 4.95-4.90 ppm (1H, m), 4.25 ppm (1H, m), 4.06 ppm (1H, m), 3.75 ppm (1H, m), 3.63-3.57 ppm (10H, d), 3.38-3.33 ppm (5H, m), 3.10-3.04 ppm (7H, m), 2.88-2.84 ppm (1H, d), 2.74-2.72 ppm (7H, broad s), 2.25-2.23 ppm (6H, t), 1.60-1.53 ppm (12H, d), 1.27 ppm (18H, broad s). MS (ESI-MS): m/z calcd for C₇₂H₉₉N₁₃O₁₂ [MH]⁺ 1338.75, found 1339.55.

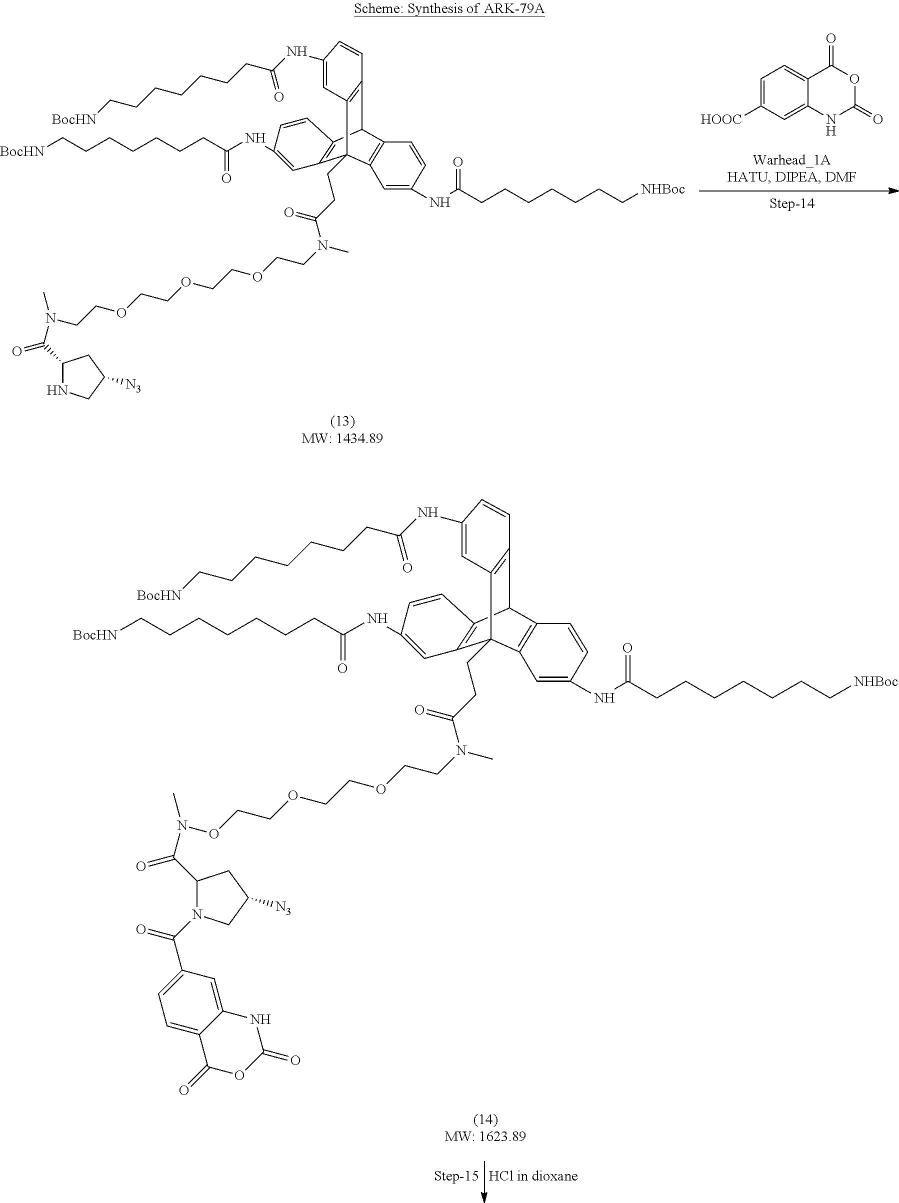 [SMILES: CN(CCOCCOCCOCCN(C)C(=O)[C@@H]1C[C@H](N=[N+]=[N-])CN1)C(=O)CCC12c3cc(NC(=O)CCCCCCCNC(=O)OC(C)(C)C)ccc3C(c3ccc(NC(=O)CCCCCCCNC(=O)OC(C)(C)C)cc31)c1ccc(NC(=O)CCCCCCCNC(=O)OC(C)(C)C)cc12 ; O=C(O)c1ccc2c(=O)oc(=O)[nH]c2c1 ; CN(CCOCCOCCON(C)C(=O)C1C[C@H](N=[N+]=[N-])CN1C(=O)c1ccc2c(=O)oc(=O)[nH]c2c1)C(=O)CCC12c3cc(NC(=O)CCCCCCCNC(=O)OC(C)(C)C)ccc3C(c3ccc(NC(=O)CCCCCCCNC(=O)OC(C)(C)C)cc31)c1ccc(NC(=O)CCCCCCCNC(=O)OC(C)(C)C)cc12]

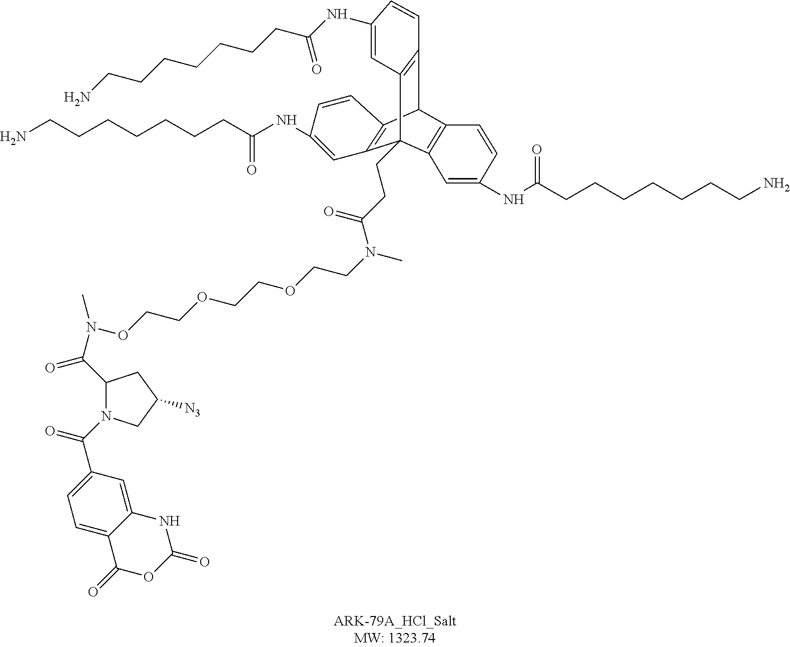 [SMILES: CN(CCOCCOCCON(C)C(=O)C1C[C@H](N=[N+]=[N-])CN1C(=O)c1ccc2c(=O)oc(=O)[nH]c2c1)C(=O)CCC12c3cc(NC(=O)CCCCCCCN)ccc3C(c3ccc(NC(=O)CCCCCCCN)cc31)c1ccc(NC(=O)CCCCCCCN)cc12]

### Tri-tert-butyl (((9-(1-((2S,4S)-4-azido-1-(2,4-dioxo-1,4-dihydro-2H-benzo[d][1,3]oxazine-7-carbonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris (8-oxooctane-8,1-diyl))tricarbamate (13) (0.2 g, 0.139 mmol) in N,N-dimethylformamide (8 mL) were sequentially added 2,4-dioxo-1,4-dihydro-2H-benzo[d][1,3]oxazine-7-carboxylic acid (Warhead_type_1A) (0.035 g, 0.167 mmol) and HATU (0.064 g, 0.167 mmol) at room temperature. The reaction mixture was stirred for 5 minutes. To this, N,N-diisopropylethylamine (0.036 g, 0.279 mmol) was added dropwise and the resulted reaction mixture was further stirred for 30 minutes at room temperature. The reaction mixture was diluted by ethyl acetate (100 mL) and washed with ice-cold water (3×30 mL). The organic layers were combined, washed with brine and concentrated under reduced pressure at 25° C. to get crude 14. The crude mixture was purified by preparative HPLC using following method to yield pure 14 (0.04 g, 17.7%) as a off white amorphous powder. The prep-fraction was concentrated by reduced pressure at 25° C. under nitrogen atmosphere. MS (ESI-MS): m/z calcd C₈₆H₁₂₁N₁₃O₁₉[MH]⁺ 1624.89, found 1525.76 (M−100; one Boc group fell off).

Method for Preparative HPLC:

(A) 100% Acetonitrile (HPLC GRADE) and (B) 100% Tetrahydrofuran (HPLC GRADE), using SUNFIRE SILICA, 150 mm*19 mm*5 μm with the flow rate of 18.0 mL/min and with the following gradient: 98% A and 2% for 20 min.

### N,N′,N″-(9-(1-((2S,4S)-4-azido-1-(2,4-dioxo-1,4-dihydro-2H-benzo[d][1,3]oxazine-7-carbonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(8-aminooctanamide), ARK-79A_HCl Salt

To a solution of tri-tert-butyl (((9-(1-((2S,4S)-4-azido-1-(2,4-dioxo-1,4-dihydro-2H-benzo[d][1,3]oxazine-7-carbonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate (14) (0.04 g, 0.024 mmol) in 1,4-Dioxane (Dry) (3 ml) was added 4 M HCl in dioxane (1.2 mL) at room temperature and the resulting reaction mixture was stirred for 30 minutes under nitrogen atmosphere. The solid material stable at the bottom of RBF, the solvent was decant under inert atmosphere, the solid material was triturating with acetonitrile (HPLC Grade) (3×3 mL). The remaining solid was concentrated by reduced pressure at 25° C. under nitrogen atmosphere to afford pure ARK-79A_HCl_Salt (0.033 g, 94.28%) as a off white amorphous powder. ¹H NMR (400 MHz, DMSO-d6) δ 11.97-11.95 ppm (1H, d), 9.90 ppm (3H, broad s), 8.02-7.98 ppm (1H, m), 7.88 ppm (8H, broad s), 7.66 ppm (3H, broad s), 7.32-7.22 ppm (7H, m), 7.16-7.08 ppm (1H, m), 5.39 ppm (1H, s), 4.96-4.91 ppm (1H, m), 4.80 ppm (1H, m), 4.28-4.20 ppm (1H, m), 4.05 ppm (1H, m), 3.75-3.73 ppm (1H, m), 3.64 ppm (3H, broad s), 3.57 ppm (11H, broad s), 3.54 ppm (2H, m), 3.39-3.38 ppm (2H, m), 3.34-3.32 ppm (3H, d), 3.16 ppm (2H, broad s), 3.08-3.02 ppm (8H, m), 2.87-2.83 ppm (1H, d), 2.76-2.68 ppm (7H, m), 2.27-2.23 ppm (6H, t), 1.60-1.53 ppm (12H, broad s), 1.27 ppm (18H, broad s). MS (ESI-MS): m/z calcd for C₇₁H₉₇N₁₃O₁₂ [MH]⁺ 1324.74, found 1325.50.

### Example 15: Synthesis of ARK-80, ARK-89, ARK-125 (Ark000024, Ark000027, and Ark000030)

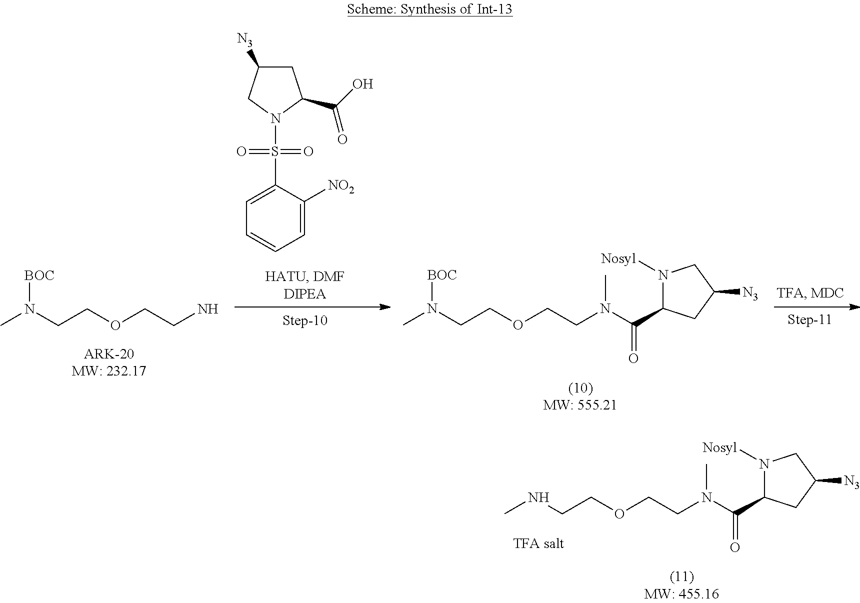

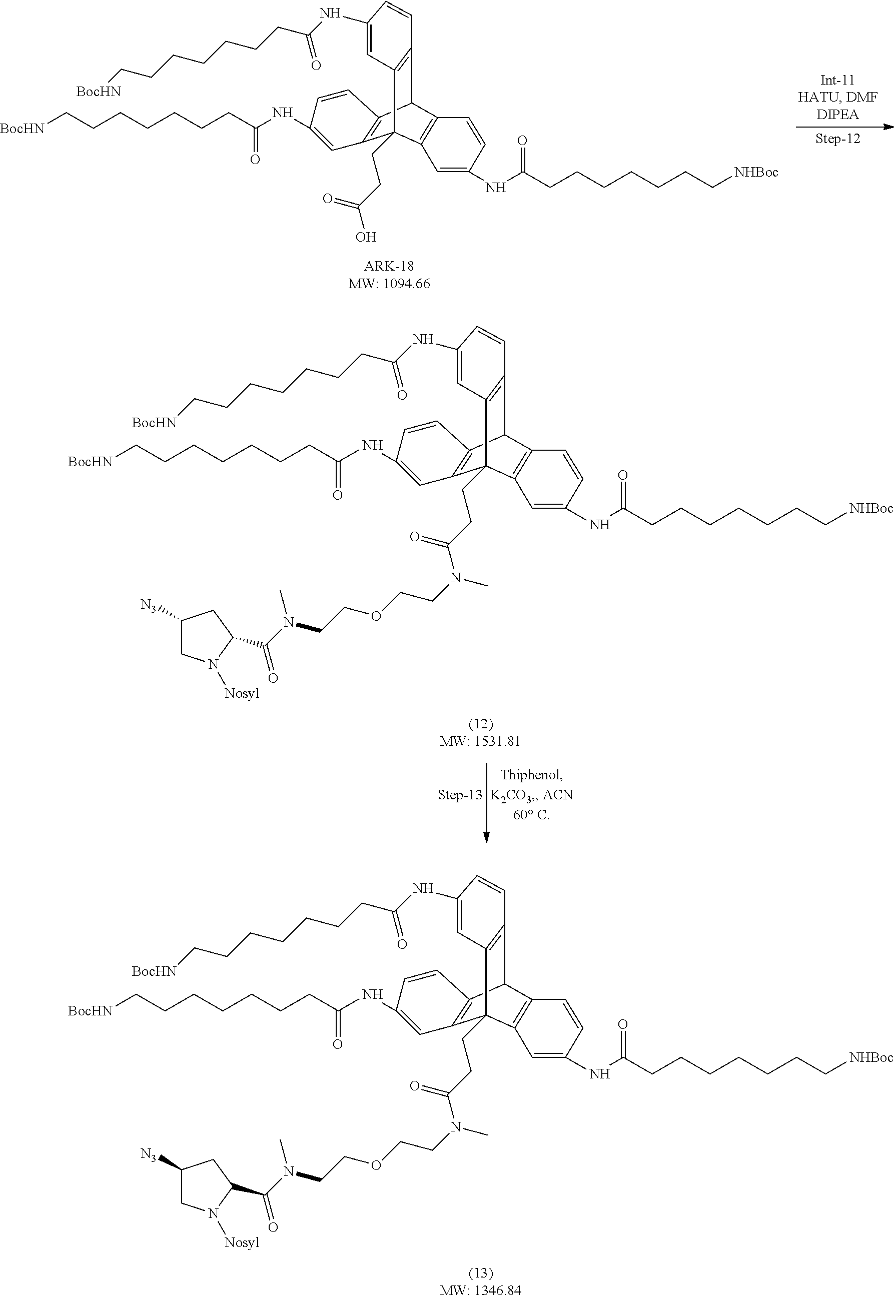

### Tert-butyl (2-(2-((2S,4S)-4-azido-N-methyl-1-((2-nitrophenyl)sulfonyl)pyrrolidine-2-carboxamido) ethoxy)ethyl)(methyl)carbamate, 10

To a solution of ARK-20 (1.0 g, 4.307 mmol) in N,N-dimethylformamide (20 mL) were sequentially added (2S,4S)-4-azido-1-((2-nitrophenyl)sulfonyl)pyrrolidine-2-carboxylic acid (1.17 g, 3.44 mmol), HATU (1.96 g, 5.17 mmol) and N,N-diisopropylethylamine (1.67 g, 12.92 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 10 (2.52 g, quantitative yield) as brown semisolid. The crude mixture was used in next step without further purification. MS (ESI-MS): m/z calcd for C₂₂H₃₃N₇O₈S [MH]⁺ 556.21, found 573.43 (M+18).

### (2S,4S)-4-azido-N-methyl-N-(2-(2-(methylamino)ethoxy)ethyl)-1-((2-nitrophenyl)sulfonyl) pyrrolidine-2-carboxamide_TFA Salt, 11

To a solution of tri tert-butyl (2-(2-((2S,4S)-4-azido-N-methyl-1-((2-nitrophenyl)sulfonyl)pyrrolidine-2-carboxamido) ethoxy)ethyl)(methyl)carbamate (10) (2.5 g, 4.50 mmol) in dichloro methane (15 mL) was added trifluoro acetic acid (1.72 mL, 22.51 mmol) at room temperature. The resulted reaction mixture was stirred at room temperature for 2 h. The reaction mixture was filtered through celite bed and filtrate thus collected was concentrated under reduced pressure to get crude 11 (4.12 g, quantitative yield) as a brown oil which was used without further purification. MS (ESI-MS): m/z calcd for C₁₇H₂₅N₇O₆S.TFA [MH]⁺ 456.16, found 456.32.

### Tri-tert-butyl (((9-(3-((2-(2-((2S,4S)-4-azido-N-methyl-1-((2-nitrophenyl)sulfonyl)pyrrolidine-2-carboxamido)ethoxy)ethyl)(methyl)amino)-3-oxopropyl)-9,10-dihydro-9,10[1,2]benzenoanthracene-2,7,15-triyl)tris(azanediyl))tris(8-oxooctane-8,1-diyl))tricarbamate, 12

To a solution of (2S,4S)-4-azido-N-methyl-N-(2-(2-(methylamino)ethoxy)ethyl)-1-((2-nitrophenyl)sulfonyl) pyrrolidine-2-carboxamide_TFA Salt (11) (1.75 g, 3.07 mmol) in N,N-dimethylformamide (30 mL) were sequentially added 3-(2,7,15-tris(8-((tert-butoxycarbonyl)amino)octanamido)-9,10-[1,2]benzenoanthracen-9(10H)-yl)propanoic acid (ARK-18) (2.8 g, 2.56 mmol), HATU (1.17 g, 3.07 mmol) and N,N-diisopropylethylamine (0.66 g, 5.12 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 12. The crude mixture was purified by column chromatography on silica gel (1.5% methanol/chloroform) to yield 12 (1.48 g, 37.8%) as a dark yellow solid. MS (ESI-MS): m/z calcd for C₇₉H₁₁₃N₁₃O₁₆S [MH]⁺ 1532.81, found 1433.19 (M−100; one Boc group fell off).

### Tri-tert-butyl (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate, 13

To a solution of tri-tert-butyl (((9-(3-((2-(2-((2S,4S)-4-azido-N-methyl-1-((2-nitrophenyl)sulfonyl) pyrrolidine-2-carboxamido)ethoxy)ethyl)(methyl)amino)-3-oxopropyl)-9,10-dihydro-9,10[1,2] benzenoanthracene-2,7,15-triyl)tris(azanediyl))tris(8-oxooctane-8,1-diyl))tricarbamate (12) (1.48 g, 0.97 mmol) in acetonitrile (15 mL) were sequentially added potassium carbonate (0.67 g, 4.83 mmol) and thiophenol (0.3 mL, 2.89 mmol) at room temperature. The resulted reaction mixture was stirred at 80° C. for 2 h. The reaction mixture was filtered through celite bed and the collected filtrate was concentrated under reduced pressure to get crude 13 as yellow oil. The crude mixture was subjected to reverse phase chromatography to yield 13 (0.76 g, 58.4%) as a light yellow solid. MS (ESI-MS): m/z calcd for C₇₃H₁₁₀N₁₂O₁₂ [MH]⁺ 1347.84, found 1349.28.

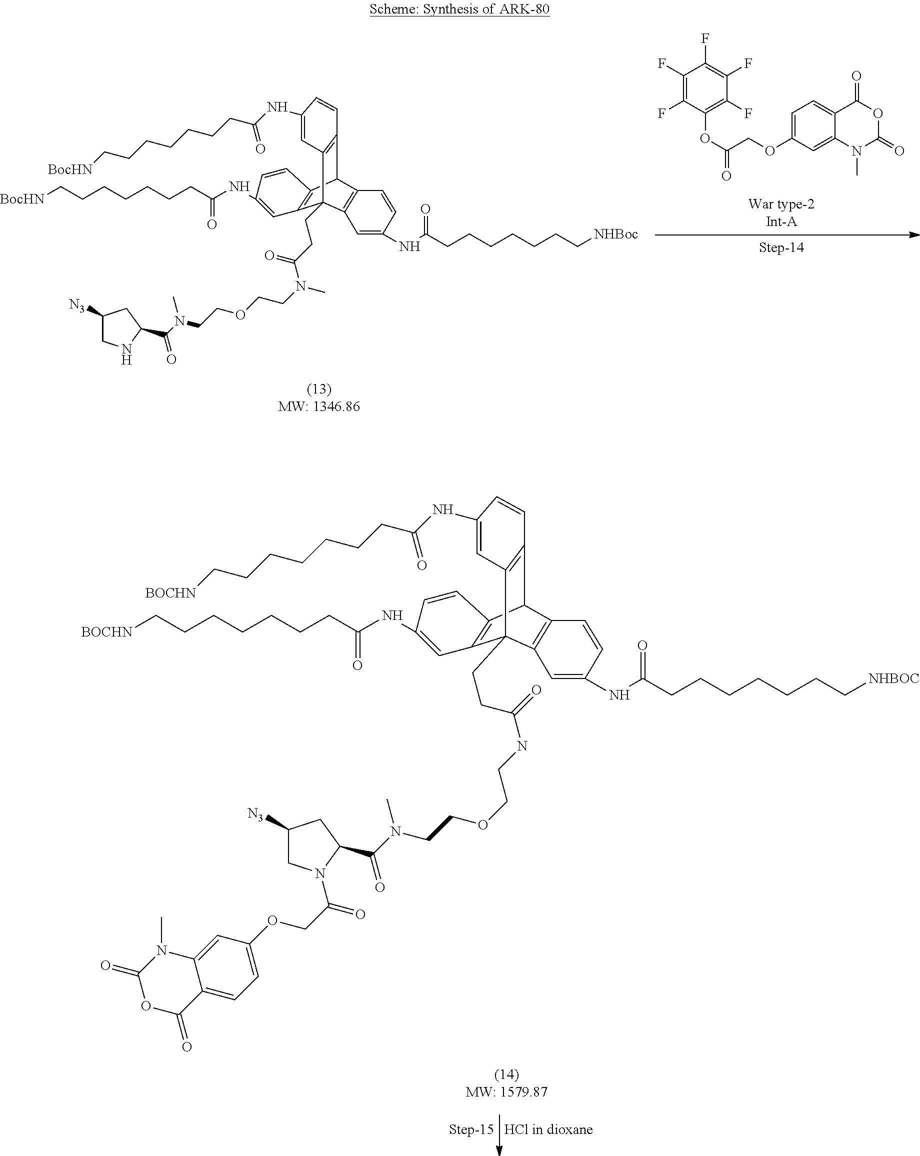

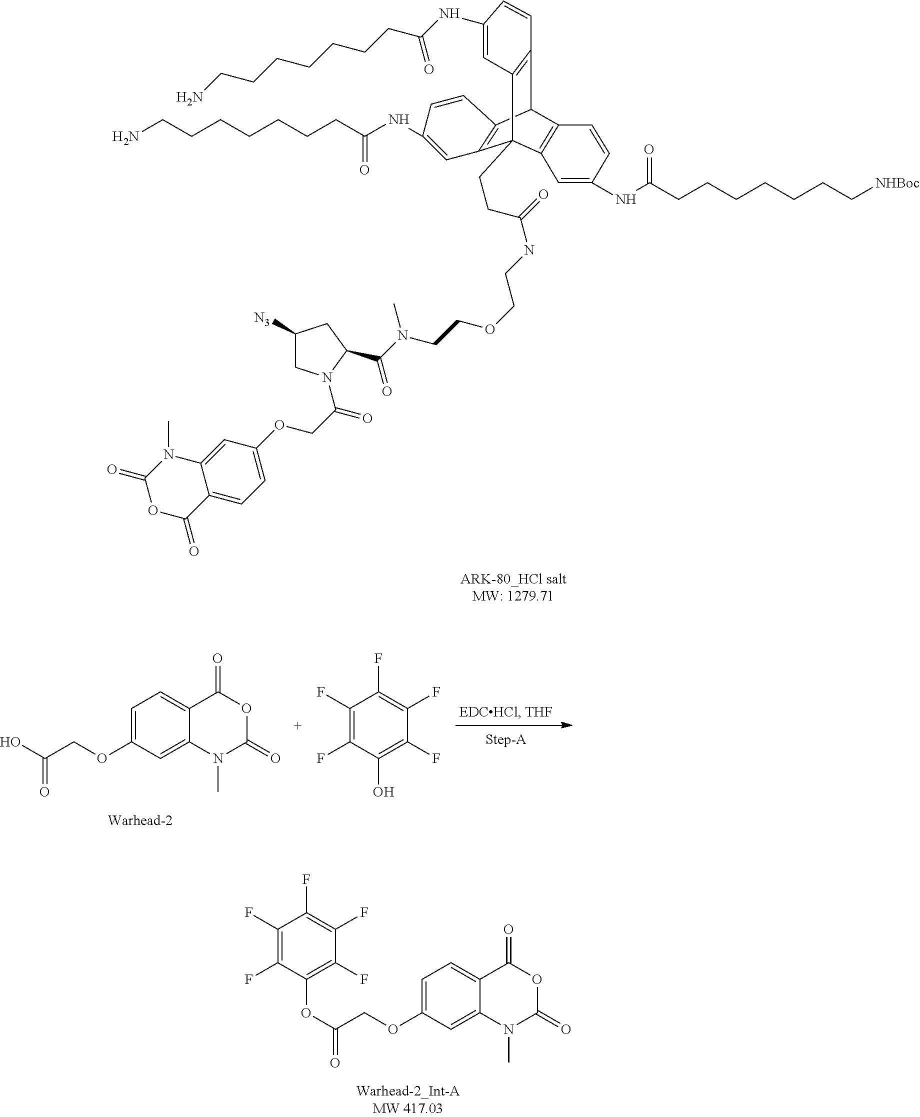

### Perfluorophenyl 2-((1-methyl-2,4-dioxo-1,4-dihydro-2H-benzo[d][1,3]oxazin-7-yl)oxy)acetate, Int-A

To a solution of Warhead-2 (0.04 g, 0.17 mmol) in tetrahydrofuran (1 mL) was added N-(3-Dimethylaminopropyl)-N-ethylcarbodiimide hydrochloride (0.035 g, 0.17 mmol) at 0° C. under nitrogen atmosphere. The reaction mixture was stirred at 0° C. for 10 min. To this, a solution of pentafluorophenol (0.03 g, 0.17 mmol) in tetrahydrofuran (0.5 mL) was added dropwise at 0° C. under nitrogen atmosphere. The resulted reaction mixture was further stirred at 0° C. for 1 h. The reaction mixture was directly used in the next step without work up and isolation. MS (ESI-MS): m/z calcd C₁₇H₈F₅NO₆ [MH]⁺ 418.03, the compound did not show mass response. Note: Intermediate-A was not isolated—the reaction mass was transferred as such to the next step reaction mass.

### Tri-tert-butyl (((9-(3-((2-(2-((2S,4S)-4-azido-N-methyl-1-(2-((1-methyl-2,4-dioxo-1,4-dihydro-2H-benzo[d][1,3]oxazin-7-yl)oxy)acetyl)pyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate (13) (0.27 g, 0.17 mmol) in tetrahydrofuran (4 mL) was added pentafluorophenyl [(1-methyl-2,4-dioxo-1,4-dihydro-2H-3,1-benzoxazin-7-yl)oxy]acetate (Warhead_type_2) (0.071 g, 0.17 mmol) and the resulted reaction mixture was further stirred for 1 h at room temperature. The reaction mixture was concentrated under reduced pressure to get crude 14 (0.38 g, Quantitative yield) as a yellow solid which was used in the next step without further purification. MS (ESI-MS): m/z calcd C₈₄H₁₁₇N₁₃O₁₇ [MH]⁺ 1580.87, found 1482.29 (M−100; one Boc group fell off).

### N,N′,N″-(9-(3-((2-(2-((2S,4S)-4-azido-N-methyl-1-(2-((1-methyl-2,4-dioxo-1,4-dihydro-2H-benzo[d][1,3]oxazin-7-yl)oxy)acetyl)pyrrolidine-2-carboxamido)ethoxy)ethyl)(methyl)amino)-3-oxopropyl)-9,10-dihydro-9,10-[1,2]benzenoanthracene-2,7,15-triyl)tris(8-aminooctanamide), ARK-80_HCl Salt

To a solution of tri-tert-butyl (((9-(3-((2-(2-((2S,4S)-4-azido-N-methyl-1-(2-((1-methyl-2,4-dioxo-1,4-dihydro-2H-benzo[d][1,3]oxazin-7-yl)oxy)acetyl)pyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate, (14) (0.38 g, 0.025 mmol) in tetrahydrofuran (5.0 mL) was added 4 M HCl in dioxane solution (2 mL) at room temperature and the resulted reaction mixture was stirred for 4 h under nitrogen atmosphere. The reaction mixture was concentrated under reduced pressure to get crude ARK-80_HCl_Salt as a yellow solid. The crude mixture was purified by preparative HPLC using following method to get pure ARK-80_HCl salt (0.012 g, 3.6%) as a white amorphous powder. ¹H NMR (400 MHz, DMSO-d6) δ 9.93-9.91 ppm (3H, broad s), 7.92-7.85 ppm (10H, broad s), 7.65 ppm (4H, broad s), 7.40 ppm (2H, broad s), 7.27-7.15 ppm (8H, m), 6.87-6.71 ppm (3H, m), 6.54 ppm (1H, s), 5.36 ppm (1H, s), 5.10-5.02 ppm (3H, m), 4.83 ppm (2H, m), 4.66-4.56 ppm (2H, m), 4.39-4.28 ppm (2H, m), 4.06-4.01 ppm (2H, m), 3.58-3.55 ppm (4H, m), 3.47-3.41 ppm (7H, m), 3.13-2.94 ppm (9H, m), 2.71-2.66 ppm (8H, m), 2.22 ppm (7H, broad s), 1.52-1.50 ppm (12H, d), 1.26 ppm (18H, s). MS (ESI-MS): m/z calcd for C₆₉H₉₃N₁₃O₁₁ [MH]⁺ 1280.71, found 1281.43.

Method for Preparative HPLC:

(A) 0.05% HCl IN WATER (HPLC GRADE) and (B) 100% Acetonitrile (HPLC GRADE), using X-BRIDGE, 250 mm*19 mm*5 μm with the flow rate of 19.0 mL/min and with the following gradient:

| Time | % A | % B |
|---|---|---|
| 0.01 | 80.0 | 20.0 |
| 7.00 | 76.0 | 24.0 |
| 23.00 | 76.0 | 24.0 |
| 23.01 | 0.0 | 100 |
| 25.00 | 0.0 | 100 |
| 25.01 | 80.0 | 20 |
| 26.00 | 80.0 | 20 |

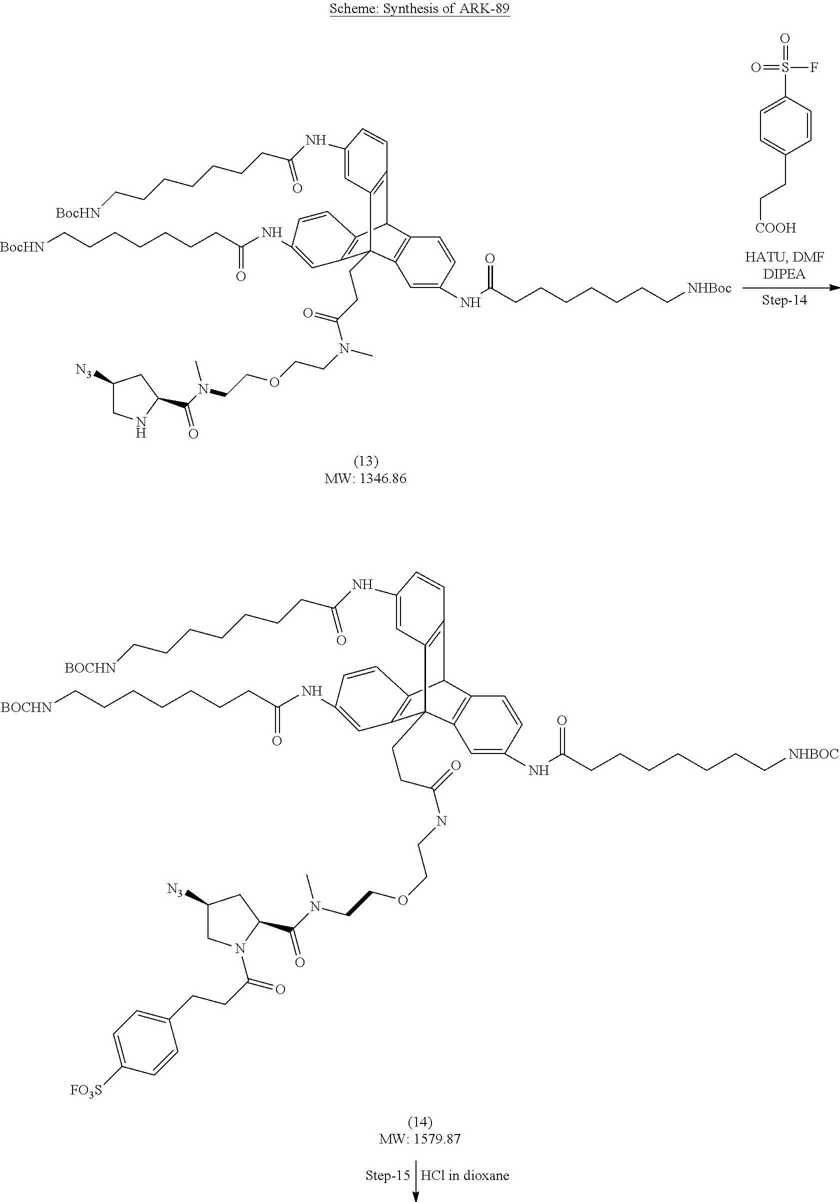

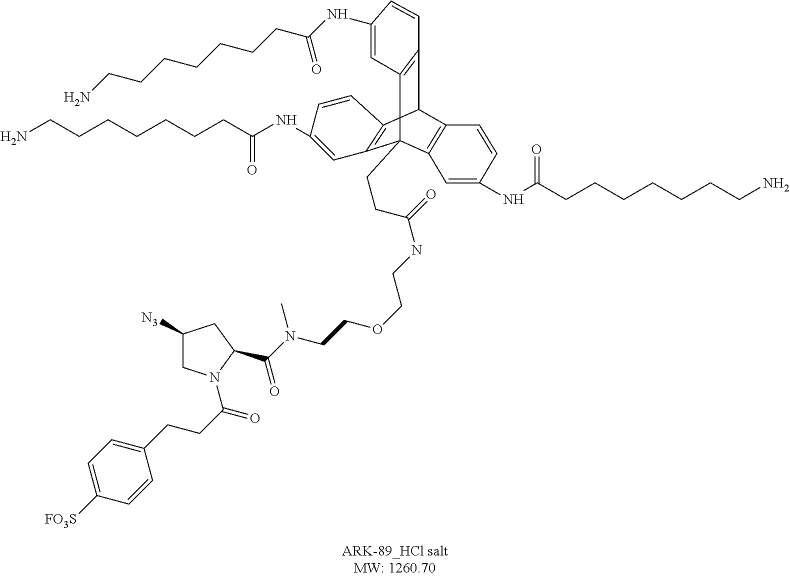

### Tri-tert-butyl (((9-(3-((2-(2-((2S,4S)-4-azido-1-(3-(4-(fluorosulfonyl)phenyl)propanoyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate (13) (0.31 g, 0.23 mmol) in N,N-dimethylformamide (6 mL) were sequentially added 3-(4-(fluorosulfonyl)phenyl)propanoic acid (0.043 g, 0.18 mmol) and HATU (0.070 g, 0.18 mmol) at room temperature. The reaction mixture was stirred for 5 minutes. To this, N,N-diisopropylethylamine (0.036 g, 0.276 mmol) was added drop wise and the resulted reaction mixture was further stirred for 1 h at room temperature. The reaction mixture was diluted by ethyl acetate (100 mL) and washed with ice-cold water (3×30 mL). The organic layers were combined, washed with brine and concentrated under reduced pressure at 25° C. to get crude 14 (0.45 g, Quantitative yield) as a dark yellow solid which was used in the next step without further purification. MS (ESI-MS): m/z calcd C₈₂H₁₁₇FN₁₂O₁₅S [MH]⁺ 1561.85, found 1463.45 (M−100, one Boc group fell off).

### N,N′N″-(9-(3-((2-(2-((2S,4S)-4-azido-1-(3-(4-(fluorosulfonyl)phenyl)propanoyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-aminooctanamide), ARK-89_HCl Salt

To a solution of tri-tert-butyl (((9-(3-((2-(2-((2S,4S)-4-azido-1-(3-(4-(fluorosulfonyl)phenyl)propanoyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate (14) (0.45 g, 0.028 mmol) in 1,4-dioxane (5.0 mL) was added 4 M HCl in dioxane (2 mL) at room temperature. The resulting reaction mixture was stirred for 4 hours. The mixture was concentrated under reduced pressure to get crude ARK-89_HCl_Salt as a yellow solid. The crude mixture was purified by preparative HPLC using following method to get pure ARK-89_HCl salt (0.053 g, 12.8%) as a yellow solid. ¹H NMR (400 MHz, DMSO-d₆) δ 9.95 ppm (3H, broad s), 8.03-7.95 ppm (10H, m), 7.67-7.62 ppm (5H, m), 7.28-7.21 ppm (6H, m), 5.38 ppm (1H, s), 4.77 ppm (0.5H, m), 4.59-4.49 ppm (1H, m), 4.31-4.21 ppm (1H, m), 4.02-3.96 ppm (2H, m), 3.62-3.44 ppm (6H, m), 3.22-3.03 ppm (8H, m), 2.98-2.88 ppm (4H, m), 2.74-2.60 ppm (10H, m), 2.24-2.23 ppm (7H, t), 1.53-1.52 ppm (12H, d), 1.26 ppm (18H, s). MS (ESI-MS): m/z calcd for C₆₇H₉₃FN₁₂O₉S [MH]⁺ 1261.70, found 1262.31.

Method for Preparative HPLC:

(A) 0.05% HCl IN WATER (HPLC GRADE) and (B) 100% Acetonitrile (HPLC GRADE), using X-SELECT FP, 250 mm*19 mm*5 μm with the following gradient:

| Time | % A | % B |
|---|---|---|
| 0.01 | 95.0 | 5.0 |
| 26.00 | 66.0 | 34.0 |
| 26.01 | 0.0 | 100 |
| 28.00 | 0.0 | 100 |
| 28.01 | 95.0 | 5.0 |
| 29.00 | 95.0 | 5.0 |

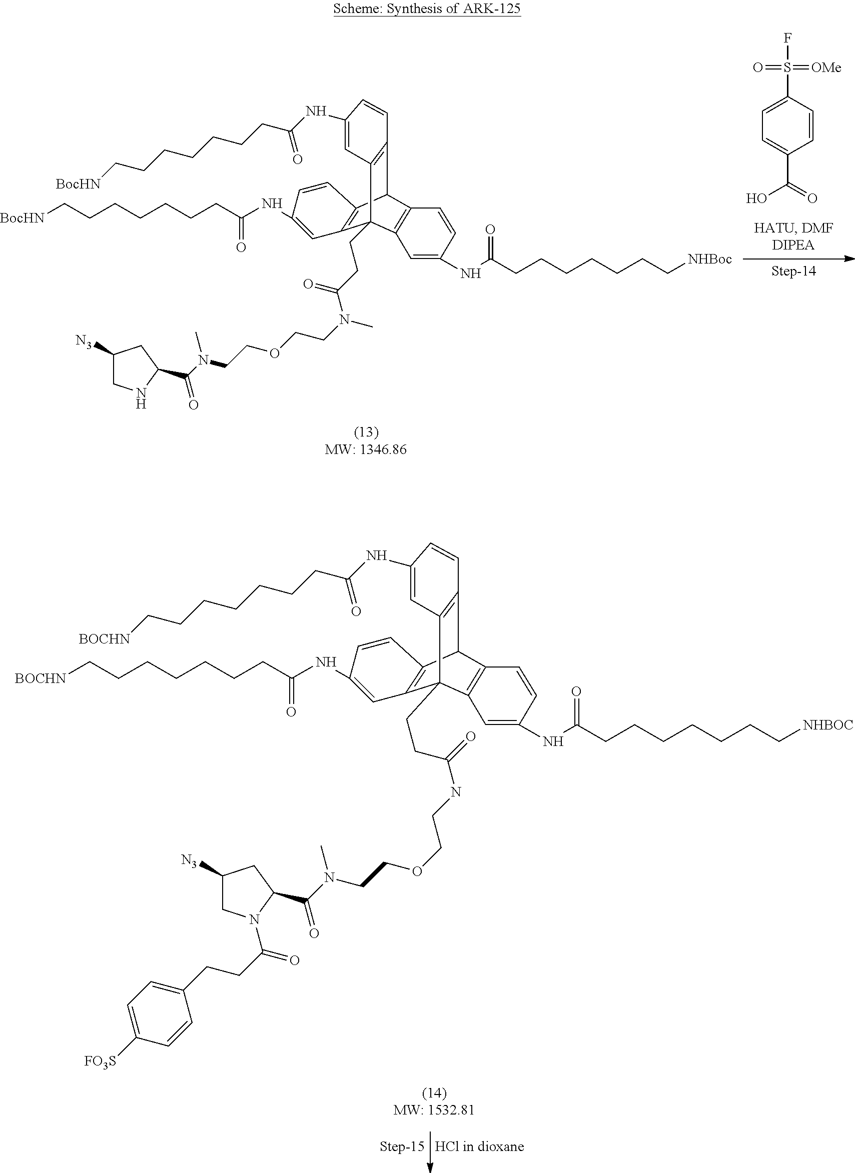

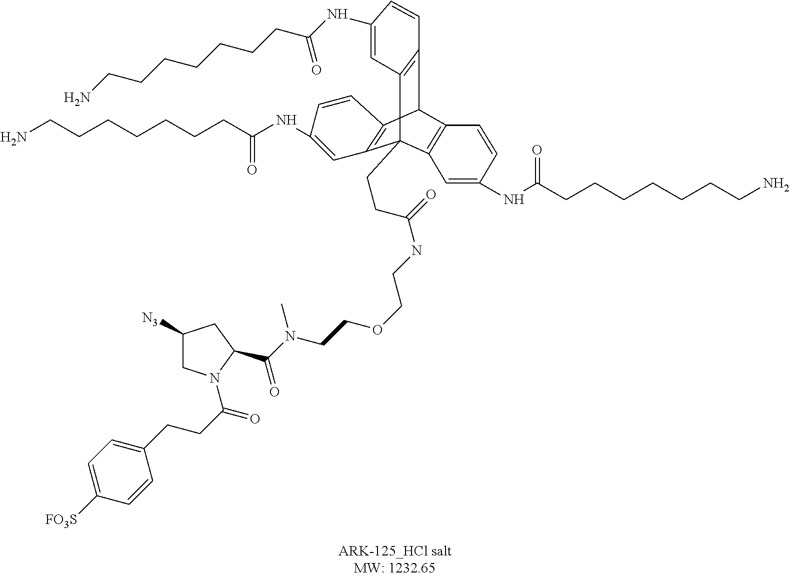

### Tri-tert-butyl (((9-(3-((2-(2-((2S,4S)-4-azido-1-(4-(fluorosulfonyl)benzoyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate (13) (0.30 g, 0.22 mmol) in N,N-dimethylformamide (10 mL) were sequentially added 4-fluorosulfonylbenzoic acid (0.054 g, 0.27 mmol) and HATU (0.101 g, 0.27 mmol) at room temperature. The reaction mixture was stirred for 5 minutes. To this, N,N-diisopropylethylamine (0.079 g, 0.45 mmol) was added drop wise and the resulted reaction mixture was further stirred for 1 h at room temperature. The reaction mixture was diluted by ethyl acetate (100 mL) and washed with ice-cold water (3×30 mL). The organic layers were combined, washed with brine and concentrated under reduced pressure at 25° C. to get crude 14 (0.388 g, Quantitative yield) as a yellow semi-solid which was used in the next step without further purification. MS (ESI-MS): m/z calcd C₈₀H₁₁₃FN₁₂O₁₅S [MH]⁺ 1532.81, found 1434.35 (M−100, one Boc group fell off).

### N,N′,N″-(9-(3-((2-(2-((2S,4S)-4-azido-1-(4-(fluorosulfonyl)benzoyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-aminooctanamide), ARK-125_HCl Salt

To a solution of tri-tert-butyl (((9-(3-((2-(2-((2S,4S)-4-azido-1-(4-(fluorosulfonyl)benzoyl)-N-methylpyrrolidine-2-carboxamido)ethoxy)ethyl)(methyl)amino)-3-oxopropyl)-9,10-dihydro-9,10-[1,2]benzenoanthracene-2,7,15-triyl)tris(azanediyl))tris(8-oxooctane-8,1-diyl))tricarbamate (14) (0.38 g, 0.0025 mmol) in 1,4-dioxane (5.0 mL) was added 4 M HCl in dioxane (2 mL) at room temperature and the resulting reaction mixture was stirred for 4 hours. The mixture was concentrated under reduced pressure to get crude ARK-125_HCl_Salt as a yellow solid. The crude mixture was purified by preparative HPLC using following method to get pure ARK-125_HCl_Salt (0.110 g, 33.0%) as a yellow solid. ¹H NMR (400 MHz, DMSO-d6) δ 9.96-9.93 ppm (3H, broad s), 8.24-8.21 ppm (2H, m), 7.97 ppm (8H, broad s), 7.87-7.82 ppm (2H, m), 7.71-7.68 ppm (3H, m), 7.32-7.19 ppm (6H, m), 5.38 ppm (1H, s), 5.05-5.01 ppm (1H, m), 4.87-4.80 ppm (1H, m), 4.30-4.20 ppm (1H, m), 3.89 ppm (18H, broad s), 3.70-3.55 ppm (5H, m), 3.48-3.38 ppm (3H, m), 3.18 ppm (1H, s), 3.08-3.04 ppm (6H, m), 2.79-2.68 ppm (7H, m), 2.25 ppm (6H, broad s), 1.54-1.52 ppm (12H, d), 1.26 ppm (18H, s). MS (ESI-MS): m/z calcd for C₆₅H₈₉FN₁₂O₉S [MH]⁺ 1233.65, found 1234.34.

Method for Preparative HPLC:

(A) 0.05% HCl IN WATER (HPLC GRADE) and (B) 100% Acetonitrile (HPLC GRADE), using X-SELECT FP, 250 mm*19 mm*5 μm with the flow rate of 19.0 mL/min and with the following gradient:

| Time | % A | % B |
|---|---|---|
| 0.01 | 90.0 | 10.0 |
| 3.00 | 85.0 | 15.0 |
| 22.00 | 80.0 | 20.0 |
| 22.01 | 0.0 | 100 |
| 23.00 | 0.0 | 100 |
| 23.01 | 90.0 | 10.0 |
| 24.00 | 90.0 | 10.0 |

### Example 16: Synthesis of ARK-81, ARK-90, and ARK-126 (Ark000025, Ark000028, and Ark000031)

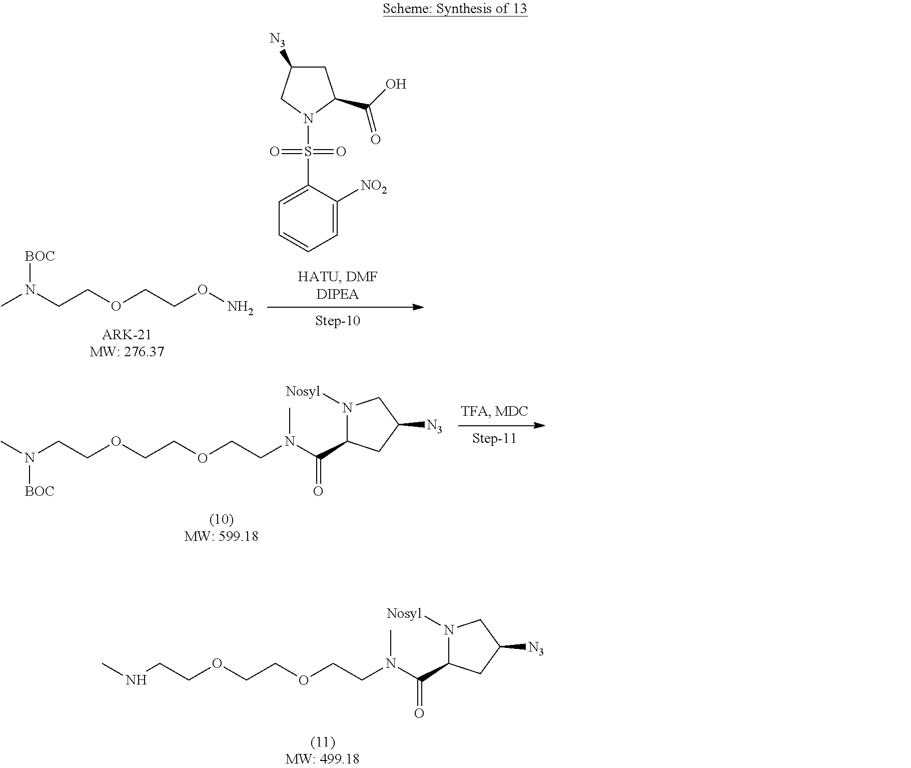

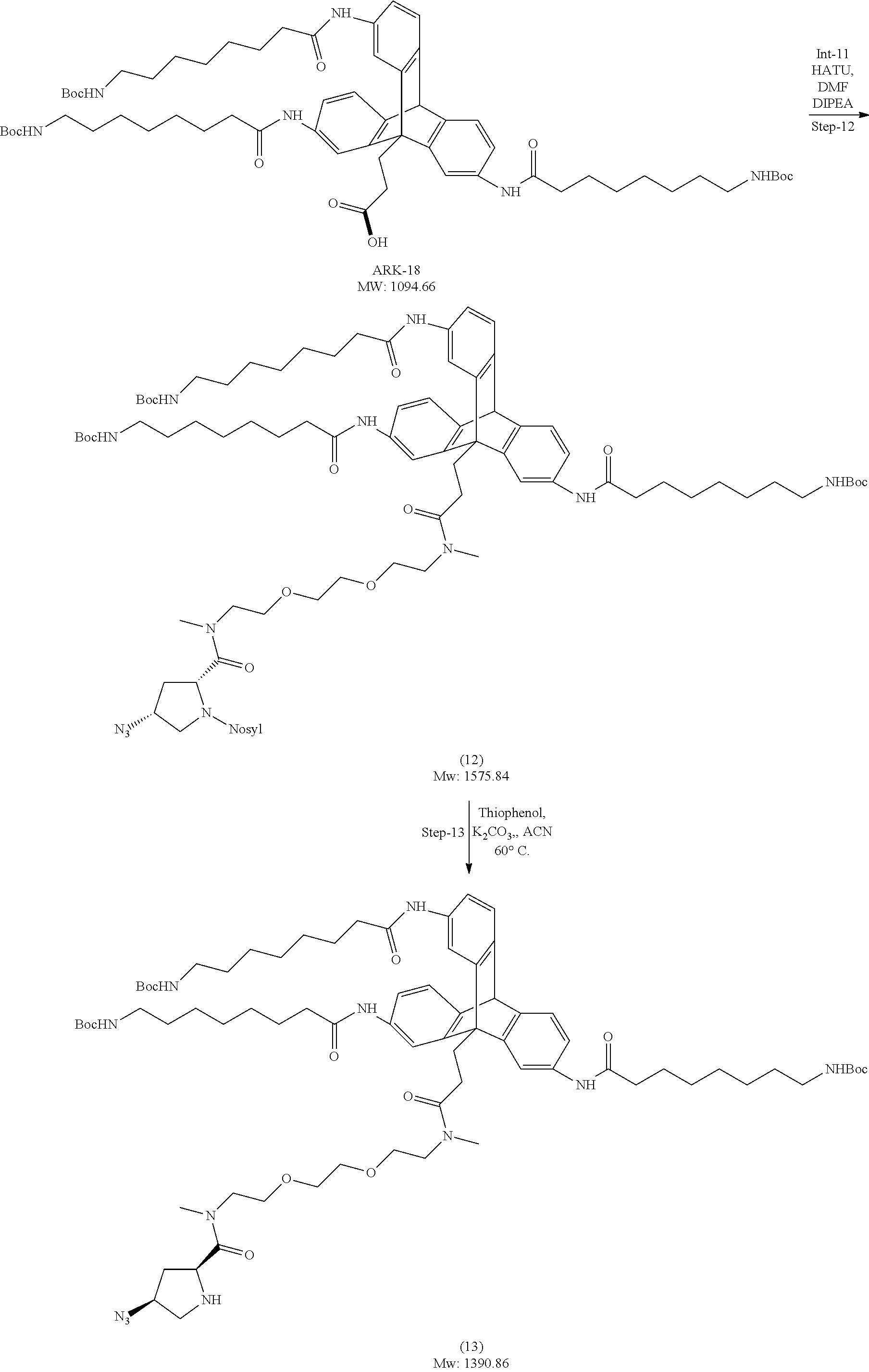

### Tert-butyl (2-(2-(2-((2S,4S)-4-azido-N-methyl-1-((2-nitrophenyl)sulfonyl)pyrrolidine-2-carboxamido)ethoxy)ethoxy)ethyl)(methyl)carbamate, 10

To a solution of ARK-21 (0.9 g, 3.04 mmol) in N,N-dimethylformamide (6 mL) were sequentially added (2S,4S)-4-azido-1-((2-nitrophenyl)sulfonyl)pyrrolidine-2-carboxylic acid (1.33 g, 3.91 mmol), HATU (1.4 g, 3.91 mmol) and N,N-diisopropylethylamine (0.85 g, 6.52 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 10 (1.5 g, 78.9%) as brown semisolid which was used in next step without further purification. MS (ESI-MS): m/z calcd for C₂₄H₃₇N₇O₉S [MH]⁺ 600.18, found 617.5 (M+18).

### (2S,4S)-4-azido-N-methyl-N-(2-(2-(2-(methylamino)ethoxy)ethoxy)ethyl)-1-((2 nitrophenyl)sulfonyl)pyrrolidine-2-carboxamide_TFA Salt, 11

To a solution of tri-tert-butyl((2R,2′R,2″R)-((9,10-dihydro-9,10-[1,2]benzenoanthracene-2,7,15-triyl)tris(azanediyl))tris(3-(1H-imidazol-4-yl)-1-oxopropane-1,2-diyl))tricarbamate (10) (1.5 g, 2.5 mmol) in dichloro methane (10 mL) was added trifluoro acetic acid (0.96 mL, 12.52 mmol) at room temperature. The resulted reaction mixture was stirred at room temperature for 2 h. The reaction mixture was filtered through celite bed and filtrate thus collected was concentrated under reduced pressure to get crude 11 (1.4 g, 91.50%) as a brown oil which was used without further purification. MS (ESI-MS): m/z calcd for C₁₉H₂₉N₇O₇S [MH]⁺ 500.18, found 500.31.

### Tri-tert-butyl (((9-(1-((2S,4S)-4-azido-1-((2-nitrophenyl)sulfonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 12

To a solution of (2S,4S)-4-azido-N-methyl-N-(2-(2-(2-(methylamino)ethoxy)ethoxy)ethyl)-1-((2-nitrophenyl)sulfonyl)pyrrolidine-2-carboxamide_TFA Salt (11) (0.56 g, 0.91 mmol) in N,N-dimethylformamide (4 mL) were sequentially added 3-(2,7,15-tris(8-((tert-butoxycarbonyl)amino)octanamido)-9,10-[1,2]benzenoanthracen-9(10H)-yl)propanoic acid (ARK-18) (0.5 g, 0.46 mmol), HATU (1.44 g, 0.55 mmol) and N,N-diisopropylethylamine (0.12 g, 0.91 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 12. The crude mixture was purified by column chromatography on silica gel (1.5% methanol/chloroform) to get 12 (0.6 g, 84.5%) as a brown solid. MS (ESI-MS): m/z calcd for Cs₁H₁₁₇N₁₃O₁₇S [MH]⁺ 1576.84, found 1578.4.

### Tri-tert-butyl (((9-(1-((2S,4S)-4-azidopyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 13

To a solution of tri-tert-butyl (((9-(1-((2S,4S)-4-azido-1-((2-nitrophenyl)sulfonyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (12) (0.6 g, 0.38 mmol) in acetonitrile (10 mL) were sequentially added potassium carbonate (0.26 g, 1.90 mmol) and thiophenol (0.12 mL, 1.14 mmol) at room temperature. The resulted reaction mixture was stirred at 80° C. for 2 h. The reaction mixture was filtered through celite bed and the collected filtrate was concentrated under reduced pressure to get crude 13 as yellow oil. The crude mixture was subjected to reverse phase chromatography to yield 13 (0.4 g, 84.9%) as a light yellow solid. MS (ESI-MS): m/z calcd for C₅₃H₆₂N₁₂O₉ [MH]⁺ 1391.86, found 1392.3.

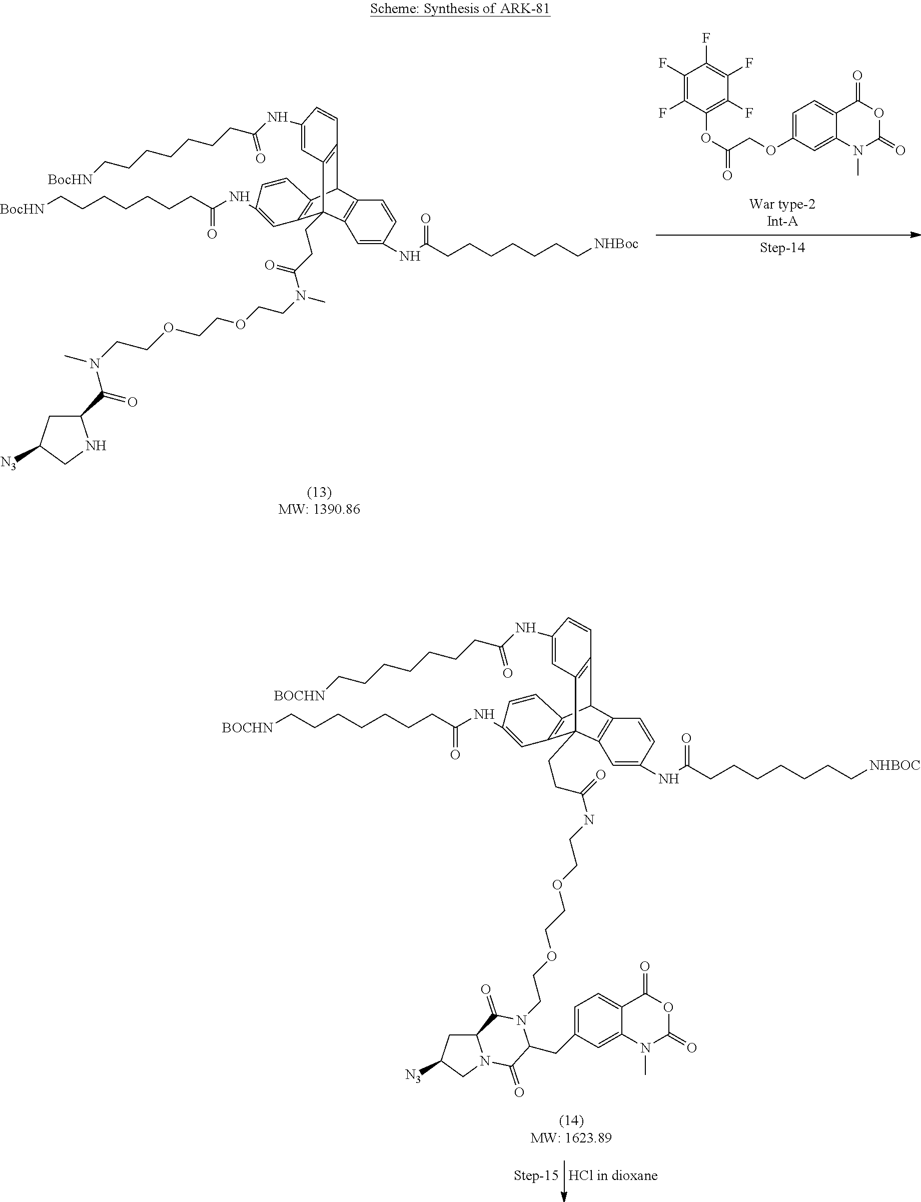

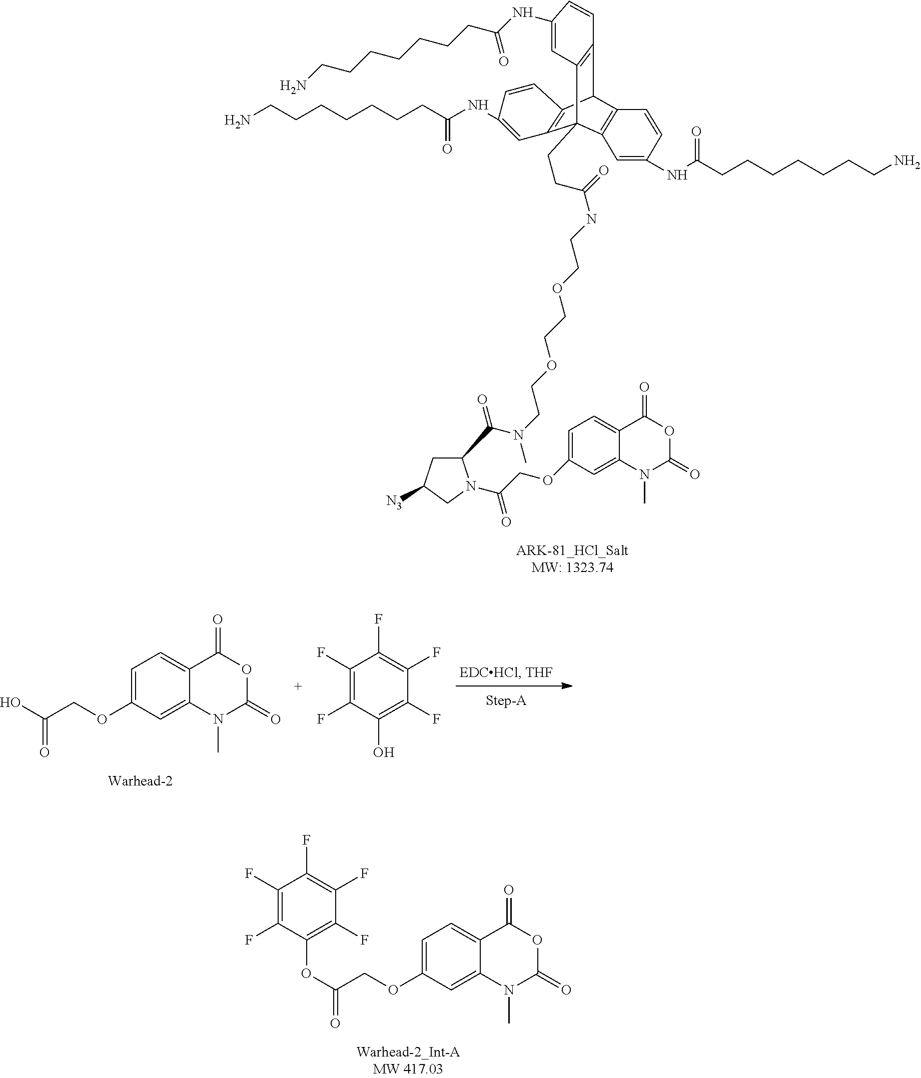 [SMILES: CN(CCOCCOCCNC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)COc1ccc2c(=O)oc(=O)n(C)c2c1 ; Cn1c(=O)oc(=O)c2ccc(OCC(=O)O)cc21 ; Oc1c(F)c(F)c(F)c(F)c1F ; Cn1c(=O)oc(=O)c2ccc(OCC(=O)Oc3c(F)c(F)c(F)c(F)c3F)cc21]

### Perfluorophenyl 2-((1-methyl-2,4-dioxo-1,4-dihydro-2H-benzo[d][1,3]oxazin-7-yl)oxy)acetate, Int-A

To a solution of Warhead-2 (0.048 g, 0.19 mmol) in tetrahydrofuran (1 mL) was added N-(3-Dimethylaminopropyl)-N-ethylcarbodiimide hydrochloride (0.037 g, 0.19 mmol) at 0° C. under nitrogen atmosphere. The reaction mixture was stirred at 0° C. for 10 min. To this, a solution of pentafluorophenol (0.036 g, 0.19 mmol) in tetrahydrofuran (0.5 mL) was added drop wise at 0° C. under nitrogen atmosphere. The resulted reaction mixture was further stirred at 0° C. for 1 h. The reaction mixture was directly used in the next step without work up and isolation. MS (ESI-MS): m/z calcd C₁₇H₈F₅NO₆ [MH]⁺ 418.03, the compound did not show mass response. Note: Intermediate-A was not isolated—the reaction mass was transferred as such to the next step reaction mass.

### Tri-tert-butyl (((9-(1-((2S,4S)-4-azido-1-(2-((1-methyl-2,4-dioxo-1,4-dihydro-2H-benzo[d][1,3]oxazin-7-yl)oxy)acetyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 14

To a solution of (((9-(3-((2-(2-((2S,4S)-4-azido-N-methylpyrrolidine-2-carboxamido)ethoxy)ethyl) (methyl)amino)-3-oxopropyl)-9,10-dihydro-9,10[1,2]benzenoanthracene-2,7,15-triyl)tris(azanediyl)) tris(8-oxooctane-8,1-diyl))tricarbamate (13) (0.27 g, 0.19 mmol) in tetrahydrofuran (4 mL) was added solution of pentafluorophenyl [(1-methyl-2,4-dioxo-1,4-dihydro-2H-3,1-benzoxazin-7-yl)oxy]acetate (Warhead_type_2_Int_A) (0.081 g, 0.19 mmol) and the resulted reaction mixture was stirred for 1 h at room temperature. The reaction mixture concentrated under reduced pressure to get crude 14 (0.3 g, 80.21%) as brown solid which was used in the next step without further purification. MS (ESI-MS): m/z calcd C₈₆H₁₂₁N₁₃O₁₉[MH]⁺ 1623.89, found 1525.46 (M−100, one Boc group fell off).

### N,N′,N″-(9-(1-((2S,4S)-4-azido-1-(2-((1-methyl-2,4-dioxo-1,4-dihydro-2H-benzo[d][1,3]oxazin-7-yl)oxy)acetyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(8-aminooctanamide), ARK-81_HCl Salt

To a solution of tri-tert-butyl (((9-(1-((2S,4S)-4-azido-1-(2-((1-methyl-2,4-dioxo-1,4-dihydro-2H-benzo[d][1,3]oxazin-7-yl)oxy)acetyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (14) (0.3 g, 0.0018 mmol) in tetrahydrofuran (5.0 mL) was added 4 M HCl in dioxane solution (2 mL) at room temperature and the resulted reaction mixture was stirred for 4 h under nitrogen atmosphere. The reaction mixture was concentrated under reduced pressure to get crude ARK-81_HCl_Salt as a yellow solid. The crude mixture was purified by preparative HPLC using following method to get pure ARK-81_HCl salt (0.034 g, 12.8%) as a yellow solid. ¹H NMR (400 MHz, DMSO-d6) δ 9.94 ppm (3H, br S), 7.79-7.86 ppm (8H, m), 7.66 ppm (2H, S), 7.43 ppm (1H, S), 7.31-7.18 ppm (7H, m), 6.88-6.82 ppm (1H, m), 6.78-6.76 ppm (1H, m), 5.38 ppm (1H, S), 5.11-5.02 ppm (1H, m), 4.80 ppm (1H, br S), 4.36-4.31 ppm (1H, m), 4.03-4.01 ppm (1H, m), 3.62-3.42 ppm (15H, m), 3.37-3.26 ppm (4H, m), 3.16 ppm (1H, s), 3.05-2.99 ppm (5H, m), 2.89 ppm (1H, s), 2.81-2.71 ppm (7H, m), 2.24 ppm (6H, S), 1.53-1.52 ppm (12H, d), 1.26 ppm (18H, S). MS (ESI-MS): m/z calcd for C₇₁H₉₇N₁₃O₁₂ [MH]⁺ 1323.74, found 1325.4.

Method for Preparative HPLC:

(A) 0.05% HCl in water (HPLC GRADE) and (B) 100% acetonitrile (HPLC GRADE), using X-SELECT C18, 250 mm*19 mm*5 μm with the flow rate of 19.0 mL/min and with the following gradient:

| Time | % A | % B |
|---|---|---|
| 0.01 | 95.0 | 5 |
| 4.00 | 85.0 | 15 |
| 20.00 | 83.0 | 17 |
| 20.01 | 0.0 | 100 |
| 23.00 | 0.0 | 100 |
| 23.01 | 95.0 | 5 |
| 24.00 | 95.0 | 5 |

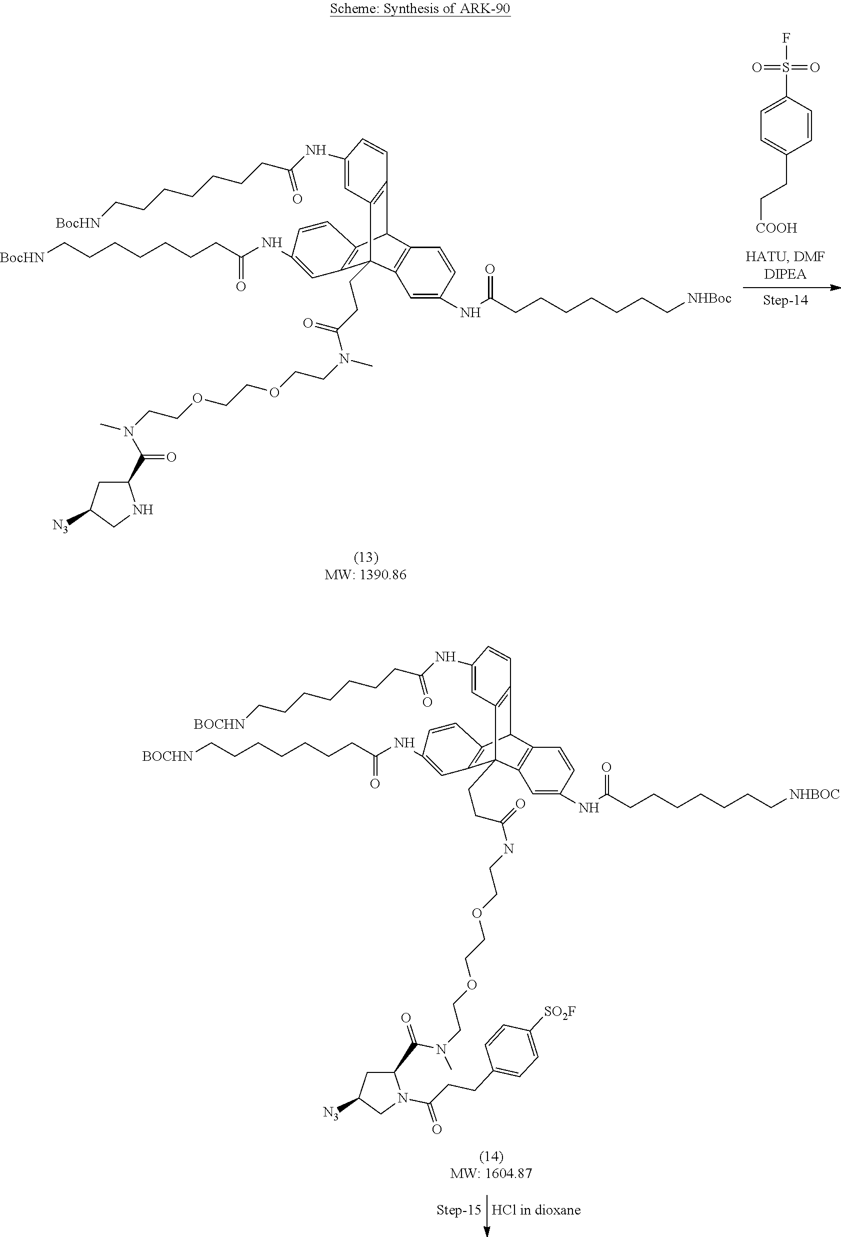

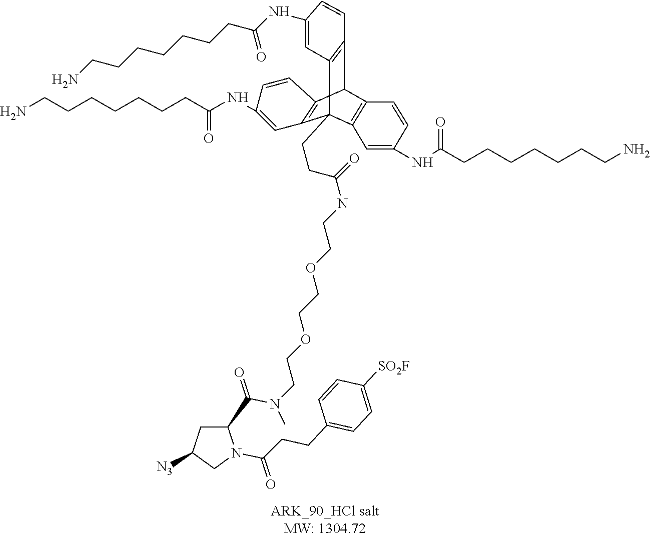 [SMILES: CN(CCOCCOCCNC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)CCc1ccc(S(=O)(=O)F)cc1]

### Tri-tert-butyl (((9-(1-((2S,4S)-4-azido-1-(3-(4-(fluorosulfonyl)phenyl)propanoyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (13) (0.4 g, 0.29 mmol) in N,N-dimethylformamide (4 mL) were sequentially added 3-(4-(fluorosulfonyl)phenyl)propanoic acid (0.07 g, 0.29 mmol) and HATU (0.13 g, 0.35 mmol) at room temperature. The reaction mixture was stirred for 5 minutes. To this, N,N-diisopropylethylamine (0.08 g, 0.56 mmol) was added drop wise and the resulted reaction mixture was further stirred for 1 h at room temperature. The reaction mixture was diluted by ethyl acetate (100 mL) and washed with ice-cold water (3×30 mL). The organic layers were combined, washed with brine and concentrated under reduced pressure at 25° C. to get crude 14 (0.4 g, 87%) as a brown solid which was used in the next step without further purification. MS (ESI-MS): m/z calcd for C₈₄H₁₂₁FN₁₂O₁₆S [MH]⁺ 1605.88, found 1506.5 (M−100, one Boc group fell off).

### N,N′,N″-(9-(1-((2S,4S)-4-azido-1-(3-(4-(fluorosulfonyl)phenyl)propanoyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-aminooctanamide), ARK-90_HCl Salt

To a solution tri-tert-butyl (((9-(1-((2S,4S)-4-azido-1-(3-(4-(fluorosulfonyl)phenyl)propanoyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (14) (0.4 g, 0.0025 mmol) in 1,4-dioxane (5.0 mL) was added 4 M HCl in dioxane (2 mL) at room temperature. The resulting reaction mixture was stirred for 4 hours. The mixture was concentrated under reduced pressure to get crude of ARK-90_HCl_Salt as yellow solid. The crude mixture was purified by preparative HPLC using following method to get pure ARK-90_HCl salt (0.035 g, 7.11%) as yellow solid. ¹H NMR (400 MHz, DMSO) δ 9.89 ppm (3H, broad s), 8.03-8.00 ppm (2H, t), 7.66-7.56 ppm (5H, m), 7.29-7.20 ppm (6H, m), 5.33 (1H, s), 3.62-3.52 ppm (6H, m) 3.49-3.44 ppm (3H, m), 3.44-3.02 ppm (6H, m), 3.05-2.99 ppm (8H, m), 2.93 ppm (3H, broad s),2.76-2.70 ppm (10H, m), 2.23 (6H, s), 1.519 (14H, s), 1.52 (21H, s). MS (ESI-MS): m/z calcd for C₆₈H₉₅FN₁₂O₁₀S [MH]⁺ 0.1304.72, found 1306.3. HPLC retention time: 10.894 min.

Method for Preparative HPLC:

0.05% HCl in water (HPLC grade) and (B) 100% Acetonitrile (HPLC grade), using WATERS X-BRIDGE C18, 250 mm*30 mm*5 μm with the flow rate of 35.0 mL/min and with the following gradient:

| Time | % A | % B |
|---|---|---|
| 0.00 | 85.0 | 15.0 |
| 5.00 | 80.0 | 20.0 |
| 25.00 | 60.0 | 40.0 |
| 25.01 | 0.0 | 100.0 |
| 26.00 | 0.0 | 100.0 |
| 26.01 | 85.0 | 15.0 |
| 27.00 | 85.0 | 15.0 |

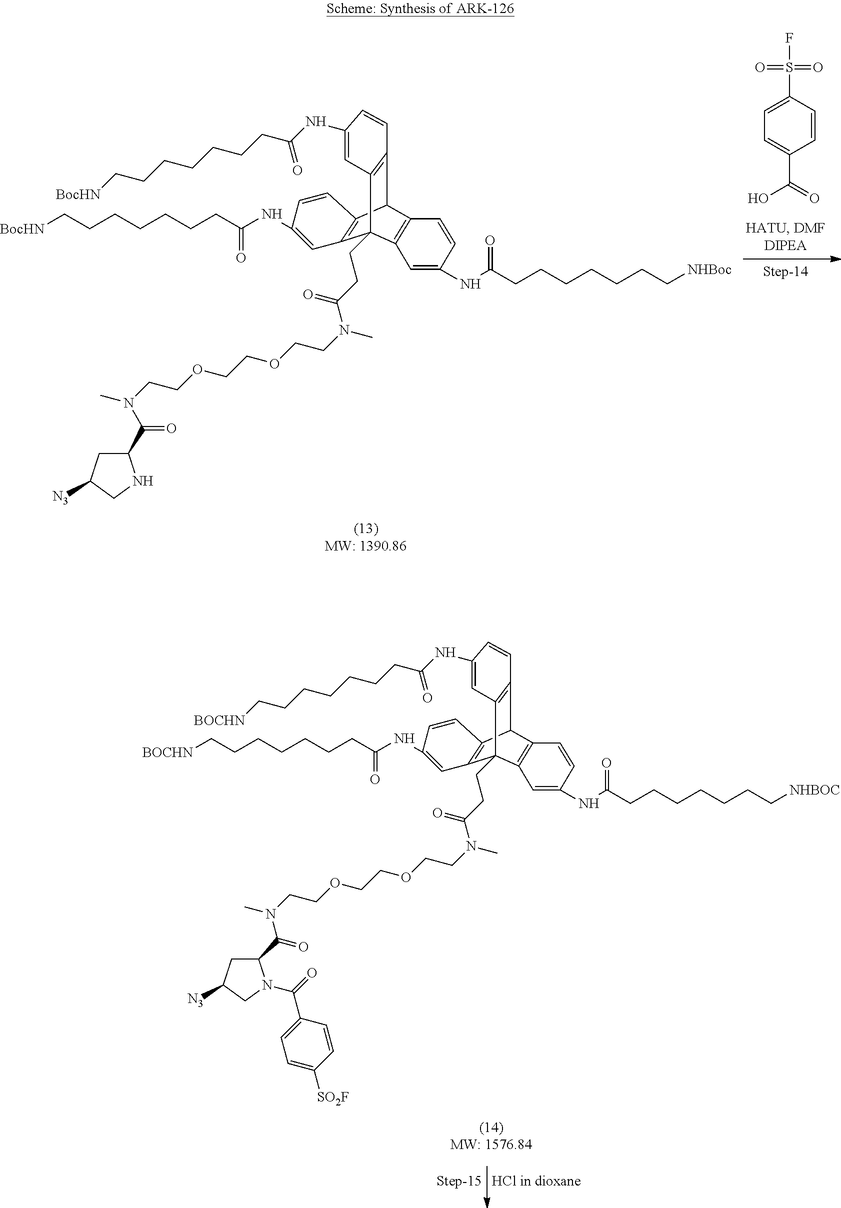

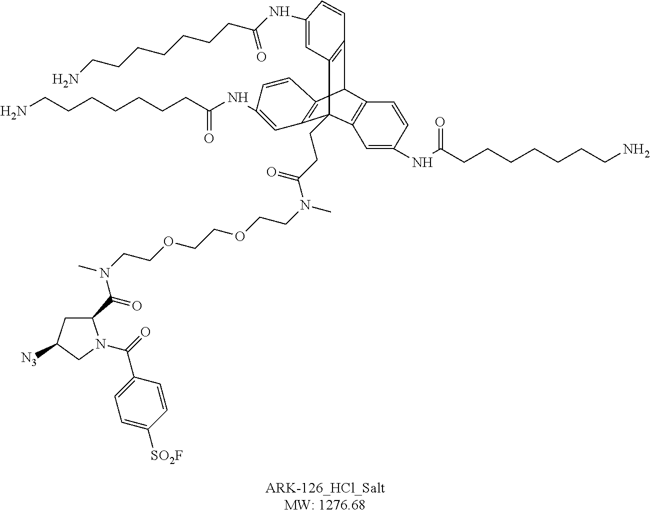 [SMILES: CN(CCOCCOCCN(C)C(=O)[C@@H]1C[C@H](N=[N+]=[N-])CN1C(=O)c1ccc(S(=O)(=O)F)cc1)C(=O)CCC12c3cc(NC(=O)CCCCCCCN)ccc3C(c3ccc(NC(=O)CCCCCCCN)cc31)c1ccc(NC(=O)CCCCCCCN)cc12]

### Tri-tert-butyl (((9-(1-((2S,4S)-4-azido-1-(3-(4-(fluorosulfonyl)benzoyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (13) (0.1 g, 0.072 mmol) in N,N-dimethylformamide (4 mL) were sequentially added 4-fluorosulfonylbenzoic acid (0.018 g, 0.09 mmol) and HATU (0.033 g, 0.09 mmol) at room temperature. The reaction mixture was stirred for 5 minutes. To this, N,N-diisopropylethylamine (0.018 g, 0.14 mmol) was added drop wise and the resulted reaction mixture was further stirred for 1 h at room temperature. The reaction mixture was diluted by ethyl acetate (100 mL) and washed with ice-cold water (3×30 mL). The organic layers were combined, washed with brine and concentrated under reduced pressure at 25° C. to get crude 14 (0.12 g, quantitative yield) as a yellow semi-solid which was used in the next step without further purification. MS (ESI-MS): m/z calcd for C₉₂H₁₁₇FN₁₂O₁₆S [MH]⁺ 1577.84, found 1478.46 (M−100, one Boc group fell off).

### N,N′,N″-(9-(1-((2S,4S)-4-azido-1-(3-(4-(fluorosulfonyl)benzoyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-aminooctanamide), ARK-126_HCl Salt

To a solution of tri-tert-butyl (((9-(1-((2S,4S)-4-azido-1-(3-(4-(fluorosulfonyl)benzoyl)pyrrolidin-2-yl)-2,11-dimethyl-1,12-dioxo-5,8-dioxa-2,11-diazatetradecan-14-yl)-9,10-dihydro-9,10-[1,2]benzenoanthracene-2,7,15-triyl)tris(azanediyl))tris(8-oxooctane-8,1-diyl))tricarbamate (14) (0.12 g, 0.0007 mmol) in 1,4-dioxane (5.0 mL) was added 4 M HCl in dioxane (2 mL) at room temperature and the resulting reaction mixture was stirred for 4 hours. The mixture was concentrated under reduced pressure to get crude ARK-126_HCl_Salt as a yellow solid. The crude mixture was purified by preparative HPLC using following method to get pure ARK-126_HCl_Salt (0.03 g, 28.57%) as a yellow solid. ¹H NMR (400 MHz, DMSO) δ 9.93-9.91 ppm (3H, broad s), 8.26-8.13 ppm (2H, m), 7.87 ppm (9H, broad s), 7.78-7.76 ppm (1H, d), 7.67 ppm (3H, broad s), 7.29-7.22 ppm (6H, m), 5.39 ppm (1H, s), 5.010-4.969 ppm (0.5H, t), 4.86-4.82 ppm (0.5H, m), 4.72-4.60 ppm (1H, m), 4.44-4.36 ppm (1H, m), 4.30-4.21 ppm (1H, m), 4.14-4.00 ppm (1H, m), 3.64-3.61 ppm (20H, m) 3.48-3.37 ppm (6H, m), 3.19-3.11 ppm (3H, m), 3.07-3.03 ppm (5H, m), 2.89-2.84 ppm (2H, broad s), 2.76-2.68 ppm (7H, m), 2.26-2.23 ppm (6H, t), 1.53 ppm (12H, s), 1.27 ppm (18H, s). MS (ESI-MS): m/z calcd for C₆₇H₉₃FN₁₂O₁₀S [MH]⁺ 0.1277.68, found 1278.35.

Method for Preparative HPLC:

(A) 0.05% HCl in water (HPLC grade) and (B) 100% Acetonitrile (HPLC grade), using SUNFIRE C18, 150 mm*19 mm*5 μm with the flow rate of 19.0 mL/min and with the following gradient:

| Time | % A | % B |
|---|---|---|
| 0.01 | 95.0 | 5.0 |
| 15.00 | 70.0 | 30.0 |
| 15.01 | 0.0 | 100.0 |
| 18.00 | 0.0 | 100.0 |
| 18.01 | 95.0 | 5.0 |
| 19.00 | 95.0 | 5.0 |

### Example 17: Synthesis of ARK-82, ARK-91, and ARK-127 (Ark000026, Ark000029, and Ark000032)

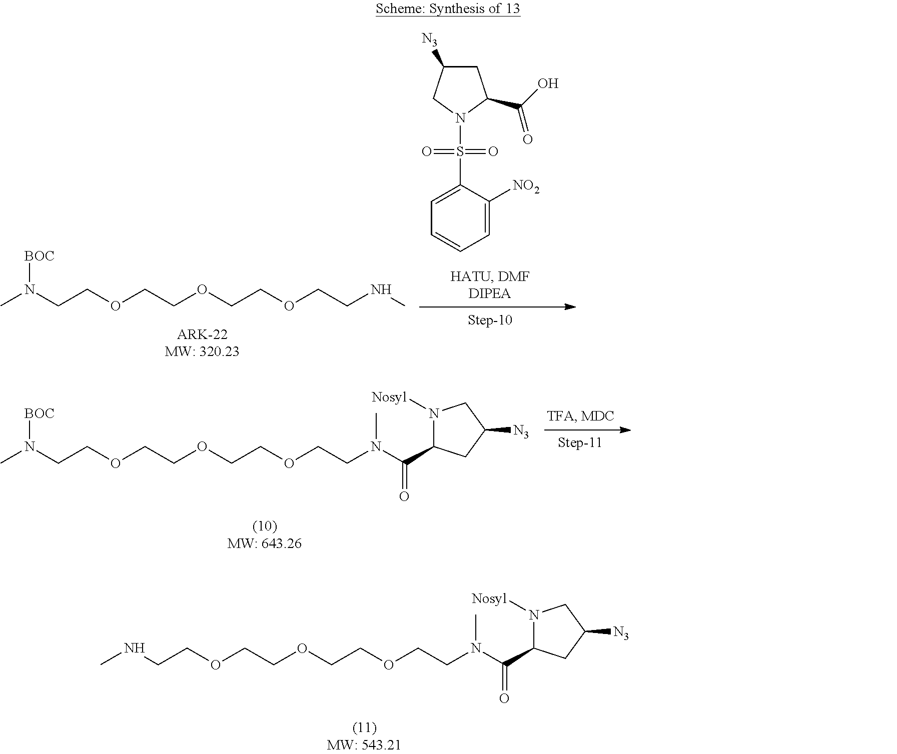

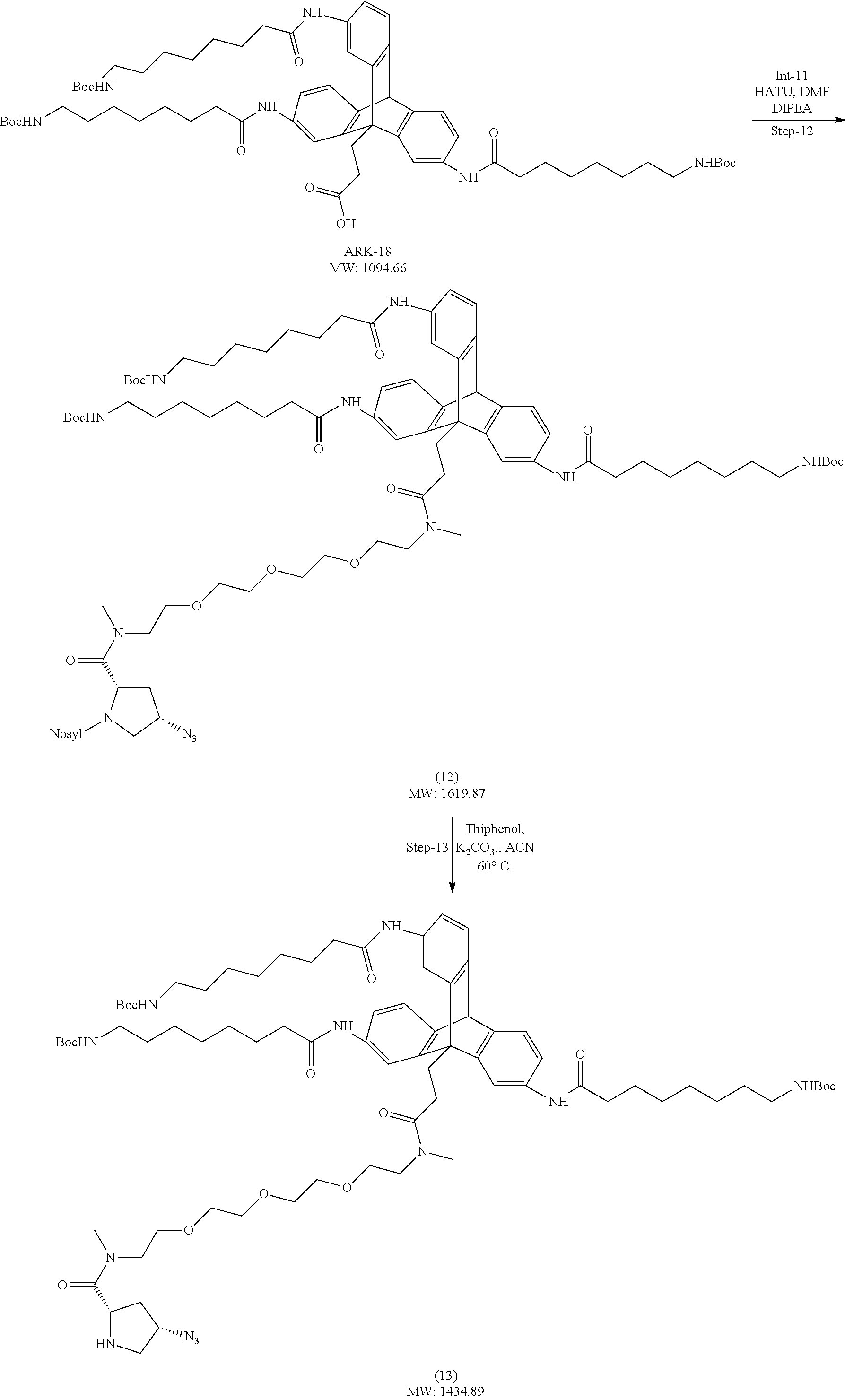

### Tert-butyl (1-((2S,4S)-4-azido-1-((2-nitrophenyl)sulfonyl)pyrrolidin-2-yl)-2-methyl-1-oxo-5,8,11-trioxa-2-azatridecan-13-yl)(methyl)carbamate, 10

To a solution of ARK-22 (0.41 g, 1.281 mmol) in N,N-dimethylformamide (10 mL) were sequentially added (2S,4S)-4-azido-1-((2-nitrophenyl)sulfonyl)pyrrolidine-2-carboxylic acid (0.52 g, 1.54 mmol), HATU (0.584 g, 1.54 mmol) and N,N-diisopropylethylamine (0.33 g, 2.56 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 10 (0.8 g, 97.2%). as brown semisolid which was used in next step without further purification. MS (ESI-MS): m/z calcd for C₂₆H₄₁N₇O₁₀S [MH]⁺ 644.26, found 544.36 (M−100).

### (2S,4S)-4-azido-N-methyl-1-((2-nitrophenyl)sulfonyl)-N-(5,8,11-trioxa-2-azatridecan-13-yl) pyrrolidine-2-carboxamide_TFA Salt, 11

To a solution of tri-tert-butyl (1-((2S,4S)-4-azido-1-((2-nitrophenyl)sulfonyl)pyrrolidin-2-yl)-2-methyl-1-oxo-5,8,11-trioxa-2-azatridecan-13-yl)(methyl) carbamate (10) (0.8 g, 1.24 mmol) in dichloro methane (10 mL) was added trifluoro acetic acid (0.48 mL, 6.21 mmol) at room temperature. The resulted reaction mixture was stirred at room temperature for 2 h. The reaction mixture was filtered through celite bed and filtrate thus collected was concentrated under reduced pressure to get crude 11 (1.05 g, Quantitative yield) as a brown oil which was used without further purification. MS (ESI-MS): m/z calcd for C₂₁H₃₃N₇O₈S.TFA [MH]⁺ 544.21, found 544.47.

### Tri-tert-butyl (((9-(1-((2S,4S)-4-azido-1-((2-nitrophenyl)sulfonyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate, 12

To a solution of (2S,4S)-4-azido-N-methyl-1-((2-nitrophenyl)sulfonyl)-N-(5,8,11-trioxa-2-azatridecan-13-yl) pyrrolidine-2-carboxamide_TFA Salt (11) (0.65 g, 0.98 mmol) in N,N-dimethylformamide (4 mL) were sequentially added 3-(2,7,15-tris(8-((tert-butoxycarbonyl)amino)octanamido)-9,10-[1,2]benzenoanthracen-9(10H)-yl)propanoic acid (ARK-18) (0.9 g, 0.822 mmol), HATU (0.375 g, 0.98 mmol) and N,N-diisopropylethylamine (0.21 g, 1.64 mmol) at room temperature. The resulted reaction mixture was stirred for 1 h at room temperature. The reaction mixture was poured in ice-cold water and extracted with ethyl acetate (3×100 mL). The organic layers were combined, washed with brine and concentrated under reduced pressure to get crude 12. The crude mixture was purified by column chromatography on silica gel (1.5% methanol/chloroform) to get 12 (1.72 g, quantitative yield) as a brown solid which was used in the next step without further purification. MS (ESI-MS): m/z calcd for C₉₃H₁₂₁N₁₃O₁₈S [MH]⁺ 1620.87, found 1522.31 (M−100).

### Tri-tert-butyl (((9-(1-((2S,4S)-4-azidopyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris (8-oxooctane-8,1-diyl))tricarbamate, 13

To a solution of tri-tert-butyl (((9-(1-((2S,4S)-4-azido-1-((2-nitrophenyl)sulfonyl)pyrrolidin-2-yl)-2,14-dimethyl-1,15-dioxo-5,8,11-trioxa-2,14-diazaheptadecan-17-yl)-9,10-dihydro-9,10-[1,2] benzenoanthracene-2,7,15-triyl)tris(azanediyl))tris(8-oxooctane-8,1-diyl))tricarbamate (12) (0.7 g, 0.43 mmol) in acetonitrile (60 mL) were sequentially added potassium carbonate (0.29 g, 2.16 mmol) and thiophenol (0.13 mL, 1.296 mmol) at room temperature. The resulted reaction mixture was stirred at 80° C. for 2 h. The reaction mixture was filtered through celite bed and the collected filtrate was concentrated under reduced pressure to get crude 13 as yellow oil. The crude mixture was subjected to reverse phase chromatography to yield 13 (0.39 g, 62.9%) as a light yellow solid. The crude was purified by trituration with n-Pentane (to remove unreacted thiophenol) to get 13 (0.39 g, 62.9%) as a yellow solid. MS (ESI-MS): m/z calcd for C₇₇H₁₁₈N₁₂O₁₄ [MH]⁺ 1435.89, found 1437.41.

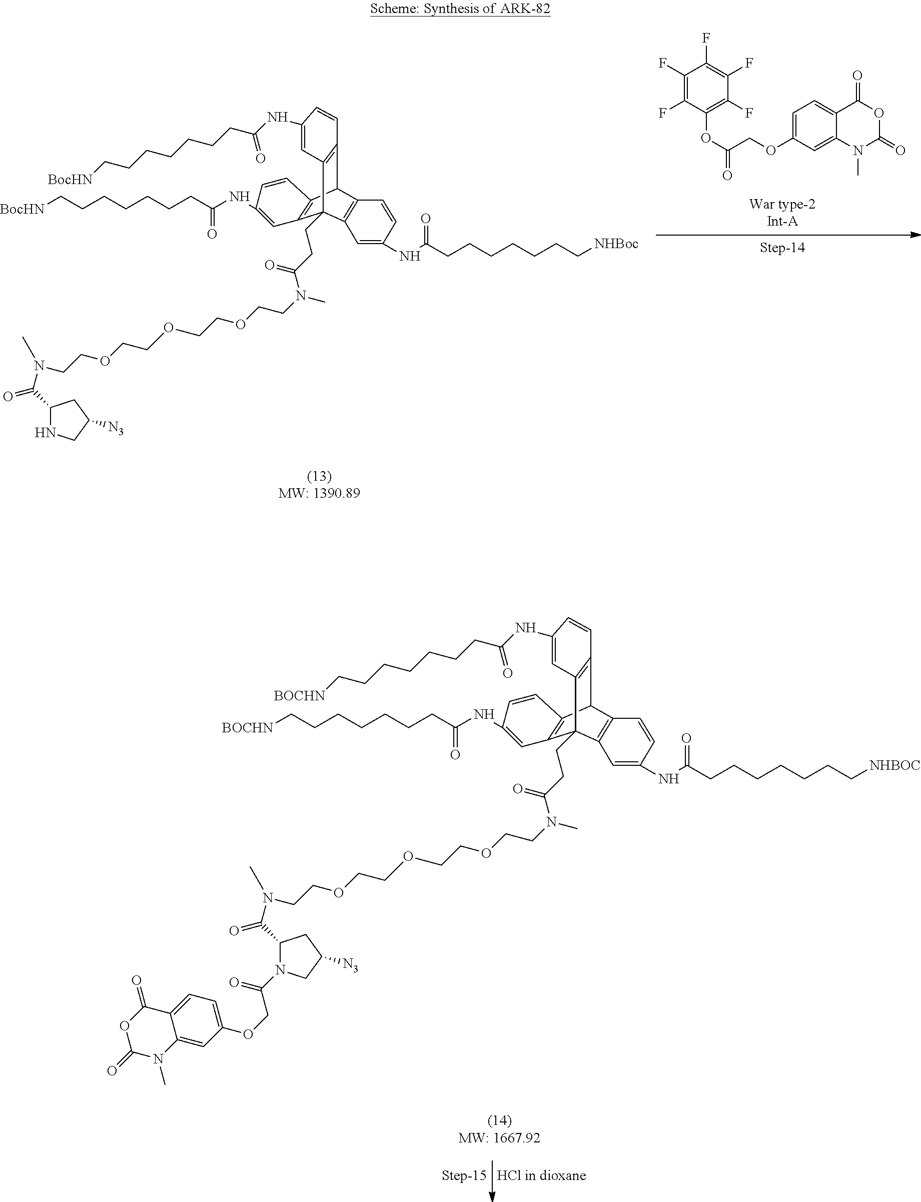

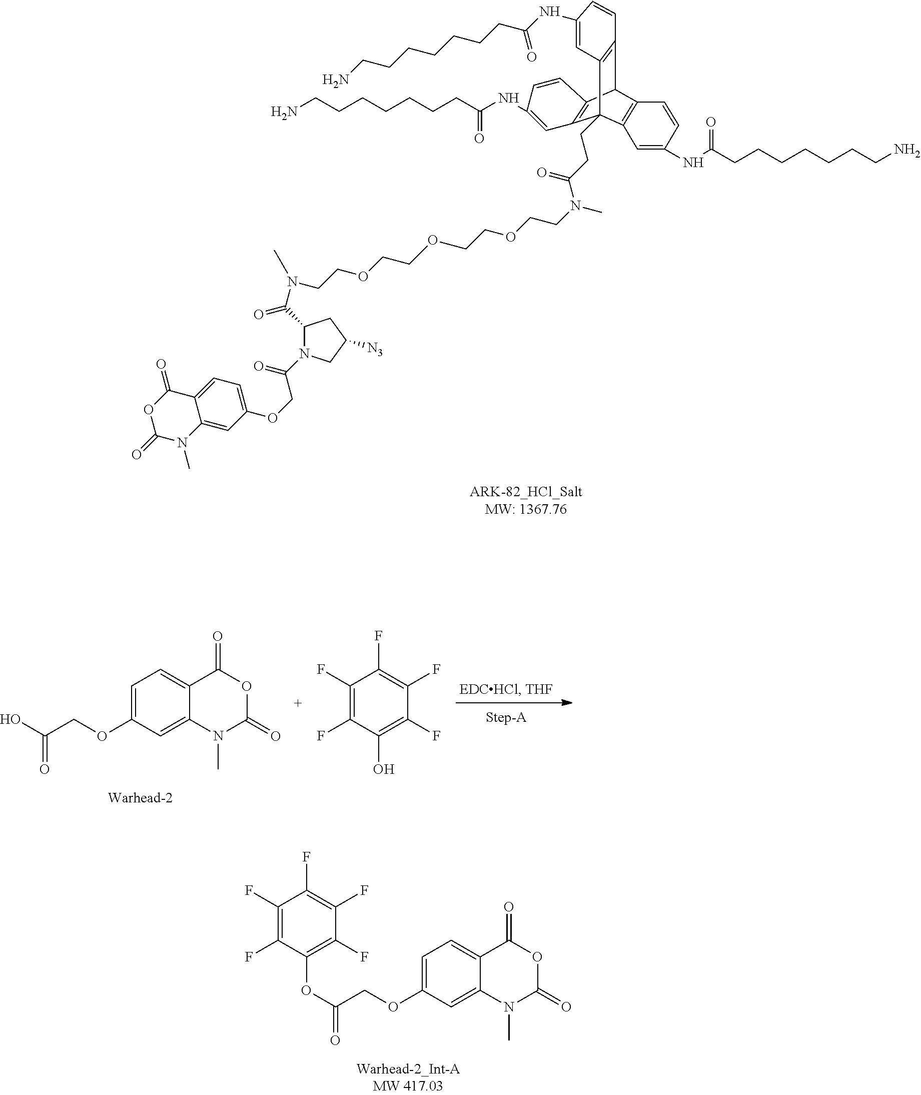 [SMILES: CN(CCOCCOCCOCCN(C)C(=O)[C@@H]1C[C@H](N=[N+]=[N-])CN1C(=O)COc1ccc2c(=O)oc(=O)n(C)c2c1)C(=O)CCC12c3cc(NC(=O)CCCCCCCN)ccc3C(c3ccc(NC(=O)CCCCCCCN)cc31)c1ccc(NC(=O)CCCCCCCN)cc12 ; Cn1c(=O)oc(=O)c2ccc(OCC(=O)O)cc21 ; Oc1c(F)c(F)c(F)c(F)c1F ; Cn1c(=O)oc(=O)c2ccc(OCC(=O)Oc3c(F)c(F)c(F)c(F)c3F)cc21]

### Perfluorophenyl 2-((1-methyl-2,4-dioxo-1,4-dihydro-2H-benzo[d][1,3]oxazin-7-yl)oxy)acetate, Int-A

To a solution of Warhead-2 (0.055 g, 0.21 mmol) in tetrahydrofuran (1 mL) was added N-(3-Dimethylaminopropyl)-N-ethylcarbodiimide hydrochloride (0.047 g, 0.21 mmol) at 0° C. under nitrogen atmosphere. The reaction mixture was stirred at 0° C. for 10 min. To this, a solution of pentafluorophenol (0.04 g, 0.21 mmol) in tetrahydrofuran (0.5 mL) was added drop wise at 0° C. under nitrogen atmosphere. The resulted reaction mixture was further stirred at 0° C. for 1 h. The reaction mixture was directly used in the next step without work up and isolation. MS (ESI-MS): m/z calcd C₁₇H₈F₅NO₆ [MH]⁺ 418.03, the compound did not show mass response. Note: Intermediate-A was not isolated—the reaction mass was transferred as such to the next step reaction mass.

### Tri-tert-butyl (((9-(1-((2S,4S)-4-azido-1-(2-((1-methyl-2,4-dioxo-1,4-dihydro-2H-benzo[d][1,3]oxazin-7-yl)oxy)acetyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris (8-oxooctane-8,1-diyl))tricarbamate (13) (0.3 g, 0.21 mmol) in tetrahydrofuran (4 mL) was added solution of pentafluorophenyl [(1-methyl-2,4-dioxo-1,4-dihydro-2H-3,1-benzoxazin-7-yl)oxy]acetate (Warhead_type_2) (0.087 g, 0.21 mmol) and the resulted reaction mixture was stirred for 1 h at room temperature. The reaction mixture concentrated under reduced pressure to get crude 14 (0.54 g, Quantitative yield) as brown solid which was used in the next step without further purification. MS (ESI-MS): m/z calcd C₈₈H₁₂₅N₁₃O₁₉ [MH]⁺ 1668.92, found 1570.41 (M−100, one Boc group fell off).

### N,N′,N″-(9-(1-((2S,4S)-4-azido-1-(2-((1-methyl-2,4-dioxo-1,4-dihydro-2H-benzo[d][1,3]oxazin-7-yl)oxy)acetyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(8-aminooctanamide), ARK-82_HCl Salt

To a solution of tri-tert-butyl (((9-(1-((2S,4S)-4-azido-1-(2-((1-methyl-2,4-dioxo-1,4-dihydro-2H-benzo[d][1,3]oxazin-7-yl)oxy)acetyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate (14) (0.54 g, 0.0032 mmol) in tetrahydrofuran (5.0 mL) was added 4 M HCl in dioxane solution (2 mL) at room temperature and the resulted reaction mixture was stirred for 4 h under nitrogen atmosphere. The reaction mixture was concentrated under reduced pressure to get crude ARK-82_HCl_Salt as a yellow solid. The crude mixture was purified by preparative HPLC using following method to get pure ARK-81_HCl salt (0.049 g, 10.2%) as a yellow solid. ¹H NMR (400 MHz, DMSO-d6) δ 9.95 ppm (3H, br S), 7.99 ppm (8H, broad s), 7.90-7.88 ppm (2H, d), 7.66 ppm (3H, broad s), 7.46 ppm (2H, broad s), 7.33 ppm (2H, broad s), 7.28-7.25 ppm (5H, m), 7.23-7.21 ppm (2H, d), 6.89-6.85 ppm (1H, m), 6.78-6.76 ppm (1H, m), 6.55 ppm (2H, broad s), 5.38 ppm (1H, s), 5.12-5.00 ppm (2H, m), 4.77 ppm (1H, m), 4.37-4.34 ppm (3H, m), 4.06-4.05 ppm (1H, m), 3.82 ppm (1H, m), 3.63-3.43 ppm (15H, m), 3.09-3.01 ppm (7H, m), 2.96-2.94 ppm (1H, d), 2.82-2.80 ppm (1H, d), 2.76-2.64 ppm (7H, m), 2.24 ppm (7H, broad s), 1.54-1.52 ppm (12H, d), 1.26 ppm (18H, s). MS (ESI-MS): m/z calcd for C₇₃H₁₀₁N₁₃O₁₃ [MH]⁺ 1368.76, found 1370.25.

Method for Preparative HPLC:

(A) 0.05% HCl in water (HPLC GRADE) and (B) 100% acetonitrile (HPLC GRADE), using KINETEX BIPHENYL, 250 mm*21.2 mm*5 μm with the flow rate of 20.0 mL/min and with the following gradient:

| Time | % A | % B |
|---|---|---|
| 0.01 | 95.0 | 5.0 |
| 3.00 | 77.0 | 23.0 |
| 24.00 | 72.0 | 28.0 |
| 24.01 | 0.0 | 100 |
| 25.00 | 0.0 | 100 |
| 25.01 | 95.0 | 5.0 |
| 26.00 | 95.0 | 5.0 |

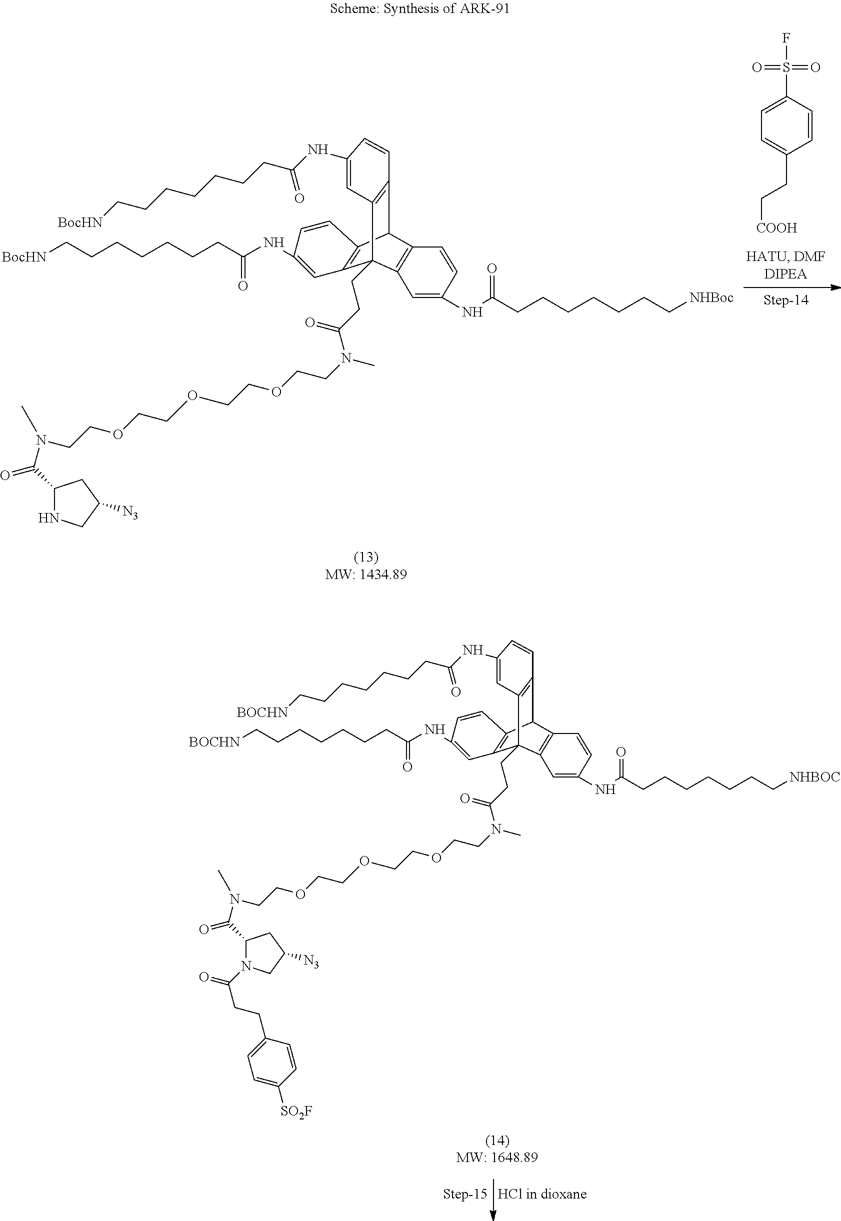

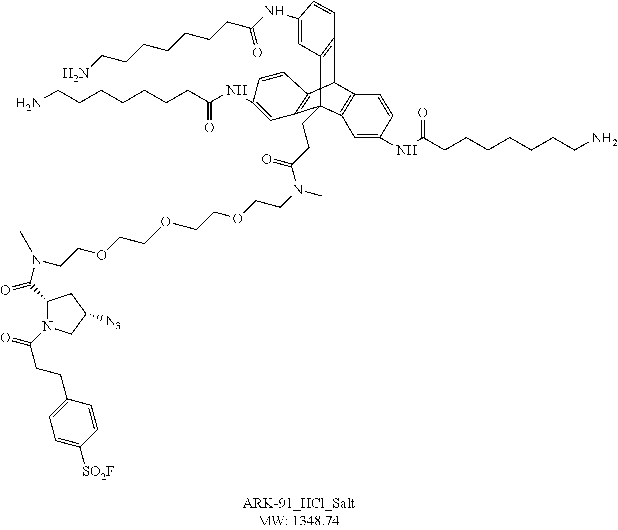

### Tri-tert-butyl (((9-(1-((2S,4S)-4-azido-1-(3-(4-(fluorosulfonyl)phenyl)propanoyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris (8-oxooctane-8,1-diyl))tricarbamate (13) (0.30 g, 0.21 mmol) in N,N-dimethylformamide (6 mL) were sequentially added 3-(4-(fluorosulfonyl)phenyl)propanoic acid (00.058 g, 0.25 mmol) and HATU (0.095 g, 0.25 mmol) at room temperature. The reaction mixture was stirred for 5 minutes. To this, N,N-diisopropylethylamine (0.054 g, 0.42 mmol) was added dropwise and the resulted reaction mixture was further stirred for 1 h at room temperature. The reaction mixture was diluted by ethyl acetate (100 mL) and washed with ice-cold water (3×30 mL). The organic layers were combined, washed with brine and concentrated under reduced pressure at 25° C. to get crude 14 (0.55 g, quantitative yield) as a brown solid which was used in the next step without further purification. MS (ESI-MS): m/z calcd C₉₆H₁₂₅FN₁₂₀₁₇S [MH]⁺ 1649.89, found 1551.29 (M−100, one Boc group fell off).

### N,N′,N″-(9-(1-((2S,4S)-4-azido-1-(3-(4-(fluorosulfonyl)phenyl)propanoyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-aminooctanamide), ARK-91_HCl Salt

To a solution tri-tert-butyl (((9-(1-((2S,4S)-4-azido-1-(3-(4-(fluorosulfonyl)phenyl) propanoyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate (14) (0.55 g, 0.0033 mmol) in 1,4-dioxane (9.0 mL) was added 4 M HCl in dioxane (4 mL) at room temperature. The resulting reaction mixture was stirred for 4 hours. The mixture was concentrated under reduced pressure to get crude of ARK-91_HCl_Salt as yellow solid. The crude mixture was purified by preparative HPLC using following method to get pure ARK-91_HCl salt (0.09 g, 18.5%) as yellow solid. ¹H NMR (400 MHz, DMSO-d6) δ 9.94 ppm (3H, broad s), 8.04-8.00 ppm (2H, m), 7.96 ppm (6H, broad s), 7.66 ppm (4H, broad s), 7.62-7.52 ppm (1H, m), 7.31-7.18 ppm (6H, broad s), 5.38 ppm (1H, s), 4.71-4.66 ppm (1H, m), 4.25 ppm (9H, m), 3.40-3.99 ppm (1H, m), 3.63-3.49 ppm (9H, m), 3.44-3.35 ppm (5H, m), 3.31-3.24 ppm (2H, m), 3.16-3.15 ppm (2H, m), 3.09-3.00 ppm (6H, m), 2.95-2.91 ppm (3H, m), 2.77-2.69 ppm (7H, m), 2.26-2.23 ppm (6H, t), 1.54-1.52 ppm (12H, d), 1.26 ppm (18H, broad s). MS (ESI-MS): m z calcd for C₇₁H₁₀₁FN₁₂O₁₁S [MH]⁺ 1349.74, found 1350.38.

Method for Preparative HPLC:

(A) 0.05% HCl in water (HPLC GRADE) and (B) 100% acetonitrile (HPLC GRADE), using X-SELECT C18, 250 mm*30 mm, 5 μm with the flow rate of 23.0 mL/min and with the following gradient:

| Time | % A | % B |
|---|---|---|
| 0.01 | 85.0 | 15.0 |
| 5.00 | 80.0 | 20.0 |
| 25.00 | 60.0 | 40.0 |
| 25.01 | 0.0 | 100 |
| 26.00 | 0.0 | 100 |
| 26.01 | 85.0 | 15.0 |
| 27.00 | 85.0 | 15.0 |

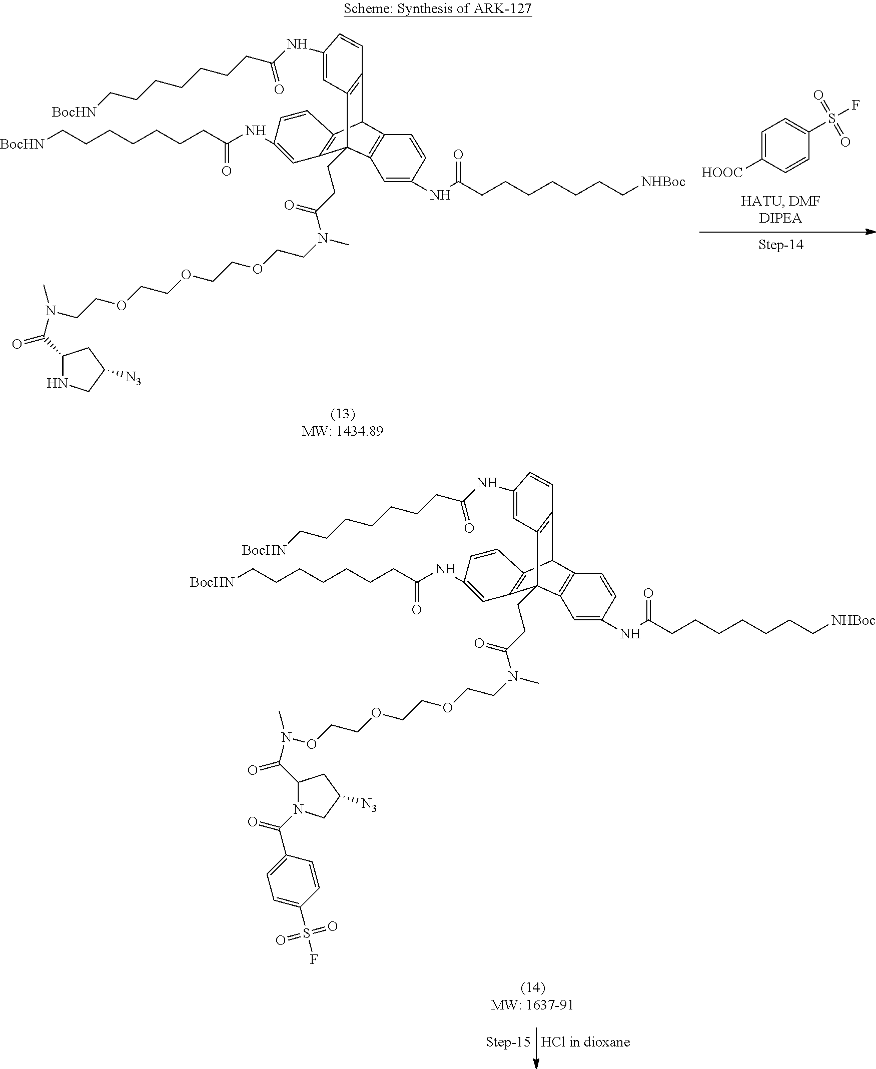 [SMILES: CN(CCOCCOCCOCCN(C)C(=O)[C@@H]1C[C@H](N=[N+]=[N-])CN1)C(=O)CCC12c3cc(NC(=O)CCCCCCCNC(=O)OC(C)(C)C)ccc3C(c3ccc(NC(=O)CCCCCCCNC(=O)OC(C)(C)C)cc31)c1ccc(NC(=O)CCCCCCCNC(=O)OC(C)(C)C)cc12 ; O=C(O)c1ccc(S(=O)(=O)F)cc1 ; CN(CCOCCOCCON(C)C(=O)C1C[C@H](N=[N+]=[N-])CN1C(=O)c1ccc(S(=O)(=O)F)cc1)C(=O)CCC12c3cc(NC(=O)CCCCCCCNC(=O)OC(C)(C)C)ccc3C(c3ccc(NC(=O)CCCCCCCNC(=O)OC(C)(C)C)cc31)c1ccc(NC(=O)CCCCCCCNC(=O)OC(C)(C)C)cc12]

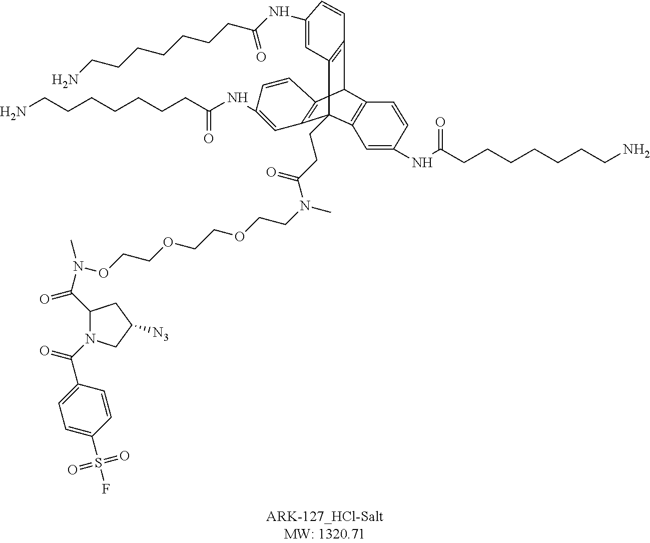 [SMILES: CN(CCOCCOCCON(C)C(=O)C1C[C@H](N=[N+]=[N-])CN1C(=O)c1ccc(S(=O)(=O)F)cc1)C(=O)CCC12c3cc(NC(=O)CCCCCCCN)ccc3C(c3ccc(NC(=O)CCCCCCCN)cc31)c1ccc(NC(=O)CCCCCCCN)cc12]

### tri-tert-butyl (((9-(1-((2S,4S)-4-azido-1-(4-(fluorosulfonyl)benzoyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate, 14

To a solution of tri-tert-butyl (((9-(1-((2S,4S)-4-azidopyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris (8-oxooctane-8,1-diyl))tricarbamate (13) (0.05 g, 0.03 mmol) in N,N-dimethylformamide (2 mL) were sequentially added 4-fluorosulfonylbenzoic acid (0.09 g, 0.04 mmol) and HATU (0.016 g, 0.04 mmol) at room temperature. The reaction mixture was stirred for 5 minutes. To this, N,N-diisopropylethylamine (0.09 g, 0.14 mmol) was added drop wise and the resulted reaction mixture was further stirred for 1 h at room temperature. The reaction mixture was diluted by ethyl acetate (100 mL) and washed with ice-cold water (3×30 mL). The organic layers were combined, washed with brine and concentrated under reduced pressure at 25° C. to get crude 14 (0.075 g, quantitative yield) as a yellow semi-solid which was used in the next step without further purification. MS (ESI-MS): m/z calcd C₈₄H₁₂₁FN₁₂O₁₇S [MH]⁺ 1621.87, found 1523.47 (M−100, one Boc group fell off).

### 4-((2S,4S)-4-azido-2-(methyl(12-methyl-13-oxo-15-(2,7,15-tris(8-aminooctanamido)-9,10-[1,2]benzenoanthracen-9(10H)-yl)-3,6,9-trioxa-12-azapentadecyl)carbamoyl)pyrrolidine-1-carbonyl)benzenesulfonyl fluoride, ARK-127_HCl Salt

To a solution of tri-tert-butyl (((9-(1-((2S,4S)-4-azido-1-(4-(fluorosulfonyl)benzoyl)pyrrolidin-2-yl)-2,14-dimethyl-1,15-dioxo-5,8,11-trioxa-2,14-diazaheptadecan-17-yl)-9,10-dihydro-9,10-[1,2]benzenoanthracene-2,7,15-triyl)tris(azanediyl))tris(8-oxooctane-8,1-diyl))tricarbamate (14) (0.075 g, 0.0005 mmol) in 1,4-dioxane (3.0 mL) was added 4 M HCl in dioxane (1 mL) at room temperature and the resulting reaction mixture was stirred for 4 hours. The mixture was concentrated under reduced pressure to get crude ARK-127_HCl_Salt as a yellow solid. The crude mixture was purified by preparative HPLC using following method to get pure ARK-127_HCl_Salt (0.014 g, 21.2%) as a yellow solid. ¹H NMR (400 MHz, DMSO-d₆) δ 9.89 ppm (3H, broad s), 8.26-8.22 ppm (1H, m), 8.16 ppm (1H, m), 7.89-7.85 ppm (9H, m), 7.75 ppm (1H, m), 7.69-7.66 ppm (3H, m), 7.29-7.22 ppm (5H, m), 5.38 ppm (1H, s), 4.99-4.87 ppm (2H, m), 4.39-4.38 ppm (1H, m), 4.28-4.16 ppm (1H, m), 4.05-4.02 ppm (1H, m), 3.81-3.74 ppm (1H, m), 3.64-3.52 ppm (9H, m), 3.38-3.28 ppm (7H, m), 3.17-2.99 ppm (8H, m), 2.76-2.65 ppm (8H, m), 2.34-2.23 ppm (5H, t), 1.54 ppm (11H, broad s), 1.27 ppm (18H, broad s). MS (ESI-MS): m/z calcd for C₆₉H₉₇FN₁₂O₁₁S [MH]⁺ 1321.71, found 1322.42.

Method for Preparative HPLC:

(A) 0.05% HCl in water (HPLC grade) and (B) 100% Acetonitrile (HPLC grade), using SUNFIRE C18, 250 mm*19 mm*5 μm with the flow rate of 20.0 mL/min and with the following gradient:

| Time | % A | % B |
|---|---|---|
| 0.01 | 86.0 | 14.0 |
| 19.00 | 70.0 | 30.0 |
| 19.01 | 100.0 | 0.0 |
| 20.00 | 100.0 | 0.0 |
| 20.01 | 86.0 | 14.0 |
| 21.00 | 86.0 | 14.0 |

### Example 18: Preparation of CPNQ Analogues and Other Quinoline-Based Ligands

Exemplary small molecule ligands based on CPNQ and other quinoline scaffolds were prepared based on the synthetic schemes shown in FIGS. 97-105. Analytical data for the prepared compounds are shown below in Table 6.

TABLE 6
Analytical Data for CPNQ Analogues and Quinoline-Based Ligands

| Target | Mol. | | HPLC | HPLC | LCMS | LCMS | |
|---|---|---|---|---|---|---|---|
| ID | Weight | MH+ | RT | Purity | RT | Purity | ¹H NMR |
| ARK-131 | 620.3 | 621.69 | 6.902 min | 97.56% | 4.378 min | 95.78% | DMSO-d6: δ 9.04-9.03 ppm |
| | | | | | | | (1H, dd, J = 4, 1.6 Hz), 8.66- |
| | | | | | | | 8.63 ppm (1H, J = 8.4, 1.2 Hz), |
| | | | | | | | 8.26-8.24 ppm (1H, d, J = 8.4 |
| | | | | | | | Hz), 7.73-7.70 ppm (1H, dd, J = |
| | | | | | | | 8.4, 4 Hz), 7.53-7.47 ppm (4H, |
| | | | | | | | m), 7.26-7.24 ppm (1H, d, J = 8.4 |
| | | | | | | | Hz), 3.96 ppm (2H, br s), 3.64 |
| | | | | | | | ppm (4H, br s), 3.51 ppm (3H, |
| | | | | | | | m), 3.43-3.39 ppm (2H, m), 3.30 |
| | | | | | | | ppm (2H, m), 3.23-3.18 ppm |
| | | | | | | | (4H, m), 2.99-2.95 (3H, m), |
| | | | | | | | 2.89-2.79 ppm (5H, m), 1.38- |
| | | | | | | | 1.37 ppm (9H, d, J = 5.6 Hz). |
| ARK-137 | 654.26 | 655.64 | 7.792 min | 97.73% | 4.630 min | 96.05% | DMSO-d6: δ 9.03 ppm (1H, br |
| | | | | | | | s), 8.76-8.74 ppm (1H, d, J = 8.4 |
| | | | | | | | Hz), 8.21-8.19 ppm (1H, d, J = 8 |
| | | | | | | | Hz), 7.74-7.72 ppm (1H, |
| | | | | | | | m), 7.58-7.53 ppm (4H, m), |
| | | | | | | | 7.31-7.30 ppm (1H, d, J = 6 Hz), |
| | | | | | | | 4.67 ppm (1H, br s), 4.28 ppm |
| | | | | | | | (1H, br s), 3.97-3.91 (1H, m), |
| | | | | | | | 3.71 ppm (3H, br s), 3.59 ppm |
| | | | | | | | (1H, br s), 3.46 ppm (1H, br s), |
| | | | | | | | 3.28-3.10 ppm (6H, m), 2.96- |
| | | | | | | | 2.94 ppm (2H, br s), 2.79-2.68 |
| | | | | | | | ppm (5H, m), 1.39-1.35 ppm |
| | | | | | | | (9H, d, J = 15.2 Hz). |
| ARK-138 | 249.18 | 250.36 | 5.761 min | 100% | 2.061 min | 100% | D20: δ 7.66-7.64 ppm (2H, d, |
| | | | | | | | J = 7.6 Hz), 7.53-7.49 ppm (1H, |
| | | | | | | | t, J = 14.8, 7.6 Hz), 7.43-7.39 |
| | | | | | | | ppm (2H, t, J = 15.2, 7.6 Hz), |
| | | | | | | | 3.40-3.36 ppm (2H, m), 3.25- |
| | | | | | | | 3.18 ppm (2H, m), 3.16-3.09 |
| | | | | | | | ppm (2H, m), 2.99-2.96 ppm |
| | | | | | | | (2H, t, J = 15.2, 7.6 Hz), 2.80 |
| | | | | | | | ppm (3H, s), 2.06-1.94 ppm |
| | | | | | | | (4H, m). |
| ARK-179 | 396.1 | 397.29 | 7.287 min | 100% | 2.297 min | 95.09% | DMSO-d6: δ 9.05-9.04 ppm |
| | | | | | | | (1H, dd, J = 4, 1.6 Hz), 8.67-8.65 |
| | | | | | | | ppm (1H, dd, J = 8.4, 1.2 Hz), |
| | | | | | | | 8.28-8.25 ppm (1H, d, J = 8.4 |
| | | | | | | | Hz), 7.75-7.71 ppm (1H, dd, |
| | | | | | | | J = 8.8, 4.4 Hz), 7.57-7.51 ppm |
| | | | | | | | (4H, m), 7.26-7.24 ppm (1H, d, |
| | | | | | | | J = 8.4 Hz), 3.94 ppm (2H, br s), |
| | | | | | | | 3.65 ppm (2H, br s), 3.21 ppm |
| | | | | | | | (4H, br s). |
| ARK-180 | 380.13 | 381.39 | 6.791 min | 96.24% | 4.185 min | 98.46% | DMSO-d6: δ 9.04-9.03 ppm |
| | | | | | | | (1H, dd, J = 4.4, 1.6 Hz), 8.65- |
| | | | | | | | 8.63 ppm (1H, dd, J = 8.8, 1.6 |
| | | | | | | | Hz), 8.25-8.23 ppm (1H, d, |
| | | | | | | | J = 8.4 Hz), 7.73-7.70 ppm (1H, |
| | | | | | | | dd, J = 8.8, 4 Hz), 7.58-7.55 ppm |
| | | | | | | | (2H, m), 7.35-7.29 ppm (2H, m), |
| | | | | | | | 7.26-7.24 ppm (1H, d, J = 8.4 |
| | | | | | | | Hz), 3.93 ppm (2H, br s), 3.67 |
| | | | | | | | ppm (2H, br s), 3.19 ppm (4H, |
| | | | | | | | br s). |
| ARK-181 | 440.04 | 441.4 | 7.404 min | 97.43% | 4.415 min | 96.33% | DMSO-d6: δ 9.04-9.03 ppm |
| | | | | | | | (1H, dd, J = 4, 1.6 Hz), 8.65-8.63 |
| | | | | | | | ppm (1H, dd, J = 8.8, 1.6 Hz), |
| | | | | | | | 8.25-8.23 ppm (1H, d, J = 8.4 |
| | | | | | | | Hz), 7.73-7.69 ppm (3H, m), |
| | | | | | | | 7.46-7.44 ppm (2H, dd, J = 6.8, |
| | | | | | | | 1.6 Hz), 7.26-7.23 ppm (1H, d, |
| | | | | | | | J = 8.4 Hz), 3.94 ppm (2H, br s), |
| | | | | | | | 3.64 ppm (2H, br s), 3.21-3.17 |
| | | | | | | | ppm (4H, m). |
| ARK-182 | 392.15 | 393.47 | 6.685 min | 96.87% | 4.190 min | 99.00% | DMSO-d6: δ 9.04-9.03 ppm |
| | | | | | | | (1H, d, J = 2.8 Hz), 8.67-8.65 |
| | | | | | | | ppm (1H, d, J = 8.8 Hz), 8.26- |
| | | | | | | | 8.24 ppm (1H, d, J = 8.4 Hz), |
| | | | | | | | 7.74-7.71 ppm (1H, dd, J = 8.8, |
| | | | | | | | 4.4 Hz), 7.47-7.44 ppm (2H, d, |
| | | | | | | | J = 8.8 Hz), 7.26-7.24 ppm (1H, |
| | | | | | | | d, J = 8.4 Hz), 7.03-7.01 ppm |
| | | | | | | | (2H, d, J = 8.4 Hz), 3.83-3.81 |
| | | | | | | | ppm (2H, d, J = 6.4 Hz), 3.19 |
| | | | | | | | ppm (4H, br s), 2.55 ppm (3H, s). |
| ARK-183 | 362.14 | 363.46 | 6.650 min | 100% | 4.158 min | 100% | DMSO-d6: δ 9.04-9.03 ppm |
| | | | | | | | (1H, dd, J = 4, 1.2 Hz), 8.66-8.63 |
| | | | | | | | ppm (1H, dd, J = 8.8, 1.6 Hz), |
| | | | | | | | 8.25-8.23 ppm (1H, d, J = 8 Hz), |
| | | | | | | | 7.73-7.70 ppm (1H, dd, J = 8.4, 4 |
| | | | | | | | Hz), 7.51-7.47 ppm(5H, br s), |
| | | | | | | | 7.26-7.24 ppm (1H, dd, J = 8.4 |
| | | | | | | | Hz), 3.95 ppm (2H, br s), 3.65 |
| | | | | | | | ppm (2H, br s), 3.20-3.19 ppm |
| | | | | | | | (4H, br s). |
| ARK-184 | 430.06 | 431.35 | 7.593 min | 96.37% | 2.453 min | 98.88% | DMSO-d6: δ 9.04-9.03 ppm |
| | | | | | | | (1H, dd, J = 4, 2.8 Hz), 8.65-8.63 |
| | | | | | | | ppm (1H, d, J = 8.4 Hz), 8.26- |
| | | | | | | | 8.24 ppm (1H, d, J = 8.4 Hz), |
| | | | | | | | 7.80-7.71 ppm (3H, m), 7.51- |
| | | | | | | | 7.48 ppm(1H, dd, J = 8, 1.6 Hz), |
| | | | | | | | 7.26-7.24 ppm (1H, d, J = 8.4 |
| | | | | | | | Hz), 3.94 ppm (2H, br s), 3.64 |
| | | | | | | | ppm (2H, br s), 3.22 (2H, S), |
| | | | | | | | 3.16 ppm (2H, S). |
| ARK-185 | 369.09 | 397.45 | 7.010 min | 96.30% | 2.291 min | 100% | DMSO-d6: δ 9.04-9.03 ppm |
| | | | | | | | (1H, dd, J = 4, 1.6 Hz), 8.65-8.63 |
| | | | | | | | ppm (1H, dd, J = 8.4, 1.6 Hz), |
| | | | | | | | 8.25-8.23 ppm (1H, d, J = 8 Hz), |
| | | | | | | | 7.73-7.70 ppm (1H, dd, J = 8.8, |
| | | | | | | | 4.4 Hz), 7.58-7.54 ppm (2H, m), |
| | | | | | | | 7.52-7.50 ppm (1H, m), 7.46- |
| | | | | | | | 7.44 ppm (1H, m), 7.26-7.24 |
| | | | | | | | ppm (1H, d J = 8.4 Hz), 3.95 ppm |
| | | | | | | | (2H, br s), 3.63 ppm (2H, br s), |
| | | | | | | | 3.23-3.17 ppm (4H, br d). |
| ARK-186 | 432.06 | 433.39 | 8.050 min | 96.44% | 4.510 min | 100% | DMSO-d6: δ 9.00-8.99 ppm |
| | | | | | | | (1H, dd, J = 4, 1.2 Hz), 8.52-8.50 |
| | | | | | | | ppm (1H, dd, J = 8.4, 1.6 Hz), |
| | | | | | | | 8.23-8.21 ppm (1H, d, J = 8.4 |
| | | | | | | | Hz), 7.86-7.79 ppm (4H, m), |
| | | | | | | | 7.64-7.61 ppm (1H, dd, J = 8.4, 4 |
| | | | | | | | Hz), 7.26-7.24 ppm (1H, d, J = 8 |
| | | | | | | | Hz), 3.24 ppm (8H, s). |
| ARK-187 | 426.11 | 427.39 | 6.319 min | 97.61% | 2.051 min | 99.40% | DMSO-d6: δ 9.03-9.02 ppm |
| | | | | | | | (1H, dd, J = 4.4, 1.6 Hz), 8.70 |
| | | | | | | | ppm (1H, br s), 8.26-8.24 ppm |
| | | | | | | | (1H, d, J = 8 Hz), 7.71-7.68 ppm |
| | | | | | | | (1H, dd, J = 8.8, 4.4 Hz), 7.56- |
| | | | | | | | 7.51 ppm (4H, m), 7.39 ppm |
| | | | | | | | (1H, br s), 4.62-4.54 ppm (1H, |
| | | | | | | | br s), 4.13-4.10 ppm (1H, m), |
| | | | | | | | 3.87 ppm (1H, br s), 3.60-3.51 |
| | | | | | | | ppm (3H, m), 3.45-3.39 ppm |
| | | | | | | | (3H, br s), 3.11 ppm (1H, br s). |
| ARK-189 | 410.11 | 411.41 | 7.229 min | 98.36% | 4.390 min | 100% | DMSO-d6: δ 9.02-8.99 ppm |
| | | | | | | | (1H, dd, J = 10.4, 3.2 Hz), 8.63- |
| | | | | | | | 8.61 ppm (1H, dd, J = 43.6, 8.4 |
| | | | | | | | Hz), 8.23-8.20 ppm (1H, m), |
| | | | | | | | 7.72-7.62 ppm (1H, m), 7.55- |
| | | | | | | | 7.47 ppm (3H, m), 7.41-7.39 |
| | | | | | | | ppm (1H, d, J = 8 Hz), 7.28-7.23 |
| | | | | | | | ppm (1H, m), 3.93 ppm (1H, br |
| | | | | | | | s), 3.85-3.82 ppm (1H, t, J = |
| | | | | | | | 11.2, 5.6 Hz), 3.56-3.53 ppm |
| | | | | | | | (3H, m), 3.46-3.42 ppm (3H, m), |
| | | | | | | | 2.17 ppm (1H, br s), 2.01 ppm |
| | | | | | | | (1H, br s). |
| ARK-190 | 408.1 | 409.16 | 6.575 min | 100% | 1.738 min | 97.92% | DMSO-d6: δ 8.97-8.96 ppm |
| | | | | | | | (1H, d, J = 2.8 Hz), 8.62-8.60 |
| | | | | | | | ppm (1H, d, J = 7.6 Hz), 8.20- |
| | | | | | | | 8.09 ppm (1H, dd, J = 36, 8.8 |
| | | | | | | | Hz), 7.58-7.47 ppm (5H, m), |
| | | | | | | | 6.98-6.81 ppm (1H, m), 4.92- |
| | | | | | | | 4.78 ppm (1H, m), 4.41-4.22 |
| | | | | | | | ppm (2H, m), 3.81-3.59 ppm |
| | | | | | | | (3H, m), 2.14-2.09 ppm (2H, m). |
| ARK-191 | 408.1 | 409.21 | 6.614 min | 98.38% | 1.719 min | 98.54% | DMSO-d6: δ 8.96 ppm (1H, br |
| | | | | | | | s), 8.61 ppm (1H, br s), 8.20- |
| | | | | | | | 8.09 ppm (1H, m), 7.52 ppm |
| | | | | | | | (5H, br s), 6.98-6.82 ppm (1H, |
| | | | | | | | m), 4.92-4.78 ppm (1H, m), |
| | | | | | | | 4.41-4.22 ppm (2H, m), 3.79- |
| | | | | | | | 3.61 ppm (3H, m), 2.14-2.09 |
| | | | | | | | ppm (2H, m). |
| ARK-194 | 352.11 | 353.44 | 6.402 min | 99.65% | 4.366 min | 99.38% | DMSO-d6: δ 8.66 ppm (1H, s), |
| | | | | | | | 8.06-8.04 ppm (1H, d, J = 8.4 |
| | | | | | | | Hz), 7.87-7.82 ppm (2H, d, |
| | | | | | | | J = 12.4, 8.8 Hz), 7.59-7.51 ppm |
| | | | | | | | (5H, m), 3.85 ppm (4H, br s), |
| | | | | | | | 3.76 ppm (3H, br s), 3.59 ppm |
| | | | | | | | (2H, br s). |
| ARK-196 | 342.11 | 343.48 | 7.873 min | 96.62% | 4.684 min | 95.57% | DMSO-d6: δ 7.55-7.47 ppm |
| | | | | | | | (4H, m), 7.04-7.00 ppm (1H, t, |
| | | | | | | | J = 15.6, 7.6 Hz), 6.45-6.42 ppm |
| | | | | | | | (2H, dd, J = 8, 3.6 Hz), , 4.50- |
| | | | | | | | 4.46 ppm (2H, t, J = 16.8, 8.4 |
| | | | | | | | Hz), 3.75 ppm (2H, br s), 3.46 |
| | | | | | | | ppm (2H, br s), 3.13-3.09 ppm |
| | | | | | | | (2H, t, J = 8.4 Hz), 3.03-2.97 |
| | | | | | | | ppm (4H, m). |

### Example 19: Exemplary Compound Data

Additional data for compounds whose preparation is described above as well as structures of further exemplary compounds are provided in Table 7 below.

TABLE 7
Exemplary Compound Structures and Data

| | Compound | Collabora- | | Parent | Batch | HPLC | LCMS |
|---|---|---|---|---|---|---|---|
| Molecule | Name | tion Code | Salt | MW | MW | (%) | (%) |
| 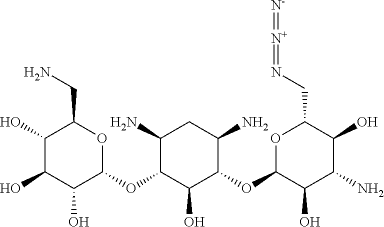 [SMILES: [N-]=[N+]=NC[C@H]1O[C@H](O[C@@H]2[C@@H](O)[C@H](O[C@H]3O[C@H](CN)[C@@H](O)[C@H](O)[C@H]3O)[C@@H](N)C[C@H]2N)[C@H](O)[C@@H](N)[C@@H]1O] | ARK000007 | ARK-1 | 4HCl | 509.5114 | 655.3514 | 97.58 | Mass confirmed |
| 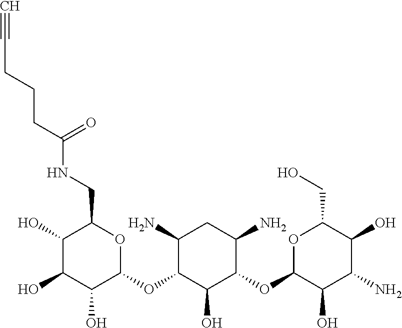 [SMILES: C#CCCCC(=O)NC[C@H]1O[C@H](O[C@H]2[C@H](O)[C@@H](O[C@H]3O[C@H](CO)[C@@H](O)[C@H](N)[C@H]3O)[C@H](N)C[C@@H]2N)[C@H](O)[C@@H](O)[C@@H]1O] | ARK000008 | ARK-2 | 3TFA | 578.60988 | 920.66988 | 96.38 | 99.5 |
| 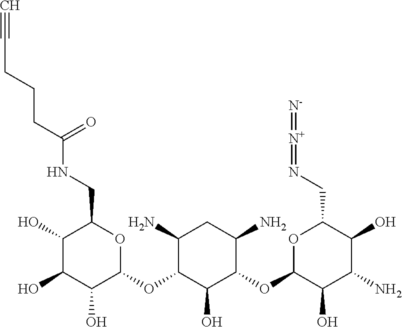 [SMILES: C#CCCCC(=O)NC[C@H]1O[C@H](O[C@H]2[C@H](O)[C@@H](O[C@H]3O[C@H](CN=[N+]=[N-])[C@@H](O)[C@H](N)[C@H]3O)[C@H](N)C[C@@H]2N)[C@H](O)[C@@H](O)[C@@H]1O] | ARK000009 | ARK-3 | 3TFA | 603.62264 | 945.68264 | 95.55 | 0.9832 |
| 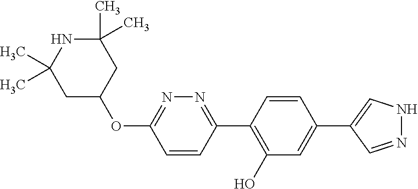 [SMILES: CC1(C)CC(Oc2ccc(-c3ccc(-c4cn[nH]c4)cc3O)nn2)CC(C)(C)N1] | ARK000010 | ARK-4 | 1HCl | 393.48208 | 429.94208 | 98.41 | 99.04 |
| 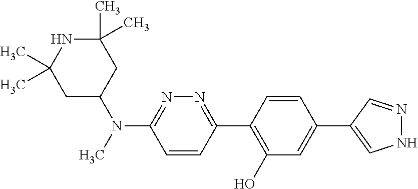 [SMILES: CN(c1ccc(-c2ccc(-c3cn[nH]c3)cc2O)nn1)C1CC(C)(C)NC(C)(C)C1] | ARK000011 | ARK-5 | 2HCl | 406.5239 | 479.4439 | 99.88 | 100 |
| 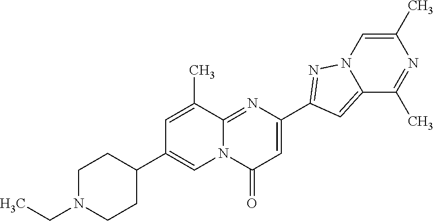 [SMILES: CCN1CCC(c2cc(C)c3nc(-c4cc5c(C)nc(C)cn5n4)cc(=O)n3c2)CC1] | ARK000012 | ARK-6 | 1HCl | 416.51872 | 452.97872 | 96.26 | 99.12 |
| 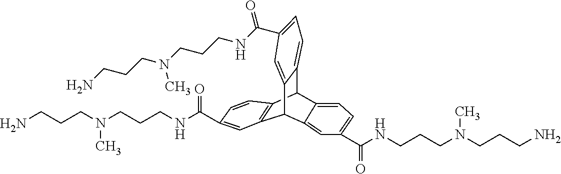 [SMILES: CN(CCCN)CCCNC(=O)c1ccc2c(c1)C1c3cc(C(=O)NCCCN(C)CCCN)ccc3C2c2ccc(C(=O)NCCCN(C)CCCN)cc21] | ARK000013 | ARK-7 | 6HCl | 768.0454 | 986.8054 | 100 | 96.15 |
| 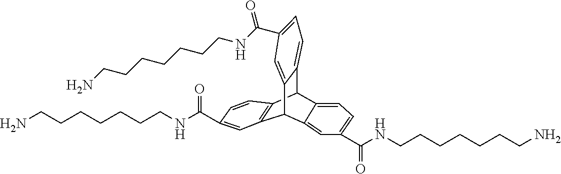 [SMILES: NCCCCCCCNC(=O)c1ccc2c(c1)C1c3cc(C(=O)NCCCCCCCN)ccc3C2c2ccc(C(=O)NCCCCCCCN)cc21] | ARK000014 | ARK-8 | 3HCl | 723.00148 | 832.38148 | 98.74 | 99.69 |
| 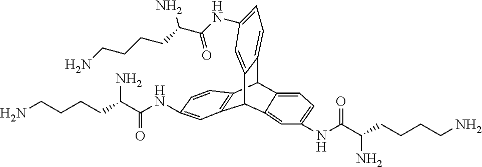 [SMILES: NCCCC[C@H](N)C(=O)Nc1ccc2c(c1)C1c3cc(NC(=O)[C@@H](N)CCCCN)ccc3C2c2ccc(NC(=O)[C@@H](N)CCCCN)cc21] | ARK000015 | ARK-9-01 | 6HCl | 683.88592 | 902.64592 | 98.56 | 98.4 |
| 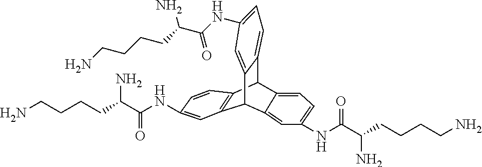 [SMILES: NCCCC[C@H](N)C(=O)Nc1ccc2c(c1)C1c3cc(NC(=O)[C@@H](N)CCCCN)ccc3C2c2ccc(NC(=O)[C@@H](N)CCCCN)cc21] | ARK000015 | ARK-9-02 | 6TFA | 683.88592 | 1368.00592 | 98.49 | 97.34 |
| 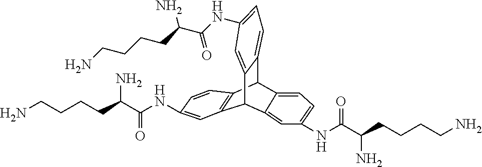 [SMILES: NCCCC[C@@H](N)C(=O)Nc1ccc2c(c1)C1c3cc(NC(=O)[C@H](N)CCCCN)ccc3C2c2ccc(NC(=O)[C@H](N)CCCCN)cc21] | ARK000016 | ARK-10 | 6HCl | 683.88592 | 902.64592 | 99.25 | 100 |
| 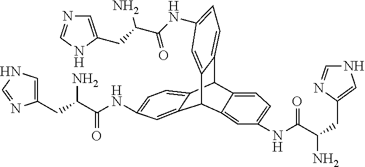 [SMILES: N[C@@H](Cc1c[nH]cn1)C(=O)Nc1ccc2c(c1)C1c3cc(NC(=O)[C@@H](N)Cc4c[nH]cn4)ccc3C2c2ccc(NC(=O)[C@@H](N)Cc3cnc[nH]3)cc21] | ARK000017 | ARK-11 | 3HCl | 710.78692 | 820.16692 | 97.95 | 94.97 |
| 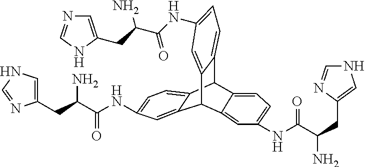 [SMILES: N[C@H](Cc1c[nH]cn1)C(=O)Nc1ccc2c(c1)C1c3cc(NC(=O)[C@H](N)Cc4c[nH]cn4)ccc3C2c2ccc(NC(=O)[C@H](N)Cc3cnc[nH]3)cc21] | ARK000018 | ARK-12 | 3HCl | 710.78692 | 820.16692 | 100 | 95.05 |
| 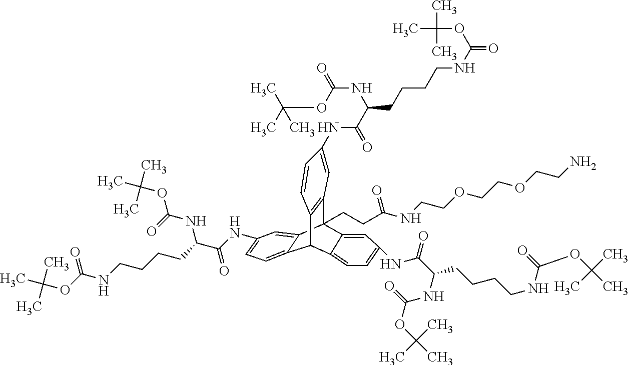 [SMILES: CC(C)(C)OC(=O)NCCCC[C@H](NC(=O)OC(C)(C)C)C(=O)Nc1ccc2c(c1)C1(CCC(=O)NCCOCCOCCN)c3cc(NC(=O)[C@H](CCCCNC(=O)OC(C)(C)C)NC(=O)OC(C)(C)C)ccc3C2c2ccc(NC(=O)[C@H](CCCCNC(=O)OC(C)(C)C)NC(=O)OC(C)(C)C)cc21] | ARK000022 | ARK-13-D | Free base | 1486.83166 | 1486.83166 | 96.31 | 95.89 |
| 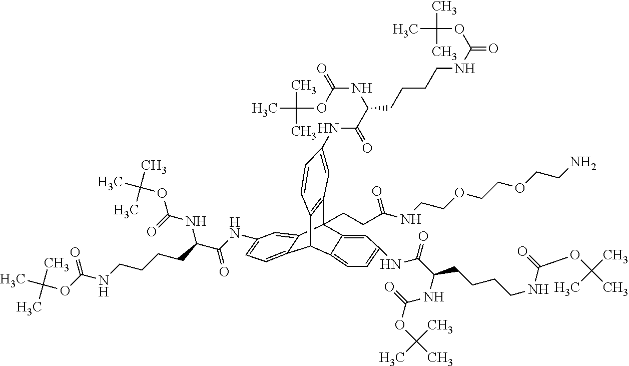 [SMILES: CC(C)(C)OC(=O)NCCCC[C@@H](NC(=O)OC(C)(C)C)C(=O)Nc1ccc2c(c1)C1(CCC(=O)NCCOCCOCCN)c3cc(NC(=O)[C@@H](CCCCNC(=O)OC(C)(C)C)NC(=O)OC(C)(C)C)ccc3C2c2ccc(NC(=O)[C@@H](CCCCNC(=O)OC(C)(C)C)NC(=O)OC(C)(C)C)cc21] | ARK000023 | ARK-13-L | Free base | 1486.83166 | 1486.83166 | 96.91 | 98.82 |
| 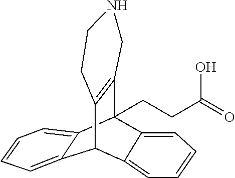 [SMILES: O=C(O)CCC12C3=C(CCNC3)C(c3ccccc31)c1ccccc12] | ARK000019 | ARK-14 | Free base | 331.40764 | 331.40764 | 96.72 | 99.58 |
| 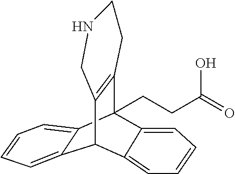 [SMILES: O=C(O)CCC12C3=C(CNCC3)C(c3ccccc31)c1ccccc12] | ARK000020 | ARK-15 | Free base | 331.40764 | 331.40764 | 92.97 | 97.11 |
| 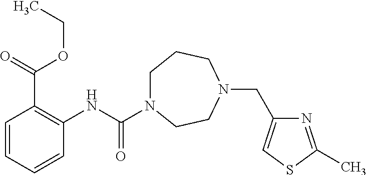 [SMILES: CCOC(=O)c1ccccc1NC(=O)N1CCCN(Cc2csc(C)n2)CC1] | ARK000021 | ARK-16 | 1HCl | 402.51044 | 438.97044 | 99.92 | 100 |
| 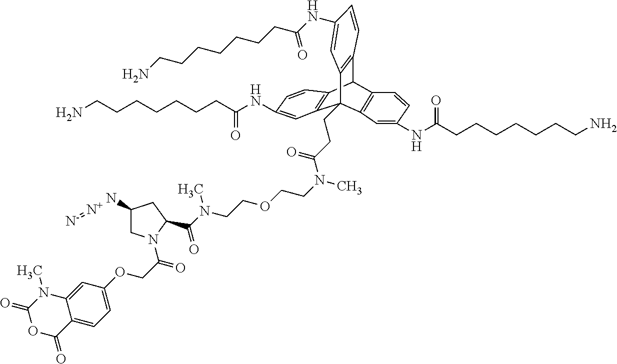 [SMILES: CN(CCOCCN(C)C(=O)[C@@H]1C[C@H](N=[N+]=[N-])CN1C(=O)COc1ccc2c(=O)oc(=O)n(C)c2c1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000024 | ARK-80 | 3HCl | 1280.55 | 1390.05 | NA | 96.45 |
| 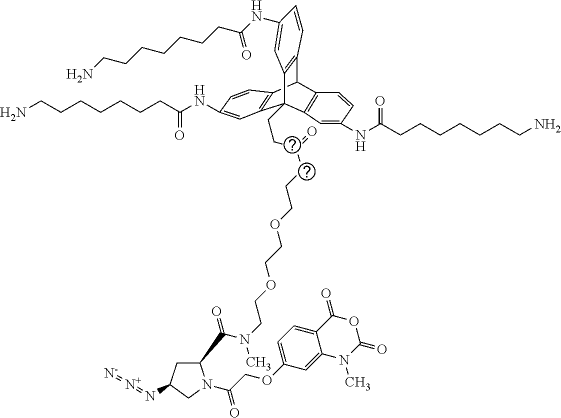 [SMILES: CN(CCOCCOCCCC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)COc1ccc2c(=O)oc(=O)n(C)c2c1] | ARK000025 | ARK-81 | 3HCl | 1324.61 | 1434.11 | NA | 95.31 |
| 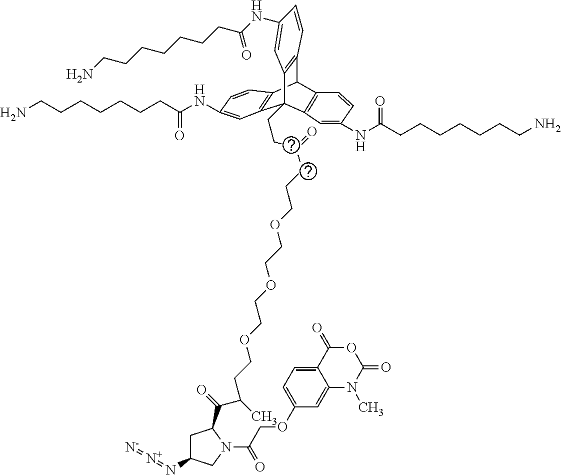 [SMILES: CC(CCOCCOCCOCCCC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)COc1ccc2c(=O)oc(=O)n(C)c2c1] | ARK000026 | ARK-82 | 3HCl | 1368.66 | 1478.16 | NA | 93.96 |
| 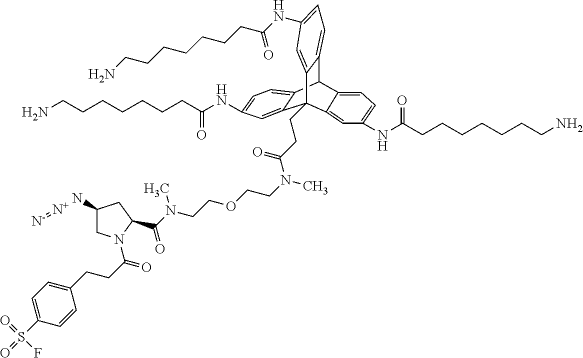 [SMILES: CN(CCOCCN(C)C(=O)[C@@H]1C[C@H](N=[N+]=[N-])CN1C(=O)CCc1ccc(S(=O)(=O)F)cc1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000027 | ARK-89 | 3HCl | 1261.59372 | 1370.97372 | NA | 96.51 |
| 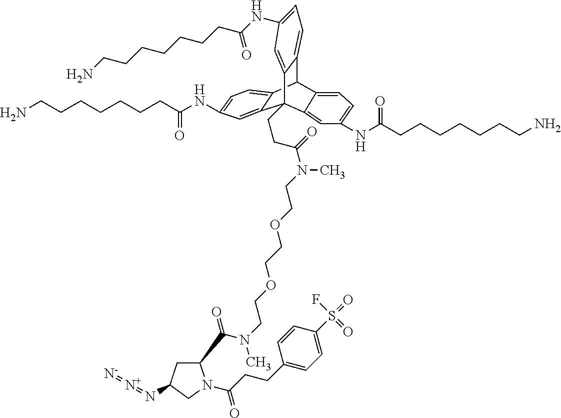 [SMILES: CN(CCOCCOCCN(C)C(=O)[C@@H]1C[C@H](N=[N+]=[N-])CN1C(=O)CCc1ccc(S(=O)(=O)F)cc1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000028 | ARK-90 | 3HCl | 1305.64628 | 1415.02628 | NA | 97.66 |
| 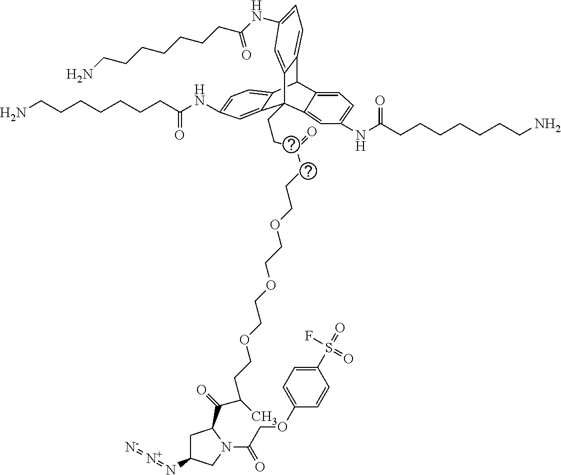 [SMILES: CC(CCOCCOCCOCCCC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)COc1ccc(S(=O)(=O)F)cc1] | ARK000029 | ARK-91 | 3HCl | 1349.69 | 1459.19 | NA | 95.66 |
| 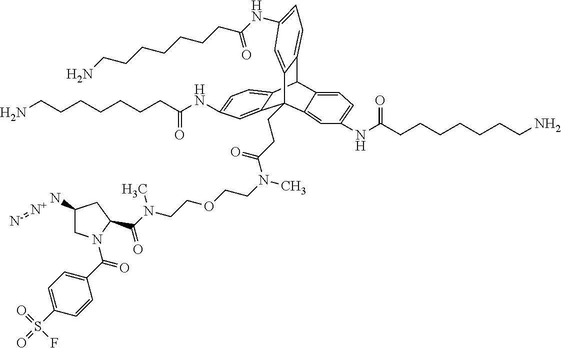 [SMILES: CN(CCOCCN(C)C(=O)[C@@H]1C[C@H](N=[N+]=[N-])CN1C(=O)c1ccc(S(=O)(=O)F)cc1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000030 | ARK-125 | 3HCl | 1232.85 | 1342.35 | NA | 94.07 |
| 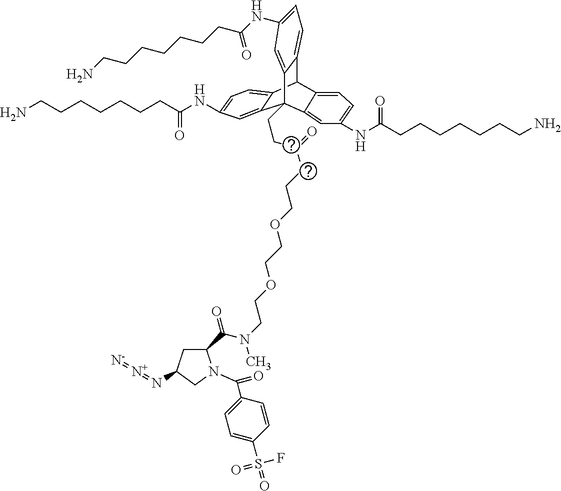 [SMILES: CN(CCOCCOCCCC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)c1ccc(S(=O)(=O)F)cc1] | ARK000031 | ARK-126 | 3HCl | 1276.68 | 1386.18 | NA | 96.89 |
| 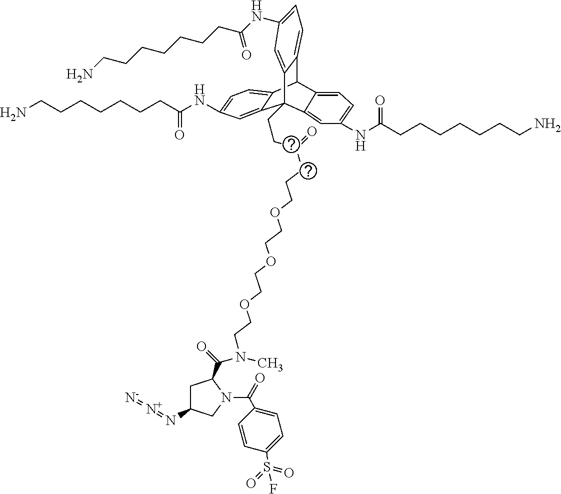 [SMILES: CN(CCOCCOCCOCCCC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)c1ccc(S(=O)(=O)F)cc1] | ARK000032 | ARK-127 | 3HCl | 1320.71 | 1430.21 | NA | 95.44 |
| 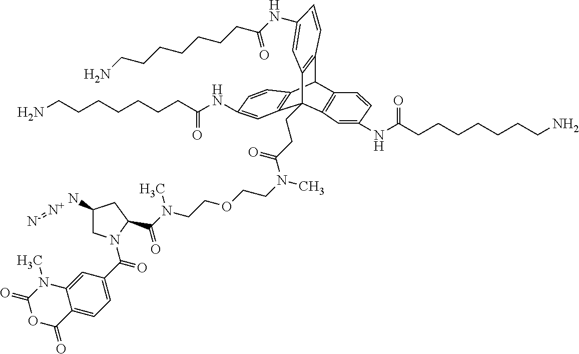 [SMILES: CN(CCOCCN(C)C(=O)[C@@H]1C[C@H](N=[N+]=[N-])CN1C(=O)c1ccc2c(=O)oc(=O)n(C)c2c1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000033 | ARK-77 | 3HCl | 1250.63 | 1360.13 | NA | 88.19 |
| 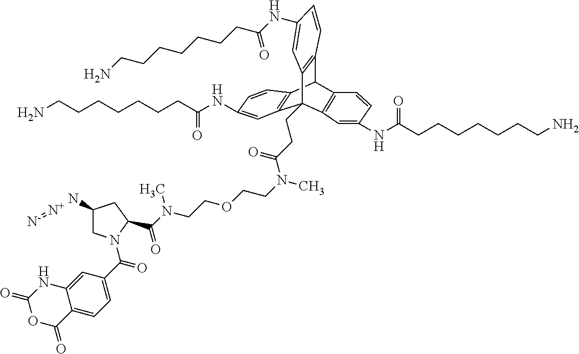 [SMILES: CN(CCOCCN(C)C(=O)[C@@H]1C[C@H](N=[N+]=[N-])CN1C(=O)c1ccc2c(=O)oc(=O)[nH]c2c1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000034 | ARK-77A | 3HCl | 1236.5 | 1346 | NA | 90.37 |
| 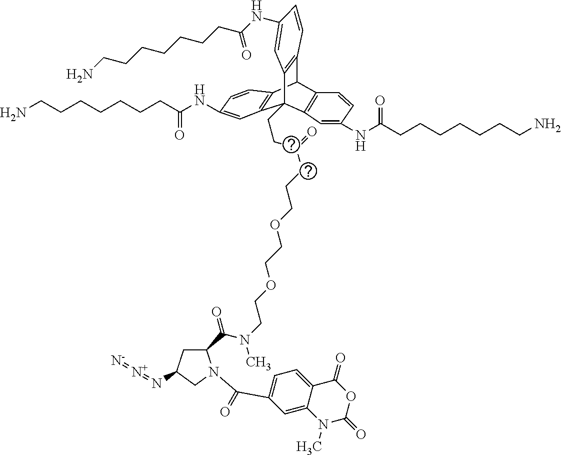 [SMILES: CN(CCOCCOCCCC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)c1ccc2c(=O)oc(=O)n(C)c2c1] | ARK000035 | ARK-78 | 3HCl | 1294.61 | 1404.11 | NA | 86.64 |
| 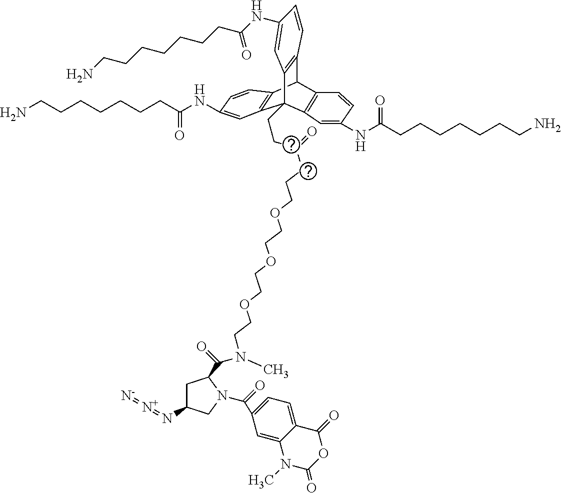 [SMILES: CN(CCOCCOCCOCCCC(=O)CCC12c3cc(NC(=O)CCCCCCCN)ccc3C(c3ccc(NC(=O)CCCCCCCN)cc31)c1ccc(NC(=O)CCCCCCCN)cc12)C(=O)[C@@H]1C[C@H](N=[N+]=[N-])CN1C(=O)c1ccc2c(=O)oc(=O)n(C)c2c1] | ARK000036 | ARK-79 | 3HCl | 1338.66 | 1448.16 | NA | 74.36 |
| 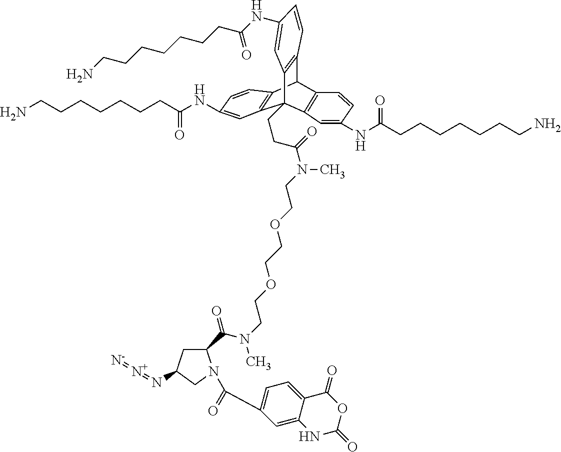 [SMILES: CN(CCOCCOCCN(C)C(=O)[C@@H]1C[C@H](N=[N+]=[N-])CN1C(=O)c1ccc2c(=O)oc(=O)[nH]c2c1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000037 | ARK-78A | 3HCl | 1280.58 | 1390.08 | NA | 95.13 |
| 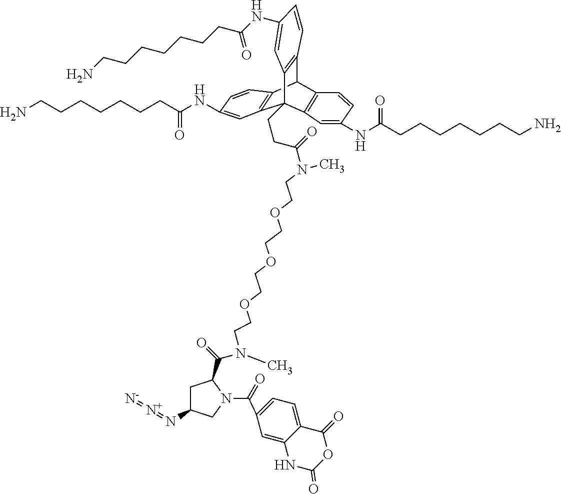 [SMILES: CN(CCOCCOCCOCCN(C)C(=O)[C@@H]1C[C@H](N=[N+]=[N-])CN1C(=O)c1ccc2c(=O)oc(=O)[nH]c2c1)C(=O)CCC12c3cc(NC(=O)CCCCCCCN)ccc3C(c3ccc(NC(=O)CCCCCCCN)cc31)c1ccc(NC(=O)CCCCCCCN)cc12] | ARK000038 | ARK-79A | 3HCl | 1324.64 | 1434.14 | NA | 95.87 |
| 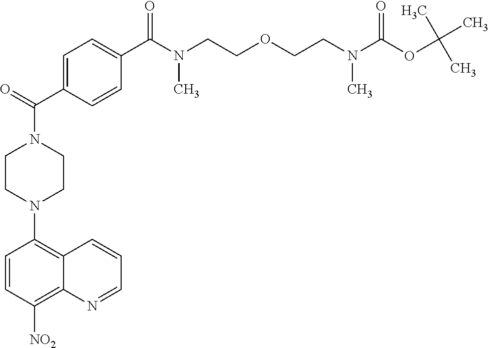 [SMILES: CN(CCOCCN(C)C(=O)c1ccc(C(=O)N2CCN(c3ccc([N+](=O)[O-])c4ncccc34)CC2)cc1)C(=O)OC(C)(C)C] | | ARK-131 | | 620.3 | | 97.56% | 95.78% |
| 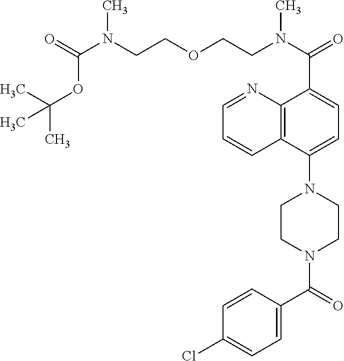 [SMILES: CN(CCOCCN(C)C(=O)c1ccc(N2CCN(C(=O)c3ccc(Cl)cc3)CC2)c2cccnc12)C(=O)OC(C)(C)C] | | ARK-132 | | | | | |
| 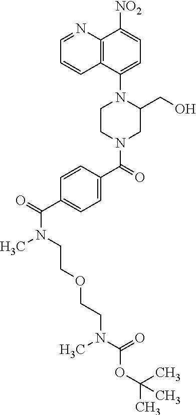 [SMILES: CN(CCOCCN(C)C(=O)c1ccc(C(=O)N2CCN(c3ccc([N+](=O)[O-])c4ncccc34)C(CO)C2)cc1)C(=O)OC(C)(C)C] | | ARK-133 | | | | | |
| 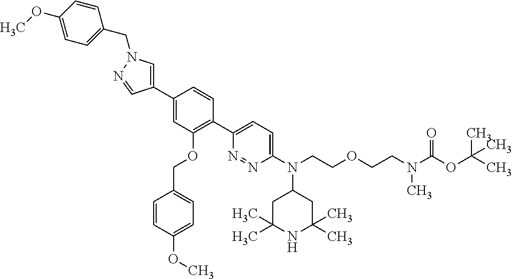 [SMILES: COc1ccc(COc2cc(-c3cnn(Cc4ccc(OC)cc4)c3)ccc2-c2ccc(N(CCOCCN(C)C(=O)OC(C)(C)C)C3CC(C)(C)NC(C)(C)C3)nn2)cc1] | | ARK-134 | | | | | |
| 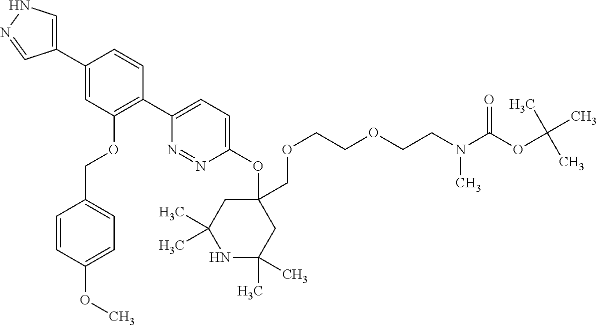 [SMILES: COc1ccc(COc2cc(-c3cn[nH]c3)ccc2-c2ccc(OC3(COCCOCCN(C)C(=O)OC(C)(C)C)CC(C)(C)NC(C)(C)C3)nn2)cc1] | | ARK-135 | | | | | |
| 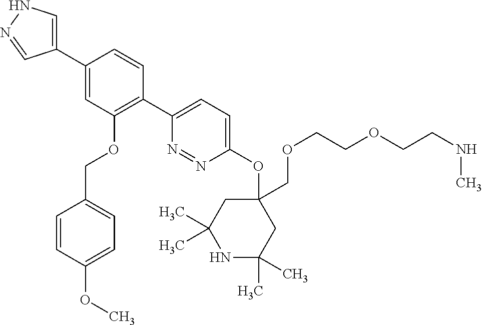 [SMILES: CNCCOCCOCC1(Oc2ccc(-c3ccc(-c4cn[nH]c4)cc3OCc3ccc(OC)cc3)nn2)CC(C)(C)NC(C)(C)C1] | | ARK-136 | | | | | |
| 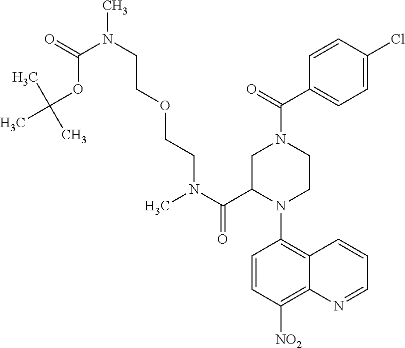 [SMILES: CN(CCOCCN(C)C(=O)C1CN(C(=O)c2ccc(Cl)cc2)CCN1c1ccc([N+](=O)[O-])c2ncccc12)C(=O)OC(C)(C)C] | | ARK-137 | | 654.26 | | 97.73% | 96.05% |
| 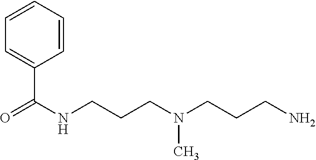 [SMILES: CN(CCCN)CCCNC(=O)c1ccccc1] | ARK000039 | ARK-138 | 2HCl | 249.35 | 322.27 | 100% | 100% |
| 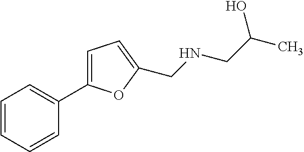 [SMILES: CC(O)CNCc1ccc(-c2ccccc2)o1] | | ARK-139 | | | | | |
| 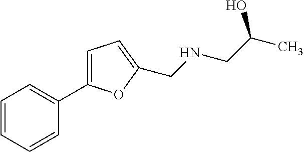 [SMILES: C[C@H](O)CNCc1ccc(-c2ccccc2)o1] | | ARK-140 | | | | | |
| 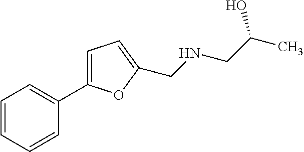 [SMILES: C[C@@H](O)CNCc1ccc(-c2ccccc2)o1] | | ARK-141 | | | | | |
| 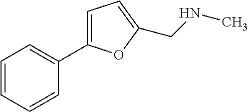 | | ARK-142 | | | | | |
| 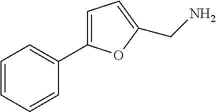 [SMILES: NCc1ccc(-c2ccccc2)o1] | | ARK-143 | | | | | |
| 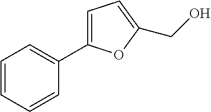 [SMILES: OCc1ccc(-c2ccccc2)o1] | | ARK-144 | | | | | |
| 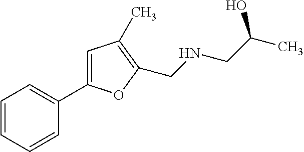 [SMILES: Cc1cc(-c2ccccc2)oc1CNC[C@H](C)O] | | ARK-145 | | | | | |
| 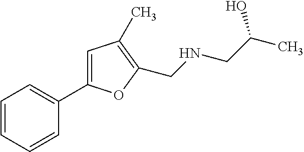 [SMILES: Cc1cc(-c2ccccc2)oc1CNC[C@@H](C)O] | | ARK-146 | | | | | |
| 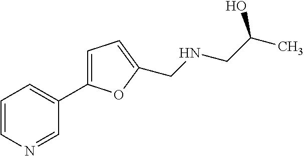 [SMILES: C[C@H](O)CNCc1ccc(-c2cccnc2)o1] | | ARK-147 | | | | | |
| 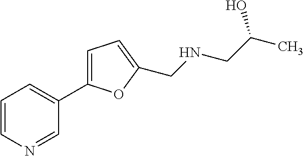 [SMILES: C[C@@H](O)CNCc1ccc(-c2cccnc2)o1] | | ARK-148 | | | | | |
| 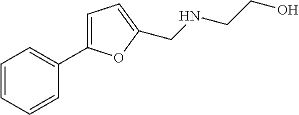 [SMILES: OCCNCc1ccc(-c2ccccc2)o1] | | ARK-149 | | | | | |
| 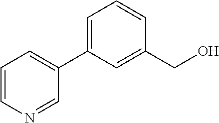 [SMILES: OCc1cccc(-c2cccnc2)c1] | | ARK-150 | | | | | |
| 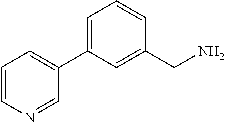 [SMILES: NCc1cccc(-c2cccnc2)c1] | | ARK-151 | | | | | |
| 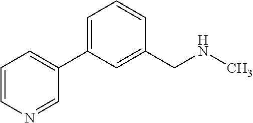 | | ARK-152 | | | | | |
| 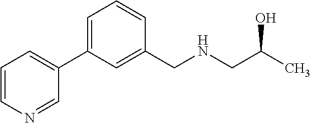 [SMILES: C[C@H](O)CNCc1cccc(-c2cccnc2)c1] | | ARK-153 | | | | | |
| 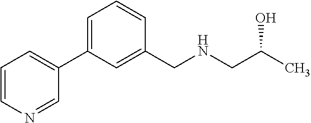 [SMILES: C[C@@H](O)CNCc1cccc(-c2cccnc2)c1] | | ARK-154 | | | | | |
| 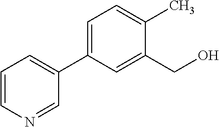 | | ARK-155 | | | | | |
| 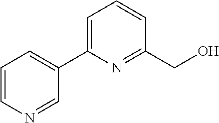 [SMILES: OCc1cccc(-c2cccnc2)n1] | | ARK-156 | | | | | |
| 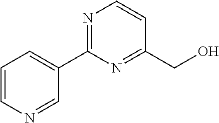 [SMILES: OCc1ccnc(-c2cccnc2)n1] | | ARK-157 | | | | | |
| 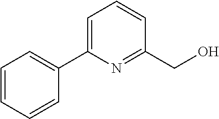 [SMILES: OCc1cccc(-c2ccccc2)n1] | | ARK-158 | | | | | |
| 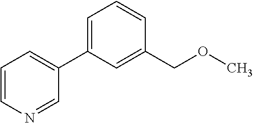 | | ARK-159 | | | | | |
| 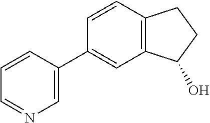 [SMILES: O[C@H]1CCc2ccc(-c3cccnc3)cc21] | | ARK-160 | | | | | |
| 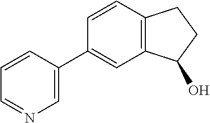 [SMILES: O[C@@H]1CCc2ccc(-c3cccnc3)cc21] | | ARK-161 | | | | | |
| 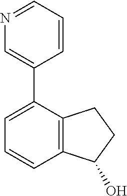 [SMILES: O[C@H]1CCc2c(-c3cccnc3)cccc21] | | ARK-162 | | | | | |
| 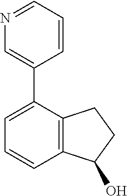 [SMILES: O[C@@H]1CCc2c(-c3cccnc3)cccc21] | | ARK-163 | | | | | |
| 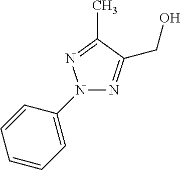 | | ARK-164 | | | | | |
| 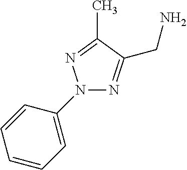 | | ARK-165 | | | | | |
| 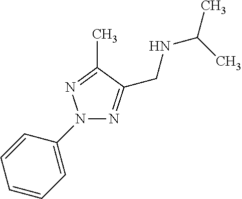 [SMILES: Cc1nn(-c2ccccc2)nc1CNC(C)C] | | ARK-166 | | | | | |
| 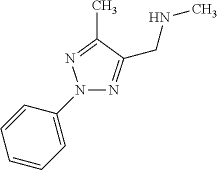 | | ARK-167 | | | | | |
| 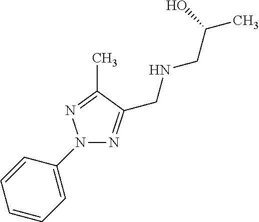 [SMILES: Cc1nn(-c2ccccc2)nc1CNC[C@@H](C)O] | | ARK-168 | | | | | |
| 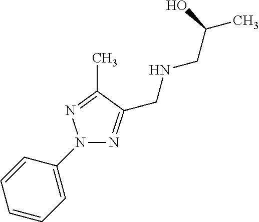 [SMILES: Cc1nn(-c2ccccc2)nc1CNC[C@H](C)O] | | ARK-169 | | | | | |
| 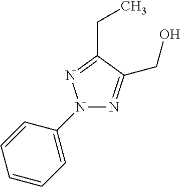 | | ARK-170 | | | | | |
| 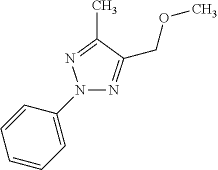 | | ARK-171 | | | | | |
| 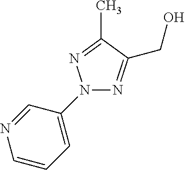 | | ARK-172 | | | | | |
| 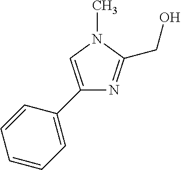 | | ARK-173 | | | | | |
| 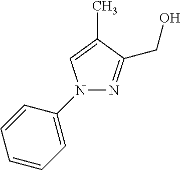 | | ARK-174 | | | | | |
| 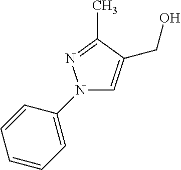 | | ARK-175 | | | | | |
| 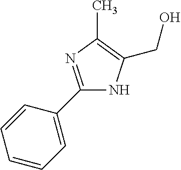 | | ARK-176 | | | | | |
| 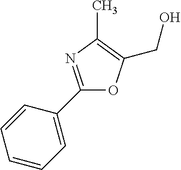 | | ARK-177 | | | | | |
| 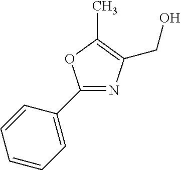 | | ARK-178 | | | | | |
| 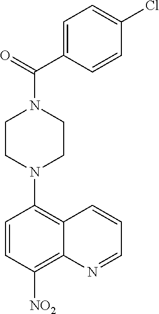 [SMILES: O=C(c1ccc(Cl)cc1)N1CCN(c2ccc([N+](=O)[O-])c3ncccc23)CC1] | | ARK-179 | 1HCl | 396.1 | 432.6 | 100% | 95.09% |
| 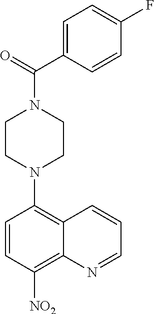 [SMILES: O=C(c1ccc(F)cc1)N1CCN(c2ccc([N+](=O)[O-])c3ncccc23)CC1] | | ARK-180 | | 380.13 | | 96.24% | 98.46% |
| 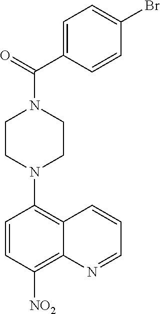 [SMILES: O=C(c1ccc(Br)cc1)N1CCN(c2ccc([N+](=O)[O-])c3ncccc23)CC1] | | ARK-181 | | 440.05 | | 97.43% | 96.33% |
| 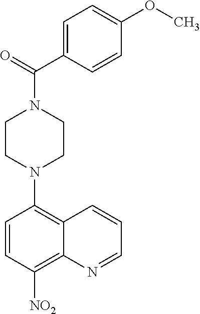 [SMILES: COc1ccc(C(=O)N2CCN(c3ccc([N+](=O)[O-])c4ncccc34)CC2)cc1] | | ARK-182 | 1HCl | 392.15 | 428.65 | 96.87% | 99.00% |
| 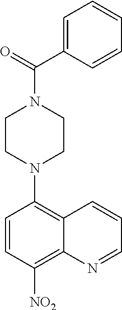 [SMILES: O=C(c1ccccc1)N1CCN(c2ccc([N+](=O)[O-])c3ncccc23)CC1] | | ARK-183 | | 362.14 | | 100% | 100% |
| 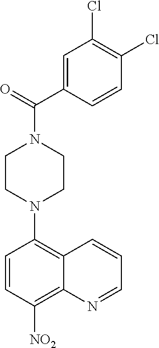 [SMILES: O=C(c1ccc(Cl)c(Cl)c1)N1CCN(c2ccc([N+](=O)[O-])c3ncccc23)CC1] | | ARK-184 | | 430.06 | | 96.36% | 98.88% |
| 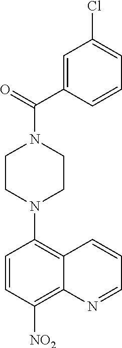 [SMILES: O=C(c1cccc(Cl)c1)N1CCN(c2ccc([N+](=O)[O-])c3ncccc23)CC1] | | ARK-185 | | 396.09 | | 96.29% | 100% |
| 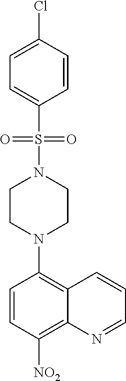 [SMILES: O=[N+]([O-])c1ccc(N2CCN(S(=O)(=O)c3ccc(Cl)cc3)CC2)c2cccnc12] | | ARK-186 | | 432.06 | | 96.44% | 100% |
| 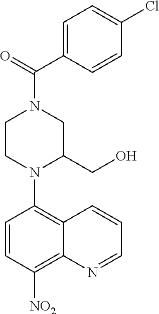 [SMILES: O=C(c1ccc(Cl)cc1)N1CCN(c2ccc([N+](=O)[O-])c3ncccc23)C(CO)C1] | | ARK-187 | | 426.11 | | 97.60% | 99.40% |
| 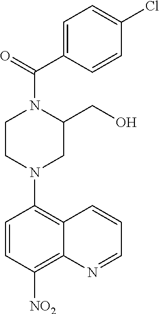 [SMILES: O=C(c1ccc(Cl)cc1)N1CCN(c2ccc([N+](=O)[O-])c3ncccc23)CC1CO] | | ARK-188 | | | | | |
| 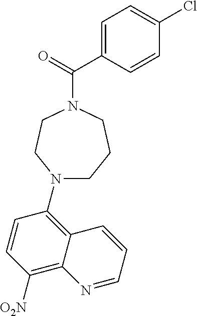 [SMILES: O=C(c1ccc(Cl)cc1)N1CCCN(c2ccc([N+](=O)[O-])c3ncccc23)CC1] | | ARK-189 | | 410.11 | | 98.36% | 100% |
| 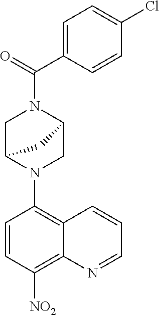 [SMILES: O=C(c1ccc(Cl)cc1)N1C[C@H]2C[C@@H]1CN2c1ccc([N+](=O)[O-])c2ncccc12] | | ARK-190 | | 408.1 | | 100% | 97.92% |
| 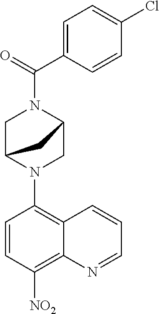 [SMILES: O=C(c1ccc(Cl)cc1)N1C[C@@H]2C[C@H]1CN2c1ccc([N+](=O)[O-])c2ncccc12] | | ARK-191 | | 408.1 | | 96.60% | 98.54% |
| 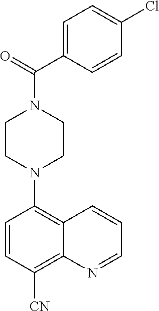 [SMILES: N#Cc1ccc(N2CCN(C(=O)c3ccc(Cl)cc3)CC2)c2cccnc12] | | ARK-192 | | | | | |
| 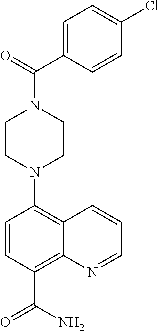 [SMILES: NC(=O)c1ccc(N2CCN(C(=O)c3ccc(Cl)cc3)CC2)c2cccnc12] | | ARK-193 | | | | | |
| 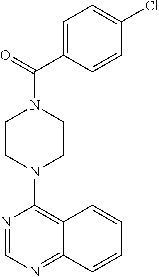 [SMILES: O=C(c1ccc(Cl)cc1)N1CCN(c2ncnc3ccccc23)CC1] | | ARK-194 | | 352.11 | | 99.65% | 99.38% |
| 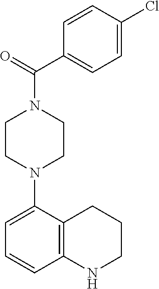 [SMILES: O=C(c1ccc(Cl)cc1)N1CCN(c2cccc3c2CCCN3)CC1] | | ARK-195 | | | | | |
| 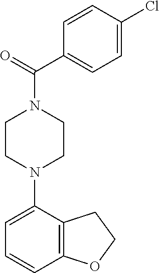 [SMILES: O=C(c1ccc(Cl)cc1)N1CCN(c2cccc3c2CCO3)CC1] | | ARK-196 | | 342.11 | | 96.62% | 95.57% |
| 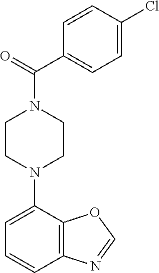 [SMILES: O=C(c1ccc(Cl)cc1)N1CCN(c2cccc3ncoc23)CC1] | | ARK-197 | | | | | |
| 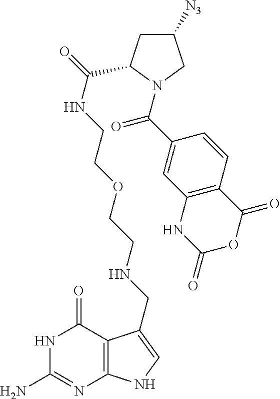 [SMILES: [N-]=[N+]=N[C@H]1C[C@@H](C(=O)NCCOCCNCc2c[nH]c3nc(N)[nH]c(=O)c23)N(C(=O)c2ccc3c(=O)oc(=O)[nH]c3c2)C1] | | ARK-198 | | | | | |
| 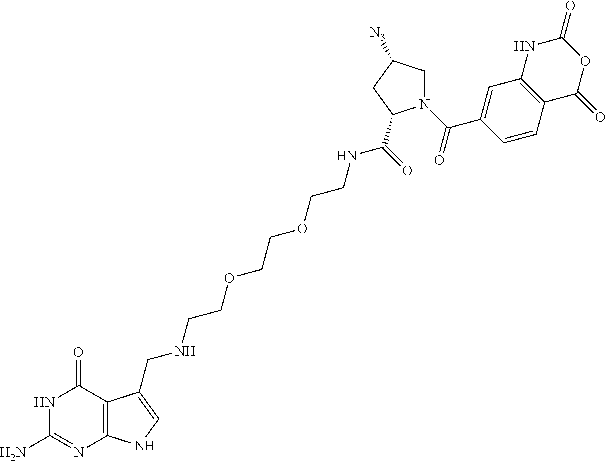 [SMILES: [N-]=[N+]=N[C@H]1C[C@@H](C(=O)NCCOCCOCCNCc2c[nH]c3nc(N)[nH]c(=O)c23)N(C(=O)c2ccc3c(=O)oc(=O)[nH]c3c2)C1] | | ARK-199 | | | | | |
| 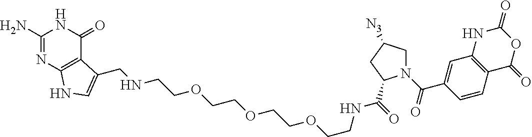 [SMILES: [N-]=[N+]=N[C@H]1C[C@@H](C(=O)NCCOCCOCCOCCNCc2c[nH]c3nc(N)[nH]c(=O)c23)N(C(=O)c2ccc3c(=O)oc(=O)[nH]c3c2)C1] | | ARK-200 | | | | | |
| 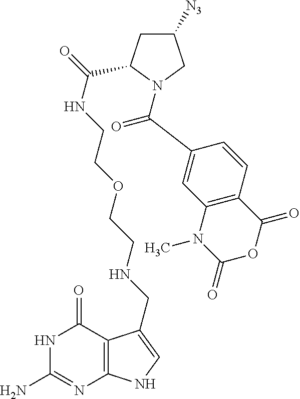 [SMILES: Cn1c(=O)oc(=O)c2ccc(C(=O)N3C[C@@H](N=[N+]=[N-])C[C@H]3C(=O)NCCOCCNCc3c[nH]c4nc(N)[nH]c(=O)c34)cc21] | | ARK-201 | | | | | |
| 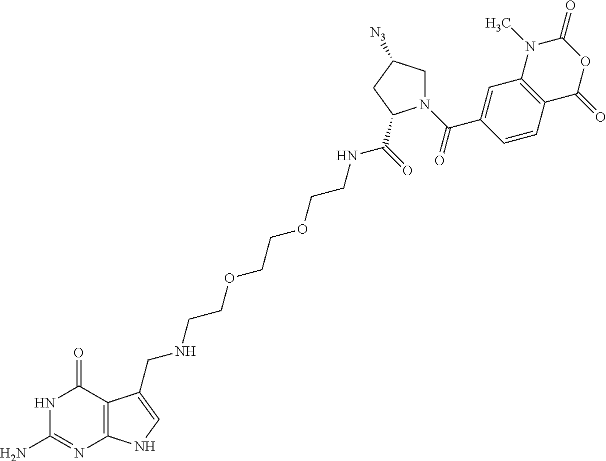 [SMILES: Cn1c(=O)oc(=O)c2ccc(C(=O)N3C[C@@H](N=[N+]=[N-])C[C@H]3C(=O)NCCOCCOCCNCc3c[nH]c4nc(N)[nH]c(=O)c34)cc21] | | ARK-202 | | | | | |
| 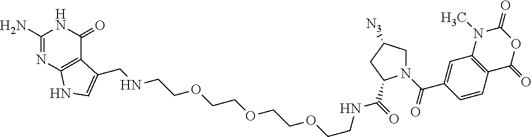 [SMILES: Cn1c(=O)oc(=O)c2ccc(C(=O)N3C[C@@H](N=[N+]=[N-])C[C@H]3C(=O)NCCOCCOCCOCCNCc3c[nH]c4nc(N)[nH]c(=O)c34)cc21] | | ARK-203 | | | | | |
| 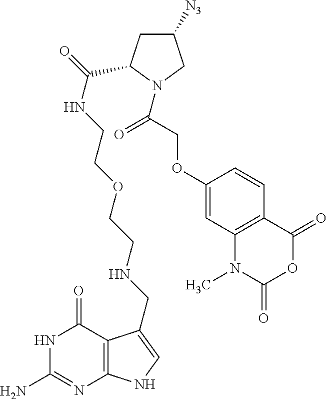 [SMILES: Cn1c(=O)oc(=O)c2ccc(OCC(=O)N3C[C@@H](N=[N+]=[N-])C[C@H]3C(=O)NCCOCCNCc3c[nH]c4nc(N)[nH]c(=O)c34)cc21] | | ARK-204 | | | | | |
| 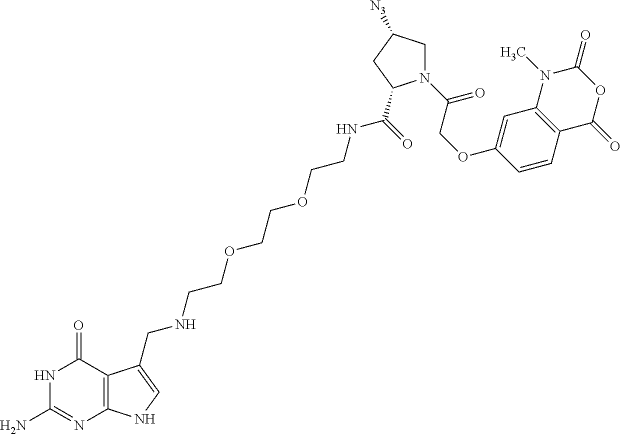 [SMILES: Cn1c(=O)oc(=O)c2ccc(OCC(=O)N3C[C@@H](N=[N+]=[N-])C[C@H]3C(=O)NCCOCCOCCNCc3c[nH]c4nc(N)[nH]c(=O)c34)cc21] | | ARK-205 | | | | | |
| 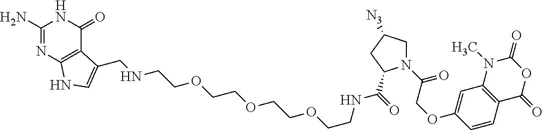 [SMILES: Cn1c(=O)oc(=O)c2ccc(OCC(=O)N3C[C@@H](N=[N+]=[N-])C[C@H]3C(=O)NCCOCCOCCOCCNCc3c[nH]c4nc(N)[nH]c(=O)c34)cc21] | | ARK-206 | | | | | |
| 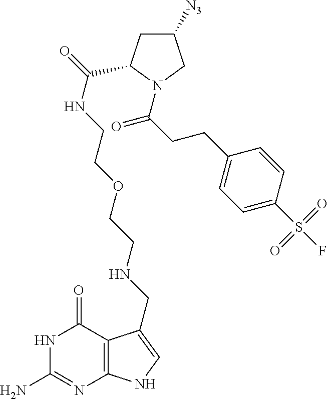 [SMILES: [N-]=[N+]=N[C@H]1C[C@@H](C(=O)NCCOCCNCc2c[nH]c3nc(N)[nH]c(=O)c23)N(C(=O)CCc2ccc(S(=O)(=O)F)cc2)C1] | | ARK-207 | | | | | |
| 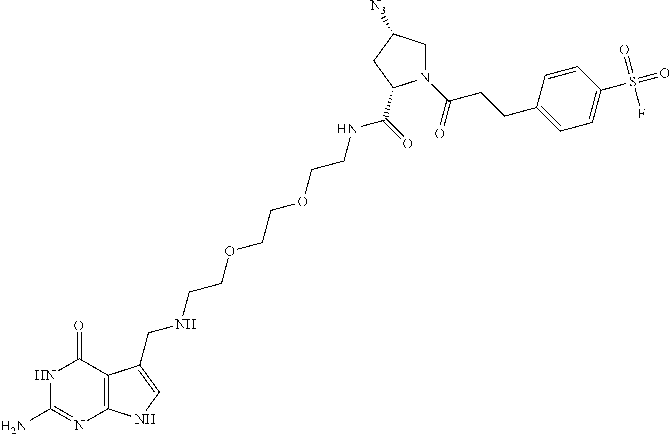 [SMILES: [N-]=[N+]=N[C@H]1C[C@@H](C(=O)NCCOCCOCCNCc2c[nH]c3nc(N)[nH]c(=O)c23)N(C(=O)CCc2ccc(S(=O)(=O)F)cc2)C1] | | ARK-208 | | | | | |
| 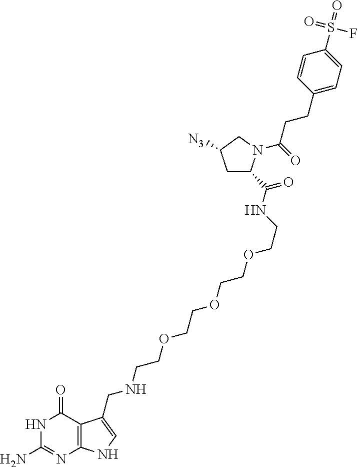 [SMILES: [N-]=[N+]=N[C@H]1C[C@@H](C(=O)NCCOCCOCCOCCNCc2c[nH]c3nc(N)[nH]c(=O)c23)N(C(=O)CCc2ccc(S(=O)(=O)F)cc2)C1] | | ARK-209 | | | | | |
| 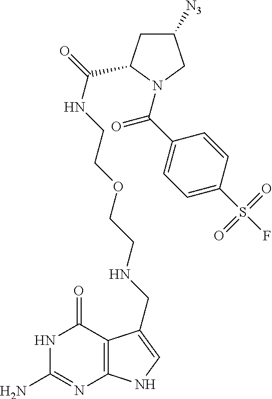 [SMILES: [N-]=[N+]=N[C@H]1C[C@@H](C(=O)NCCOCCNCc2c[nH]c3nc(N)[nH]c(=O)c23)N(C(=O)c2ccc(S(=O)(=O)F)cc2)C1] | | ARK-210 | | | | | |
| 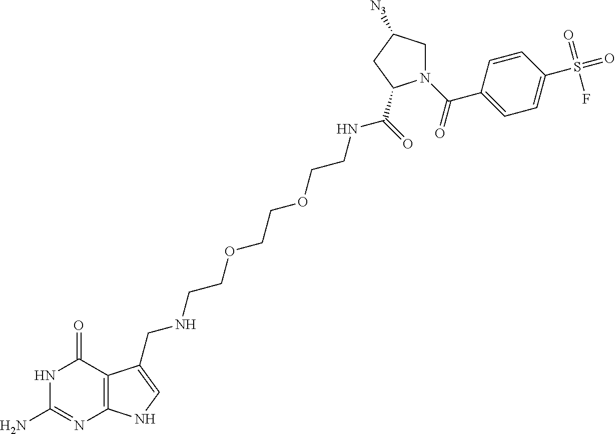 [SMILES: [N-]=[N+]=N[C@H]1C[C@@H](C(=O)NCCOCCOCCNCc2c[nH]c3nc(N)[nH]c(=O)c23)N(C(=O)c2ccc(S(=O)(=O)F)cc2)C1] | | ARK-211 | | | | | |
| 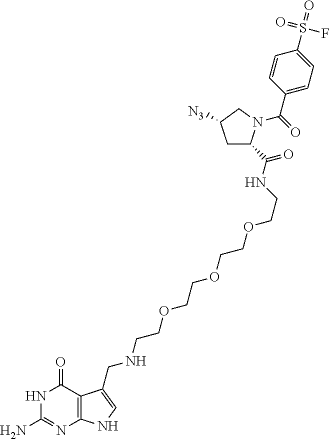 [SMILES: [N-]=[N+]=N[C@H]1C[C@@H](C(=O)NCCOCCOCCOCCNCc2c[nH]c3nc(N)[nH]c(=O)c23)N(C(=O)c2ccc(S(=O)(=O)F)cc2)C1] | | ARK-212 | | | | | |
| 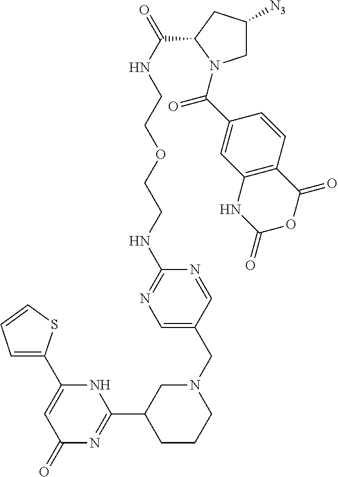 [SMILES: [N-]=[N+]=N[C@H]1C[C@@H](C(=O)NCCOCCNc2ncc(CN3CCCC(c4nc(=O)cc(-c5cccs5)[nH]4)C3)cn2)N(C(=O)c2ccc3c(=O)oc(=O)[nH]c3c2)C1] | | ARK-213 | | | | | |
| 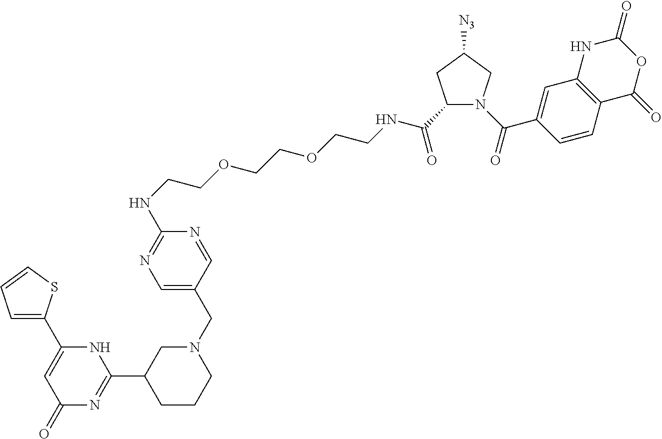 [SMILES: [N-]=[N+]=N[C@H]1C[C@@H](C(=O)NCCOCCOCCNc2ncc(CN3CCCC(c4nc(=O)cc(-c5cccs5)[nH]4)C3)cn2)N(C(=O)c2ccc3c(=O)oc(=O)[nH]c3c2)C1] | | ARK-214 | | | | | |
| 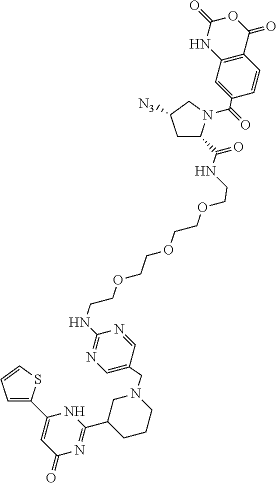 [SMILES: [N-]=[N+]=N[C@H]1C[C@@H](C(=O)NCCOCCOCCOCCNc2ncc(CN3CCCC(c4nc(=O)cc(-c5cccs5)[nH]4)C3)cn2)N(C(=O)c2ccc3c(=O)oc(=O)[nH]c3c2)C1] | | ARK-215 | | | | | |
| 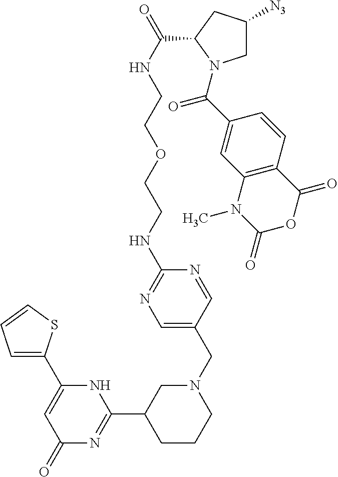 [SMILES: Cn1c(=O)oc(=O)c2ccc(C(=O)N3C[C@@H](N=[N+]=[N-])C[C@H]3C(=O)NCCOCCNc3ncc(CN4CCCC(c5nc(=O)cc(-c6cccs6)[nH]5)C4)cn3)cc21] | | ARK-216 | | | | | |
| 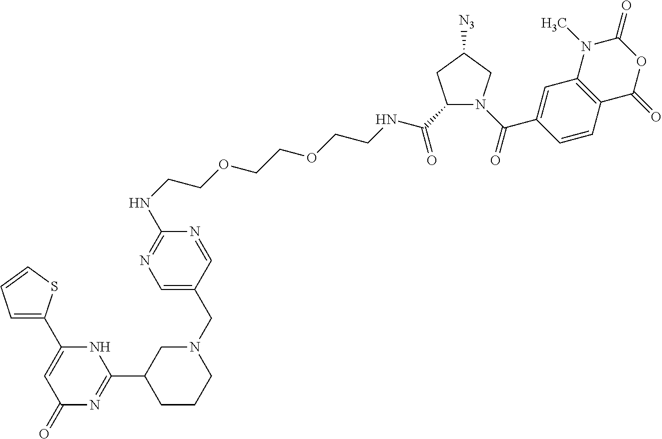 [SMILES: Cn1c(=O)oc(=O)c2ccc(C(=O)N3C[C@@H](N=[N+]=[N-])C[C@H]3C(=O)NCCOCCOCCNc3ncc(CN4CCCC(c5nc(=O)cc(-c6cccs6)[nH]5)C4)cn3)cc21] | | ARK-217 | | | | | |
| 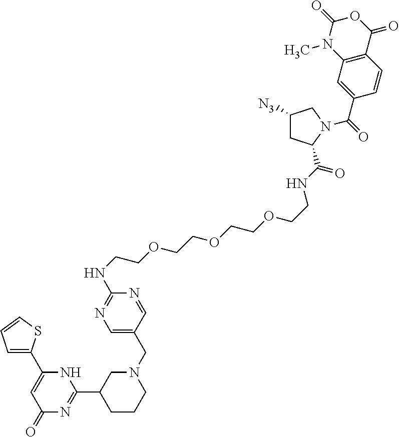 [SMILES: Cn1c(=O)oc(=O)c2ccc(C(=O)N3C[C@@H](N=[N+]=[N-])C[C@H]3C(=O)NCCOCCOCCOCCNc3ncc(CN4CCCC(c5nc(=O)cc(-c6cccs6)[nH]5)C4)cn3)cc21] | | ARK-218 | | | | | |
| 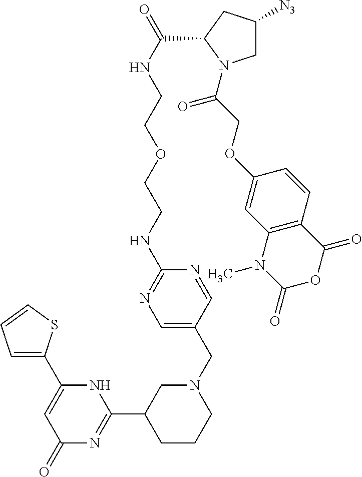 [SMILES: Cn1c(=O)oc(=O)c2ccc(OCC(=O)N3C[C@@H](N=[N+]=[N-])C[C@H]3C(=O)NCCOCCNc3ncc(CN4CCCC(c5nc(=O)cc(-c6cccs6)[nH]5)C4)cn3)cc21] | | ARK-219 | | | | | |
| 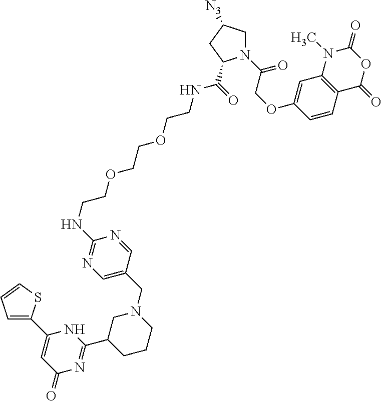 [SMILES: Cn1c(=O)oc(=O)c2ccc(OCC(=O)N3C[C@@H](N=[N+]=[N-])C[C@H]3C(=O)NCCOCCOCCNc3ncc(CN4CCCC(c5nc(=O)cc(-c6cccs6)[nH]5)C4)cn3)cc21] | | ARK-220 | | | | | |
| 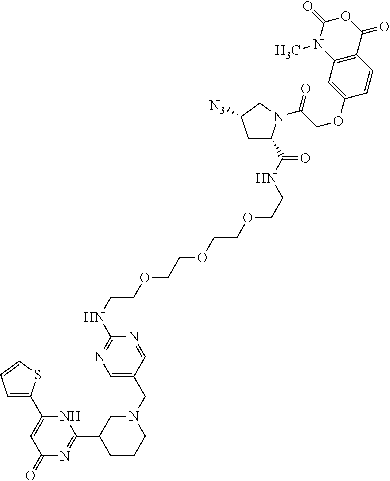 [SMILES: Cn1c(=O)oc(=O)c2ccc(OCC(=O)N3C[C@@H](N=[N+]=[N-])C[C@H]3C(=O)NCCOCCOCCOCCNc3ncc(CN4CCCC(c5nc(=O)cc(-c6cccs6)[nH]5)C4)cn3)cc21] | | ARK-221 | | | | | |
| 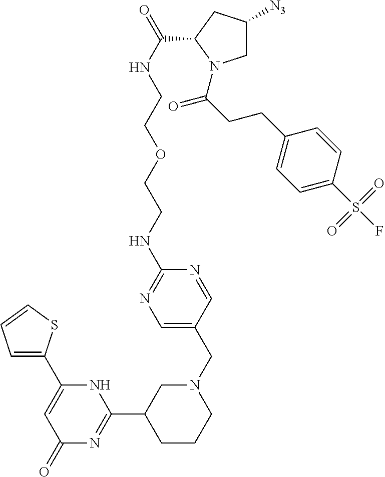 [SMILES: [N-]=[N+]=N[C@H]1C[C@@H](C(=O)NCCOCCNc2ncc(CN3CCCC(c4nc(=O)cc(-c5cccs5)[nH]4)C3)cn2)N(C(=O)CCc2ccc(S(=O)(=O)F)cc2)C1] | | ARK-222 | | | | | |
| 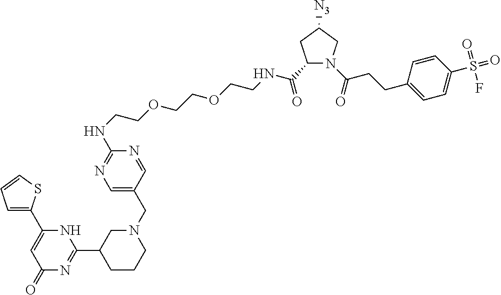 [SMILES: [N-]=[N+]=N[C@H]1C[C@@H](C(=O)NCCOCCOCCNc2ncc(CN3CCCC(c4nc(=O)cc(-c5cccs5)[nH]4)C3)cn2)N(C(=O)CCc2ccc(S(=O)(=O)F)cc2)C1] | | ARK-223 | | | | | |
| 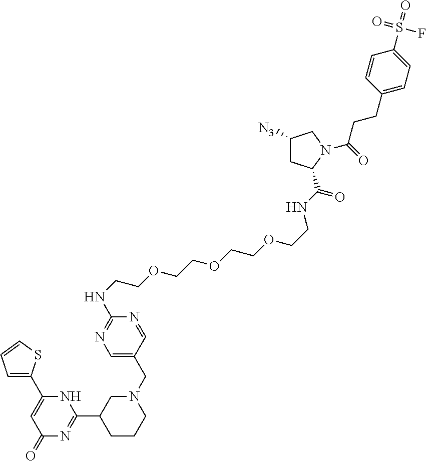 [SMILES: [N-]=[N+]=N[C@H]1C[C@@H](C(=O)NCCOCCOCCOCCNc2ncc(CN3CCCC(c4nc(=O)cc(-c5cccs5)[nH]4)C3)cn2)N(C(=O)CCc2ccc(S(=O)(=O)F)cc2)C1] | | ARK-224 | | | | | |
| 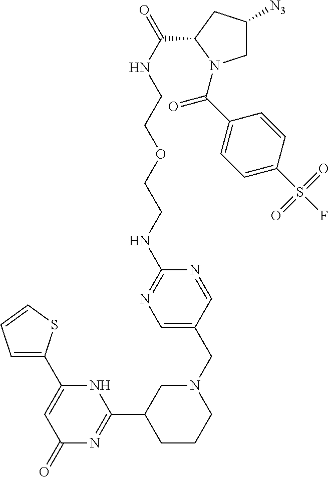 [SMILES: [N-]=[N+]=N[C@H]1C[C@@H](C(=O)NCCOCCNc2ncc(CN3CCCC(c4nc(=O)cc(-c5cccs5)[nH]4)C3)cn2)N(C(=O)c2ccc(S(=O)(=O)F)cc2)C1] | | ARK-225 | | | | | |
| 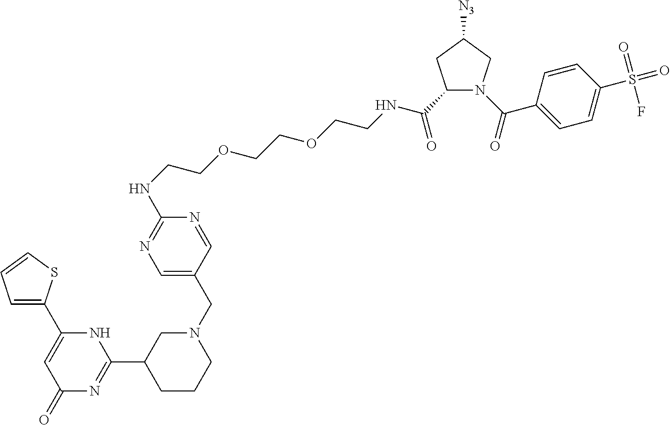 [SMILES: [N-]=[N+]=N[C@H]1C[C@@H](C(=O)NCCOCCOCCNc2ncc(CN3CCCC(c4nc(=O)cc(-c5cccs5)[nH]4)C3)cn2)N(C(=O)c2ccc(S(=O)(=O)F)cc2)C1] | | ARK-226 | | | | | |
| 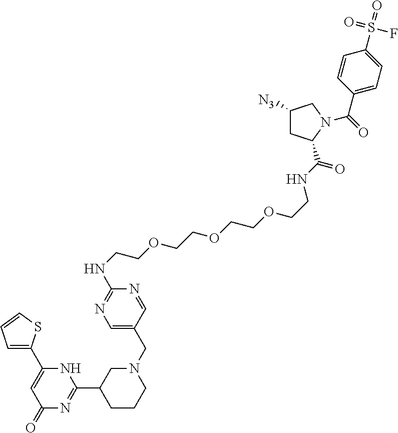 [SMILES: [N-]=[N+]=N[C@H]1C[C@@H](C(=O)NCCOCCOCCOCCNc2ncc(CN3CCCC(c4nc(=O)cc(-c5cccs5)[nH]4)C3)cn2)N(C(=O)c2ccc(S(=O)(=O)F)cc2)C1] | | ARK-227 | | | | | |

### Example 20: RNA Sequences Prepared

The following RNA sequences were designed and prepared for use in testing compound binding (including verifying the expected binding mode or identifying the binding mode, when not known) and validating the methods of the present invention.

TABLE 8
RNA Sequences Prepared

| RNA | | | | | SEQ |
|---|---|---|---|---|---|
| Desig- | Length | Modifi- | Descrip- | Sequence | ID |
| nation | (nt) | cations | tion | (5′ to 3′) | NO: |
| HTT. | 474 | none | Exon 1 | GCUGCCGGGACGGGUC | 25 |
| Exon1. | | | of | CAAGAUGGACGGCCGCU | |
| 41CAG | | | the HTT | CAGGUUCUGCUUUUACC | |
| | | | mRNA | UGCGGCCCAGAGCCCCA | |
| | | | with 41 | UUCAUUGCCCCGGUGCU | |
| | | | CAG | GAGCGGCGCCGCGAGU | |
| | | | repeats | CGGCCCGAGGCCUCCG | |
| | | | (HD | GGGACUGCCGUGCCGG | |
| | | | disease) | GCGGGAGACCGCCAUGG | |
| | | | | CGACCCUGGAAAAGCUG | |
| | | | | AUGAAGGCCUUCGAGUC | |
| | | | | CCUCAAGUCCUUCCAGC | |
| | | | | AGCAGCAGCAGCAGCAG | |
| | | | | CAGCAGCAGCAGCAGCA | |
| | | | | GCAGCAGCAGCAGCAGC | |
| | | | | AGCAGCAGCAGCAGCAG | |
| | | | | CAGCAGCAGCAGCAGCA | |
| | | | | GCAGCAGCAGCAGCAGC | |
| | | | | AGCAGCAGCAGCAGCAG | |
| | | | | CAACAGCCGCCACCGCC | |
| | | | | GCCGCCGCCGCCGCCG | |
| | | | | CCUCCUCAGCUUCCUCA | |
| | | | | GCCGCCGCCGCAGGCAC | |
| | | | | AGCCGCUGCUGCCUCAG | |
| | | | | CCGCAGCCGCCCCCGCC | |
| | | | | GCCGCCCCCGCCGCCAC | |
| | | | | CCGGCCCGGCUGUGGC | |
| | | | | UGAGGAGCCGCUGCACC | |
| | | | | GACC | |
| HTT. | 474 | 5′-Biotin | Exon 1 | GCUGCCGGGACGGGUC | 25 |
| Exon1. | | | of | CAAGAUGGACGGCCGCU | |
| 41CAG_ | | | the HTT | CAGGUUCUGCUUUUACC | |
| 5Bio | | | mRNA | UGCGGCCCAGAGCCCCA | |
| | | | with 41 | UUCAUUGCCCCGGUGCU | |
| | | | CAG | GAGCGGCGCCGCGAGU | |
| | | | repeats | CGGCCCGAGGCCUCCG | |
| | | | (HD | GGGACUGCCGUGCCGG | |
| | | | disease) | GCGGGAGACCGCCAUGG | |
| | | | | CGACCCUGGAAAAGCUG | |
| | | | | AUGAAGGCCUUCGAGUC | |
| | | | | CCUCAAGUCCUUCCAGC | |
| | | | | AGCAGCAGCAGCAGCAG | |
| | | | | CAGCAGCAGCAGCAGCA | |
| | | | | GCAGCAGCAGCAGCAGC | |
| | | | | AGCAGCAGCAGCAGCAG | |
| | | | | CAGCAGCAGCAGCAGCA | |
| | | | | GCAGCAGCAGCAGCAGC | |
| | | | | AGCAGCAGCAGCAGCAG | |
| | | | | CAACAGCCGCCACCGCC | |
| | | | | GCCGCCGCCGCCGCCG | |
| | | | | CCUCCUCAGCUUCCUCA | |
| | | | | GCCGCCGCCGCAGGCAC | |
| | | | | AGCCGCUGCUGCCUCAG | |
| | | | | CCGCAGCCGCCCCCGCC | |
| | | | | GCCGCCCCCGCCGCCAC | |
| | | | | CCGGCCCGGCUGUGGC | |
| | | | | UGAGGAGCCGCUGCACC | |
| | | | | GACC | |
| HTT. | 402 | none | Exon 1 | GCUGCCGGGACGGGUC | 26 |
| Exon1. | | | of | CAAGAUGGACGGCCGCU | |
| 17CAG | | | the HTT | CAGGUUCUGCUUUUACC | |
| | | | mRNA | UGCGGCCCAGAGCCCCA | |
| | | | with 17 | UUCAUUGCCCCGGUGCU | |
| | | | CAG | GAGCGGCGCCGCGAGU | |
| | | | repeats | CGGCCCGAGGCCUCCG | |
| | | | (healthy) | GGGACUGCCGUGCCGG | |
| | | | | GCGGGAGACCGCCAUGG | |
| | | | | CGACCCUGGAAAAGCUG | |
| | | | | AUGAAGGCCUUCGAGUC | |
| | | | | CCUCAAGUCCUUCCAGC | |
| | | | | AGCAGCAGCAGCAGCAG | |
| | | | | CAGCAGCAGCAGCAGCA | |
| | | | | GCAGCAGCAGCAGCAAC | |
| | | | | AGCCGCCACCGCCGCCG | |
| | | | | CCGCCGCCGCCGCCUCC | |
| | | | | UCAGCUUCCUCAGCCGC | |
| | | | | CGCCGCAGGCACAGCCG | |
| | | | | CUGCUGCCUCAGCCGCA | |
| | | | | GCCGCCCCCGCCGCCGC | |
| | | | | CCCCGCCGCCACCCGGC | |
| | | | | CCGGCUGUGGCUGAGG | |
| | | | | AGCCGCUGCACCGACC | |
| HTT. | 402 | 5′-Biotin | Exon 1 | GCUGCCGGGACGGGUC | 26 |
| Exon1. | | | of | CAAGAUGGACGGCCGCU | |
| 17CAG_ | | | the HTT | CAGGUUCUGCUUUUACC | |
| 5Bio | | | mRNA | UGCGGCCCAGAGCCCCA | |
| | | | with 41 | UUCAUUGCCCCGGUGCU | |
| | | | CAG | GAGCGGCGCCGCGAGU | |
| | | | repeats | CGGCCCGAGGCCUCCG | |
| | | | (healthy) | GGGACUGCCGUGCCGG | |
| | | | | GCGGGAGACCGCCAUGG | |
| | | | | CGACCCUGGAAAAGCUG | |
| | | | | AUGAAGGCCUUCGAGUC | |
| | | | | CCUCAAGUCCUUCCAGC | |
| | | | | AGCAGCAGCAGCAGCAG | |
| | | | | CAGCAGCAGCAGCAGCA | |
| | | | | GCAGCAGCAGCAGCAAC | |
| | | | | AGCCGCCACCGCCGCCG | |
| | | | | CCGCCGCCGCCGCCUCC | |
| | | | | UCAGCUUCCUCAGCCGC | |
| | | | | CGCCGCAGGCACAGCCG | |
| | | | | CUGCUGCCUCAGCCGCA | |
| | | | | GCCGCCCCCGCCGCCGC | |
| | | | | CCCCGCCGCCACCCGGC | |
| | | | | CCGGCUGUGGCUGAGG | |
| | | | | AGCCGCUGCACCGACC | |
| HTT41C | 68 | 5′-Biotin | Portion of | GCAGCAGCAGCAGCAGC | 27 |
| AG_ | | | the | AGCAGCAGCAGCAGCAG | |
| 3WJ_ | | | 41 CAG | CAGCAGCAGCAGCAACA | |
| 5Bio | | | HTT RNA | GCCGCCACCGCCGCCGC | |
| | | | having | | |
| | | | the 3- | | |
| | | | way | | |
| | | | junction | | |
| HTT17C | 64 | 5′-Biotin | Portion of | GCAGCAGCAGCAGCAGC | 28 |
| AG_ | | | the | AGCAGCAGCAGCAACAG | |
| internal | | | 17CAG | CCGCCACCGCCGCCGCC | |
| bulge_ | | | HTT RNA | GCCGCCGCCGCCU | |
| 5Bio | | | having | | |
| | | | the | | |
| | | | internal | | |
| | | | bulge | | |
| 22CAG_ | 66 | 5′-Biotin | A hairpin | CAGCAGCAGCAGCAGCA | 29 |
| hairpin_ | | | consisting | GCAGCAGCAGCAGCAGC | |
| 5Bio | | | of a | AGCAGCAGCAGCAGCAG | |
| | | | pure | CAGCAGCAGCAGCAG | |
| | | | stretch of | | |
| | | | 22 CAGs | | |
| HTT41C | 68 | none | Portion of | GCAGCAGCAGCAGCAGC | 27 |
| AG_3WJ | | | the | AGCAGCAGCAGCAGCAG | |
| | | | 41 CAG | CAGCAGCAGCAGCAACA | |
| | | | HTT RNA | GCCGCCACCGCCGCCGC | |
| | | | having | | |
| | | | the 3- | | |
| | | | way | | |
| | | | junction | | |
| HTT17C | 64 | none | Portion of | GCAGCAGCAGCAGCAGC | 28 |
| AG_inter | | | the | AGCAGCAGCAGCAACAG | |
| nalbulge | | | 17CAG | CCGCCACCGCCGCCGCC | |
| | | | HTT RNA | GCCGCCGCCGCCU | |
| | | | having | | |
| | | | the | | |
| | | | internal | | |
| | | | bulge | | |
| 22CAG_ | 66 | none | A hairpin | CAGCAGCAGCAGCAGCA | 29 |
| hairpin | | | consisting | GCAGCAGCAGCAGCAGC | |
| | | | of a | AGCAGCAGCAGCAGCAG | |
| | | | pure | CAGCAGCAGCAGCAG | |
| | | | stretch of | | |
| | | | 22 CAGs | | |
| Tetracycline | 57 | none | Tetracycline | GAGCCUAAAACAUACCA | 30 |
| Aptamer | | | binding | GAGAAAUCUGGAGAGGU | |
| | | | RNA | GAAGAAUACGACCACCU | |
| | | | | AGGCUC | |
| RNA3WJ_ | 38 | none | 0.0.0 | GGCACAAAUGCAACACU | 31 |
| 0.0.0 | | | Triptycene | GCAUUACCAUGCGGUUG | |
| | | | 3-Way | UGCC | |
| | | | Junction | | |
| RNA3WJ_ | 38 | 5′ Iowa | 0.0.0 | GGCACAAAUGCAACACU | 31 |
| 0.0.0_51 | | Black; 3′ | Triptycene | GCAUUACCAUGCGGUUG | |
| B_3FAM | | 6FAM | 3-Way | UGCC | |
| | | | Junction | | |
| | | | with | | |
| | | | fluorophore | | |
| | | | & | | |
| | | | quencher | | |
| RNA3WJ_ | 38 | 3′ 6FAM | 0.0.0 | GGCACAAAUGCAACACU | 31 |
| 0.0.0_3 | | | Triptycene | GCAUUACCAUGCGGUUG | |
| FAM | | | 3-Way | UGCC | |
| | | | Junction | | |
| | | | with | | |
| | | | fluorophore | | |
| | | | but no | | |
| | | | quencher | | |
| RNA3WJ | 39 | 5′ Iowa | 1.0.0 | GGCACACAAUGCAACAC | 32 |
| 1.0.0_51 | | Black; 3′ | Triptycene | UGCAUUACCAUGCGGUU | |
| B_3FAM | | 6FAM | 3-Way | GUGCC | |
| | | | Junction | | |
| | | | with | | |
| | | | fluorophore | | |
| | | | & | | |
| | | | quencher | | |
| RNA3WJ | 40 | 5′ Iowa | 1.1.0 | GGCACACAAUGCAACAC | 33 |
| 1.1.0_51 | | Black; 3′ | Triptycene | UGCAUUGACCAUGCGGU | |
| B_3FAM | | 6FAM | 3-Way | UGUGCC | |
| | | | Junction | | |
| | | | with | | |
| | | | fluorophore | | |
| | | | & | | |
| | | | quencher | | |
| RNA3WJ | 40 | 5′ Iowa | 2.0.0 | GGCACACGAAUGCAACA | 34 |
| 2.0.0_51 | | Black; 3′ | Triptycene | CUGCAUUACCAUGCGGU | |
| B_3FAM | | 6FAM | 3-Way | UGUGCC | |
| | | | Junction | | |
| | | | with | | |
| | | | fluorophore | | |
| | | | & | | |
| | | | quencher | | |
| RNA3WJ_ | 41 | 5′ Iowa | 1.1.1 | GGCACACAAUGCAACAC | 35 |
| 1.1.1 51 | | Black; 3′ | Triptycene | UGCAUUGACCAUGCGGU | |
| B_3FAM | | 6FAM | 3-Way | AUGUGCC | |
| | | | Junction | | |
| | | | with | | |
| | | | fluorophore | | |
| | | | & | | |
| | | | quencher | | |
| RNA3WJ_ | 41 | 5′ Iowa | 2.1.0 | GGCACACGAAUGCAACA | 36 |
| 2.1.0_51 | | Black; 3′ | Triptycene | CUGCAUUGACCAUGCGG | |
| B_3FAM | | 6FAM | 3-Way | UUGUGCC | |
| | | | Junction | | |
| | | | with | | |
| | | | fluorophore | | |
| | | | & | | |
| | | | quencher | | |
| RNA3WJ_ | 41 | 5′ Iowa | 3.0.0 | GGCACACAGAAUGCAAC | 37 |
| 3.0.0_51 | | Black; 3′ | Triptycene | ACUGCAUUACCAUGCGG | |
| B_3FAM | | 6FAM | 3-Way | UUGUGCC | |
| | | | Junction | | |
| | | | with | | |
| | | | fluorophore | | |
| | | | & | | |
| | | | quencher | | |
| Split3WJ. | 14 | 5′ Iowa | 0.0.0 | GGCACAAAUGCAAC | 38 |
| 1_up_5IB | | Black | Triptycene | | |
| | | | 3-Way | | |
| | | | Junction | | |
| | | | split at | | |
| | | | first loop; | | |
| | | | 5′ end | | |
| Split3WJ. | 24 | 3′ 6FAM | 0.0.0 | ACUGCAUUACCAUGCGG | 39 |
| 1_down_ | | | Triptycene | UUGUGCC | |
| 3FAM | | | 3-Way | | |
| | | | Junction | | |
| | | | split at | | |
| | | | first loop; | | |
| | | | 3′ end | | |
| Split3WJ. | 27 | 5′ Iowa | 0.0.0 | GGCACAAAUGCAACACU | 40 |
| 2_up_5IB | | Black | Triptycene | GCAUUACCAU | |
| | | | 3-Way | | |
| | | | Junction | | |
| | | | split at | | |
| | | | second | | |
| | | | loop; 5′ | | |
| | | | end | | |
| Split3WJ. | 11 | 3′ 6FAM | 0.0.0 | GCGGUUGUGCC | 41 |
| 2_down_ | | | Triptycene | | |
| 3FAM | | | 3-Way | | |
| | | | Junction | | |
| | | | split at | | |
| | | | second | | |
| | | | loop; 3′ | | |
| | | | end | | |

### Example 21: Fluorescence Quenching Binding Assay

This assay will be used to test binding of compounds for RNA three way junction (such as a 38 nt construct). This is a fluorescence quenching assay utilizing FAM as fluorescence tag and Iowa Black as quencher. Tags are attached at the 3′ and 5′ end, respectively. Stable formation of MW upon compound binding would lead to quenching of the FAM fluorescence due to close proximity of the Iowa Black tag. Assay readout: FAM (485-520 nm) Fluorescence Intensity.

Nucleic acid junctions are ubiquitous structural motifs, occurring in both DNA and RNA. They represent important and sometimes transient structures in biological processes, such as replication and recombination, while also occurring in triplet repeat expansions, which are associated with a number of neurodegenerative diseases. Nucleic acid junctions are ubiquitous in viral genomes and are important structural motifs in riboswitches. Three-way junctions are key building blocks present in many nanostructures, soft materials, multichromophore assemblies, and aptamer-based sensors. In the case of aptamer based sensors, DNA three-way junctions serve as an important structural motif.

This assay can serve as a part of the toolkit for discovering RNA-binding small molecules by testing binding to a 3WJ in the context of a controlled system with a readily observable readout. PEARL-seq or other methods disclosed herein may then be used to further screen compounds.

Assay sample buffer used: 10 mM CacoK pH 7.2, 30 mM NaCl. Buffer preparation in Dnase/Rnase Free distilled water (Gibco Life Technologies).

Compound Preparation

Tool compounds provided as dry powder are prepared as 50 mM stock solution in 100% d₆-DMSO. Stock solutions of 50 mM concentration in d₆-DMSO are stored at RT.

Hardware

Sample plate: Greiner cat #784076, black, 384 (Dilution plate: Greiner REF 781101, PS-Microplate, 384 well, clear). Fluorescence Intensity device: Envision 1040285

Assay Protocol

### Assay Buffer Preparation

Daily fresh (10 ml): 1 ml 100 mM CacoK pH 7.2 and 0.3 ml 1 M NaCl filled up to 10 ml with Dnase/Rnase Free distilled water

### RNA Preparation (RNA Sample Homogenization)

Dilute the RNA 1:10 (final 10 μM) in Assay Buffer.

Heat up the diluted RNA up to 90° C. for 5 min (sealed Eppendorf Tube).

Cool down the RNA probe slowly to RT.

### Compound Preparation

Dilute the compounds to 800 μM in DMSO (Assay: 8 μM).

### Sample Preparation

71.2-78.4 μL Assay Buffer are pipetted into Greiner REF 781101, PS-Microplate, 384 (each well needed).

Add 0.8-8 μL of the RNA-Solution (100 mM).

Add 0.8 μL Compound-Solution (800 mM).

Mix gently with Multi-Channel Pipette.

Final concentrations in the sample: 1-10 μM RNA, 8 μM compound, 1% DMSO

Thermal Shift measurement (LightCycler480)

Pipet 25 μL Sample Solution into Greiner cat #784076, black, 384

When sample transfer is finished put lid on top.

Measure the 96 plate with the LightCycler480 (Channel: 485/520 nm).

Readout

Software used was PerkinElmer Envision Manager.

Basic assay information

| | |
|---|---|
| Assay ID: | 12697 |
| Protocol ID: | 100279 |
| Protocol Name: | Copy 2 of |
| | FI_picogreen_filter_LV opt |
| Picogreen_filter_LV_opt | 4000045 |
| Top mirror | FITC |
| Exc. filter | FITC 485 |
| Using of excitation filter | Top |
| Ems. filter | TRF Emission 520 |
| Filters: | |
| FITC 485 | 102 |
| Filter type | Excitation |
| Description | X485 CWL = 485 nm BW = 14 nm |
| | Tmin = 60% |
| Used with | DELFIA - Time-resolved |
| | Fluorescence |
| TRF Emission 520 | 275 |
| Filter type | Emission |
| Description | M520 CWL = 520 nm BW = 25 nm |
| | Tmin = 80% |
| Used with | DELFIA - Time-resolved |
| | Fluorescence |

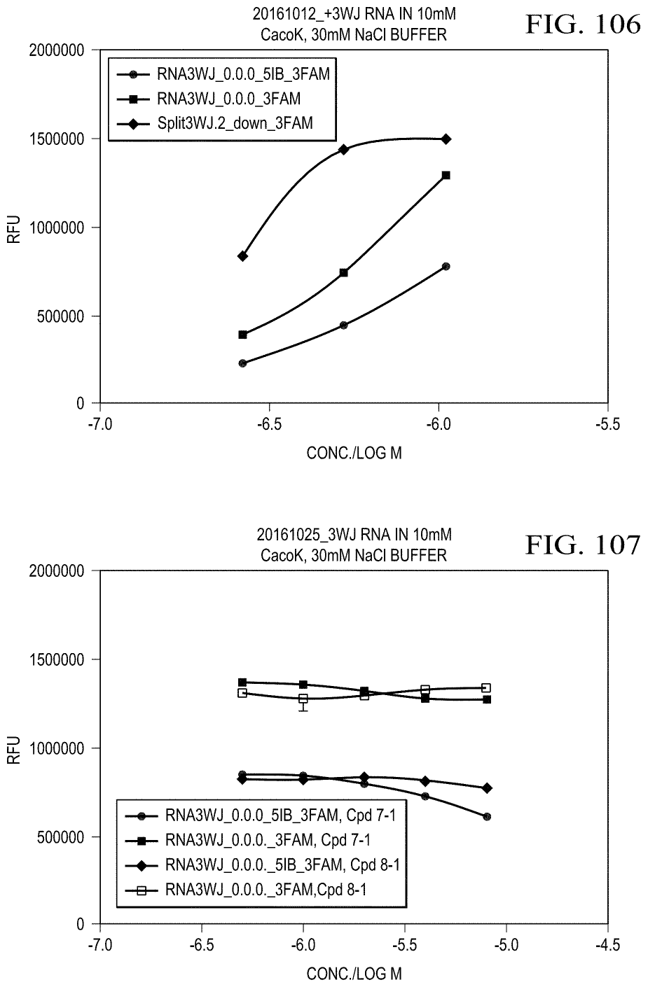

Calibration of the expected fluorescence signal at various RNA concentrations in either CacoK or NaPO₄ buffers was performed first. Experiments in buffers containing salt show distinct fluorescence quenching behavior. A calibration experiment for the CacoK buffer is shown in FIG. 106. Similar results were obtained for the NaPO₄ buffer (results not shown).

First, two compounds (i.e. Ark000007 & Ark000008) were tested in the fluorescence quenching assay to assess concentration dependent influence on the fluorescence signal. Only Ark000007 showed an increase of quenching effect vs. 3WJ_0.0.0_5IB_3FAM construct at conc. >5 μM (FIG. 107). Remaining buffer and sample conditions did not show significant influence of the compound on the fluorescence signal.

The fluorescence quenching experiment was repeated for compounds Ark0000013 and Ark0000014 to measure binding with:

A) RNA3WJ_1.0.0_5IB_3FAM (cis 3WJ with one unpaired nucleotide)

B) Split3WJ.1_up_5IB+Split3WJ.1_down_3FAM (trans 3WJ as 1:1 mix)

C) Split3WJ.2 up_5IB+Split3WJ.2_down_3FAM (trans 3WJ as 1:1 mix)

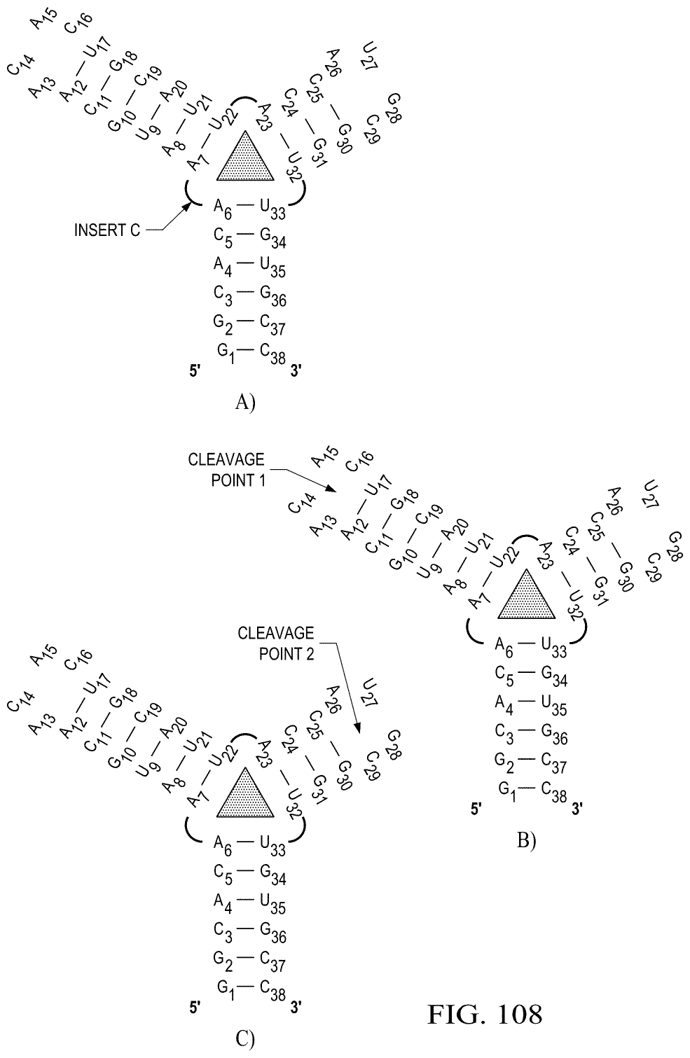

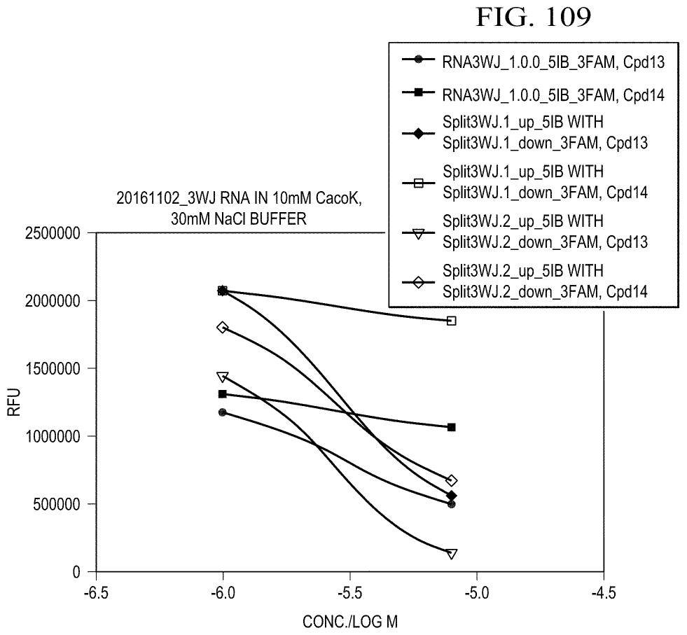

Likely structures for the sequences are illustrated in FIG. 108, and the results of the experiments are shown in FIG. 109. Both cmpds were tested at two concentration points in the fluorescence quenching assays to assess effect upon RNA constructs utilized in the study. Ark000013 (curves associated with Cpd 13 in the Figure) shows a significant concentration dependent effect upon all three RNA constructs used (least effect for cis 3WJ and equal effects for trans 3WJs). The data suggest specific interaction of Ark000013 with the 3WJ constructs. Ark000014 (Cpd14) shows a smaller effect on the RNA constructs (Split3WJ_2 shows larger effect). The compound does appear to be interacting with the RNA target species.

### Example 22: Thermal Shift Binding Assay

Purpose: Test binding of compounds for RNA three way junction (for example, a construct of 38 nt). Thermal shift assay based on established fluorescence quenching assay utilizing FAM as fluorescence tag and Iowa Black as quencher. Tags were attached at the 3′ and 5′ end, respectively. Stable formation of 3WJ upon compound binding would lead to quenching of the FAM fluorescence due to close proximity of the Iowa Black tag. Thermal unfolding leads to increase of fluorescence emission. Assay readout: FAM (465-510 nm) Thermal Shift.

This assay can serve as a part of the toolkit for discovering RNA-binding small molecules by testing binding to a 3WJ in the context of a controlled system with a readily observable readout. PEARL-seq or other methods disclosed herein may then be used to further screen compounds.

Assay sample buffer used: 10 mM CacoK pH 7.2, 30 mM NaCl. Buffer preparation in Dnase/Rnase Free distilled water (Gibco Life Technologies).

Compound Preparation

Tool compounds provided as dry powder are prepared as 50 mM stock solution in 100% d₆-DMSO. Stock solutions of 50 mM concentration in d₆-DMSO are stored at RT.

Hardware

Sample plate: Roche, Light Cycler480 Multiwell Plate96, white, REF 04729692001. (Dilution plate: Greiner REF 781101, PS-Microplate, 384 well, clear). Thermal Shift device: Roche, Light Cycler480.

Assay Protocol

### Assay Buffer Preparation

Daily fresh (10 ml): 1 ml 100 mM CacoK pH 7.2 and 0.3 ml 1 M NaCl filled up to 10 ml with Dnase/Rnase Free distilled water.

### RNA Preparation (RNA Sample Homogenization)

Dilute the RNA 1:10 (final 10 μM) in Assay Buffer.

Heat up the diluted RNA up to 90° C. for 5 min (sealed Eppendorf Tube).

Cool down the RNA probe slowly to RT.

### Compound Preparation

Dilute the compounds to 800 μM in DMSO (Assay: 8 μM).

### Sample Preparation

78.4 μL Assay Buffer are pipetted into Greiner REF 781101, PS-Microplate, 384 (each well needed).

Add 0.8 μL of the RNA-Solution (100 mM).

Add 0.8 μL Compound-Solution (800 mM).

Mix gentle with Multi-Channel Pipette.

Final concentrations in the sample: 1 μM RNA, 8 μM compound, 1% DMSO

### Thermal Shift Measurement (LightCycler480)

Pipet 20 μL Sample Solution into Roche, Light Cycler480 Multiwell Plate96, white, REF 04729692001.

When sample transfer is finished, seal the plate with a clear topseal (part of REF 04729692001).

Centrifuge the plate with a table-top device to spin down the samples.

Measure the 96 plate with the LightCycler480 (Channel: 480/510 nm; Temperature: 41-91° C.).

Analyse measurement-data with the MeltingCurveGenotyping Mode.

### Software

LightCycler480 LCS480 1.5.1.62 LightCycler ThermalShift Analysis

Settings: Acquisition mode: continuous; Ramp rate: 0.1 C.°/sec; Acquisition: 6/C.° Melt Curve Genotyping for All Samples

Channel 480/510 nm

Program Name Program

Stds Settings Auto-Group

Sensitivity normal

Temp Range 41-91° C.

Score 0.7

Res. 0.1

Curves are fitted with raw and normalized data.

Results

Melting curves analysis show melting temperature (Tₘ) of ˜51° C. Range of RNA concentrations was tested and assay window was determined (conc. range of 0.5-1 μM yields best results). The choice of buffer also affected the Tₘ. RNA constructs were tested under different buffer conditions (especially in presence of salt) in the thermal shift assay. Increase of salt concentration shows a tendency to increase melting temperature. However, as seen already for the fluorescence quenching assay, this observation is strongly dependent on buffer conditions. CacoK with 30 mM salt at 1 μM RNA conc. was used to assess compound effects on 3WJ stability. RNA constructs were tested under different buffer conditions (especially in presence of salt) in the thermal shift assay. As expected, an increase of salt concentration shows a tendency to increase melting temperature. However, as seen already for the fluorescence quenching assay, this observation is strongly dependent on buffer conditions. The RNA construct was folded in presence of higher salt concentration and had a melting temperature of 61° C. rather than the 51° C. at lower salt concentration. These conditions were used for screening test compounds.

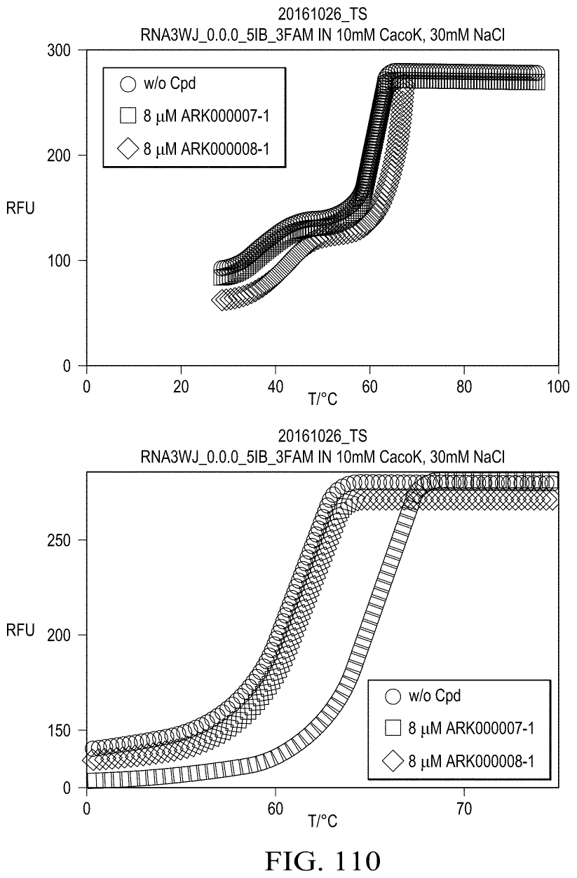

Compounds Ark000007 & Ark000008 were tested in the thermal shift assay with the 3WJ_0.0.0_5IB_3FAM RNA construct (FIG. 110). Data analysis shows a significant effect for Ark000007 with melting temperature shift of ˜5° C. (i.e. from 61.2° C. to 65.6° C.). In contrast, only a very small effect for Ark000008 was observed. These data suggest that the presence of Ark000007 increases stability of the 3WJ.

Compounds Ark0000013 and Ark0000014 were also tested in the thermal shift assay against three RNA 3WJ constructs, A) RNA3WJ_1.0.0_5IB_3FAM (cis 3WJ with one unpaired nucleotide); B) Split3WJ.1_up_5IB+Split3WJ.1_down_3FAM (trans 3WJ as 1:1 mix); and C) Split3WJ.2_up_5IB+Split3WJ.2_down_3FAM (trans 3WJ as 1:1 mix).

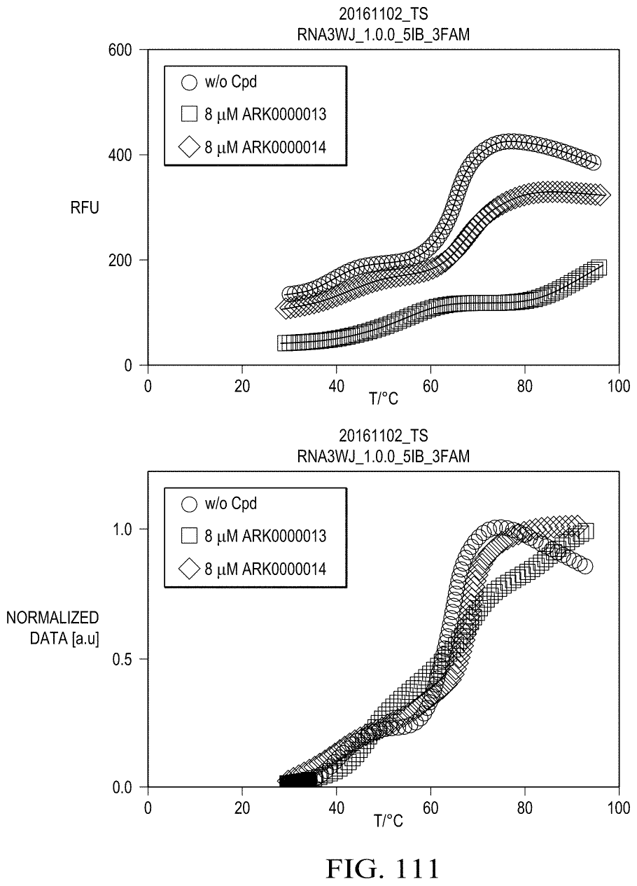

When the compounds were tested with RNA3WJ_1.0.0_5IB_3FAM, data analysis showed a significant effect for Ark000013 in the melting curves with a significantly lower fluorescence signal in presence of the compound (FIG. 111).

Normalized data showed no proper melting curve in the presence of Ark000013 and the algorithm of data analysis software was unable to determine a meaningful melting point. A weaker effect was observed for Ark000014, with a melting temperature shift of ˜3° C. (i.e. from 65.6° C. to 68.4° C.). The data suggest that the presence of Ark000013 increases stability of the 3WJ fold upon binding, whereas Ark000014 shows a much less pronounced effect. These results are in line with the fluorescence quenching assay.

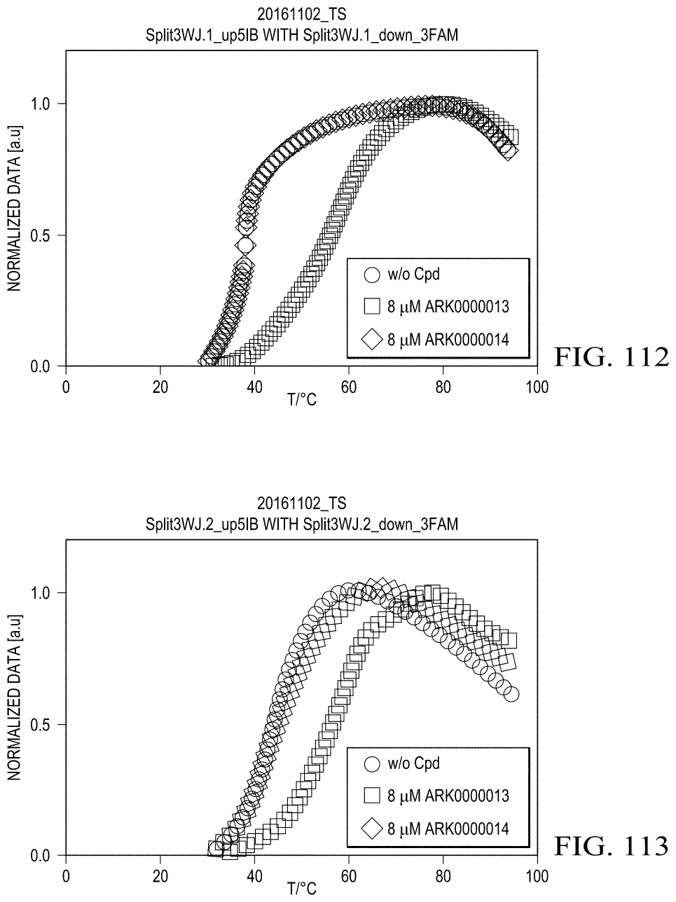

In the presence of the B) RNA above, Split3WJ.1_up_5IB+Split3WJ.1_down_3FAM, data analysis showed a significant effect for Ark000013, with a melting temperature shift of ˜21° C. (i.e. from 37.5° C. to 58.2° C.) (FIG. 112). Only a minor effect was observed for Ark000014 with a melting temperature shift of only ˜1° C. (i.e. from 37.5° C. to 38.8° C.). The data suggested that the presence of Ark000013 increased the stability of the 3WJ fold upon binding, whereas Ark000014 showed a much less pronounced effect. The 3WJ formed in trans from 2 RNA molecules shows a significantly lower stability than the cis folded 3WJ (in absence and presence of cmpd). Especially in absence of a compound, a stem-loop structure with a larger bulge is possibly the most populated conformation.

In the presence of the C) RNA above, Split3WJ.2 up_5IB+Split3WJ.2_down_3FAM, data analysis showed a significant effect for Ark000013, with a melting temperature shift of ˜13° C. (i.e. from 44.0° C. to 56.9° C.) (FIG. 113). Only a minor effect was observed for Ark000014, with melting temperature shift of only ˜1° C. (i.e. from 44.0° C. to 44.7° C.). The data suggest that the presence of Ark000013 increases the stability of the 3WJ fold upon binding, whereas Ark000014 shows a much less pronounced effect. The trans 3WJs studied seem to show lower stability than the cis 3WJs, however, the Split_2 3WJ adopts a more stable conformation than Split_1 (in absence of a compound). In the presence of a compound, the melting temperature for both trans 3WJ Split_1 & Split_2 is similar, suggesting the formation of a 3WJ fold in the presence of the compound.

Ark0000013 and Ark0000014 were tested with a number of RNA constructs. The results are shown below in Tables 9 and 10. Compound Ark000039 was also tested in the thermal shift assay vs. the cis folded RNA 3WJs at different RNA:ligand ratios (i.e. 1:1, 1:3). For construct 3WJ_0.0.0_5IB_3FAM the raw data shows no significant effect for Ark000039 in the melting curves (neither at equimolar concentrations nor 3× molar excess). Also, normalized data show no significant effect for cmpd Ark000039. It appears that Ark000039 does not significantly influence stability of the 3WJ fold and hence no indication of binding for Ark000039 was observed. The same lack of effect was noted in tests with sequences RNA3WJ_3.0.0_5IB_3FAM and RNA3WJ_1.0.0_5IB 3FAM.

TABLE 9
Ark0000013 Thermal Shift Data

| | Melting | Melting | |
|---|---|---|---|
| | temp | temp | Temp. |
| | [° C.] − | [° C.] + | Shift |
| 3WJ construct | cmpd | cmpd | [° C.] |
| RNA3WJ_0.0.0_5IB_3FAM | 61.2 | 84.1 | 24.2 |
| RNA3WJ_1.0.0_5IB_3FAM | 65.6 | 87.0 | 21.4 |
| RNA3WJ_1.1.0_5IB_3FAM | 63.3 | 85.5 | 22.2 |
| RNA3WJ_1.1.1_5IB_3FAM | 62.2 | 82.9 | 20.7 |
| RNA3WJ_2.0.0_5IB_3FAM | 62.2 | 84.3 | 22.1 |
| RNA3WJ_2.1.0_5IB_3FAM | 41.9 | 45.7 | 3.8 |
| RNA3WJ_3.0.0_5IB_3FAM | 62.0 | 83.7 | 21.7 |
| Split3WJ_1 | 37.8 | 58.2 | 20.4 |
| Split3WJ_2 | 44.7 | 56.9 | 12.2 |

TABLE 10
Ark0000014 Thermal Shift Data

| | Melting | Melting | |
|---|---|---|---|
| | temp | temp | Temp. |
| | [° C.] − | [° C.] + | Shift |
| 3WJ construct | cmpd | cmpd | [° C.] |
| RNA3WJ_0.0.0_5IB_3FAM | 59.9 | 61.5 | 1.6 |
| RNA3WJ_1.0.0_5IB_3FAM | 65.6 | 68.1 | 2.5 |
| RNA3WJ_1.1.0_5IB_3FAM | 63.3 | 65.1 | 1.8 |
| RNA3WJ_1.1.1_5IB_3FAM | 62.2 | 64.3 | 2.1 |
| RNA3WJ_2.0.0_5IB_3FAM | 62.2 | 64.4 | 2.2 |
| RNA3WJ_2.1.0_5IB_3FAM | 41.9 | 42.0 | 0.1 |
| RNA3WJ_3.0.0_5IB_3FAM | 62.0 | 63.9 | 1.9 |
| Split3WJ_1 | 37.5 | 37.8 | 0.3 |
| Split3WJ_2 | 44.3 | 44.0 | −0.3 |

TABLE 11
Thermal Shift Data for Additional Compounds Tested
with RNA Sequence 3WJ_0.0.0_5IB_FAM

| | | Melt. | Melt. | | |
|---|---|---|---|---|---|
| | | Temp. | Temp. | Shift | |
| | | without | with | melt. | |
| Compound | Collaboration | cmpd | cmpd | Temp. | |
| No. | Code | [° C.] | [° C.] | [° C.] | Remarks |
| ARK000007 | ARK-1 | 61.2 | 65.6 | +4.4 | |
| ARK000008 | ARK-2 | 61.2 | 61.8 | +0.6 | |
| ARK000009 | ARK-3 | 62.3 | 62.9 | +0.6 | |
| ARK000010 | ARK-4 | 61.6 | 61.4 | −0.2 | |
| ARK000011 | ARK-5 | 61.7 | 60.3 | −1.4 | |
| ARK000012 | ARK-6 | N/A | N/A | N/A | |
| ARK000013 | ARK-7 | 59.9 | 84.1 | +24.2 | |
| ARK000014 | ARK-8 | 59.9 | 61.5 | +1.6 | |
| ARK000015-1 | ARK-9-01 | 61.2 | 78.8 | +17.6 | Enantiomer of target |
| | | | | | ARK-10, TFA Salt of |
| | | | | | same material is |
| | | | | | registered as |
| | | | | | ARK000015-2 |
| | | | | | (ARK-9-02) |
| ARK000015-2 | ARK-9-02 | 60.8 | 79.8 | +19.0 | Enantiomer of target |
| | | | | | ARK-10, HCl Salt of |
| | | | | | same material is |
| | | | | | registered as |
| | | | | | ARK000015-1 (ARK-9-01) |
| ARK000016 | ARK-10 | 62.5 | 83.8 | +21.3 | Enantiomer of target |
| | | | | | ARK000015 (ARK-9) |
| ARK000017 | ARK-11 | 61.2 | 60.6 | −0.6 | Enantiomer of target |
| | | | | | ARK000018-1 (ARK-12) |
| ARK000018 | ARK-12 | 60.6 | 60.8 | +0.2 | Enantiomer of target |
| | | | | | ARK000017-1 (ARK-11) |
| ARK000022 | ARK-13-D | 59.8 | 60.2 | +0.4 | Enantiomer of target |
| | | | | | ARK000023-1 (ARK-13-L) |
| ARK000023 | ARK-13-L | 60.1 | 60.7 | +0.6 | Enantiomer of target |
| | | | | | ARK000022-1 (ARK-13-D) |
| ARK000019 | ARK-14 | 60.3 | 60.0 | −0.3 | |
| ARK000020 | ARK-15 | 59.9 | 60.2 | +0.3 | |
| ARK000021 | ARK-16 | 59.5 | 40.5 | −19.0 | |
| ARK000024 | ARK-80 | 60.4 | 60.7 | +0.3 | LCMS carried out in a |
| | | | | | long 16 min run time |
| | | | | | method hence HPLC is |
| | | | | | not recorded separately. |
| ARK000025 | ARK-81 | 60.6 | 50.4 | −10.2 | LCMS carried out in a |
| | | | | | long 16 min run time |
| | | | | | method hence HPLC is |
| | | | | | not recorded separately. |
| ARK000026 | ARK-82 | 59.7 | 60.9 | −1.2 | LCMS carried out in a |
| | | | | | long 16 min run time |
| | | | | | method hence HPLC is |
| | | | | | not recorded separately. |
| ARK000027 | ARK-89 | 59.7 | 83.4 | +23.7 | LCMS carried out in a |
| | | | | | long 16 min run time |
| | | | | | method hence HPLC is |
| | | | | | not recorded separately. |
| ARK000028 | ARK-90 | 60.3 | 82.3 | +22.0 | LCMS carried out in a |
| | | | | | long 16 min run time |
| | | | | | method hence HPLC is |
| | | | | | not recorded separately. |
| ARK000029 | ARK-91 | 60.2 | 63.2 | +3.0 | LCMS carried out in a |
| | | | | | long 16 min run time |
| | | | | | method hence HPLC is |
| | | | | | not recorded separately. |
| ARK000030 | ARK-125 | 60.0 | 64.8 | +4.8 | LCMS carried out in a |
| | | | | | long 16 min run time |
| | | | | | method hence HPLC is |
| | | | | | not recorded separately. |
| ARK000031 | ARK-126 | 60.0 | 84.1 | +24.1 | LCMS carried out in a |
| | | | | | long 16 min run time |
| | | | | | method hence HPLC is |
| | | | | | not recorded separately. |
| ARK000032 | ARK-127 | 60.1 | 75.1 | +15.0 | LCMS carried out in a |
| | | | | | long 16 min run time |
| | | | | | method hence HPLC is |
| | | | | | not recorded separately. |
| ARK000033 | ARK-77 | 61.5 | 62.4 | +0.9 | LCMS carried out in a |
| | | | | | long 16 min run time |
| | | | | | method hence HPLC is |
| | | | | | not recorded separately. |
| ARK000034 | ARK-77A | 59.9 | 60.2 | +0.3 | LCMS carried out in a |
| | | | | | long 16 min run time |
| | | | | | method hence HPLC is |
| | | | | | not recorded separately. |
| ARK000039 | ARK-138 | 61.5 | 60.3 | −1.2 | |

Interestingly, hook and click compounds (PEARL-seq compounds) bearing ligand, tether, warhead, and click-ready group, such as ARK000031 and ARK000032, showed large thermal shift values of +24.1 and +15.0° C., indicating strong binding to the RNA target sequence.

### Example 23: Ligand Observed NMR Binding Assay

Purpose: Test direct binding of compounds for RNA three way junction (3WJ). This ligand observed NMR assay is used to test direct binding of compounds to an RNA target, for example a 38 nt synthetic RNA 3WJ and others as described below. Ligand observed assay was used for hit validation studies of single compounds. Established experiments were eventually used to perform group epitope mapping, described below.

Assay Reagents and Hardware

Sample buffer: 10 mM Cacodylate, pH 7.1; 0.68 g [MW: 137.99 g/mol]; fill up to 500 ml with Millipore H₂O.

### Compound Preparation

Compound Stocks: Tool compounds provided as dry powder were prepared as 50 mM stock solution in 100% d₆-DMSO. Test compounds provided as dry powder were prepared as 50 mM stock solution in 100% d₆-DMSO. Stock solutions of 50 mM concentration in d₆-DMSO were stored at 4° C.

### Hardware

Sample tube: NMR tube; Norell, article #ST500-7 for NMR sample measurement

NMR spectrometer: Bruker AVANCE600 spectrometer operating at 600.0 MHz for ¹H. 5-mm z-gradient TXI Cryoprobe.

Assay Procedure

### RNA Preparation (RNA Sample Homogenization)

Dried RNA pellet is solubilized in sample buffer 10 mM Cacodylate pH 7.1.

RNA aliquot at 200 μM (stock concentration) is denatured at 95° C. for 3 min and snap-cooled on ice for 3 min.

### Sample Preparation

23 μL d₆-DMSO are pipetted into a 1.5 mL eppendorf tube to ensure 5% d₆-DMSO present in the sample as a locking agent.

Add 2 μL of each fragment (50 mM stock solution).

Add 450 μL assay buffer.

Add 25 μL homogenized RNA stock solution of the RNA 3WJ (200 μM stock solution).

Sample is vortexed to ensure proper mixing and placed into NMR spectrometer to start measurement of the sample.

Final concentrations in the sample: 200 μM each compound and 10 μM RNA target molecule.

### NMR Measurement

Sample is placed into magnet and temperature adjusted to 288 K. Spectrometer frequency at 600 MHz is matched and tuned. Magnetic field is shimmed to homogenize magnetic field around the sample.

Proton 900 pulse is determined and water resonance frequency is adjusted to ensure maximal water suppression. The determined values are transferred to the NMR experiments that will be recorded for the respective sample.

Sequence of experiments includes a Proton 1D experiment with a Watergate sequence for water suppression, a WaterLOGSY (WLOGSY) and a 1D Saturation transfer difference (STD) experiment to test for direct binding of the compounds to the RNA.

Details 1D Watergate experiment: For each 1D WATERGATE spectrum a total of 8192 complex points in fl (¹H) with 128 scans were acquired (experiment time 4 min.). The spectral width was set to 16.66 ppm.

Details WLOGSY experiment: For the WLOGSY-spectrum, a total of 1024 complex points in fl (¹H) with 256 scans were acquired (experiment time 25 min.). The carrier frequency for ¹H was set at the water resonance (˜4.7 ppm). The spectral width was set to 16.66 ppm in the direct dimension (¹H).

Details STD experiment: For the STD-spectrum, a total of 1024 complex points in fl (¹H) with 1024 scans were acquired (experiment time 65 min.). The carrier frequency for ¹H was set at the water resonance (˜4.7 ppm). The spectral width was set to 16.66 ppm in the direct dimension (¹H). For the on-resonance experiments saturation is set to 2.0 sec at a saturation frequency of −2500 Hz. For the off-resonance experiment saturation frequency is set at 10200 Hz.

### Readout

Software: Topspin™ version: 2.1 (Oct. 24, 2007)

Measurement mode: ID

Python scripts are used to process all recorded spectra in the assay setup, screening and deconvolution process.

Spectra were analyzed for direct binding signals of the compounds. Identified single compound hits were reported.

Ligand Observed NMR Binding Assay for CAG Repeat RNA

Following the above procedure, various tool and test compounds were assayed for binding. In a first series of experiments, compounds were tested for binding to 17CAG or 41CAG (sample was 3 μM in RNA). Compounds HP-AC008001-A08, HP-AC008002-A06, HP-AC008002-D10, and most of an initial screen of 41 small molecule fragments did not show significant difference in binding signals for both RNA target species 17CAG and 41CAG. However, several of the compounds did show significant changes in their signals in the presence of the two RNA target species.

| Structure | ID | STD signal |
|---|---|---|
| 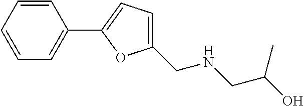 [SMILES: CC(O)CNCc1ccc(-c2ccccc2)o1] | HP-AT005003-C03 | distinct |
| 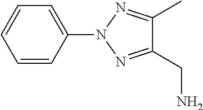 | HP-AC008001-E02 | distinct |
| 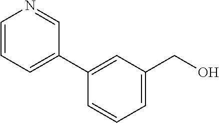 [SMILES: OCc1cccc(-c2cccnc2)c1] | HP-AC008002-E01 | distinct |
| 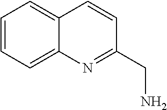 [SMILES: NCc1ccc2ccccc2n1] | HP-AC008004-C07 | weak |
| 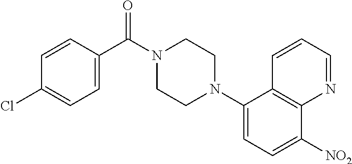 [SMILES: O=C(c1ccc(Cl)cc1)N1CCN(c2ccc([N+](=O)[O-])c3ncccc23)CC1] | CPNQ | distinct |

ARK0000013 was also tested in the NMR binding assay. Test Sample: 10 μM RNA3WJ_0.0.0_5IB_3FAM+/−200 Ark000013. ¹H 1D Watergate & WaterLOGSY spectra recorded of Ark000013 were used as a reference (Note: aromatic signals observed between 7.4-7.9 ppm and due to symmetry of the center triptycene scaffold all 9 protons are magnetically equivalent). In the presence of RNA a clear reduction of negative sign signals occurred for the Ark000013 resonances. Data suggested binding of Ark000013 to the 3 WJ RNA as the target species. STD experiments showed small signals that were sufficient to qualitatively confirm binding.

Epitope Mapping

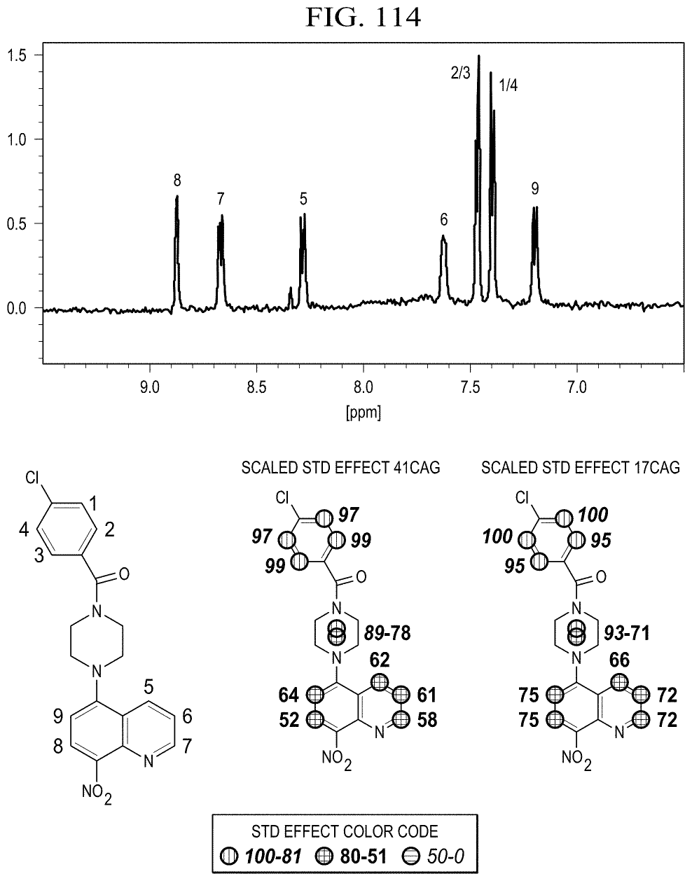

Epitope mapping was performed on a number of compounds. As a first example, compound CPNQ was analyzed at a concentration of 50 μM. A ¹H 1D Watergate spectrum with zoom to aromatic region of the spectrum was obtained. Preliminary assignment of ¹H resonances for this and the following examples was based on chemical shift distribution, coupling pattern and simulation of NMR spectrum (www.nmrdb.org). The structure of CPNQ, assigned proton resonances, NMR spectrum, and epitope mapping results are shown in FIG. 114. Due to signal overlap no individual assignment of the piperazine ring system was possible. Conditions: 10 mM Tris pH 8.0, 5 mM DTT, 5% DMSO-d₆; T=288.1 K. Epitope mapping experiments were performed in the presence of 41CAG and 17CAG sequences using the STD experimental conditions described above. In the case of CPNQ, data suggests for both RNA constructs the tendency that protons of the chlorophenyl moiety are in closer proximity to RNA than the nitroquinoline.

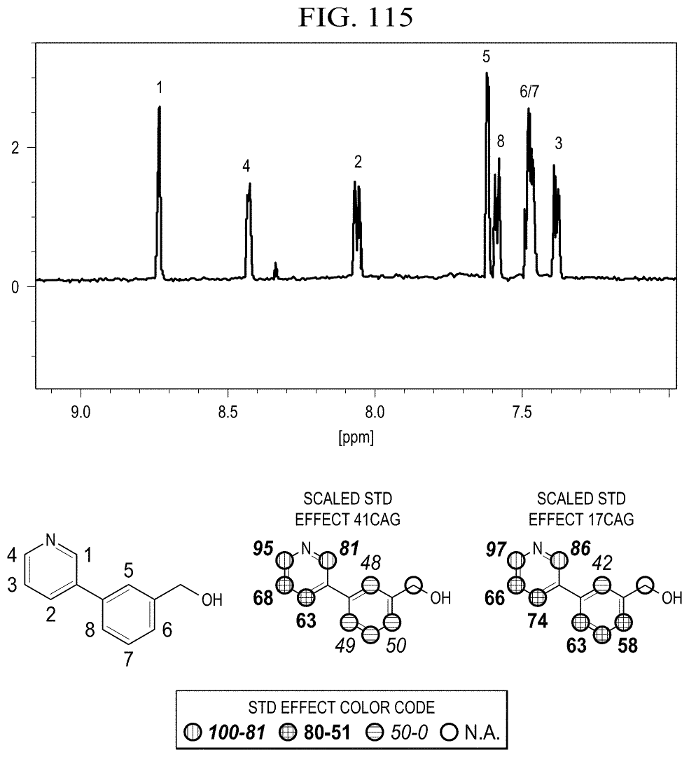

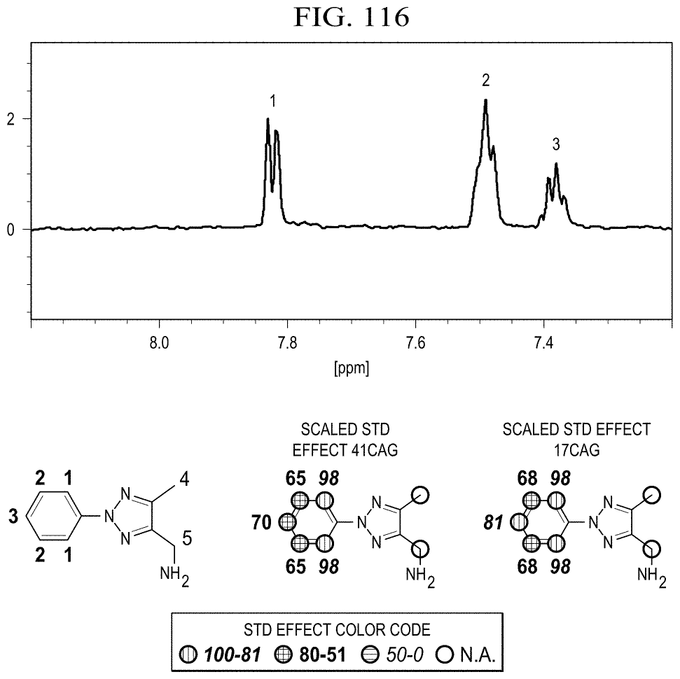

The same experiment was performed for compound HP-AC008002-E01 under similar conditions (see FIG. 115). The scaled STD effect was plotted onto the molecule according to the preliminary assignments. The data suggests for both RNA constructs that protons of the pyridine ring are in closer proximity to RNA than the benzene ring. The aliphatic CH₂ group could not be observed due to buffer signal overlap in that region.

The same experiment was performed for compound HP-AC008001-E02 under similar conditions (see FIG. 116). The scaled STD effect was plotted onto the molecule according to the preliminary assignments. The data suggest for both RNA constructs that aromatic protons closest to the heterocycle are in closer proximity to RNA protons. Aliphatic proton resonances could not be assessed by STD due to direct saturation artifacts/buffer signal overlap in that region (epitope mapping by WaterLOGSY).

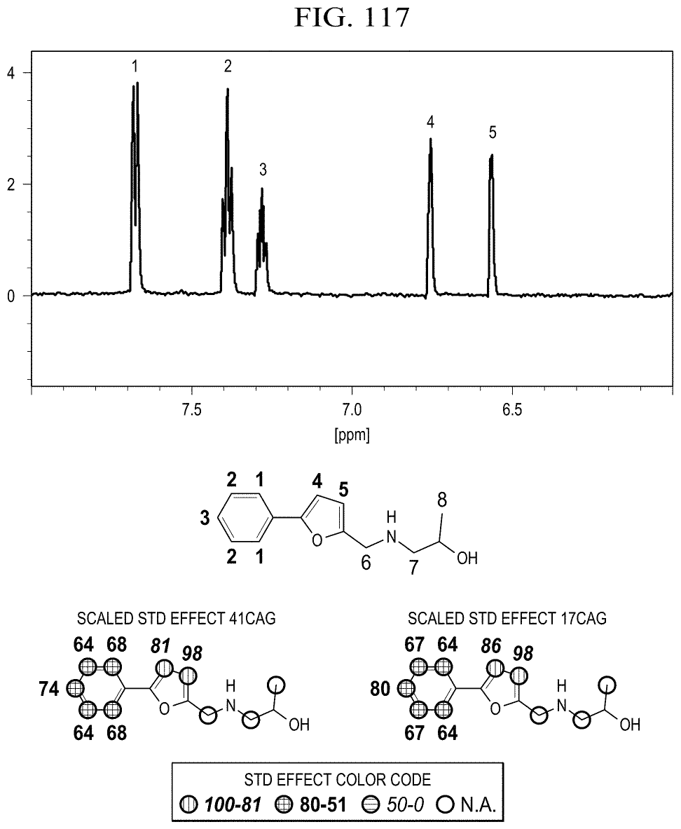

The same experiment was performed for compound HP-AT005003-C03 under similar conditions (see FIG. 117). The scaled STD effect was plotted onto the molecule according to the preliminary assignments. Due to signal overlap no individual assignment of the CH₂ groups was possible. The data suggest for both RNA constructs that protons of the furan moiety are in closer proximity to RNA protons than the phenyl.

NMR Competition Experiments

Competition experiments were also performed. Test Sample: 2.5 μM 41CAG RNA (476 nt) was combined with the following: 100 μM HP-AC008002-E01 (A); +/−200-400 μM HP-AC008001-E02 (B); and +/−200-400 μM HP-AT005003-C03 (C). ¹H 1D Watergate & WaterLOGSY spectra recorded of HP-AC008002-E01 are used as a reference. In the presence of competitor (i.e. either HP-AT005003-C03 or HP-AC008001-E02) the WaterLOGSY signals of HP-AC008002-E01 were still observed, even at a 1:4 ratio of compound vs. competitor. The experiments did not reveal any indication of competitive behavior in the utilized compound mixtures. Data suggests that compounds do not compete for the same single binding site.

In a further experiment, as Test Sample 2.5 μM 41CAG RNA (476 nt) was used in the presence of: 100 μM HP-AC008001-E02 (B) or 100 μM HP-AT005003-C03 (C); +/−200-400 μM HP-AC008002-E01 (A). ¹H 1D Watergate and WaterLOGSY spectra recorded single cmpds were used as a reference. In the presence of a competitor (i.e. HP-AC008002-E01 (A)) the WaterLOGSY signals of HP-AC008001-E02 (B) or HP-AT005003-C03 (C) were still observed even at a 1:4 ratio of cmpd vs. competitor. Experiments did not reveal any indication of competitive behavior in the utilized compound mixtures. The data suggest that compounds do not compete for the same single binding site.

In a further experiment, as Test Sample: 2.5 μM 41CAG RNA (476 nt) was used in the presence of: 100 μM HP-AC008001-E02 (B)+/−200-400 μM HP-AT005003-C03 (C). ¹H 1D Watergate & WaterLOGSY spectra recorded of single cmpd HP-AC008001-E02 were used as a reference. In the presence of competitor (i.e. HP-AT005003-C03 (C)) the WaterLOGSY signals of HP-AC008001-E02 (B) were still observed even at a 1:4 ratio of cmpd vs. competitor. The experiments did not reveal any indication of competitive behavior in the utilized compound mixture. The data suggest that compounds do not compete for the same single binding site.

### Example 24: Ligand Observed NMR Binding Assay for CAG Repeat RNA

Purpose: Test direct binding of compounds for httmRNA (construct with 41 CAG repeats 474 nt) and others as described below. Ligand observed NMR assay to test direct binding of fragments to RNA target (e.g. construct with 41 CAG repeats 474 nt). Single compound hits were identified for further characterization by orthogonal assay (e.g. surface plasmon resonance, SPR). Ligand observed assay was used for primary screen and deconvolution into single fragment hits. Established experiments were eventually used for group epitope mapping.

CAG repeat expansions in protein coding portions of specific genes are classified as Category I repeat expansion diseases. Currently, nine neurologic disorders are known to be caused by an increased number of CAG repeats, typically in coding regions of otherwise unrelated proteins. During protein synthesis, the expanded CAG repeats are translated into a series of uninterrupted glutamine residues forming what is known as a polyglutamine tract (“polyQ”).

This assay tests for direct binding of compounds to httmRNA and may be adapted for other repeat RNAs. Compounds are tested in pools (i.e. pool size of 12 fragments in each sample in the primary screen and smaller pool sizes during deconvolution and eventually single compound measurements).

Assay Reagents and Hardware

Sample buffer: 10 mM Tris-HCl, pH 8.0, 0.78 g [MW: 157.56 g/mol]; 75 mM KCl, 2.79 g [MW: 74.55 g/mol]; 3 mM MgCl₂, 0.14 g [MW: 95.21 g/mol]; fill up to 500 mL with Millipore H₂O.

### Compound Preparation

Compound Stocks: Fragment library stock solutions are provided at 100 mM concentration in 100% d₆-DMSO. Tool compounds provided as dry powder are prepared as 100 mM stock solution in 100% d₆-DMSO. Stock solutions of 100 mM concentration in d₆-DMSO are stored at 4° C.

### Hardware

Sample tube: NMR tube; Norell, article #ST500-7 for NMR sample measurement.

NMR spectrometer: Bruker AVANCE600 spectrometer operating at 600.0 MHz for ¹H. 5-mm z-gradient TXI Cryoprobe.

Assay Procedure

### RNA Preparation (RNA Sample Homogenization)

Dried RNA pellet is solubilized in sample buffer 10 mM Tris-HCl pH 8.0, 75 mM KCl, 3 mM MgCl₂. RNA aliquot at 13.9 μM (stock concentration) is denatured at 95° C. for 3 min and snap-cooled on ice for 3 min, and refolded at 37° C. for 30 min.

### Sample Preparation

13-24 μL d₆-DMSO are pipetted into an 1.5 mL eppendorf tube to ensure 5% d₆-DMSO present in the sample as a locking agent (depending on pool size of the prepared sample). Add 1 μL of each fragment (100 mM stock solution).

Add 367 μL assay buffer.

Add 108 μL homogenized RNA stock solution of the httmRNA (13.9 μM stock solution).

Sample is vortexed to ensure proper mixing and placed into NMR spectrometer to start measurement of the sample.

Final concentrations in the sample: 200 μM each fragment and 3 μM RNA target molecule.

### NMR Measurement

Sample is placed into magnet and temperature adjusted to 288 K. Spectrometer frequency at 600 MHz is matched and tuned. Magnetic field is shimmed to homogenize magnetic field around the sample.

Proton 900 pulse is determined and water resonance frequency is adjusted to ensure maximal water suppression. The determined values are transferred to the NMR experiments that will be recorded for the respective sample.

Sequence of experiments includes a Proton 1D experiment with a Watergate sequence for water suppression, a WaterLOGSY (WLOGSY) and a 1D Saturation transfer difference (STD) experiment to test for direct binding of the compounds to the RNA.

Details for 1D Watergate experiment: For each 1D WATERGATE spectrum a total of 8192 complex points in fl (¹H) with 128 scans were acquired (experiment time 4 min.). The spectral width was set to 16.66 ppm.

Details WLOGSY experiment: For the WLOGSY-spectrum, a total of 1024 complex points in fl (¹H) with 256 scans were acquired (experiment time 25 min.). The carrier frequency for ¹H was set at the water resonance (˜4.7 ppm). The spectral width was set to 16.66 ppm in the direct dimension (¹H).

Details STD experiment: For the STD-spectrum, a total of 1024 complex points in fl (¹H) with 1024 scans were acquired (experiment time 65 min.). The carrier frequency for ¹H was set at the water resonance (˜4.7 ppm). The spectral width was set to 16.66 ppm in the direct dimension (¹H). For the on-resonance experiments saturation is set to 2.0 sec at a saturation frequency of −2500 Hz. For the off-resonance experiment saturation frequency is set at 10200 Hz. Readout

Software: Topspin™ version: 2.1 (Oct. 24, 2007)

Measurement mode: 1D

Python scripts were used to process all recorded spectra in the assay setup, screening and deconvolution process. Spectra were analyzed for direct binding signals of the compounds. Identified single compound hits were reported.

### Example 25: Illumina Small RNA-Seq Library Preparation Using T4 RNA Ligase 1 Adenylated Adapters

Purpose: Enable deep sequencing of a short synthetic RNA after treatment with SHAPE reagents or PEARL-seq compounds. The herein-described library preparation protocol describes a method to generate next generation sequencing libraries from small synthetic RNAs by ligating adapters to both ends. The ligation is required in order to allow cDNA synthesis from the ligated adapters—hence sequencing the whole target RNA. The technique represents one step in the process of SHAPE sequencing. SHAPE sequencing aims at analysing RNA secondary structure by determination of a mutation frequency after treatment with conformation selective SHAPE reagents.

Target name for this example: targetRNA oligonucleotide “RNA3WJ_0.0.0_noLab”, sequence rGrGrCrArCrArArArUrGrCrArArCrArCrUrGrCrArUrUrArCrCrArUrGrCrGrGrUrU rGrUrGrCrC (SEQ ID NO: 31). Physiological role: Synthetic RNA oligonucleotide capable of forming a three way junction secondary structure. Assay principle: 1) Ligation of 3′-adapter to target RNA; 2) Phosphorylation of 5′ end of target RNA; 3) 1st and 2nd strand cDNA synthesis from ligated adapters; 4) Incorporation and amplification of barcoded Illumina primers by PCR. Assay readout: Agarose Gel Electrophoresis, Sanger-sequencing.

Assay Reagents and Hardware
T4 RNA Ligase 2, truncated KQ (NEB #M0373S)
50% PEG8000 (supplied with NEB #M0373 S)
RNaseOUT (Invitrogen)
T4 RNA Ligase 1 (ssRNA Ligase) (NEB #M0204S)
10 mM ATP (supplied with NEB #M0204S)
SuperScriptIII Reverse Transcriptase (Invitrogen)
Phusion® Hot Start Flex DNA Polymerase (NEB M0535)
MinElute Gel Extraction kit (Qiagen)
Quant-iT HS DNA assay kit (Inivtrogen)
0.2 M Cacodylic acid

| 0.1M Potassium Cacodylate pH 7.2 (CacoK-stock) | | stock | final |
|---|---|---|---|
| 25.0 ml | 0.2M Cacodylic acid | 200 mM | 100 mM |
| ~25.0 ml - adjust pH to 7.2 | 0.2M KOH | 200 mM | 100 mM |
| Adjust to 50 ml | Millipore H₂O | | |
| Storage: 4° C. | | | |

| 10 mM CacoK pH 7.2 | | stock | final |
|---|---|---|---|
| 1 ml | CacoK pH 7.2 | 100 mM | 10 mM |
| 9.00 ml | Millipore H₂O | | |

| Buffer 1 (10 mM CacoK pH 7.2; 30 mM NaCl) | | | stock | final |
|---|---|---|---|---|
| 1 | ml | CacoK pH 7.2 | 100 mM | 10 mM |
| 0.3 | ml | NaCl | 1000 mM | 30 mM |
| 8.7 | ml | Millipore H₂O | | |

Oligonucleotides

targetRNA oligonucleotide “RNA3WJ_0.0.0_noLab” (IDT custom synthese)

(SEQ ID NO: 31)
5′ rGrGrCrArCrArArArUrGrCrArArCrArCrUrGrCr
ArUrUrArCrCrArUrGrCrGrGrUrUrGrUrGrCrC 3′
3′ adapter DNA oligo “Universal miRNA Cloning
Linker” (NEB S1315S)
(SEQ ID NO: 42)
5′ rAppCTGTAGGCACCATCAAT-NH₂ 3′
5′ adapter RNA oligo
(SEQ ID NO: 43)
5′ rGrUrUrCrArGrArGrUrUrCrUrArCrArGrUrCrCr
GrArCrGrArUrC 3′

Reverse transcription primers: (NNNNNN indicates an 8 base “unique molecular identifier” tag)

1st strand synthesis Primer (P7 RT-Anti UCL)
(SEQ ID NO: 44)
5′ GTGACTGGAGTTCAGACGTGTGCTCTTCCGATCTNNNNNNN
NATTGATGGTGCCTACAG 3′
2nd strand synthesis Primer (P5 2nd strand)
(SEQ ID NO: 45)
5′ TCTTTCCCTACACGACGCTCTTCCGATCTNNNNNNNNGTTC
AGAGTTCTACAGTCCGACGATC 3′

Library PCR amplification primers: All primers contain specific 8 nt index sequence tag (INDEX) required for library deconvolution.

Several forward PCR primers
(SEQ ID NOS 46-47, respectively)
5′ AATGATACGGCGACCACCGAGATCTACAC
(INDEX)TCTTTCCCTACACGACGCTCTTCCG
ATCT 3′
Several reverse PCR primers
(SEQ ID NOS 48-49, respectively)
5′CAAGCAGAAGACGGCATACGAGAT(INDEX)
GTGACTGGAGTTCAGACGTGTGCTCTTCCGAT
CT 3′
qPCR/Sequencing Primers:
Quanti qPCR 1_fw
(SEQ ID NO: 50)
5′ GATACGGCGACCACCGAG 3′
Quanti qPCR l_rv
(SEQ ID NO: 51)
5′ GCAGAAGACGGCATACGAGAT 3′

Assay Procedure

### Preparation

Dissolve target RNA with RNase free water to 100 μM.

Pipette 3 aliquots a 180 μl and additional small volume aliquots (5 μl). Storage: −80° C.

For ligation, resuspend the lyophilized Universal miRNA Cloning Linker (UCL) in RNAse-free water to 100 μM stock concentration. 1 μl UCL has a concentration of 100 pmol (100 μM).

Adjust the adapter concentration to 10 pmol/μl (10 μM) with RNase-free water (1:10 dilution).

### RNA Folding

Dilute the dissolved target RNA 1:10 with Buffer 1 to get a 10 μM solution for ligation.

Incubate at 90° C. for 5 min, cool down slowly to RT and store on ice.

### 3′ Adapter Ligation

Denature the 3′ adapter (UCL) at 65° C. for 30 sec, immediately chill on ice.

Ligations are carried out with T4 RNA Ligase 2 in the absence of ATP.

Setup the ligation reaction with:

| | |
|---|---|
| 1 μl RNA | 10 μM |
| 4 μl 3′ adapter “Universal miRNA | 40 μM |
| Cloning Linker” | |
| 2 μL 10 × T4 RNA ligase buffer | 1× |
| without ATP | |
| 4 μl PEG8000 | 10% (w/v) |
| 0.5 μL RNase inhibitor | 20 U |
| 0.5 μL T4 RNA ligase 2, truncated | 100 U |
| 8.5 μl RNase-free H₂O | |
| Ad 20 μl | |

| | |
|---|---|
| 1 μl RNA | 10 μM |
| 2 μl 3′ adapter “Universal miRNA | 20 μM |
| Cloning Linker” | |
| 2 μL 10 × T4 RNA ligase buffer | 1× |
| without ATP | |
| 4 μl PEG8000 | 10% (w/v) |
| 0.5 μL RNase inhibitor | 20 U |
| 0.5 μL T4 RNA ligase 2, truncated | 100 U |
| 10.5 μl RNase-free H₂O | |
| Ad 20 μl | |

| | |
|---|---|
| 2 μl RNA | 20 μM |
| 2 μl 3′ adapter “Universal miRNA | 20 μM |
| Cloning Linker” | |
| 2 μL 10 × T4 RNA ligase buffer | 1× |
| without ATP | |
| 4 μl PEG8000 | 10% (w/v) |
| 0.5 μL RNase inhibitor | 20 U |
| 0.5 μL T4 RNA ligase 2, truncated | 100 U |
| 9.5 μl RNase-free H₂O | |
| Ad 20 μl | |

| | |
|---|---|
| 4 μl RNA | 40 μM |
| 2 μl 3′ adapter “Universal miRNA | 20 μM |
| Cloning Linker” | |
| 2 μL 10 × T4 RNA ligase buffer | 1× |
| without ATP | |
| 4 μl PEG8000 | 10% (w/v) |
| 0.5 μL RNase inhibitor | 20 U |
| 0.5 μL T4 RNA ligase 2, truncated | 100 U |
| 7.5 μl RNase-free H₂O | |
| Ad 20 μl | |

The reaction is incubated at 25° C. for 4 h or 18° C. overnight. Note: ligation reaction must be performed in the absence of ATP. Heat inactivation: 65° C. 20 min.

### 5′ Adapter Ligation

Denature the 5′ adapter RNA oligo (10 μM, in RNase-free water) at 65° C. for 30 sec, immediately chill on ice.

Add to 20 μl 3′ Adapter-RNA-Mix to:

| | |
|---|---|
| 4 bzw. 2 μl 5′ Adapter RNA oligo | 20 μM |
| 1 μL 10 × T4 RNA ligase buffer | 1× |
| 3 μl 10 mMATP | 0.6 mM |
| 2 μl PEG8000 | 10% (w/v) |
| 0.5 μL RNase inhibitor | 20 U |
| 1 μL T4 RNA ligase 1 | 10 U |
| Ad 30 μl | |

The reaction is incubated at 25° C. for 4 h or 18° C. overnight. Heat inactivation: 65° C. for 15 minutes. Note: the 3′ end of the small RNA has already been ligated to the 3′ adapter that has an amine group at the 3′ end, and could no longer take part in the ligation reaction; thus its 5′ end could be ligated to an RNA oligo in the presence of ATP.

Reverse Transcription (1st Strand cDNA Synthesis)

Mix and briefly centrifuge each component before use.

Combine the following in a 0.2-ml PCR tube:

| | |
|---|---|
| Adapter-ligated targetRNA | 15 μl |
| P7 RT-Anti UCL primer 2 μM | 2 μl |
| 10 mM dNTP mix | 2 μl |
| DEPC-treated water to 20 μl | 1 μl |

Incubate at 65° C. for 5 min, then place on ice for at least 1 min.

Prepare the following cDNA Synthesis Mix, adding each component in the indicated order.

| | |
|---|---|
| 10 × RT buffer | 4 μl |
| 25 mM MgCl₂ | 8 μl |
| 0.1M DTT | 4 μl |
| RNaseOUT (40 U/μl) | 2 μl |
| Superscript III RT (200 U/μl) | 2 μl |

Add 20 μl of cDNA Synthesis Mix to each RNA/primer mixture, mix gently, and collect by brief centrifugation. Incubate at: 50 min at 50° C. Terminate the reactions at 85° C. for 5 min. Chill on ice. Collect the reactions by brief centrifugation. cDNA synthesis reaction can be stored at −20° C. or used for PCR immediately.

2nd Strand cDNA Synthesis

Prepare the following PCR Mix:

| Component | Amount | Final concentration |
|---|---|---|
| 1st strand cDNA | 18 μl | |
| 10 × PCR Buffer, —Mg | 3 μl | 1× |
| 50 mM MgCl₂ | 0.9 μl | 1.5 mM |
| 10 mM dNTP mix | 1.5 μl | 0.5 mM |
| P5 2nd strand primer 2 μM | 1.5 μl | 0.1 μM |
| Taq DNA Polymerase (5 U/μL) | 0.2 μl | 1U |
| RNase-free H₂O ad 30 μl | 4.9 μl | |

Place samples in PCR analyzer and execute the following cycling program:

Denature: 95° C., 3 min

Annealing: 65° C. 10 sec, Decrease 65° C.-55° C. at 0.1° C./sec

Elongation: 72° C. 3 min

Cool to 4° C. ∞

Store at −20° C. until PCR enrichment.

PCR enrichment

Prepare the following PCR Mix:

| Component | Amount | Final concentration |
|---|---|---|
| 5 × Phusion HF buffer | 5 μl | 1× |
| 10 mM dNTPs | 0.5 μl | 200 μM |
| 10 μM forward primer (indexed) | 1.25 μl | 0.5 μM |
| 10 μM reverse primer (indexed) | 1.25 μl | 0.5 μM |
| RT product (cDNA) | 10 μl | |
| Phusion Hot Start Flex DNA Polymerase | 0.25 μl | 1 unit/50 μl |
| Nuclease-free water ad 25 μl | 6.75 μl | |

Place samples in PCR analyzer and execute the following cycling program

Initiation: Denature 98° C., 30 Seconds

15 Cycles:
1. Denature 98° C., 10 Seconds
2. Annealing 72° C., 20 Seconds*
3. Elongation 72° C., 15 Seconds

Final Extension 72° C., 3 minutes

Hold 4-10° C. *To determine the optimal annealing temperature for a given set of primers, use of the NEB Tm Calculator is highly recommended.

The remaining RT product can be stored at −20° C.

Readout

Separate the PCR product on a 2% agarose gel using an appropriate molecular weight marker. Note: The accurate ligated and amplified Library has a size of 233 bases. Cut the band and gel-purify the product with Qiagen MinElute kit.

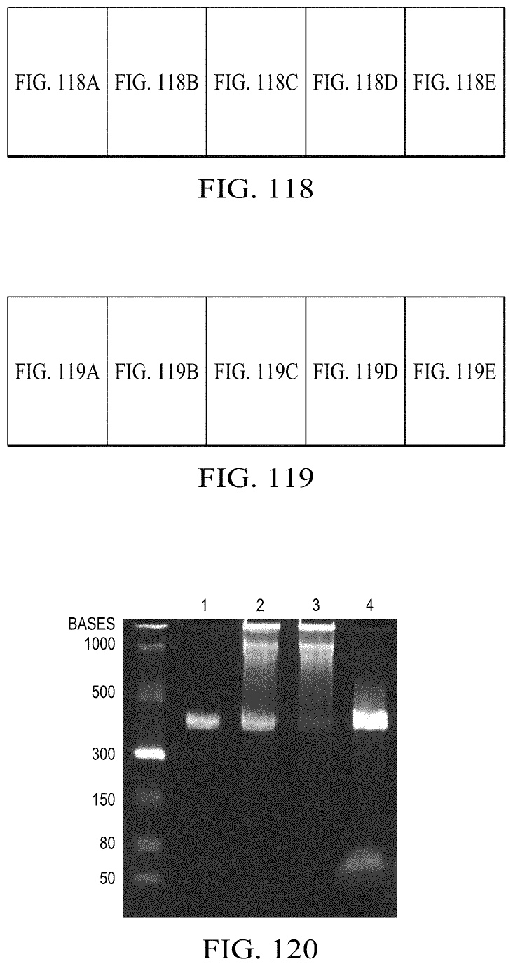

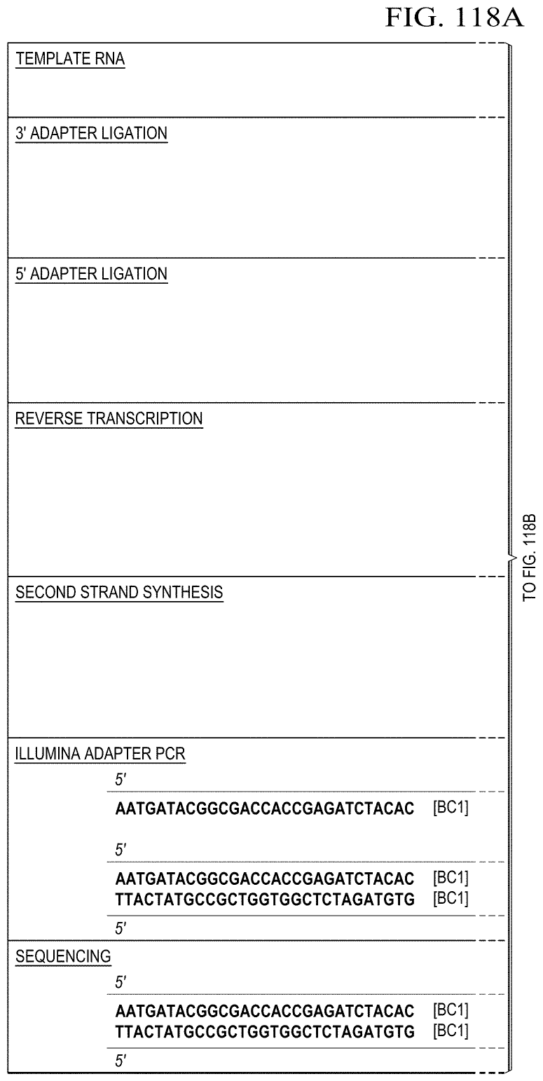

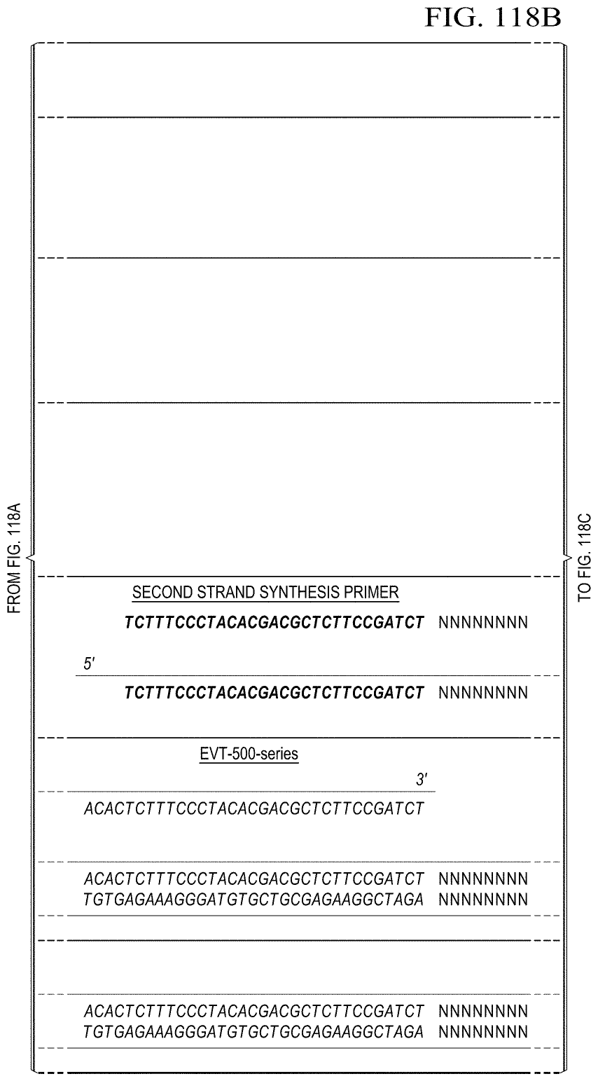

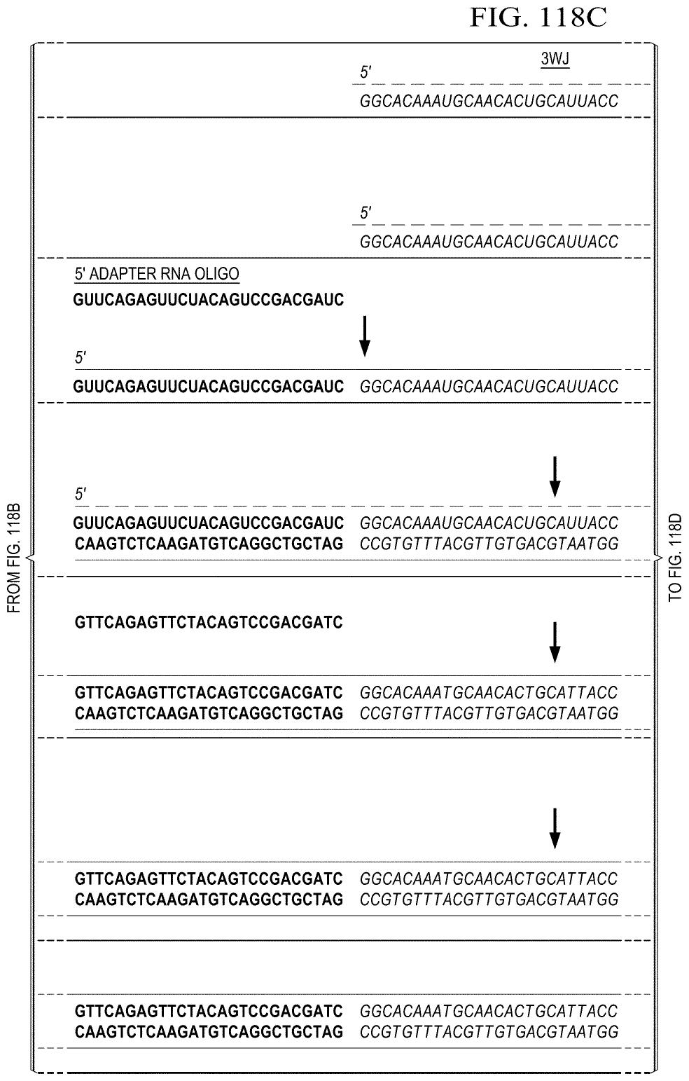

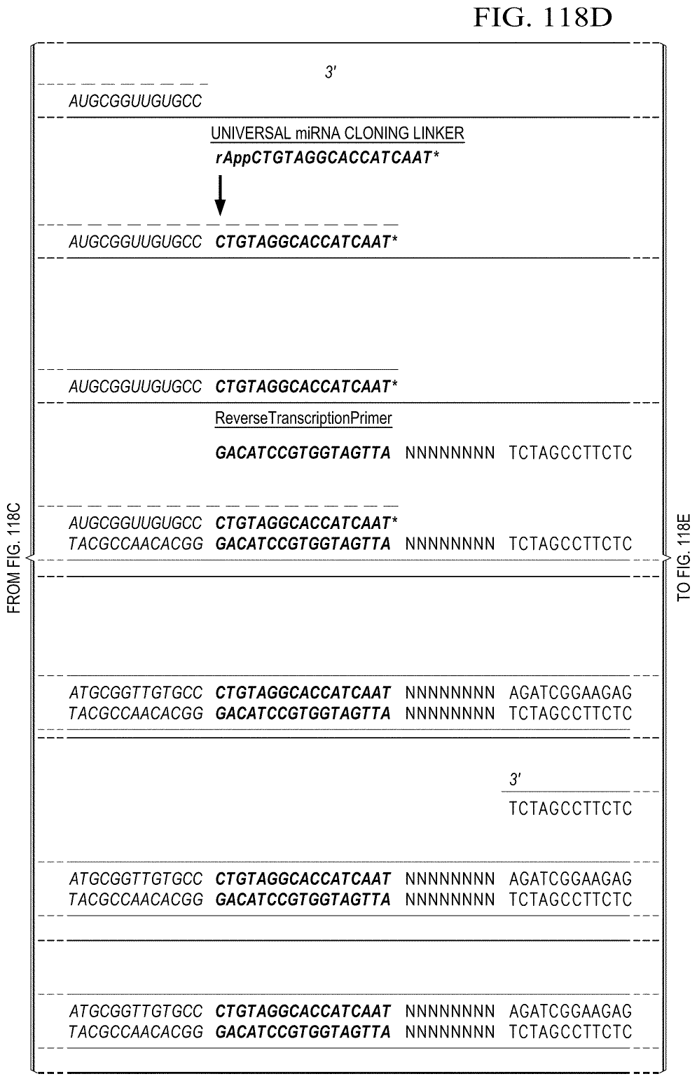

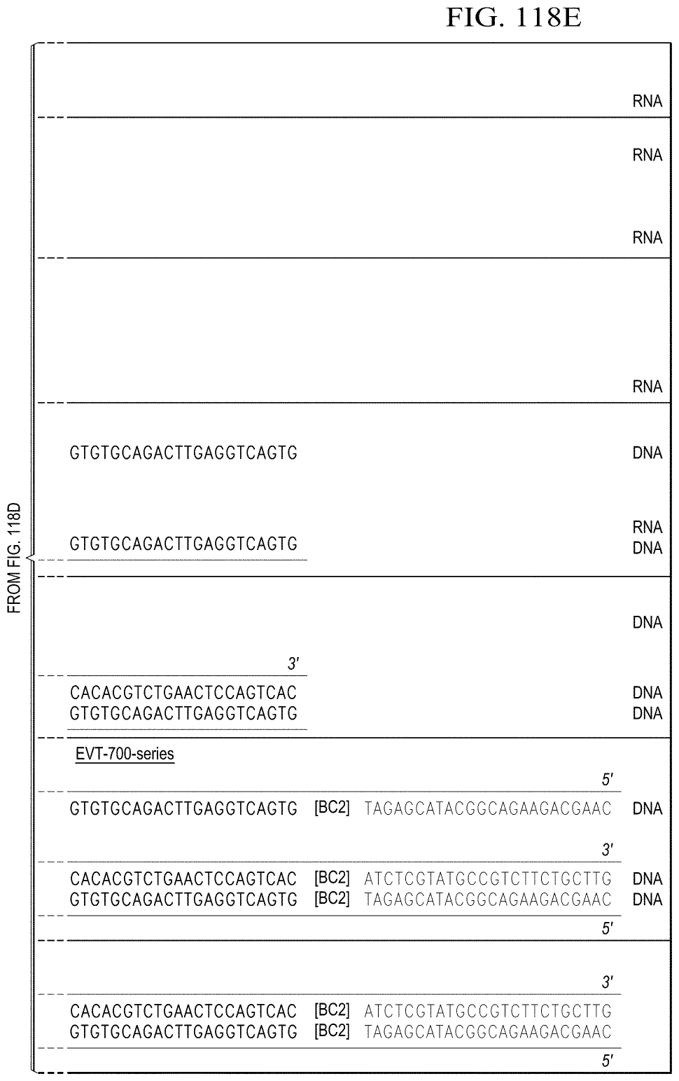

Subject the purified fragment to direct Sanger Sequencing (at a Provider of Choice) using either “Quanti qPCR 1_fw” or “Quanti qPCR 1_rv” primers. The steps and sequences involved are shown in FIG. 118.

### Example 26: Alternate Procedure for Producing Illumina Small RNA-Seq Library

An alternate procedure for producing the desired RNA library was developed that included the further step of ligating the 5′ adapter to the target RNA. The principal steps of the alternate method were: 1) Ligation of 3′-adapter to target RNA; 2) Phosphorylation of 5′ end of target RNA; 3) Ligation of 5′-adapter to target RNA; 4) 1st and 2nd strand cDNA synthesis from ligated adapters; 5) Incorporation and amplification of barcoded Illumina primers by PCR.

To effect this additional step, T4 Polynucleotide Kinase (NEB) was included among the reagents. The additional phosphorylation step was performed as follows:

Phosphorylation with T4 Polynucleotide Kinase

For non-radioactive phosphorylation, use up to 300 pmol of 5′ termini

| 20 μl 3′ Adapter-RNA-Mix | 200/400/800 pmol | |
|---|---|---|
| 4 μL 10 × T4 RNA ligase buffer | 1 × (1 mM DTT) | |
| 4 μl 10 mM ATP | 1 | mM |
| 3.6 μl DTT 0.1M | 9 | mM |
| 1 μl T4 Polynucleotide Kinase | 10 | U |

7.4 μl RNase-free H2O
Ad 40 μl

Incubate at 37° C. for 30 minutes. Fresh buffer is required for optimal activity (loss of DTT due to oxidation lowers activity).

Also, during the subsequent 5′ adapter ligation step, 40 μl phosphorylated 3′ Adapter-RNA-Mix instead of 20 μl was used.

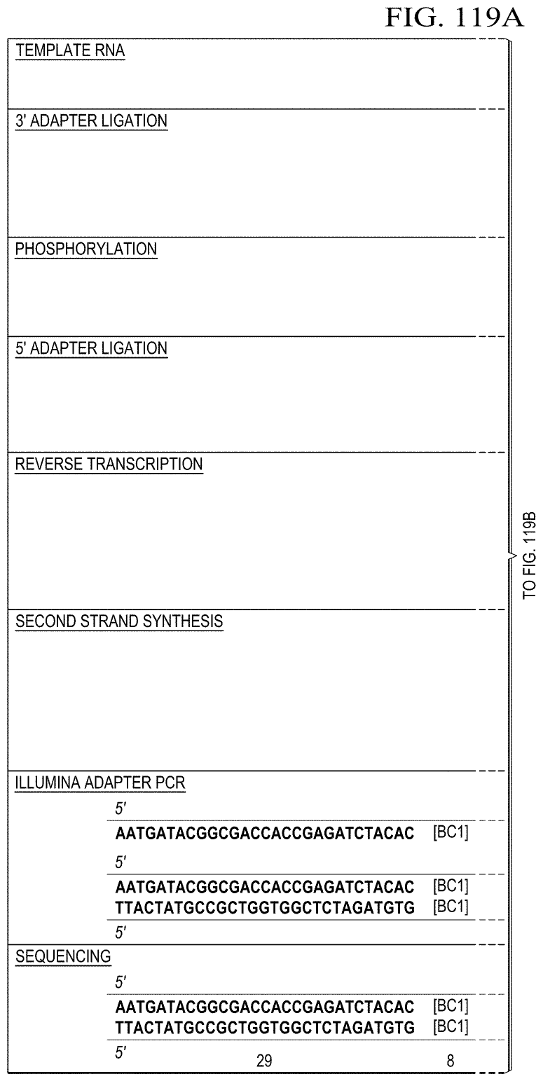

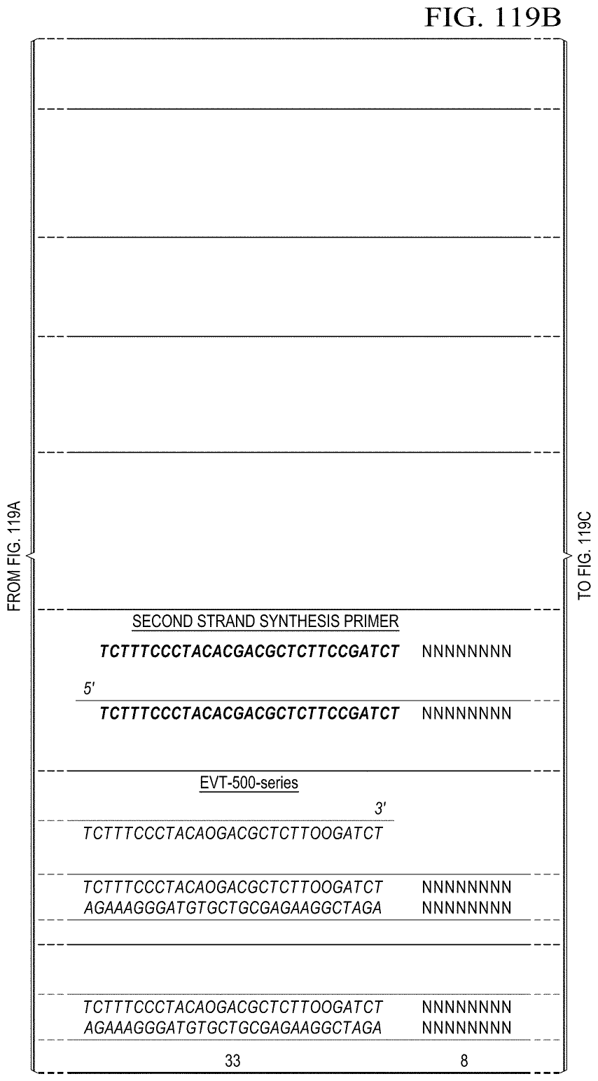

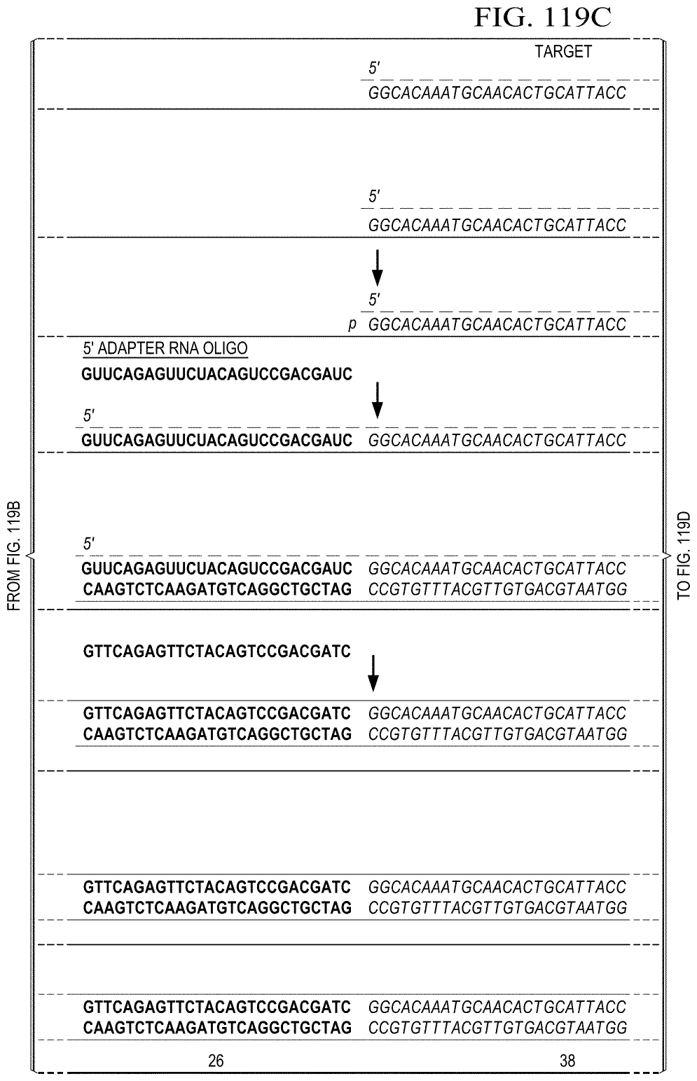

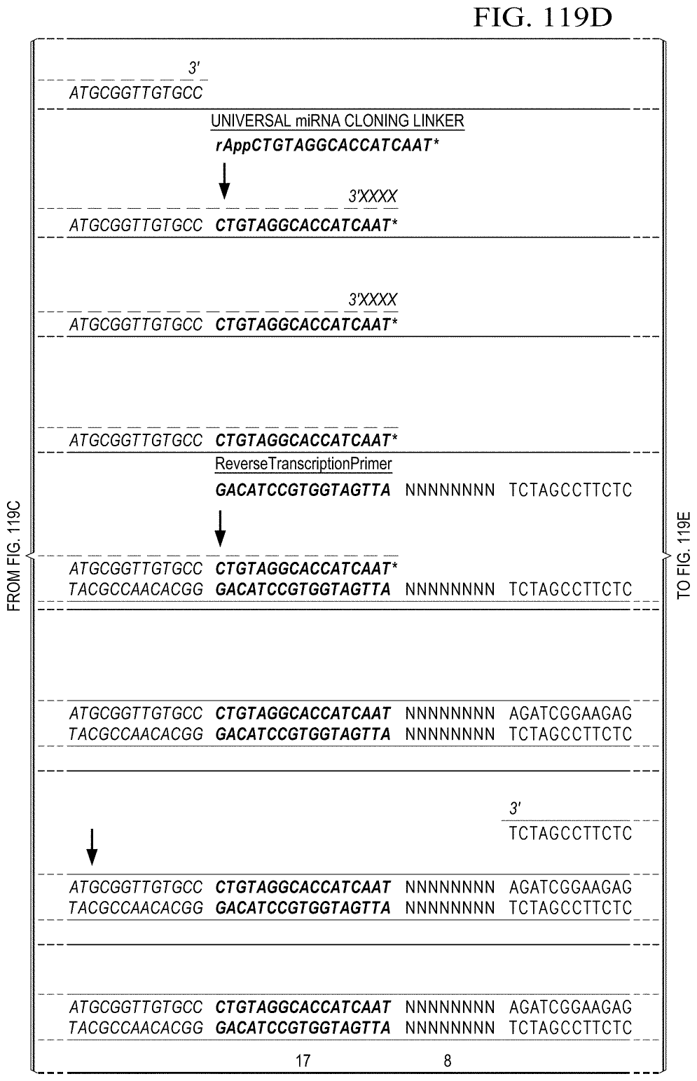

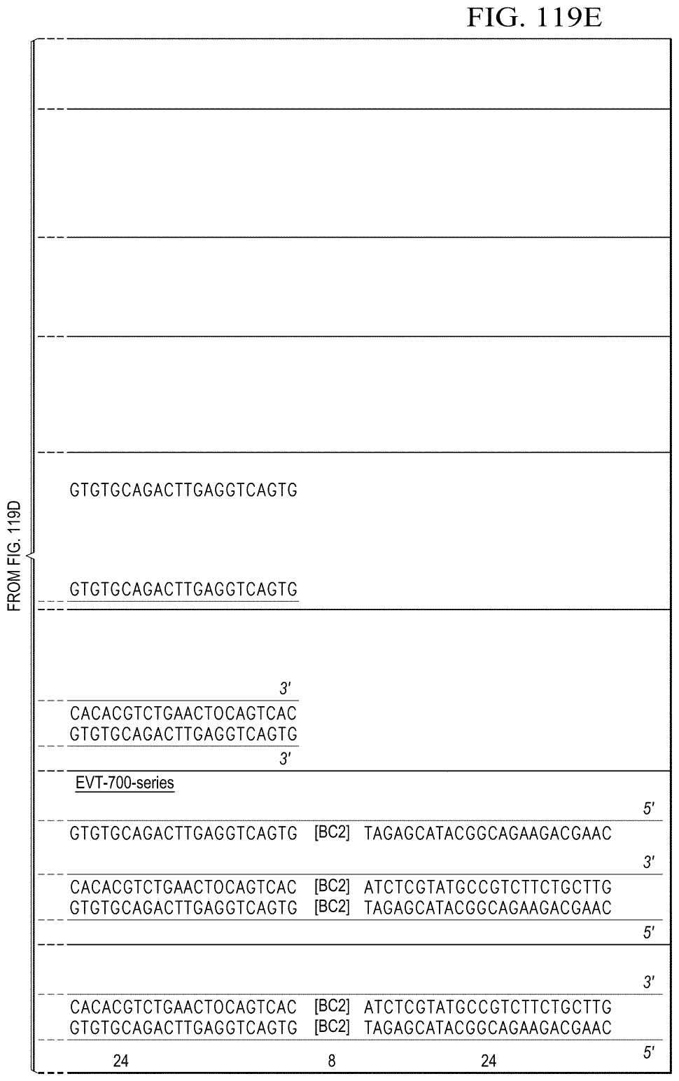

The steps and sequences involved in two methods of production of the library are shown in FIGS. 118 and 119.

### Example 27: Preparation and Immobilization of DELs (DNA-Encoded Libraries)

Sequences HTT41CAG and HTT17CAG were successfully synthesized and refolded after incubation for 2 h in the selection buffer described below. This was confirmed by native PAGE (results not shown). Native PAGE: Denatured at 95° C. for 3 min, snap cooled on ice for 3 min and refolded at 37° C. for 30 min (10 mM Tris-HCl, pH 8.0, 75 mM KCl, and 3 mM MgCl₂). About 50% of the RNA targets were immobilized on neutravidin resin. The RNA targets were stable under selection conditions after the following improvements: apply stain after gel electrophoresis. Decreasing the concentration of ssDNA and Rnase inhibitor during immobilization also helped.

### Selection Conditions

DEL specifics: DEL Set 1=610 DEL libraries, 5.521 billion compounds in total; DEL Set 2=205 DEL libraries, 70 million compounds in total (sets screened separately)

Selection rounds: 3-4

Selection mode: Target immobilized

Capture resin: Neutravidin resin

Target amount: 100 pmol

Immobilization buffer composition: NMR buffer, 0.1% Tween-20, 0.03 mg/ml ssDNA, 2 mM Vanadyl ribonucleoside complexes.

Selection buffer composition: 50 mM Tris-HCl (pH 8), 75 mM KCl, 3 mM or 10 mM MgCl₂, 0.1% Tween-20, 0.3 mg/ml ssDNA, 20 mM Vanadyl ribonucleoside complexes.

Volume, temperature, and time: 100 uL, RT, 1 hour

### Wash Conditions

Buffer composition: 50 mM Tris-HCl (pH 8), 75 mM KCl, 3 mM or 10 mM MgCl₂.

Number and volume: 2×200 uL

Temperature and time: RT, fast

### Elution Conditions:

Elution mode: Heat elution

Buffer composition: 50 mM Tris-HCl (pH 8), 75 mM KCl, 3 mM or 10 mM MgCl₂.

Volume, temperature, and time: 80 uL; 80° C.; 15 minutes.

Stability of the RNA complexes was confirmed by incubation in the selection buffer for 2 h at room temperature. The refolded RNA was successfully immobilized on the resin.

| | RNA Input | RNA Flow | RNA on | % of total |
|---|---|---|---|---|
| Sample | (ng) | Through (ng) | resin (ng) | immobilized |
| HTT17CAG | 2000 | 802.5 | 1197.5 | 60% |
| HTT41CAG | 500 | 138.5 | 361.5 | 72% |

Conclusions:

After decreasing the concentration of ssDNA and Rnase inhibitor during immobilization: 50% refolded HTT17CAG was adsorbed on Neutravidin resin; after incubation with DEL compounds, refolded HTT17CAG was recovered from Neutravidin resin; the target is now ready for affinity selection.

### Example 28: Surface Plasmon Resonance Experiments

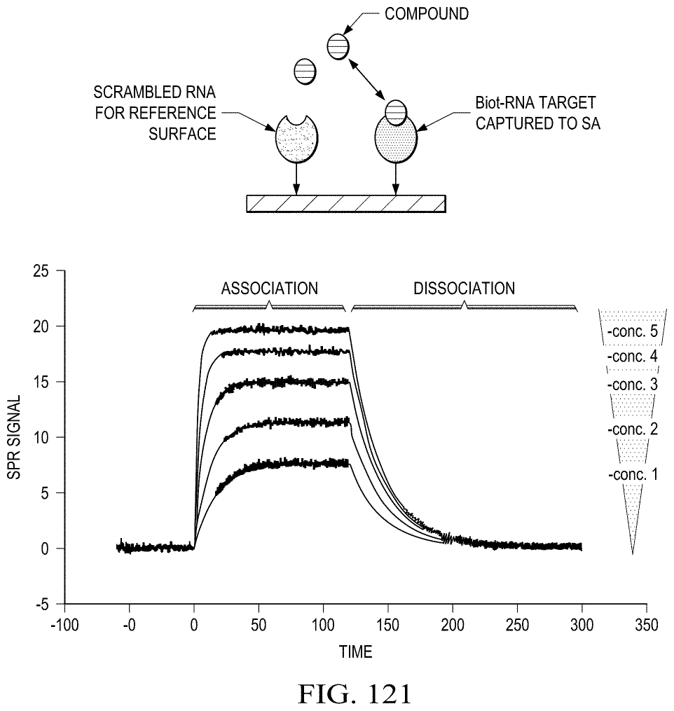

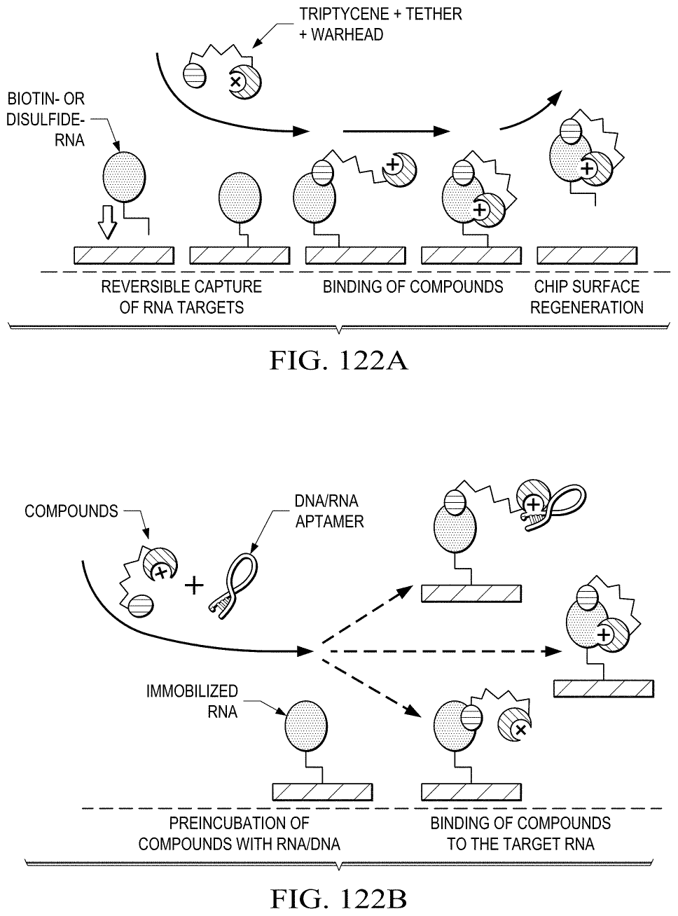

FIGS. 121 and 122 show possible methods of employing surface plasmon resonance (SPR) to screen ligands and hook and click constructs for binding to a target RNA of interest. SPR is especially useful for monitoring biomolecular interactions in real time. Typically, target species and unrelated control are immobilized to a sensor chip, then analytes (compounds/fragments) are flowed over the surface. Binding of the compound to target species results in increase of SPR signal (association phase). Washing away bound compound with buffer results in a decrease of SPR signal (dissociation phase). Fitting of sensorgrams recorded at different compound concentrations is performed to an appropriate interaction model. The method allows extraction of kinetic parameters (kₐ, k_{d}→K_{D}). Requirements/limitations include that the kₐ/k_{d} values be in reasonable ranges; and the target size must not be too large (<100 kDa). It is an excellent method to screen fragments and profile or validate hits. BC4000 may be used for primary screening (up to 4,000 data pts/week). Biacore T200 is suitable for hit profiling and validation.

In the PEARL-seq context, SPR allows monitoring binding of “hooks” to DNA/RNA aptamers. The target species is immobilized to sensor chip, analytes (i.e. hooks) are flowed over surface (association phase), DNA/RNA aptamer is flowed over surface (plateau phase), competitor compound is washed over surface (dissociation phase), thus yielding binding data. The requirements/limitations are that, again, kₐ/k_{d} values must be in reasonable ranges and fitting for their respective purpose. Furthermore, the target size must be <100 kDa. In addition, steps 1 and 2 need to be in place (tested first) in order to enable setup. A competitor with fitting affinity will also be needed.

With the goal of identifying interaction partners (RNA/DNA) that bind to capture RNA (3WJ), the following steps are contemplated:

Use biotinylated capture-RNA (bio3WJ) to fold into secondary structure;

Allow binding of warhead triptycene ligands;

Fish interacting RNA/DNA's by covalent linking to warhead;

Precipitate complexes via binding of bio3WJ to streptavidin beads;

Wash and elute; and

Library generation from eluate and sequencing.

A protocol for smooth generation of cell lysates or RNA preps will be required. One exemplary protocol would involve the following steps:

Preparation of RT-qPCR-Ready Cell Lysates:

MDCK-London cells in 24-well plates were washed once with PBS (1 mL/well). Cell lysatesare prepared by exposing cell monolayers to 200 mL/well of Cell-Lysis (CL) Buffer. The final formulation of CL Buffer consist of 10 mM Tris-HCl pH 7.4, 0.25% Igepal CA-630, and 150 mM NaCl. CL Buffer is freshly prepared from appropriate stock solutions. All reagents are molecular biology grade and dilutions are made with DEPC-treated water (351-068-721; Quality Biological, Inc.). For certain experiments, CL Buffer also includes MgCl₂ (M1028; Sigma) or RNasin Plus RNase Inhibitor (N2615; Promega). Cells are exposed for appropriate times (typically 5 min for CL Buffer). The resulting lysates are carefully collected without disturbing the cell monolayer remnants and either analyzed immediately or stored frozen. See, e.g. Shatzkes et al., “A simple, inexpensive method for preparing cell lysates suitable for downstream reverse transcription quantitative PCR,” Scientific Reports 4, Article number: 4659 (2014).

Simple Lysis buffer: using Igepal CA-630 and 150 mM NaCl; generates crude cell lysate, contains still everything (no polyA-enrichment or protein removal).

Different Protocols possible: smallRNA workflow: Adapter ligation, cDNA-synthesis, Library (small clusters); or total RNA workflow: random primed w/wo RiboZero, standard library prep (normal clusters).

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1-17. (canceled)

18. A method of identifying a small molecule that binds to and modulates the function of a target RNA, comprising the steps of: screening a compound for binding to the target RNA; and analyzing the results by an RNA binding assay to identify a location of a binding site of the compound in a primary sequence of the target RNA; wherein the compound is of Formula I: 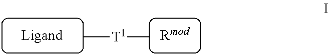
or a pharmaceutically acceptable salt thereof, wherein:
Ligand is a small molecule RNA binder selected from an optionally substituted aminoglycoside or an analog thereof,
T¹ is a bivalent tethering group; and
R^{mod} is an RNA-modifying moiety; wherein R^{mod} reacts with an unconstrained 2′-hydroxyl group of a target RNA to which Ligand binds to produce a 2′-covalently modified RNA.

19. The method of claim 18, wherein the Ligand is a kanamycin, a paromomycin, or a neomycin, or an analog thereof, wherein the Ligand is optionally substituted with 1, 2, or 3 substituents.

20. The method of claim 18, wherein the Ligand is kanamycin A, paromomycin, or neomycin B, or an analog thereof, wherein the Ligand is optionally substituted with 1, 2, or 3 substituents.

21. The method of claim 18, wherein T¹ is selected from the following: 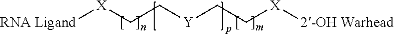
X is —CO—, —SO₂—, —NH—, —N(alkyl)-, —S—, —O—, -triazole-, -arylene-, or -heteroarylene-;
n is 1, 2, 3, 4, 5; n can be 0 when X is CO or SO₂ or -arylene-;
Y is a bond, —O—, —S—, —SO—, —SO₂—, —NH—, —N(alkyl)-, —CH₂—, -arylene-, or -heteroarylene-;
p is 0, 1, 2, 3, 4, 5, 6, 7, 8, 9, 10, 11, or 12;
m is 1, 2, 3, 4, or 5; m can be 0 when X is CO or SO₂ or -arylene- or -heteroarylene-; 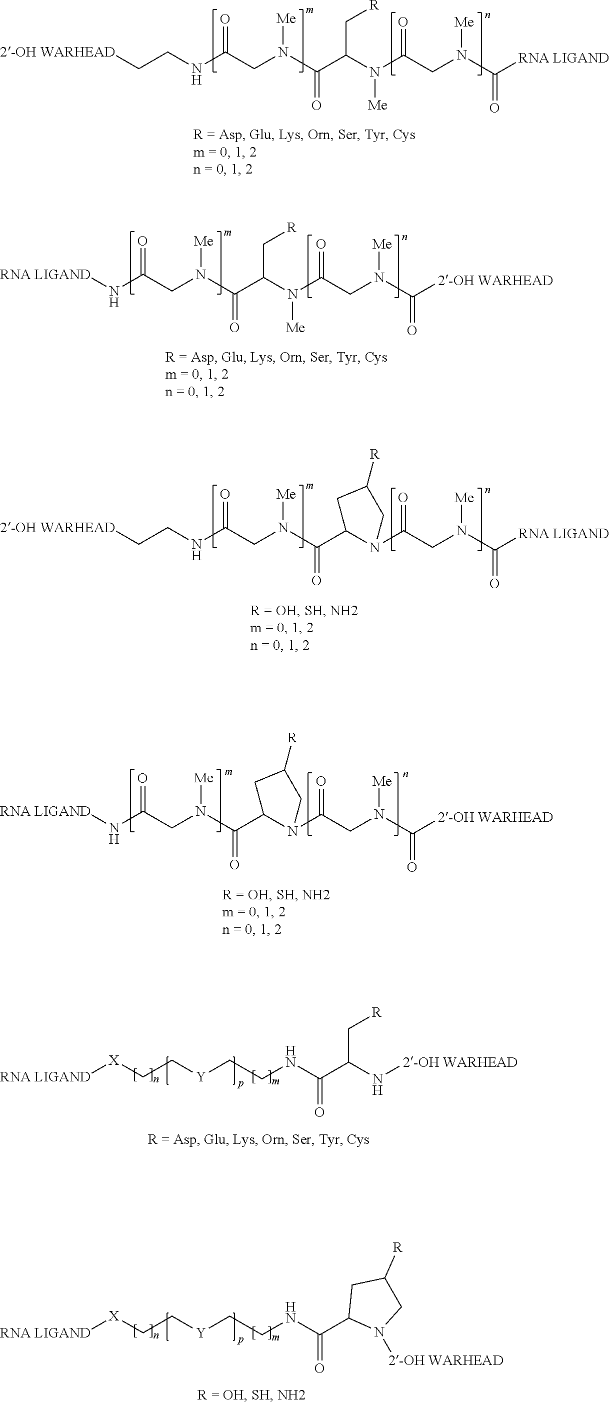 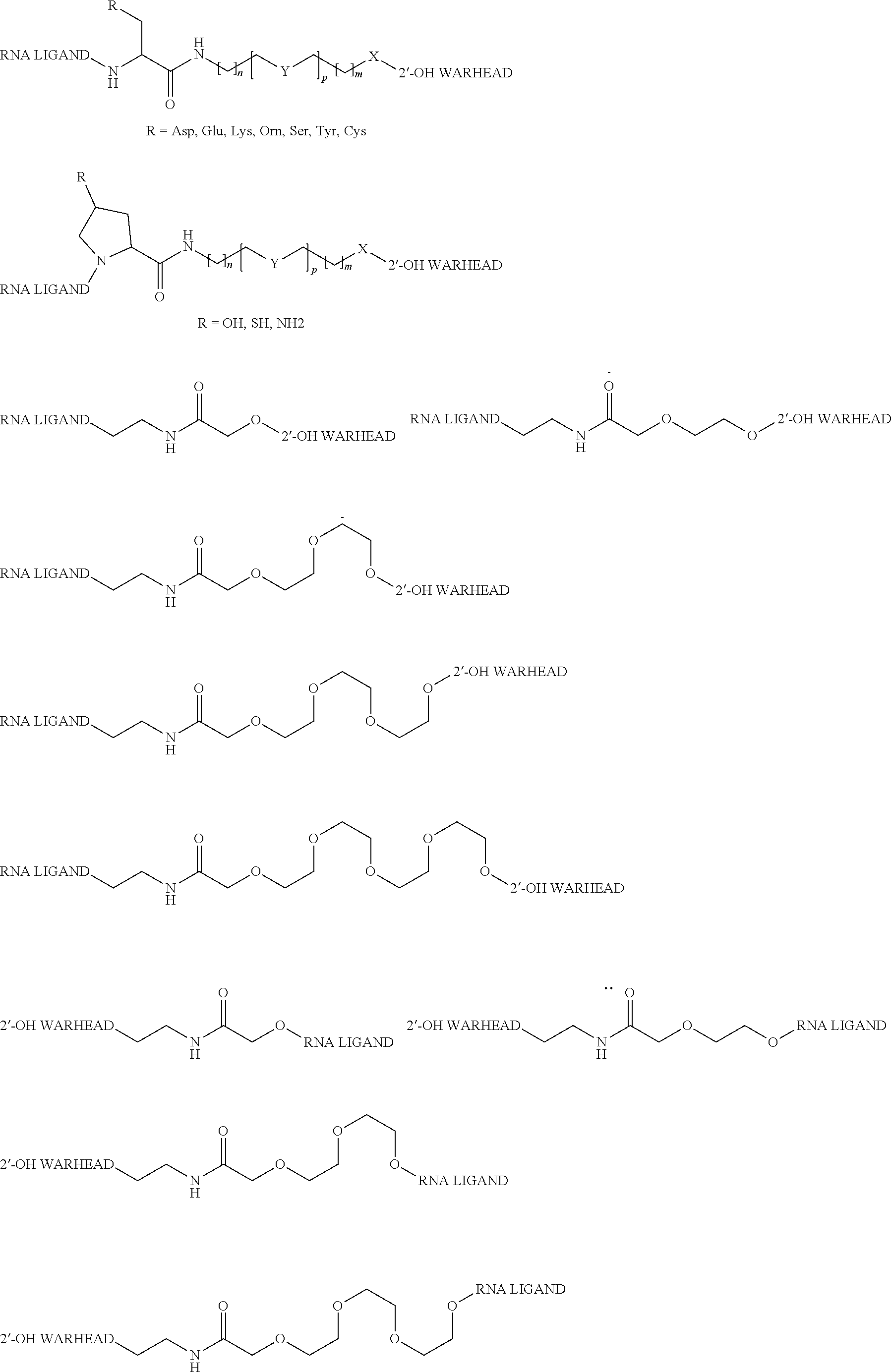 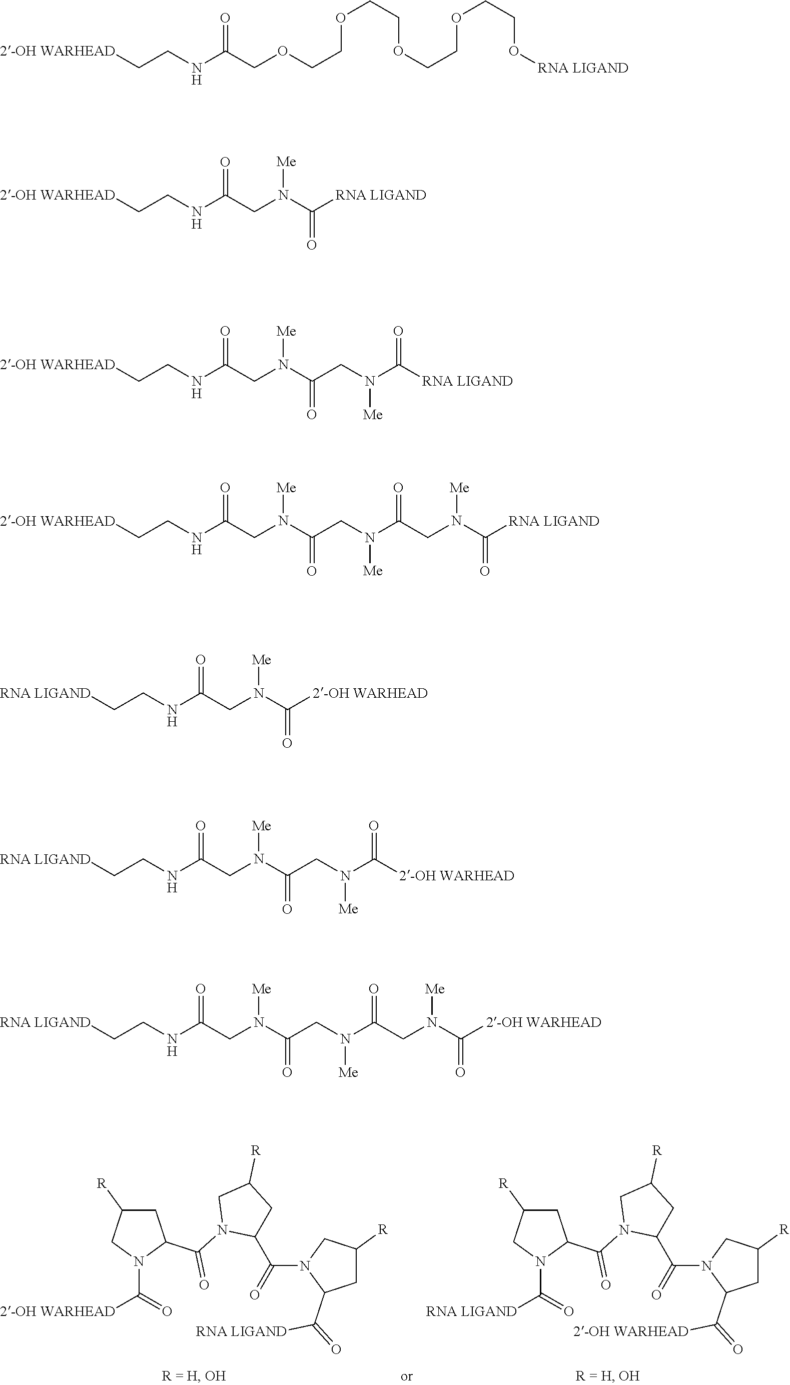
wherein RNA LIGAND indicates a covalent bond to Ligand and 2′-OH WARHEAD indicates a covalent bond to R^{mod}.

22. The method of claim 18, wherein T¹ is selected from a polyethylene glycol (PEG) group, an optionally substituted C₁₋₁₂ aliphatic group, or a peptide comprising 1-8 amino acids.

23. The method of claim 18, wherein R^{mod} is selected from sulfonyl halides, acyl imidazoles, aryl esters, heteroaryl esters, epoxides, alkyl halides, benzyl halides, and isocyanates.

24. The method of claim 18, wherein R^{mod} is selected from 1-methyl-7-nitroisatoic anhydride (1M7), benzoyl cyanide (BzCN), 2-methylnicotinic acid imidazolide (NAI), and 2-methyl-3-furoic acid imidazolide (FAI).

25. A method of modulating the activity of a target RNA in a biological sample, comprising the step of contacting the biological sample with a compound of Formula I: 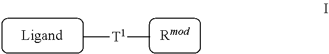
or a pharmaceutically acceptable salt thereof, wherein:
Ligand is a small molecule RNA binder selected from an optionally substituted aminoglycoside or an analog thereof,
T¹ is a bivalent tethering group; and
R^{mod} is an RNA-modifying moiety; wherein R^{mod} reacts with an unconstrained 2′-hydroxyl group of a target RNA to which Ligand binds to produce a 2′-covalently modified RNA.

26. The method of claim 25, wherein the target RNA is an mRNA, and the method comprises modulating the downstream protein expression associated with the mRNA.

27. The method of claim 26, wherein the compound irreversibly inhibits the activity of the mRNA.

28. The method of claim 26, wherein the Ligand is a kanamycin, a paromomycin, or a neomycin, or an analog thereof, wherein the Ligand is optionally substituted with 1, 2, or 3 substituents.

29. The method of claim 26, wherein the Ligand is kanamycin A, paromomycin, or neomycin B, or an analog thereof, wherein the Ligand is optionally substituted with 1, 2, or 3 substituents.

30. The method of claim 26, wherein T¹ is selected from the following: 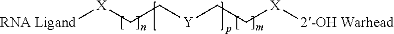
X is —CO—, —SO₂—, —NH—, —N(alkyl)-, —S—, —O—, -triazole-, -arylene-, or -heteroarylene-;
n is 1, 2, 3, 4, 5; n can be 0 when X is CO or SO₂ or -arylene-;
Y is a bond, —O—, —S—, —SO—, —SO₂—, —NH—, —N(alkyl)-, —CH₂—, -arylene-, or -heteroarylene-;
p is 0, 1, 2, 3, 4, 5, 6, 7, 8, 9, 10, 11, or 12;
m is 1, 2, 3, 4, or 5; m can be 0 when X is CO or SO₂ or -arylene- or -heteroarylene-; 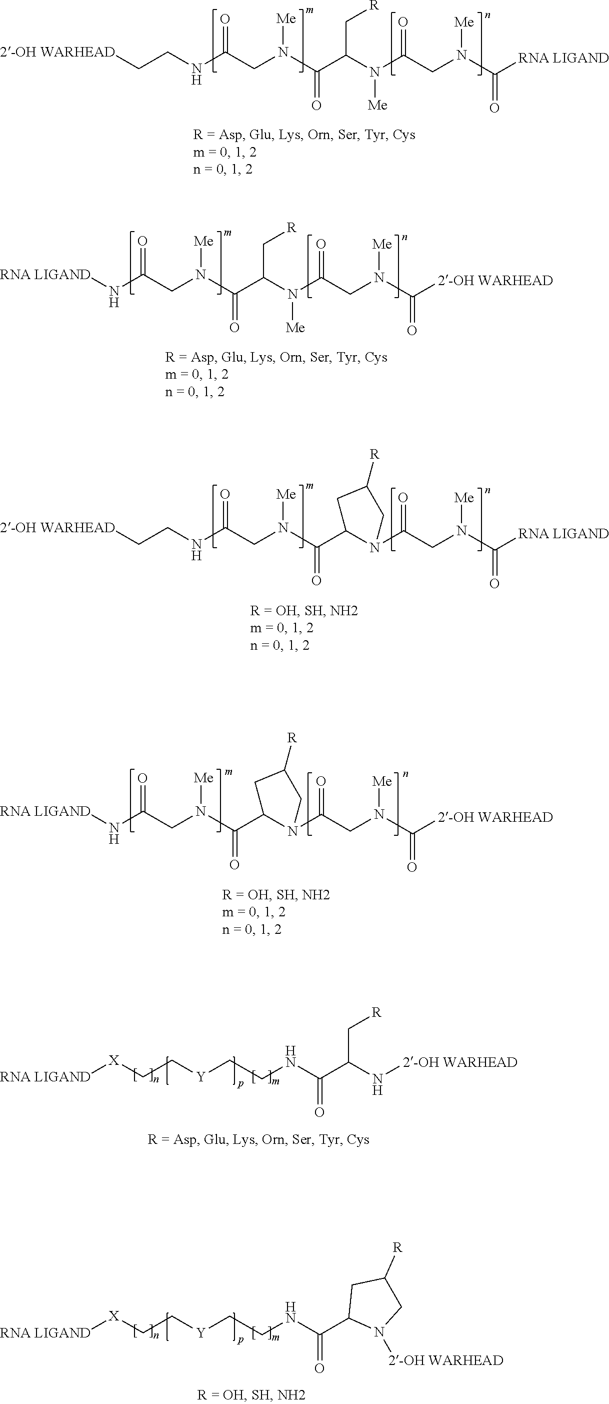 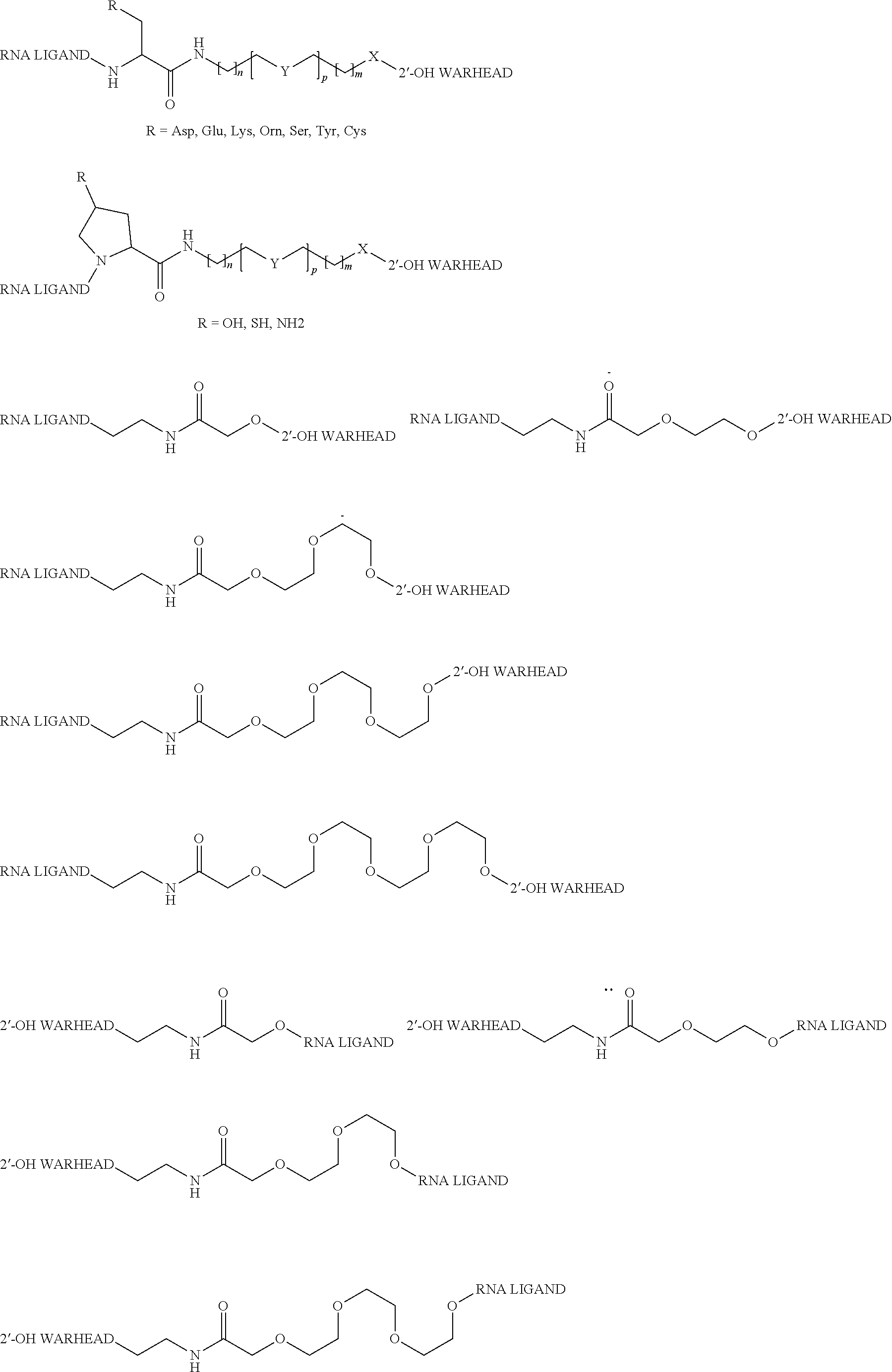 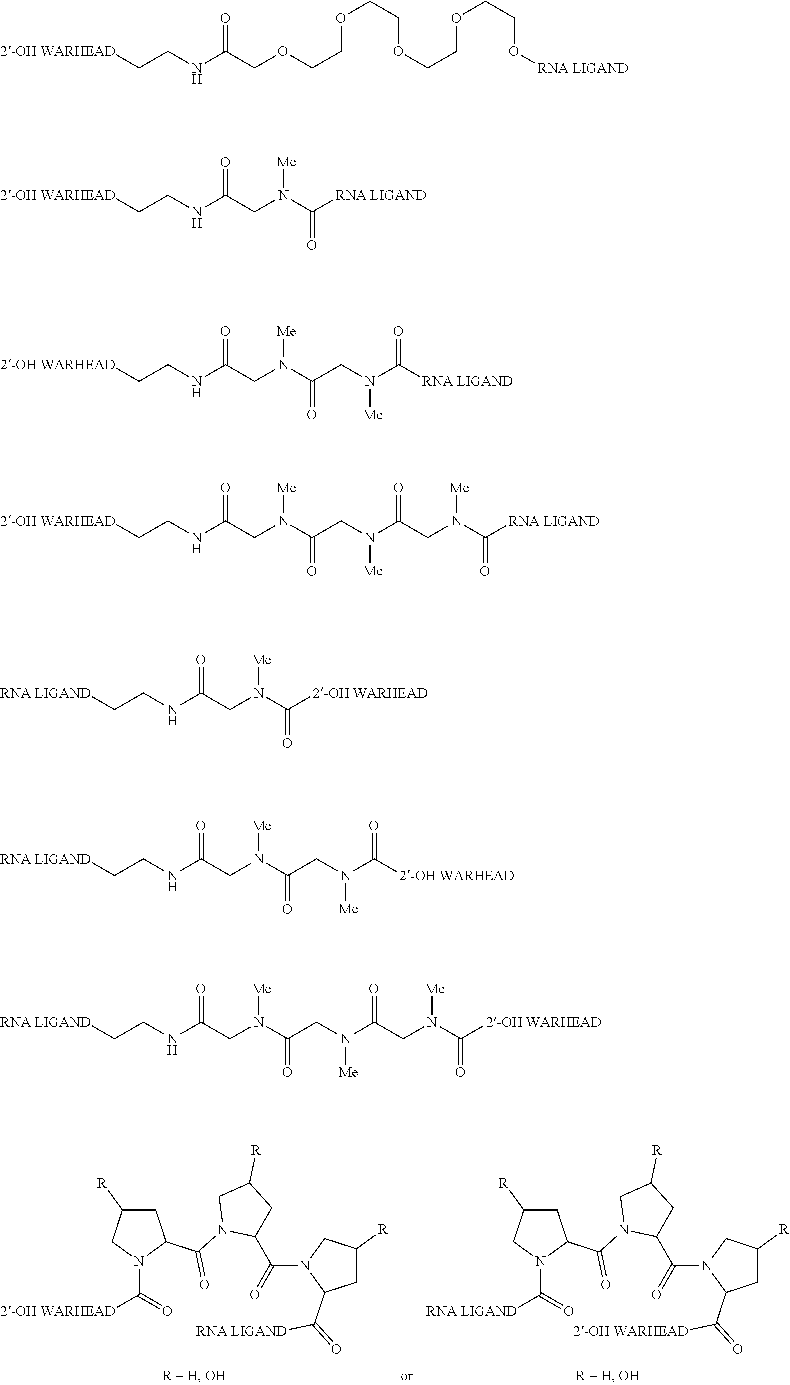
wherein RNA LIGAND indicates a covalent bond to Ligand and 2′-OH WARHEAD indicates a covalent bond to R^{mod}.

31. The method of claim 26, wherein T¹ is selected from a polyethylene glycol (PEG) group, an optionally substituted C₁₋₁₂ aliphatic group, or a peptide comprising 1-8 amino acids.

32. The method of claim 26, wherein R^{mod} is selected from sulfonyl halides, acyl imidazoles, aryl esters, heteroaryl esters, epoxides, alkyl halides, benzyl halides, and isocyanates.

33. The method of claim 26, wherein R^{mod} is selected from 1-methyl-7-nitroisatoic anhydride (1M7), benzoyl cyanide (BzCN), 2-methylnicotinic acid imidazolide (NAI), and 2-methyl-3-furoic acid imidazolide (FAI).


SEQUENCE LISTING (65 sequences)

SEQ ID NO: 1 - DNA, 105 bp, Artificial Sequence
  Description of Artificial Sequence Synthetic polynucleotide
  misc_feature (1)..(105): This sequence may encompass 6-35 ‘cag’ repeating units
cagcagcagcagcagcagcagcagcagcagcagcagcagcagcagcagcagcagcagcagcagcagcagcagcagcagcagcagcagcagcagcagcagcagcag

SEQ ID NO: 2 - DNA, 108 bp, Artificial Sequence
  Description of Artificial Sequence Synthetic polynucleotide
  misc_feature (1)..(108): This sequence may encompass 9-36 ‘cag’ repeating units
cagcagcagcagcagcagcagcagcagcagcagcagcagcagcagcagcagcagcagcagcagcagcagcagcagcagcagcagcagcagcagcagcagcagcagcag

SEQ ID NO: 3 - DNA, 96 bp, Artificial Sequence
  Description of Artificial Sequence Synthetic oligonucleotide
  misc_feature (1)..(96): This sequence may encompass 14-32 ‘cag’ repeating units
cagcagcagcagcagcagcagcagcagcagcagcagcagcagcagcagcagcagcagcagcagcagcagcagcagcagcagcagcagcagcagcag

SEQ ID NO: 4 - DNA, 120 bp, Artificial Sequence
  Description of Artificial Sequence Synthetic polynucleotide
  misc_feature (1)..(120): This sequence may encompass 12-40 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 5 - DNA, 54 bp, Artificial Sequence
  Description of Artificial Sequence Synthetic oligonucleotide
  misc_feature (1)..(54): This sequence may encompass 4-18 ‘cag’ repeating units
cagcagcagcagcagcagcagcagcagcagcagcagcagcagcagcagcagcag

SEQ ID NO: 6 - DNA, 51 bp, Artificial Sequence
  Description of Artificial Sequence Synthetic oligonucleotide
  misc_feature (1)..(51): This sequence may encompass 7-17 ‘cag’ repeating units
cagcagcagcagcagcagcagcagcagcagcagcagcagcagcagcagcag

SEQ ID NO: 7 - DNA, 126 bp, Artificial Sequence
  Description of Artificial Sequence Synthetic polynucleotide
  misc_feature (1)..(126): This sequence may encompass 25-42 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 8 - DNA, 750 bp, Artificial Sequence
  Description of Artificial Sequence Synthetic polynucleotide
  misc_feature (1)..(750): This sequence may encompass 36-250 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 9 - DNA, 264 bp, Artificial Sequence
  Description of Artificial Sequence Synthetic polynucleotide
  misc_feature (1)..(264): This sequence may encompass 49-88 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 10 - DNA, 186 bp, Artificial Sequence
  Description of Artificial Sequence Synthetic polynucleotide
  misc_feature (1)..(186): This sequence may encompass 38-62 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 11 - DNA, 231 bp, Artificial Sequence
  Description of Artificial Sequence Synthetic polynucleotide
  misc_feature (1)..(231): This sequence may encompass 33-77 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 12 - DNA, 258 bp, Artificial Sequence
  Description of Artificial Sequence Synthetic polynucleotide
  misc_feature (1)..(258): This sequence may encompass 55-86 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 13 - DNA, 90 bp, Artificial Sequence
  Description of Artificial Sequence Synthetic oligonucleotide
  misc_feature (1)..(90): This sequence may encompass 21-30 ‘cag’ repeating units
cagcagcagcagcagcagcagcagcagcagcagcagcagcagcagcagcagcagcagcagcagcagcagcagcagcagcagcagcagcag

SEQ ID NO: 14 - DNA, 360 bp, Artificial Sequence
  Description of Artificial Sequence Synthetic polynucleotide
  misc_feature (1)..(360): This sequence may encompass 38-120 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 15 - DNA, 189 bp, Artificial Sequence
  Description of Artificial Sequence Synthetic polynucleotide
  misc_feature (1)..(189): This sequence may encompass 47-63 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 16 - DNA, 159 bp, Artificial Sequence
  Description of Artificial Sequence Synthetic polynucleotide
  misc_feature (1)..(159): This sequence may encompass 6-53 ‘cgg’ repeating units
cggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcggcgg

SEQ ID NO: 17 - DNA, 111 bp, Artificial Sequence
  Description of Artificial Sequence Synthetic polynucleotide
  misc_feature (1)..(111): This sequence may encompass 5-37 ‘ctg’ repeating units
ctgctgctgctgctgctgctgctgctgctgctgctgctgctgctgctgctgctgctgctgctgctgctgctgctgctgctgctgctgctgctgctgctgctgctgctgctg

SEQ ID NO: 18 - DNA, 102 bp, Artificial Sequence
  Description of Artificial Sequence Synthetic polynucleotide
  misc_feature (1)..(102): This sequence may encompass 7-34 ‘gaa’ repeating units
gaagaagaagaagaagaagaagaagaagaagaagaagaagaagaagaagaagaagaagaagaagaagaagaagaagaagaagaagaagaagaagaagaagaa

SEQ ID NO: 19 - DNA, 111 bp, Artificial Sequence; identical to SEQ ID NO: 17
  Description of Artificial Sequence Synthetic polynucleotide
  misc_feature (1)..(111): This sequence may encompass 16-37 ‘ctg’ repeating units

SEQ ID NO: 20 - DNA, 160 bp, Artificial Sequence
  Description of Artificial Sequence Synthetic polynucleotide
  misc_feature (1)..(160): This sequence may encompass 9-32 ‘attct’ repeating units
attctattctattctattctattctattctattctattctattctattctattctattctattctattctattctattctattctattctattctattctattctattctattctattctattctattctattctattctattctattctattctattct

SEQ ID NO: 21 - DNA, 84 bp, Artificial Sequence
  Description of Artificial Sequence Synthetic oligonucleotide
  misc_feature (1)..(84): This sequence may encompass 7-28 ‘cag’ repeating units
cagcagcagcagcagcagcagcagcagcagcagcagcagcagcagcagcagcagcagcagcagcagcagcagcagcagcagcag

SEQ ID NO: 22 - DNA, 750 bp, Artificial Sequence
  Description of Artificial Sequence Synthetic polynucleotide
  misc_feature (1)..(750): This sequence may encompass 110-250 ‘ctg’ repeating units
ctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctgctg

SEQ ID NO: 23 - DNA, 22500 bp, Artificial Sequence
  Description of Artificial Sequence Synthetic polynucleotide
  misc_feature (1)..(22500): This sequence may encompass 800-4500 ‘attct’ repeating units
attctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattctattct

SEQ ID NO: 24 - DNA, 234 bp, Artificial Sequence
  Description of Artificial Sequence Synthetic polynucleotide
  misc_feature (1)..(234): This sequence may encompass 66-78 ‘cag’ repeating units
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 25 - RNA, 474 nt, Artificial Sequence
  Description of Artificial Sequence Synthetic polynucleotide
gcugccgggacggguccaagauggacggccgcucagguucugcuuuuaccugcggcccagagccccauucauugccccggugcugagcggcgccgcgagucggcccgaggccuccggggacugccgugccgggcgggagaccgccauggcgacccuggaaaagcugaugaaggccuucgagucccucaaguccuuccagcagcagcagcagcagcagcagcagcagcagcagcagcagcagcagcagcagcagcagcagcagcagcagcagcagcagcagcagcagcagcagcagcagcagcagcagcagcagcagcagcaacagccgccaccgccgccgccgccgccgccgccuccucagcuuccucagccgccgccgcaggcacagccgcugcugccucagccgcagccgcccccgccgccgcccccgccgccacccggcccggcuguggcugaggagccgcugcaccgacc

SEQ ID NO: 26 - RNA, 402 nt, Artificial Sequence
  Description of Artificial Sequence Synthetic polynucleotide
gcugccgggacggguccaagauggacggccgcucagguucugcuuuuaccugcggcccagagccccauucauugccccggugcugagcggcgccgcgagucggcccgaggccuccggggacugccgugccgggcgggagaccgccauggcgacccuggaaaagcugaugaaggccuucgagucccucaaguccuuccagcagcagcagcagcagcagcagcagcagcagcagcagcagcagcagcagcaacagccgccaccgccgccgccgccgccgccgccuccucagcuuccucagccgccgccgcaggcacagccgcugcugccucagccgcagccgcccccgccgccgcccccgccgccacccggcccggcuguggcugaggagccgcugcaccgacc

SEQ ID NO: 27 - RNA, 68 nt, Artificial Sequence
  Description of Artificial Sequence Synthetic oligonucleotide
gcagcagcagcagcagcagcagcagcagcagcagcagcagcagcagcaacagccgccaccgccgccgc

SEQ ID NO: 28 - RNA, 64 nt, Artificial Sequence
  Description of Artificial Sequence Synthetic oligonucleotide
gcagcagcagcagcagcagcagcagcagcaacagccgccaccgccgccgccgccgccgccgccu

SEQ ID NO: 29 - RNA, 66 nt, Artificial Sequence
  Description of Artificial Sequence Synthetic oligonucleotide
cagcagcagcagcagcagcagcagcagcagcagcagcagcagcagcagcagcagcagcagcagcag

SEQ ID NO: 30 - RNA, 57 nt, Artificial Sequence
  Description of Artificial Sequence Synthetic oligonucleotide
gagccuaaaacauaccagagaaaucuggagaggugaagaauacgaccaccuaggcuc

SEQ ID NO: 31 - RNA, 38 nt, Artificial Sequence
  Description of Artificial Sequence Synthetic oligonucleotide
ggcacaaaugcaacacugcauuaccaugcgguugugcc

SEQ ID NO: 32 - RNA, 39 nt, Artificial Sequence
  Description of Artificial Sequence Synthetic oligonucleotide
ggcacacaaugcaacacugcauuaccaugcgguugugcc

SEQ ID NO: 33 - RNA, 40 nt, Artificial Sequence
  Description of Artificial Sequence Synthetic oligonucleotide
ggcacacaaugcaacacugcauugaccaugcgguugugcc

SEQ ID NO: 34 - RNA, 40 nt, Artificial Sequence
  Description of Artificial Sequence Synthetic oligonucleotide
ggcacacgaaugcaacacugcauuaccaugcgguugugcc

SEQ ID NO: 35 - RNA, 41 nt, Artificial Sequence
  Description of Artificial Sequence Synthetic oligonucleotide
ggcacacaaugcaacacugcauugaccaugcgguaugugcc

SEQ ID NO: 36 - RNA, 41 nt, Artificial Sequence
  Description of Artificial Sequence Synthetic oligonucleotide
ggcacacgaaugcaacacugcauugaccaugcgguugugcc

SEQ ID NO: 37 - RNA, 41 nt, Artificial Sequence
  Description of Artificial Sequence Synthetic oligonucleotide
ggcacacagaaugcaacacugcauuaccaugcgguugugcc

SEQ ID NO: 38 - RNA, 14 nt, Artificial Sequence
  Description of Artificial Sequence Synthetic oligonucleotide
ggcacaaaugcaac

SEQ ID NO: 39 - RNA, 24 nt, Artificial Sequence
  Description of Artificial Sequence Synthetic oligonucleotide
acugcauuaccaugcgguugugcc

SEQ ID NO: 40 - RNA, 27 nt, Artificial Sequence
  Description of Artificial Sequence Synthetic oligonucleotide
ggcacaaaugcaacacugcauuaccau

SEQ ID NO: 41 - RNA, 11 nt, Artificial Sequence
  Description of Artificial Sequence Synthetic oligonucleotide
gcgguugugcc

SEQ ID NO: 42 - DNA, 17 bp, Artificial Sequence
  Description of Artificial Sequence Synthetic oligonucleotide
ctgtaggcaccatcaat

SEQ ID NO: 43 - RNA, 26 nt, Artificial Sequence
  Description of Artificial Sequence Synthetic oligonucleotide
guucagaguucuacaguccgacgauc

SEQ ID NO: 44 - DNA, 59 bp, Artificial Sequence
  Description of Artificial Sequence Synthetic oligonucleotide
  modified_base (35)..(42): a, c, t, g, unknown or other
gtgactggagttcagacgtgtgctcttccgatctnnnnnnnnattgatggtgcctacag

SEQ ID NO: 45 - DNA, 63 bp, Artificial Sequence
  Description of Artificial Sequence Synthetic oligonucleotide
  modified_base (30)..(37): a, c, t, g, unknown or other
tctttccctacacgacgctcttccgatctnnnnnnnngttcagagttctacagtccgacgatc

SEQ ID NO: 46 - DNA, 29 bp, Artificial Sequence
  Description of Artificial Sequence Synthetic oligonucleotide
aatgatacggcgaccaccgagatctacac

SEQ ID NO: 47 - DNA, 29 bp, Artificial Sequence
  Description of Artificial Sequence Synthetic oligonucleotide
tctttccctacacgacgctcttccgatct

SEQ ID NO: 48 - DNA, 24 bp, Artificial Sequence
  Description of Artificial Sequence Synthetic oligonucleotide
caagcagaagacggcatacgagat

SEQ ID NO: 49 - DNA, 34 bp, Artificial Sequence
  Description of Artificial Sequence Synthetic oligonucleotide
gtgactggagttcagacgtgtgctcttccgatct

SEQ ID NO: 50 - DNA, 18 bp, Artificial Sequence
  Description of Artificial Sequence Synthetic oligonucleotide
gatacggcgaccaccgag

SEQ ID NO: 51 - DNA, 21 bp, Artificial Sequence
  Description of Artificial Sequence Synthetic oligonucleotide
gcagaagacggcatacgagat

SEQ ID NO: 52 - RNA, 13 nt, Artificial Sequence
  Description of Artificial Sequence Synthetic oligonucleotide
ggcacaaaugcaa

SEQ ID NO: 53 - RNA, 23 nt, Artificial Sequence
  Description of Artificial Sequence Synthetic oligonucleotide
cugcauuaccaugcgguugugcc

SEQ ID NO: 54 - RNA, 26 nt, Artificial Sequence
  Description of Artificial Sequence Synthetic oligonucleotide
ggcacaaaugcaacacugcauuacca

SEQ ID NO: 55 - RNA, 10 nt, Artificial Sequence
  Description of Artificial Sequence Synthetic oligonucleotide
cgguugugcc

SEQ ID NO: 56 - DNA, 55 bp, Artificial Sequence
  Description of Artificial Sequence Synthetic oligonucleotide
  Description of Combined DNA/RNA Molecule Synthetic oligonucleotide
ggcacaaaugcaacacugcauuaccaugcgguugugccctgtaggcaccatcaat

SEQ ID NO: 57 - DNA, 81 bp, Artificial Sequence
  Description of Artificial Sequence Synthetic oligonucleotide
  Description of Combined DNA/RNA Molecule Synthetic oligonucleotide
guucagaguucuacaguccgacgaucggcacaaaugcaacacugcauuaccaugcgguugugccctgtaggcaccatcaat

SEQ ID NO: 58 - DNA, 123 bp, Artificial Sequence
  Description of Artificial Sequence Synthetic polynucleotide
  modified_base (35)..(42): a, c, t, g, unknown or other
gtgactggagttcagacgtgtgctcttccgatctnnnnnnnnattgatggtgcctacagggcacaaccgcatggtaatgcagtgttgcatttgtgccgatcgtcggactgtagaactctgaac

SEQ ID NO: 59 - DNA, 160 bp, Artificial Sequence
  Description of Artificial Sequence Synthetic polynucleotide
  modified_base (30)..(37): a, c, t, g, unknown or other
  modified_base (119)..(126): a, c, t, g, unknown or other
tctttccctacacgacgctcttccgatctnnnnnnnngttcagagttctacagtccgacgatcggcacaaatgcaacactgcattaccatgcggttgtgccctgtaggcaccatcaatnnnnnnnnagatcggaagagcacacgtctgaactccagtcac

SEQ ID NO: 60 - DNA, 33 bp, Artificial Sequence
  Description of Artificial Sequence Synthetic oligonucleotide
acactctttccctacacgacgctcttccgatct

SEQ ID NO: 61 - DNA, 164 bp, Artificial Sequence
  Description of Artificial Sequence Synthetic polynucleotide
  modified_base (34)..(41): a, c, t, g, unknown or other
  modified_base (123)..(130): a, c, t, g, unknown or other
acactctttccctacacgacgctcttccgatctnnnnnnnngttcagagttctacagtccgacgatcggcacaaatgcaacactgcattaccatgcggttgtgccctgtaggcaccatcaatnnnnnnnnagatcggaagagcacacgtctgaactccagtcac

SEQ ID NO: 62 - DNA, 24 bp, Artificial Sequence
  Description of Artificial Sequence Synthetic oligonucleotide
atctcgtatgccgtcttctgcttg

SEQ ID NO: 63 - DNA, 38 bp, Artificial Sequence
  Description of Artificial Sequence Synthetic oligonucleotide
ggcacaaatgcaacactgcattaccatgcggttgtgcc

SEQ ID NO: 64 - DNA, 55 bp, Artificial Sequence
  Description of Artificial Sequence Synthetic oligonucleotide
ggcacaaatgcaacactgcattaccatgcggttgtgccctgtaggcaccatcaat

SEQ ID NO: 65 - DNA, 81 bp, Artificial Sequence
  Description of Artificial Sequence Synthetic oligonucleotide
  Description of Combined DNA/RNA Molecule Synthetic oligonucleotide
guucagaguucuacaguccgacgaucggcacaaatgcaacactgcattaccatgcggttgtgccctgtaggcaccatcaat